(19) United States
(12) Patent Application Publication
(10) Pub. No.: US 2026/0109972 A1
(43) Pub. Date: Apr. 23, 2026

(54) High-Throughput Discovery of Multi-Partite CRISPR-Based Editors

(71) Applicant: THE REGENTS OF THE UNIVERSITY OF CALIFORNIA, Oaklang, CA (US)

(72) Inventors: Luke A. Gilbert, San Francisco, CA (US); Greg Pommier, San Francisco, CA (US); Jonathan Weissman, San Francisco, CA (US); Caroline Wilson, San Francisco, CA (US)

(21) Appl. No.: 19/127,007

(22) PCT Filed: Nov. 9, 2023
(86) PCT No.: PCT/US2023/079280

§ 371 Date: May 2, 2025

Related U.S. Application Data
(60) Provisional application No. 63/424,049, filed on Nov. 9, 2022.
(60) Provisional application No. 63/463,832, filed on May 3, 2023.

(51) Int. Cl. C12N 15/10

(52) U.S. Cl. CPC C12N 15/1082

(57) ABSTRACT

Provided are methods for determining that a candidate polypeptide in a library of polypeptides has editing activity comprising expressing in a plurality of cells: a library of first expression cassettes each comprising a nucleic acid encoding a polypeptide wherein each polypeptide in the library comprises, a first editing domain, a first linker, a nuclease-deficient RNA guided endonuclease domain, a second linker, and a second editing domain; and a second expression cassette comprising a nucleic acid encoding a guide RNA directed to a target of interest, wherein said first and second expression cassettes are expressed in the plurality of cells; separating the plurality of cells based on the expression level of the target of interest; extracting genomic DNA (gDNA) from the plurality of cells; and sequencing the gDNA of cells that have a high level expression or a low level of expression of the target of interest.

### CROSS-REFERENCE TO RELATED APPLICATIONS

Pursuant to 35 U.S.C. § 119 (e), this application claims priority to the filing date of U.S. Provisional Patent Application Ser. No. 63/424,049 filed Nov. 9, 2022, and the filing date of U.S. Provisional Patent Application Ser. No. 63/463,832 filed May 3, 2023 the disclosure of which applications are herein incorporated by reference.

### STATEMENT REGARDING FEDERALLY SPONSORED RESEARCH

This invention was made with government support under Grant Number HRO0111920007 awarded by the National Institutes of Health. The government has certain rights in the invention.

### REFERENCE TO AN ELECTRONIC SEQUENCE LISTING

The contents of the electronic sequence listing (UCSF-692WO_Seq_List.xml; Size: 1,652,402 bytes; and Date of Creation: Nov. 8, 2023) is herein incorporated by reference in its entirety.

### INTRODUCTION

Although considered a promising therapeutic approach for the treatment of disease, genome editing carries inherent risks due to the potential for genotoxicity from double-strand breaks. Further, genome editing often is associated with an all-or-none effect on the target gene (i.e., it produces a full knockout). In contrast, targeted epigenome engineering does not carry the risk of DSB-induced genotoxicity; further, it affords the opportunity to create a more graded effect on gene expression and thus function from a complete silencing through a less pronounced effect. The discovery of novel epigenomic editing proteins is needed. Provided herein are methods for identifying novel epigenomic editing proteins.

### SUMMARY

The present disclosure provides methods for determining that a candidate polypeptide in a library of polypeptides has editing activity comprising expressing in a plurality of cells: a library of first expression cassettes each comprising a nucleic acid encoding a polypeptide wherein each polypeptide in the library comprises, in order, a first editing domain, a first linker, a nuclease-deficient RNA guided endonuclease domain, a second linker, and a second editing domain; and a second expression cassette comprising a nucleic acid encoding a guide RNA directed to a target of interest, wherein said first and second expression cassettes are expressed in the plurality of cells; separating the plurality of cells based on the expression level of the target of interest; extracting genomic DNA (gDNA) from the plurality of cells; and sequencing the gDNA of cells that have a high level expression or a low level of expression of the target of interest thereby determining that the candidate polypeptide has editing activity.

### BRIEF DESCRIPTION OF THE DRAWINGS

FIG. 1. Schematic representation of library generation. Top: Design for combinatorial domain library generation, with 37×37 combinations for library 1 (L1) and 347×347 elements for library 2 (L2), with two library elements listed in positions 1 and 2. Bottom: Construct design for dual transcriptional effectors, with N and C termini elements fused to dCas9 via two linker sequences.

FIG. 2. Experimental workflow. N and C-termini DNA elements were synthesized and assembled into dual-dCas9 effector domains. Constructs were packed into lentivirus and infected into sgRNA expressing cells, followed by selection for library integration. Target gene of interest (CD55) expression was assayed at the final timepoints (day 8) and cells were sorted based on CD55 expression, followed by gDNA extraction, dual-domain amplification by PCR, library preparation, and PacBio long read sequencing.

FIG. 3. Library 1 (37×37) targeting CD55 in K562s replicates. Log transformed domain counts (fraction of total reads) per replicate for each sample; unsorted (left), CD55^{high} (middle), and CD55^{low} (right).

FIG. 4. Library 1 screen at CD55 identifies 187 significant hits. Colored points (red, green, blue) represent significant hits (pvalue<=0.05), including significant activators (blue, log 2FC>=1) and significant CD55 repressors (red, log 2FC<=−1).

FIG. 5A-FIG. 5B. Library 1 targeting CD55 showing replicates plotted against each other with Log 2(CD55^{High}/CD55^{LOW}), where x-axis=Replicate 1 and y-axis=Replicate 2. Points are colored by fusion protein length (base pairs). “VPR_JKNp64” emerged on the activating side (FIG. 5B), as this was the activation control, and “Gilbert_Lab_CRISPRi_KRAB” (CRISPR inhibition) domain emerged on the repressive plot (FIG. 5A).

FIG. 6A-FIG. 6B. Library 2 replicates are highly correlated. Library 2 replicates for CD55^{HIGH} (FIG. 6A) and CD55^{LOW} (FIG. 6B) samples. Pearson correlation coefficient (R): CD55^{HIGH}=0.94, CD5^{LOW} 0.90.

FIG. 7. Library 2 screen at CD55 identifies 3709 significant hits. Colored points (red, green, blue) represent significant hits (pvalue<=0.05), including significant activators (blue, log 2FC>=1) and significant CD55 repressors (red, log 2FC<=−1).

FIG. 8. Library 2 screen at CD55 identifies 3709 significant hits. Colored points indicate domain length (N-dCa9-C total length, bp). A subset of domains are labeled.

FIG. 9. SETD1A-KDM5A effector combinations exhibit position specific effects at CD55.

FIG. 10. Workflow for processing long read sequencing data. Pacbio subread BAM files are exported from Sequel II and ccs is run to generate HiFi reads. Reads are demultiplexed using lima and aligned to the domain library reference. Alignments are counted and counts per domain are normalized for downstream analyses.

FIG. 11. Gel electrophoresis analysis of pooled and processed gDNAs.

### DEFINITIONS

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine. Coded amino acids (followed in parentheses by their corresponding three-letter codes and one-letter codes) include: alanine (Ala; A), arginine (Arg; R), asparagine (Asn; N), aspartic acid (Asp; D), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F); proline (Pro; P), serine (Ser; S), threonine (Thr; T), tryptophan (Trp; W), tyrosine (Tyr; Y), or valine (Val; V)

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, Phyre2, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/, http://www.sbg.bio.ic.ac.uk/-phyre2/. See, e.g., Altschul et al. (1990), J. Mol. Bioi. 215:403-10.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., protein coding) and/or regulate translation of an encoded polypeptide.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, above). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

“Heterologous,” as used herein, refers to a nucleotide or polypeptide sequence that is not found in the native nucleic acid or protein, respectively. For example, relative to a variant type V CRISPR/Cas effector polypeptide of the present disclosure, a heterologous polypeptide comprises an amino acid sequence from a protein other than the variant type V CRISPR/Cas effector polypeptide. As another example, a variant type V CRISPR/Cas effector polypeptide of the present disclosure can be fused to an active domain from a non-CRISPR/Cas effector protein (e.g., a histone deacetylase), and the sequence of the active domain could be considered a heterologous polypeptide (it is heterologous to the variant type V CRISPR/Cas effector polypeptide). As another example, a guide sequence of a guide RNA that is heterologous to a protein-binding sequence of a guide RNA is a guide sequence that is not found in nature together with the protein-binding sequence.

The term “nuclease-deficient RNA-guided DNA endonuclease domain” and the like refer, in the usual and customary sense, to an RNA-guided DNA endonuclease (e.g. a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., a guide RNA or a single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., gRNA or sgRNA), but lacks significant endonuclease activity (e.g. any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCpf1, ddCpf1, Cas-phi, a nuclease-deficient Cas9 variant, a nuclease-deficient Class II CRISPR endonuclease, a zinc finger domain, a transcription activator-like effector (TALE), a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a zinc finger domain, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain. In aspects, the nuclease-deficient RNA-guided DNA endonuclease enzyme is a winged helix domain. In aspects, the nuclease-deficient RNA-guided DNA endonuclease enzyme is a helix-turn-helix motif. In aspects, the nuclease-deficient RNA-guided DNA endonuclease enzyme is a helix-loop-helix domain. In aspects, the nuclease-deficient RNA-guided DNA endonuclease enzyme is an HMB-box domain. In aspects, the nuclease-deficient RNA-guided DNA endonuclease enzyme is a Wor3 domain. In aspects, the nuclease-deficient RNA-guided DNA endonuclease enzyme is an OB-fold domain. In aspects, the nuclease-deficient RNA-guided DNA endonuclease enzyme is an immunoglobulin domain. In aspects, the nuclease-deficient RNA-guided DNA endonuclease enzyme is a B3 domain. In aspects, the nuclease-deficient RNA-guided DNA endonuclease enzyme is dCas9, ddCpf1, Cas-phi, a nuclease-deficient Cas9 variant, or a nuclease-deficient Class II CRISPR endonuclease. In aspects, the nuclease-deficient RNA-guided DNA endonuclease enzyme is dCas9. In aspects, the nuclease-deficient RNA-guided DNA endonuclease enzyme is dCas9 from S. pyogenes. In aspects, the nuclease-deficient RNA-guided DNA endonuclease enzyme is dCas9 from S. aureus. In aspects, the nuclease-deficient RNA-guided DNA endonuclease enzyme is dCas 12a from Lachnospiracea (dLbCas12a). In aspects, the nuclease-deficient RNA-guided DNA endonuclease enzyme is dCas12a from Lachnospiracea bacterium. In aspects, the nuclease-deficient RNA-guided DNA endonuclease enzyme is dCas12a. In aspects, the nuclease-deficient RNA-guided DNA endonuclease enzyme is dCas12. In aspects, the nuclease-deficient RNA-guided DNA endonuclease enzyme is ddCas 12a. In aspects, the nuclease-deficient RNA-guided DNA endonuclease enzyme is Cas-phi.

In embodiments, the nuclease-deficient RNA-guided DNA endonuclease enzyme is dCas9. The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity. In aspects, dCas9 includes the amino acid sequence of SEQ ID NO:1. In aspects, dCas9 has the amino acid sequence of SEQ ID NO:1. In aspects, dCas9 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1. In aspects, dCas9 has an amino acid sequence that has at least 75% sequence identity to SEQ ID NO: 1. In aspects, dCas9 has an amino acid sequence that has at least 80% sequence identity to SEQ ID NO:1. In aspects, dCas9 has an amino acid sequence that has at least 85% sequence identity to SEQ ID NO: 1. In aspects, dCas9 has an amino acid sequence that has at least 90% sequence identity to SEQ ID NO: 1. In aspects, dCas9 has an amino acid sequence that has at least 95% sequence identity to SEQ ID NO:1. In embodiments, the nuclease-deficient RNA-guided DNA endonuclease enzyme is dCas9 from S. pyogenes. In embodiments, the nuclease-deficient RNA-guided DNA endonuclease enzyme is dCas9 from S. aureus.

(SEQ ID NO: 1)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLONGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In embodiments, the nuclease-deficient RNA-guided DNA endonuclease enzyme is “ddCpf1” or “ddCas 12a”. The terms “DNAse-dead Cpf1” or “ddCpf1” refer to mutated Acidaminococcus sp. Cpf1 (AsCpf1) resulting in the inactivation of Cpf1 DNAse activity. In aspects, ddCpf1 includes an E993A mutation in the RuvC domain of AsCpf1. In aspects, the ddCpf1 has substantially no detectable endonuclease (e.g., endodeoxyribonuclease) activity. In aspects, ddCpf1 includes the amino acid sequence of SEQ ID NO:2. In aspects, ddCpf1 has the amino acid sequence of SEQ ID NO:2. In aspects, ddCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:2. In aspects, ddCpf1 has an amino acid sequence that has at least 75% sequence identity to SEQ ID NO:2. In aspects, ddCpf1 has an amino acid sequence that has at least 80% sequence identity to SEQ ID NO:2. In aspects, ddCpf1 has an amino acid sequence that has at least 85% sequence identity to SEQ ID NO:2. In aspects, ddCpf1 has an amino acid sequence that has at least 90% sequence identity to SEQ ID NO:2. In aspects, ddCpf1 has an amino acid sequence that has at least 95% sequence identity to SEQ ID NO:2. In aspects, the nuclease-deficient RNA-guided DNA endonuclease enzyme is dCas 12a from Lachnospiracea bacterium.

(SEQ ID NO: 2)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLANLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In embodiments, the nuclease-deficient RNA-guided DNA endonuclease enzyme is dLbCpf1. The term “dLbCpf1: refers to mutated Cpf1 from Lachnospiraceae bacterium ND2006 (LbCpf1) that lacks DNAse activity. In aspects, dLbCpf1 includes a D832A mutation. In spects, the dLbCpf1 has substantially no detectable endonuclease (e.g., endodeoxyribo-nuclease) activity. In aspects, dLbCpf1 includes the amino acid sequence of SEQ ID NO:3. In aspects, dLbCpf1 has the amino acid sequence of SEQ ID NO:3. In aspects, dLbCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:3. In aspects, dLbCpf1 has an amino acid sequence that has at least 75% sequence identity to SEQ ID NO:3. In aspects, dLbCpf1 has an amino acid sequence that has at least 80% sequence identity to SEQ ID NO:3. In aspects, dLbCpf1 has an amino acid sequence that has at least 85% sequence identity to SEQ ID NO:3. In aspects, dLbCpf1 has an amino acid sequence that has at least 90% sequence identity to SEQ ID NO:3. In aspects, dLbCpf1 has an amino acid sequence that has at least 95% sequence identity to SEQ ID NO:3.

(SEQ ID NO: 3)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIARGERNLLYIVVVDGKGNI
VEQYSLNEIINNFNGIRIKTDYHSLLDKKEKERFEARQNWTSIENIKELK
AGYISQVVHKICELVEKYDAVIALADLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH

In embodiments, the nuclease-deficient RNA-guided DNA endonuclease enzyme is dFnCpf1. The term “dFnCpf1” refers to mutated Cpf1 from Francisella novicida UI 12 (FnCpf1) that lacks DNAse activity. In aspects, dFnCpf1 includes a D917 A mutation. In aspects, the dFnCpf1 has substantially no detectable endonuclease (e.g., endodeoxyribo-nuclease) activity. In aspects, dFnCpf1 includes the amino acid sequence of SEQ ID NO: 4. In aspects, dFnCpf1 has the amino acid sequence of SEQ ID NO: 4. In aspects, dFnCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:4. In aspects, dFnCpf1 has an amino acid sequence that has at least 75% sequence identity to SEQ ID NO:4. In aspects, dFnCpf1 has an amino acid sequence that has at least 80% sequence identity to SEQ ID NO:4. In aspects, dFnCpf1 has an amino acid sequence that has at least 85% sequence identity to SEQ ID NO:4. In aspects, dFnCpf1 has an amino acid sequence that has at least 90% sequence identity to SEQ ID NO:4. In aspects, dFnCpf1 has an amino acid sequence that has at least 95% sequence identity to SEQ ID NO:4.

(SEQ ID NO: 4)
MYPYDVPDYASGSGMSIYQEFVNKYSLSKTLRFELIPQGKTLENIKARGL
ILDDEKRAKDYKKAKQIIDKYHQFFIEEILSSVCISEDLLQNYSDVYFKL
KKSDDDNLQKDFKSAKDTIKKQISEYIKDSEKFKNLFNQNLIDAKKGQES
DLILWLKQSKDNGIELFKANSDITDIDEALEIIKSFKGWTTYFKGFHENR
KNVYSSNDIPTSIIYRIVDDNLPKFLENKAKYESLKDKAPEAINYEQIKK
DLAEELTFDIDYKTSEVNQRVESLDEVFEIANFNNYLNQSGITKFNTIIG
GKFVNGENTKRKGINEYINLYSQQINDKTLKKYKMSVLFKQILSDTESKS
FVIDKLEDDSDVVTTMQSFYEQIAAFKTVEEKSIKETLSLLFDDLKAQKL
DLSKIYFKNDKSLTDLSQQVFDDYSVIGTAVLEYITQQIAPKNLDNPSKK
EQELIAKKTEKAKYLSLETIKLALEEFNKHRDIDKQCRFEEILANFAAIP
MIFDEIAQNKDNLAQISIKYQNQGKKDLLQASAEDDVKAIKDLLDQTNNL
LHKLKIFHISQSEDKANILDKDEHFYLVFEECYFELANIVPLYNKIRNYI
TQKPYSDEKFKLNFENSTLANGWDKNKEPDNTAILFIKDDKYYLGVMNKK
NNKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWKD
FGFRFSDTQRYNSIDEFYREVENQGYKLTFENISESYIDSVVNQGKLYLF
QIYNKDFSAYSKGRPNLHTLYWKALFDERNLQDVVYKLNGEAELFYRKQS
IPKKITHPAKEAIANKNKDNPKKESVFEYDLIKDKRFTEDKFFFHCPITI
NFKSSGANKFNDEINLLLKEKANDVHILSIARGERHLAYYTLVDGKGNII
KQDTFNIIGNDRMKTNYHDKLAAIEKDRDSARKDWKKINNIKEMKEGYLS
QVVHEIAKLVIEYNAIVVFEDLNFGFKRGRFKVEKQVYQKLEKMLIEKLN
YLVFKDNEFDKTGGVLRAYQLTAPFETFKKMGKQTGIIYYVPAGFTSKIC
PVTGFVNQLYPKYESVSKSQEFFSKFDKICYNLDKGYFEFSFDYKNFGDK
AAKGKWTIASFGSRLINFRNSDKNHNWDTREVYPTKELEKLLKDYSIEYG
HGECIKAAICGESDKKFFAKLTSVLNTILQMRNSKTGTELDYLISPVADV
NGNFFDSRQAPKNMPQDADANGAYHIGLKGLMLLGRIKNNQEGKKLNLVI
KNEEYFEFVQNRNN

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In aspects, the Cpf1 protein is substantially identical to the protein identified by the UniProt reference number U2UMQ6 or a variant or homolog having substantial identity thereto. In aspects, the Cpf1 protein is identical to the protein identified by the UniProt reference number U2UMQ6. In aspects, the Cpf1 protein has at least 75% sequence identity to the amino acid sequence of the protein identified by the UniProt reference number U2UMQ6. In aspects, the Cpf1 protein has at least 80% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 85% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 90% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 95% sequence identity to the amino acid sequence of the protein identified by the UniProt reference number U2UMQ6.

In embodiments, the nuclease-deficient RNA-guided DNA endonuclease enzyme is a nuclease-deficient Cas9 variant. The term “nuclease-deficient Cas9 variant” refers to a Cas9 protein having one or more mutations that increase its binding specificity to PAM compared to wild type Cas9 and further include mutations that render the protein incapable of or having severely impaired endonuclease activity. Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but P AMs are typically 2-5 base pair sequences adjacent the protospacer (that is, the target sequence). The binding specificity of nuclease-deficient Cas9 variants to PAM can be determined by any method known in the art. Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8. 2017. Exemplary Cas9 variants are listed in the Table 1 below.

TABLE 1

| Cas9 Variants | PAM domains | References |
|---|---|---|
| Strep pyogenes (Sp) | NGG | Hsu et al. 2014 Cell |
| Cas9 | | |
| Staph aureus (Sa) Cas9 | NNGRRT or NNGRR | Ran et al. 2015 Nature |
| | NNGGGT, NNGAAT, | |
| | NNGAGT (Zetsche) | |
| SpCas9 VQR mutant | NGAG > NGAT = | Kleinstiver et al. 2015 |
| (D1135V, R1335Q, | NGAA > NGAC | Nature |
| T1337R) | NGCG | |
| SpCas9 VRER mutant | NGCG | Kleinstiver et al. 2015 |
| (D1135V/G1218R/ | | Nature |
| R1335E/T1337R) | | |
| SpCas9 D1135E | NGG, greater fidelity, | Kleinstiver et al. 2015 |
| | less cutting at NAG | Nature |
| | and NGA sites | |
| eSpCas9 1.1 mutant | NGG | Slaymaker et al. |
| (K848A/K1003A/ | | Science 2015 |
| R1060A) | | |
| SpCas9 HFl | NGG | Kleinstiver et al. 2016 |
| (Q695A, Q926A, | | Nature |
| N497A, R661A) | | |
| AsCpfl | TTTN (5′ of sgRNA) | Zetsche et al. 2015 |
| HypaCas9 (N692A, | | Cell Chen et al., |
| M694A, Q695A, | | Nature volume 550, |
| H698A) | | pages 407-410 (19 |
| | | Oct. 2017) |

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a nuclease-deficient CRISPR/Cas effector polypeptide” includes a plurality of such a nuclease-deficient CRISPR/Cas effector polypeptides and reference to “the guide nucleic acid” includes reference to one or more guide nucleic acid and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for determining that a candidate polypeptide in a library of polypeptides has editing activity comprising expressing in a plurality of cells: a library of first expression cassettes each comprising a nucleic acid encoding a polypeptide wherein each polypeptide in the library comprises, in order, a first editing domain, a first linker, a nuclease-deficient RNA guided endonuclease domain, a second linker, and a second editing domain; and a second expression cassette comprising a nucleic acid encoding a guide RNA directed to a target of interest, wherein said first and second expression cassettes are expressed in the plurality of cells; separating the plurality of cells based on the expression level of the target of interest; extracting genomic DNA (gDNA) from the plurality of cells; and sequencing the gDNA of cells that have a high level expression or a low level of expression of the target of interest thereby determining that the candidate polypeptide has epigenetic editing activity. In some embodiments, the polypeptide comprises a third editing domain. In some embodiments, the polypeptide comprises a fourth editing domain. The present disclosure also provided libraries containing the first expression cassettes of the present disclosure.

Methods for Determining that a Candidate Polypeptide in a Library of Polypeptide has Editing Activity

The present disclosure provides methods for determining that a candidate polypeptide in a library of polypeptides has editing activity comprising expressing in a plurality of cells: a library of first expression cassettes each comprising a nucleic acid encoding a polypeptide wherein each polypeptide in the library comprises, in order, a first editing domain, a first linker, a nuclease-deficient RNA guided endonuclease domain, a second linker, and a second editing domain; and a second expression cassette comprising a nucleic acid encoding a guide RNA directed to a target of interest, wherein said first and second expression cassettes are expressed in the plurality of cells; separating the plurality of cells based on the expression level of the target of interest; extracting genomic DNA (gDNA) from the plurality of cells; and sequencing the gDNA of cells that have a high level expression or a low level of expression of the target of interest thereby determining that the candidate polypeptide has editing activity. In some embodiments, the polypeptide comprises a third editing domain. In some embodiments, the polypeptide comprises a fourth editing domain.

The methods of the present disclosure comprise expressing a first and second expression cassette in a cell. In general, the expression involves introducing an expression cassette into a host cell. Methods of introducing a nucleic acid into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like.

The methods disclosed herein comprise expressing a first and a second expression cassette in a plurality of cells. The plurality of cells may be any cell of interest. Cells that find use in the present disclosure include, without limitation, mammalian cells, plant cells, insect cells, eukaryotic cells, yeast cells, bacterial cells, fungal cells, etc.

The library utilized in the methods of the present application comprise a collection of first expression cassettes each comprising a nucleic acid encoding a polypeptide. In some embodiments, the nucleic acid in the first expression cassette is operably linked to a promoter. The promoter may be any promoter described below. In some embodiments, the first expression cassette does not comprise a nucleic acid encoding a barcode sequence.

The libraries of the present disclosure contain a large number of unique polypeptides. The number of unique polypeptides in the libraries include greater than 500, greater than 1000, greater than 1500, greater than 2000, greater than 3000, greater than 4000, greater than 5000, greater than 6000, greater than 7000, greater than 8000, greater than 9000, greater than 10000, greater than 20000, greater than 30000, greater than 40000, greater than 50000, greater than 60000, greater than 70000, greater than 80000, greater than 90000, or greater than 100000 unique polypeptides.

The polypeptides of the present application comprise a first editing domain, a first linker, a nuclease-deficient RNA guided endonuclease domain, a second linker, and a second editing domain. In some embodiments, the editing domain is the enzymatic domain of a polypeptide related to epigenetic modifications. Epigenetic modifications may be a specific modification of DNA (e.g. genomic DNA) or may be modification of amino acids present in histones. Epigenetic modifications of DNA include, without limitation, methylation, demethylation, deamination, etc. Epigenetic modifications of histones include, without limitation, acetylation, deacetylation, ubiquitination, deubiquitination, sumoylation, desumolyation, ADP-Ribosylation, phosphorylation, etc. In some embodiments, the epigenetic modification is modification of DNA. In some embodiments, the epigenetic modification is modification of histones. The editing domain may be any enzymatic domain of a polypeptide associated with epigenetic modification. The editing domain may also be a truncation or a modified variant of any enzymatic domain of a polypeptide associated with epigenetic modification. Polypeptides involved in epigenetic modification are known in the art and have been described in, for example, Medvedeva et al. (Database (Oxford). 2015 Jul. 7; 2015:bav067) which is specifically incorporated by reference herein. In some embodiments, the first and second editing domain is an enzymatic domain selected from any of the polypeptides listed in table 2. In some embodiments, the first and second editing domain is selected from any of the polypeptides encoded by the nucleic acids listed in table 4. In some embodiments, the first and second editing domains are different from each other. In some embodiments, the first and second editing domains are the same as each other.

TABLE 2
Polypeptides associated with epigenetic modification

| | | | | | | | |
|---|---|---|---|---|---|---|---|
| A1CF | CBX5 | EID2 | IKZF3 | MORF4L2 | PRDM1 | SETD1A | TEX10 |
| ACINU | CBX6 | EID2B | ING1 | MOV10 | PRDM11 | SETD1B | TFDP1 |
| ACTB | CBX7 | EIF4A3 | ING2 | MPHOSPH8 | PRDM12 | SETD2 | TFPT |
| ACTL6A | CBX8 | ELP2 | ING3 | MPND | PRDM13 | SETD3 | THRAP3 |
| ACTL6B | CCDC101 | ELP3 | ING4 | MRGBP | PRDM14 | SETD5 | TLE1 |
| ACTR3B | CDC6 | ELP4 | ING5 | MSH6 | PRDM16 | SETD6 | TLE2 |
| ACTR5 | CDC73 | ELP5 | INO80 | MSL1 | PRDM2 | SETD7 | TLE4 |
| ACTR6 | CDK1 | ELP6 | INO80B | MSL2 | PRDM4 | SETD8 | TLK1 |
| ACTR8 | CDK17 | ENY2 | INO80C | MSL3 | PRDM5 | SETDB1 | TLK2 |
| ADNP | CDK2 | EP300 | INO80D | MST1 | PRDM6 | SETDB2 | TNP1 |
| AEBP2 | CDK3 | EP400 | INO80E | MTA1 | PRDM7 | SETMAR | TNP2 |
| AICDA | CDK5 | EPC1 | JADE1 | MTA2 | PRDM8 | SF3B1 | TONSL |
| AIRE | CDK7 | EPC2 | JADE2 | MTA3 | PRDM9 | SF3B3 | TOP2A |
| ALKBH1 | CDK9 | ERBB4 | JADE3 | MTF2 | PRKAA1 | SFMBT1 | TOP2B |
| ALKBH1 | CDY1 | ERCC6 | JAK2 | MUM1 | PRKAA2 | SFMBT2 | TP53 |
| ALKBH4 | CDY1B | EXOSC1 | JARID2 | MYBBP1A | PRKAB1 | SFPQ | TP53BP1 |
| ALKBH5 | CDY2A | EXOSC2 | JDP2 | MYO1C | PRKAB2 | SFSWAP | TRA2B |
| ANKRD32 | CDY2B | EXOSC3 | JMJD1C | MYSM1 | PRKAG1 | SHPRH | TRIM16 |
| ANP32A | CDYL | EXOSC4 | JMJD6 | NAA60 | PRKAG2 | SIN3A | TRIM24 |
| ANP32B | CDYL2 | EXOSC5 | KANSL1 | NAP1L1 | PRKAG3 | SIN3B | TRIM27 |
| ANP32E | CECR2 | EXOSC6 | KANSL2 | NAP1L2 | PRKCA | SIRT1 | TRIM28 |
| APBB1 | CELF1 | EXOSC7 | KANSL3 | NAP1L4 | PRKCB | SIRT2 | TRIM33 |
| APEX1 | CELF2 | EXOSC8 | KAT2A | NASP | PRKCD | SIRT6 | TRRAP |
| APOBEC1 | CELF3 | EXOSC9 | KAT2B | NAT10 | PRKDC | SIRT7 | TRUB2 |
| APOBEC2 | CELF4 | EYA1 | KAT5 | NAT10 | PRMT1 | SKP1 | TSSK6 |
| APOBEC3A | CELF5 | EYA2 | KAT6A | NBN | PRMT2 | SLU7 | TTK |
| APOBEC3B | CELF6 | EYA3 | KAT6B | NCL | PRMT5 | SMARCA1 | TYW5 |
| APOBEC3C | CENPC | EYA4 | KAT7 | NCOA1 | PRMT6 | SMARCA2 | U2AF2 |
| APOBEC3D | CHAF1A | EZH1 | KAT8 | NCOA2 | PRMT7 | SMARCA4 | UBE2A |
| APOBEC3F | CHAF1B | EZH2 | KDM1A | NCOA3 | PRMT8 | SMARCA5 | UBE2B |
| APOBEC3G | CHD1 | FAM175A | KDM1B | NCOA6 | PRMT9 | SMARCAD1 | UBE2D1 |
| APOBEC3H | CHD1L | FAM175B | KDM2A | NCOR1 | PRPF31 | SMARCAL1 | UBE2D3 |
| ARID1A | CHD2 | FBL | KDM2B | NCOR2 | PRR14 | SMARCB1 | UBE2E1 |
| ARID1B | CHD3 | FBRS | KDM3A | NEK6 | PSIP1 | SMARCC1 | UBE2H |
| ARID2 | CHD4 | FBRSL1 | KDM3B | NEK9 | PTBP1 | SMARCC2 | UBE2N |
| ARID4A | CHD5 | FOXA1 | KDM4A | NFRKB | PTBP1 | SMARCD1 | UBE2T |
| ARID4B | CHD6 | FOXO1 | KDM4B | NFYB | PUF60 | SMARCD2 | UBN1 |
| ARNTL | CHD7 | FOXP1 | KDM4C | NFYC | RAD51 | SMARCD3 | UBR2 |
| ARRB1 | CHD8 | FOXP2 | KDM4D | NIPBL | RAD54B | SMARCE1 | UBR5 |
| ASF1A | CHD9 | FOXP3 | KDM4E | NOC2L | RAD54L | SMEK1 | UBR7 |
| ASF1B | CHEK1 | FOXP4 | KDM5A | NPAS2 | RAD54L2 | SMEK2 | UCHL5 |
| ASH1L | CHRAC1 | FTO | KDM5B | NPM1 | RAG1 | SMYD1 | UHRF1 |
| ASH2L | CHTOP | GADD45A | KDM5C | NPM2 | RAG2 | SMYD2 | UHRF2 |
| ASXL1 | CHUK | GADD45B | KDM5D | NSD1 | RAI1 | SMYD3 | UIMC1 |
| ASXL2 | CIR1 | GADD45G | KDM6A | NSL1 | RARA | SMYD4 | USP11 |
| ASXL3 | CIT | GATAD1 | KDM6B | NSRP1 | RB1 | SNAI2 | USP12 |
| ATAD2 | CLNS1A | GATAD2A | KDM7A | NSUN2 | RBBP4 | SP1 | USP15 |
| ATAD2B | CLOCK | GATAD2B | KDM8 | NSUN6 | RBBP5 | SP100 | USP16 |
| ATF2 | CRB2 | GFI1 | KEAP1 | NTMT1 | RBBP7 | SP140 | USP17L2 |
| ATF7IP | CREBBP | GFI1B | KHDRBS1 | NUP98 | RBFOX1 | SPEN | USP21 |
| ATM | CSNK2A1 | GLYR1 | KLF18 | OGT | RBM11 | SPOP | USP22 |
| ATN1 | CSRP2BP | GSE1 | KMT2A | OIP5 | RBM15 | SRCAP | USP3 |
| ATR | CTBP1 | GSG2 | KMT2B | PADI1 | RBM15B | SRRM4 | USP36 |
| ATRX | CTBP2 | GTF21 | KMT2C | PADI2 | RBM17 | SRSF1 | USP44 |
| ATXN7 | CTCF | GTF3C4 | KMT2D | PADI3 | RBM24 | SRSF10 | USP46 |
| ATXN7L3 | CTCFL | HAT1 | KMT2E | PADI4 | RBM25 | SRSF12 | USP49 |
| AURKA | CTR9 | HCFC1 | L3MBTL1 | PAF1 | RBM4 | SRSF3 | USP7 |
| AURKB | CUL1 | HCFC2 | L3MBTL2 | PAGR1 | RBM5 | SRSF6 | UTY |
| AURKC | CUL2 | HDAC1 | L3MBTL3 | PAK2 | RBM7 | SS18L1 | VDR |
| BABAM1 | CUL3 | HDAC10 | L3MBTL4 | PARG | RBM8A | SS18L2 | VIRMA |
| BAHD1 | CUL4A | HDAC11 | LAS1L | PARP1 | RBMY1A1 | SSRP1 | VPS72 |
| BANP | CUL4B | HDAC2 | LBR | PARP2 | RBX1 | STK4 | VRK1 |
| BAP1 | CUL5 | HDAC3 | LEO1 | PARP3 | RCC1 | SUDS3 | WAC |
| BARD1 | CXXC1 | HDAC4 | LRWD1 | PAXIP1 | RCOR1 | SUPT16H | WDR5 |
| BAZ1A | DAPK3 | HDAC5 | MAGOH | PBK | RCOR3 | SUPT3H | WDR77 |
| BAZ1B | DAXX | HDAC6 | MAP3K7 | PBRM1 | REST | SUPT6H | WDR82 |
| BAZ2A | DDB1 | HDAC7 | MAPKAPK3 | PCGF1 | RFOX1 | SUPT7L | WHSC1 |
| BAZ2B | DDB2 | HDAC8 | MASTL | PCGF2 | RING1 | SUV39H1 | WHSC1L1 |
| BCOR | DDX17 | HDAC9 | MAX | PCGF3 | RLIM | SUV39H2 | WSB2 |
| BCORL1 | DDX21 | HDGF | MAZ | PCGF5 | RMI1 | SUV420H1 | WTAP |
| BMI1 | DDX5 | HDGFL2 | MBD1 | PCGF6 | RNF168 | SUV420H2 | YAF2 |
| BPTF | DDX50 | HELLS | MBD2 | PCNA | RNF2 | SUZ12 | YEATS2 |
| BRCA1 | DEK | HIF1AN | MBD3 | PDP1 | RNF20 | SYNCRIP | YEATS4 |
| BRCA2 | DHX9 | HINFP | MBD4 | PELP1 | RNF40 | TADA1 | YTHDC1 |
| BRCC3 | DMAP1 | HIRA | MBD5 | PHC1 | RNF8 | TADA2A | YWHAB |
| BRD1 | DNAJC1 | HIRIP3 | MBD6 | PHC2 | RNPS1 | TADA2B | YWHAE |
| BRD2 | DNAJC2 | HJURP | MBIP | PHC3 | RPS6KA3 | TADA3 | YWHAZ |
| BRD3 | DND1 | HLCS | MBNL1 | PHF1 | RPS6KA4 | TAF1 | YY1 |
| BRD4 | DNMT1 | HLTF | MBNL3 | PHF10 | RPS6KA5 | TAF10 | ZBTB16 |
| BRD7 | DNMT3A | HMG20A | MBTD1 | PHF12 | RPUSD3 | TAF12 | ZBTB33 |
| BRD8 | DNMT3B | HMG20B | MCRS1 | PHF13 | RRP8 | TAF1L | ZBTB7A |
| BRD9 | DNMT3L | HMGB1 | MDC1 | PHF14 | RSF1 | TAF2 | ZBTB7C |
| BRDT | DNTTIP2 | HMGN1 | MEAF6 | PHF19 | RSRC1 | TAF3 | ZC3H13 |
| BRE | DOT1L | HMGN2 | MECP2 | PHF2 | RUVBL1 | TAF4 | ZCWPW1 |
| BRMS1 | DPF1 | HMGN3 | MEN1 | PHF20 | RUVBL2 | TAF5 | ZFP57 |
| BRMS1L | DPF2 | HMGN4 | METTL11B | PHF20L1 | RYBP | TAF5L | ZGPAT |
| BRPF1 | DPF3 | HMGN5 | METTL14 | PHF21A | SAFB | TAF6 | ZHX1 |
| BRPF3 | DPPA3 | HNRNPU | METTL16 | PHF8 | SAP130 | TAF6L | ZMYM2 |
| BRWD1 | DPY30 | HNRPL | METTL21A | PHIP | SAP18 | TAF7 | ZMYM3 |
| BRWD3 | DR1 | HNRPM | METTL3 | PIWIL4 | SAP25 | TAF8 | ZMYND11 |
| BUB1 | DTX3L | HP1BP3 | METTL4 | PKM | SAP30 | TAF9 | ZMYND8 |
| C11orf30 | DZIP3 | HR | MGA | PKN1 | SAP30L | TAF9B | ZNF217 |
| C14orf169 | E2F6 | HSFX3 | MGEA5 | POGZ | SATB1 | TBL1XR1 | ZNF516 |
| C17orf49 | EED | HSPA1A | MINA | POLE3 | SATB2 | TDG | ZNF532 |
| CARM1 | EEF1AKMT3 | HSPA1A | MIS18A | PPARGC1A | SCMH1 | TDRD3 | ZNF541 |
| CBLL1 | EEF1AKMT4 | HSPA1B | MIS18BP1 | PPM1G | SCML2 | TDRD7 | ZNF592 |
| CBX1 | EEF1AKNMT | HSPA1B | MLLT1 | PPP2CA | SCML4 | TDRKH | ZNF687 |
| CBX2 | EHMT1 | HUWE1 | MLLT10 | PPP4C | SENP1 | TET1 | ZNF711 |
| CBX3 | EHMT2 | IKBKAP | MLLT6 | PPP4R2 | SENP3 | TET2 | ZNHIT1 |
| CBX4 | EID1 | IKZF1 | MORF4L1 | PQBP1 | SET | TET3 | ZRANB3 |

In some embodiments, the editing domain is a transcriptional editing domain. The transcriptional editing domain may be a transcriptional editing domain that enhances transcription or represses transcription. The transcriptional editing domain may be any transcriptional editing domain deemed useful. Transcriptional editing domains include, without limitation, VP64, KRAB, VP16, MeCP2, HSF1, etc. In some embodiments, the transcriptional editing domain is a transcriptional editing domain found in Table 4.

In some embodiments, the first or second editing domain is a nanobody or scFv. Domains that are nanbodies or scFvs are known in the art and have been described by, for example, Van et al. (Nat Commun. 2021 Jan. 22; 12(1):537) which is specifically incorporated by reference herein.

The polypeptides of the present disclosure comprise a nuclease-deficient RNA guided endonuclease domain. The nuclease-deficient RNA guided endonucleasc domain utilized in the methods disclosed herein is any nuclease-deficient RNA guided endonuclease domain that is able to bind to specific genomic site without cleaving said genomic site. In some embodiments, the nuclease-deficient RNA guided endonuclease domain is a nuclease-deficient CRISPR/Cas effector polypeptide. In some embodiments, the CRISPR/Cas effector polypeptide is a class 2 nuclease-deficient CRISPR/Cas effector polypeptide. Class 2 nuclease-deficient CRISPR/Cas effector polypeptides include, without limitation, type II, type V, type VI nuclease-deficient CRISPR/Cas effector polypeptides, etc. Type II nuclease-deficient CRISPR/Cas effector polypeptides include, without limitation, nuclease-deficient Cas9 (e.g. dCas9), etc. Type V nuclease-deficient CRISPR/Cas effector polypeptides include, without limitation, nuclease-deficient Cpf1 (e.g. dCas12a), nuclease-deficient C2c1, nuclease-deficient C2c3, etc. Type VI nuclease-deficient CRISPR/Cas effector polypeptides include, without limitation, nuclease-deficient C2c2 (e.g. dCas13a), etc. In a preferred embodiment, the class 2 nuclease-deficient CRISPR/Cas effector polypeptide is a CRISPR/dCas9 effector polypeptide. In some embodiments, the CRISPR/dCas9 effector polypeptide further comprises further modification that alter PAM site specificity such as those disclosed in Table 1. In some embodiments, the class 2 nuclease-deficient CRISPR/Cas effector polypeptide is a CRISPR/dCas12 effector polypeptide.

In some embodiments, in place of the nuclease-deficient RNA guided endonuclease domain, a naturally DNase-free CRISPR enzyme domain. Any naturally DNase-free CRISPR enzyme domain may be used. In some embodiments, the naturally DNase-free CRISPR enzyme domain is a Cas12c enzyme domain. Cas12c enzyme domains are known in the art and have been described by, for example, Huang et al. (Mol Cell. 2022 Jun. 2; 82(11):2148-2160.e4) which is incorporated by reference herein.

In some embodiments, a DNA binding domain is used in place of a nuclease-deficient RNA guided endonuclease domain. The DNA binding domain may be any DNA binding domain deemed useful. DNA binding domains that find use in the present disclosure include, without limitation, transcription activator-like effector (TALE), zinc finger DNA binding domain, etc. TALE is known in the art and has been described in, for example, Becker et al. (Gene Genome Ed. 2021; 2:100007) which is specifically incorporated by reference herein. Zinc finger binding domains are known in the art and have been described by, for example, Kim et al. (Mol Cells. 2017 Aug. 31; 40(8): 533-54), which is specifically incorporated by reference herein. In some embodiments, when a DNA binding domain is used in place of a nuclease-deficient RNA guided endonuclease domain, the method does not comprise the second expression cassette.

The polypeptides of the present disclosure comprise a first and second linker. The linker may be any linker deemed useful. In some embodiments, the peptide linker has 2, 3, 4, 5, 6, 7, 8, 9, 10, 11, 12, 13, 14, 15, 16, 17, 18, 19, 20 or greater than 20 amino acids. In some embodiments, the peptide linker is between 1 to 5, 1 to 10, 1 to 15, 1 to 20, 5 to 10, 5 to 15 or 5 to 20 amino acids in length. Exemplary linkers include linear peptides having at least two amino acid residues such as Gly-Gly, Gly-Ala-Gly, Gly-Pro-Ala, Gly-Gly-Gly-Gly-Ser (SEQ ID NO: 5). Suitable linear peptides include poly glycine, polyserine, polyproline, polyalanine and oligopeptides consisting of alanyl and/or serinyl and/or prolinyl and/or glycyl amino acid residues. In some embodiments, the peptide linker has the amino acid sequence selected from the group consisting of Gly₉ (SEQ ID NO: 6), Glug (SEQ ID NO: 7), Serg (SEQ ID NO: 8), Gly₅-Cys-Pro2-Cys (SEQ ID NO: 9), (Gly₄-Ser)₃ (SEQ ID NO: 10), Ser-Cys-Val-Pro-Leu-Met-Arg-Cys-Gly-Gly-Cys-Cys-Asn (SEQ ID NO: 11), Pro-Ser-Cys-Val-Pro-Leu-Met-Arg-Cys-Gly-Gly-Cys-Cys-Asn (SEQ ID NO: 12), Gly-Asp-Leu-Ile-Tyr-Arg-Asn-Gln-Lys (SEQ ID NO: 13), and Gly₉-Pro-Ser-Cys-Val-Pro-Leu-Met-Arg-Cys-Gly-Gly-Cys-Cys-Asn (SEQ ID NO: 14). In some embodiment, the linker is a Gly-Thr-Gly-Ala (SEQ ID NO: 15) linker. The linker may be an XTEN linker such as the XTEN linkers described in U.S. Pat. No. 11,434,491, specifically incorporated by reference herein.

The methods of the present disclosure comprise expressing a second expression cassette in a cell. The second expression cassette comprises a nucleic acid encoding a guide RNA directed to a target of interest. The target of interest may be any target of interest deemed useful. In some embodiments, the guide RNA is a single guide RNA. In some embodiments, the nucleic acid in the second expression is operably linked to a promoter.

The expression cassette of the present disclosure may comprise any promoter deemed useful. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter.

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to variant type V CRISPR/Cas effector polypeptide of the present disclosure, thus resulting in a fusion polypeptide.

In some cases, a nucleotide sequence encoding a variant type V CRISPR/Cas effector polypeptide of the present disclosure, or a fusion polypeptide of the present disclosure, is operably linked to an inducible promoter. In some cases, a nucleotide sequence encoding a variant type V CRISPR/Cas effector polypeptide of the present disclosure is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20. 497-500 (2002)), an enhanced U6 promoter (e.g., Xia et al., Nucleic Acids Res. 2003 Sep. 1; 31(17)), a human H1 promoter (H1), and the like.

The method of the present disclosure comprise separating the plurality of cells based on the expression level of the target of interest. The separating may be any separating deemed useful for the methods disclosed herein. In some embodiments, the separating comprising sorting the cells using a flow cytometer. In some embodiments, the separating comprises magnetically separating the cells. In some embodiments, the separating comprises the use of drug selection to remove undesired cells. In some embodiments, the target of interest is a gene. In some embodiments, the expression level refers to amount of gene product produced by the gene. In some embodiments, the gene product is a protein. In some embodiments, the plurality of cells are separated into groups that have a high level of expression or a low level of expression. High and low levels of expression are relative to the expression levels of the target of interest in the population as a whole. In some embodiments, cells that have high a level of expression are in the top 40% of the plurality of cells that have the highest level of expression and cells that have low expression are the bottom 40% of the plurality of cells that have the lowest expression. In some embodiments, cells that have high a level of expression are in the top 30% of the plurality of cells that have the highest level of expression and cells that have low expression are bottom 30% of the plurality of cells that have the lowest expression. In some embodiments, cells that have high a level of expression are in the top 25% of the plurality of cells that have the highest level of expression and cells that have low expression are the bottom 25% of the plurality of cells that have the lowest expression. In some embodiments, cells that have high a level of expression are in the top 15% of the plurality of cells that have the highest level of expression and cells that have low expression are bottom 15% of the plurality of cells that have the lowest expression. In some embodiments, cells that have high a level of expression are in the top 10% of the plurality of cells that have the highest level of expression and cells that have low expression are the bottom 10% of the plurality of cells that have the lowest expression.

The target of interest of the present disclosure may be any target of interest to which the editing is desired. In some embodiments, the target of interest is a gene. In some embodiments, the gene is operably linked to a sequence encoding a fluorescent protein. In some embodiments, the gene is not operably linked to a sequence encoding a fluorescent protein. In some embodiments, when the gene is not operably linked to a sequence encoding a fluorescent protein, the product of the gene, also known as the gene product, (e.g. a protein) may be labeled with an antibody conjugated to a fluorophore.

The method of the present disclosure comprise preforming a sequencing reaction on genomic DNA (gDNA) in each cell in the plurality of cells. In some embodiments, only the gDNA of cells that have a high level of expression of the target of interest or have a low level of expression of the target of interest is sequenced. In some embodiments, the sequence reaction is a long read sequencing reaction. Long read sequencing reaction are known in the art and have been described in, for example, Amarasinghe et al. (Genome Biol. 2020 Feb. 7; 21(1):30) which is specifically incorporated by reference herein. The genomic DNA used the sequencing reaction may be any region of genomic DNA of interest. In some embodiments, the genomic DNA comprises a nucleic acid sequence corresponding to the first expression cassette.

In some embodiments, in place of extracting and sequencing gDNA, perturb-seq may be used. Peturb-seq is known in the art and has been described by, for example, Schraivogel et al. (Nat Methods. 2020 June; 17(6):629-635) which is incorporated by reference herein.

The first and/or second expression cassette of the present disclosure may be contained in an expression vector. Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like, and transposons such as sleeping beauty or piggyback. In some cases, a recombinant expression vector of the present disclosure is a recombinant adeno-associated virus (AAV) vector.

In some embodiments, the first expression cassette is contained in a lentivirus expression vector. In some embodiments, the second expression cassette is contained in a lentivirus expression vector. In some embodiments, the first and the second expression cassettes are contained in the same lentivirus expression vector.

### Libraries

The present disclosure provides libraries comprise a plurality of expression cassettes each comprising a nucleic acid encoding a polypeptide wherein each polypeptide comprises, in order, a first editing domain, a first linker, a nuclease-deficient RNA guided endonuclease domain, a second linker, and a second editing domain; wherein the plurality of expression cassettes comprise at least 1000 different combinations of first and second editing domain combinations.

The first and second editing domain may any combination of the editing domains disclosed in Table 4. In some embodiments, the first editing domain is any one of N1-N349 as defined by Table 4. In some embodiments, the second editing domain is any one of C1-C349 as defined by Table 4. In some embodiments, the first and second linker is an XTEN linker. In some embodiments, the nuclease-deficient RNA guided endonuclease domain is a nuclease-deficient CRISPR/Cas effector polypeptide. In some embodiments, the CRISPR/Cas effector polypeptide is a class 2 nuclease-deficient CRISPR/Cas effector polypeptide. In some embodiments, the class 2 nuclease-deficient CRISPR/Cas effector polypeptide is a CRISPR/dCas9 effector polypeptide. In some embodiments, the class 2 nuclease-deficient CRISPR/Cas effector polypeptide is a CRISPR/dCas12 effector polypeptide.

The libraries of the present disclosure comprise a range of different unique combinations of first and second editing domains. For instance, the libraries may comprise at least 1000, at least 2000, at least 4000, at least 6000, at least 8000, at least 10000, at least 20000, at least 30000, at least 40000, at least 50000, at least 60000, at least 70000, at least 80000, at least 90000, at least 100000 or greater than 100000 unique combinations of first and second editing domains.

### EXAMPLES

### Example 1

A novel platform for identifying and characterizing programmable gene modulators was developed. The approach involves screening libraries of proteins composed of catalytically inactivated CRISPR associated protein 9 (dCas9) fused to protein domains from human gene products involved in epigenetic processes. Each element of the libraries encodes dCas9 and a discrete domain at each terminus (N and C) of dCas9, resulting in a dual terminus dCas9 fusion protein that can be screened for functional activity in mammalian cells in a high-throughput and unbiased manner. To date two libraries have been constructed composed of 1369 or ˜110,000 unique proteins. Long-read sequencing and analysis method were employed to uncover causative constructs in relation to the phenotype of interest. The use of dCas9, which has been widely shown to be programmable to any region of the genome, allows for the platform to be used to target any genetic element in the genome with ease, including, for example, transcribed or untranscribed genes which could be protein coding or non-coding as well as gene regulatory elements such as enhancers, insulators and topology modulators. Moreover, the format of this platform allows it to be utilized in a variety of biological models, including but not limited to, mammalian cell lines, primary cells, and in vivo models.

This approach differs from conventional methods of gene editor discovery by bypassing time- and labor-intensive construct development, which is most frequently done in an arrayed format and at low throughput. Moreover, the sequencing and analysis methods allow for the identification of active editors without relying on construct barcodes, which are easily decoupled from their cogent construct through lentiviral recombination, thus recovering truly active constructs.

### Example 2

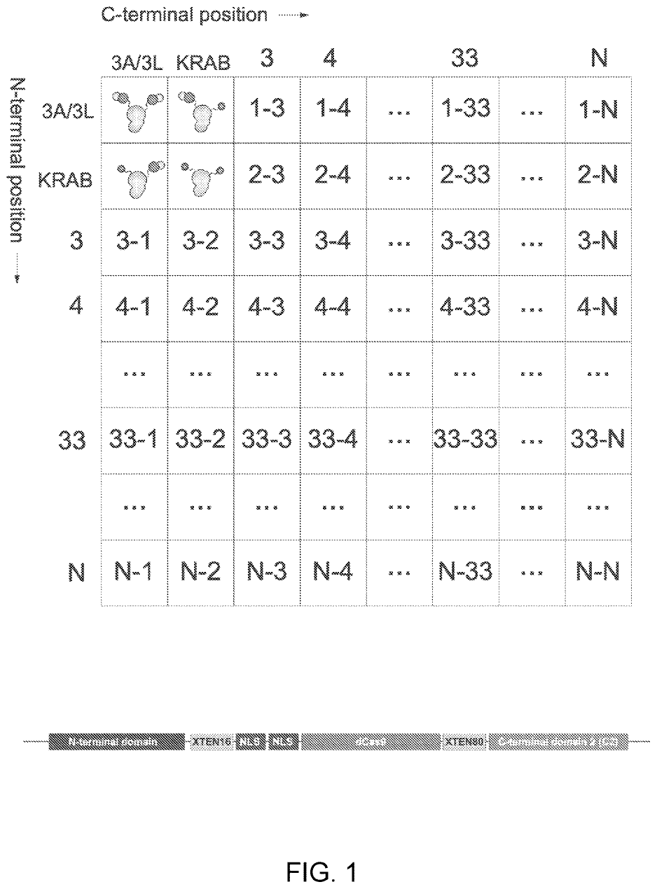

Proof-of-concept combinatorial transcriptional effector library modulates CD55 expression. We report the generation of RNA-guided dual transcriptional effector libraries. We generated a proof-of-concept dual-fusion transcriptional effector domain library consisting of 37 protein domains fused on either terminus of a catalytically inactive Cas9 (dCas9). Of the 37 protein domains used in the preliminary library, 32 domains were extracted from 20 genes (see methods) and 5 domains come from previously characterized gene modulators(1-4). The 20 genes represented in the small-scale library were selected because they have been thoroughly characterized as genes whose products modify gene expression via epigenetic or transcriptional modulation. The inclusion of domains on both the N- and C-termini of dCas9 generated 1,369 dual terminus fusion proteins, hereby referred to as Library 1 (L1) (FIG. 1).

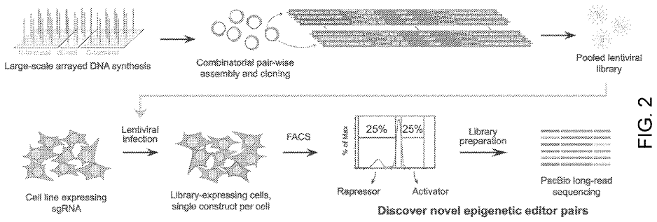

Using a K562 cell line expressing a sgRNA targeting CD55, we transduced library 1 and selected for domain construct integration via puromycin selection (FIG. 2, see methods), with two independent technical replicates. On Day 8 post infection. >20e6 cells per replicate were analyzed via FACS and sorted based on CD55 expression (CD55-high and -low populations), along with an unsorted population, maintaining greater than 10,000-fold coverage of L1. Full length effector constructs were amplified from genomic DNA and sequenced using long read sequencing.

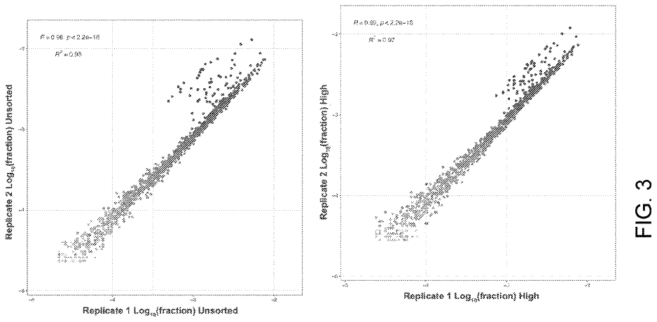

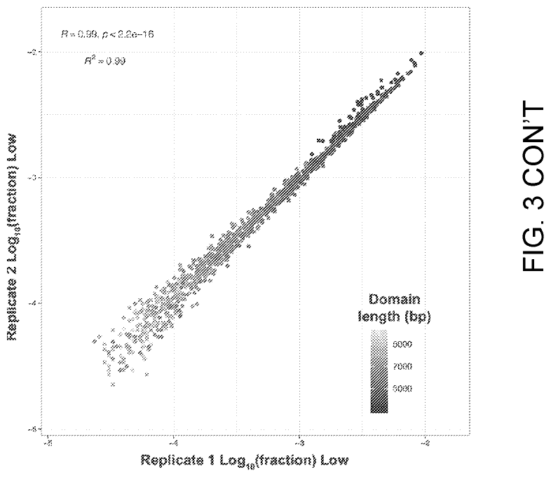

92.8-93.4% of Library 1 was successfully represented in the screen (1270 to 1278 of 1369 possible combinations, replicate dependent) in the unsorted population of infected cells, with slight drop out of library elements that were not recoded for both termini prior to synthesis. Library 1 replicates were highly correlated for all 3 conditions [Pearson correlation coefficient (R): CD55^{high}=0.99 (p<2.2c-16), CD55^{low}=0.99 (p<2.2c-16), unsorted=0.98 (p<2.2e-16)](FIG. 3), suggesting that our combinatorial transcriptional effector platform is robust and reproducible across replicates.

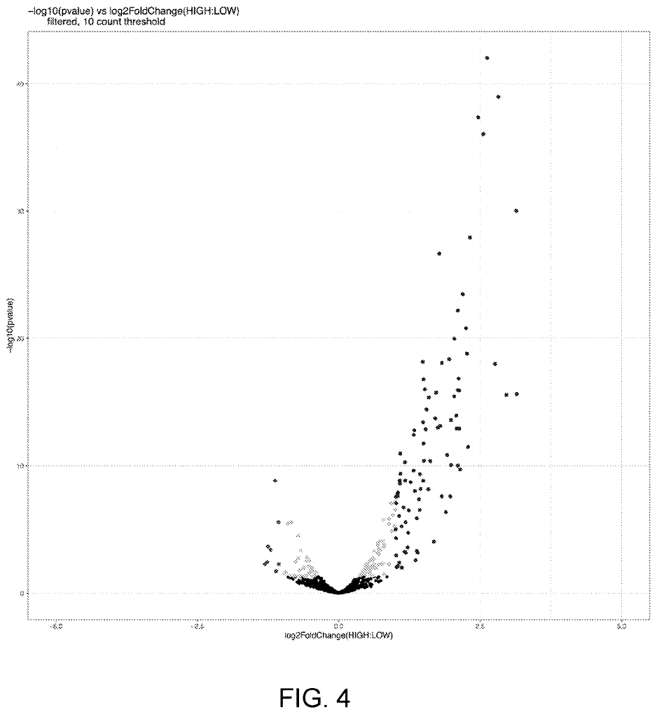

L1 targeting CD55 in K562s, nominated 187 significantly enriched dual-transcriptional effectors (18.57%, p<=0.05) of 1007 fusion proteins that were detected after count filtering. Enriched hits consisted of both activators and repressors of CD55, with many more activators identified compared to repressors (146 and 41 effector combinations, respectively) highlighted by the long tail of activation (FIG. 4). We identified both significant activators (n=91) and repressors (n=8) that are 2-fold more enriched in their respective populations [abs(Log 2FoldChange)>1].

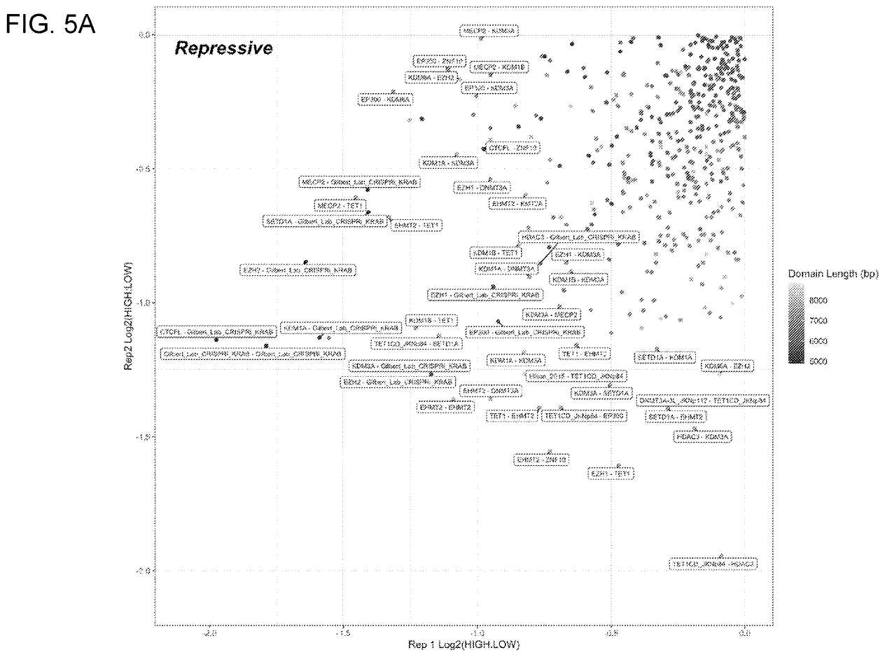

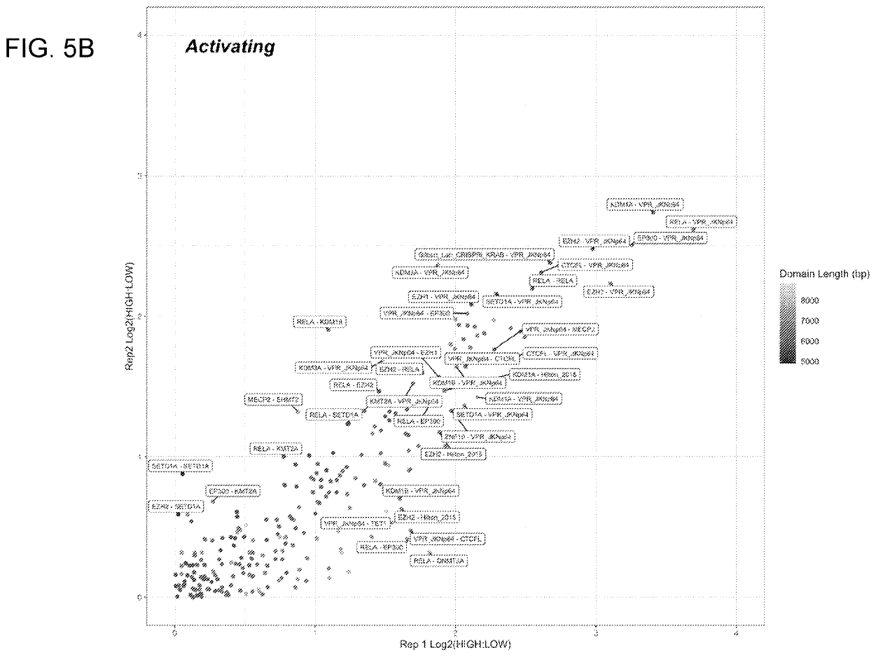

As further proof-of-method, known dCas9-guided transcriptional regulators developed in the Gilbert and Weissman laboratories(1,5) or by other labs in the field(2) including dCas9-VPR and dCas9-KRAB containing constructs, were identified in the CD55 activating and repressing categories, respectively (FIG. 5).

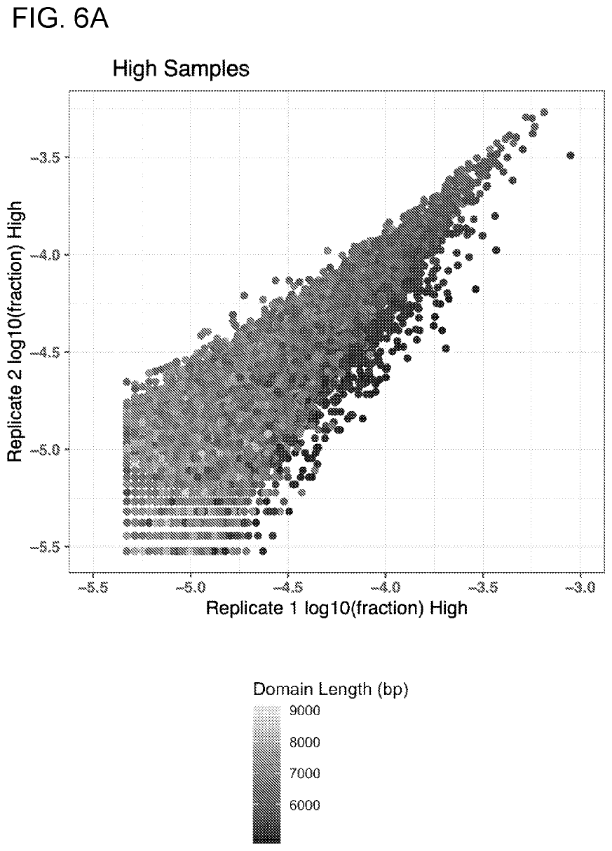

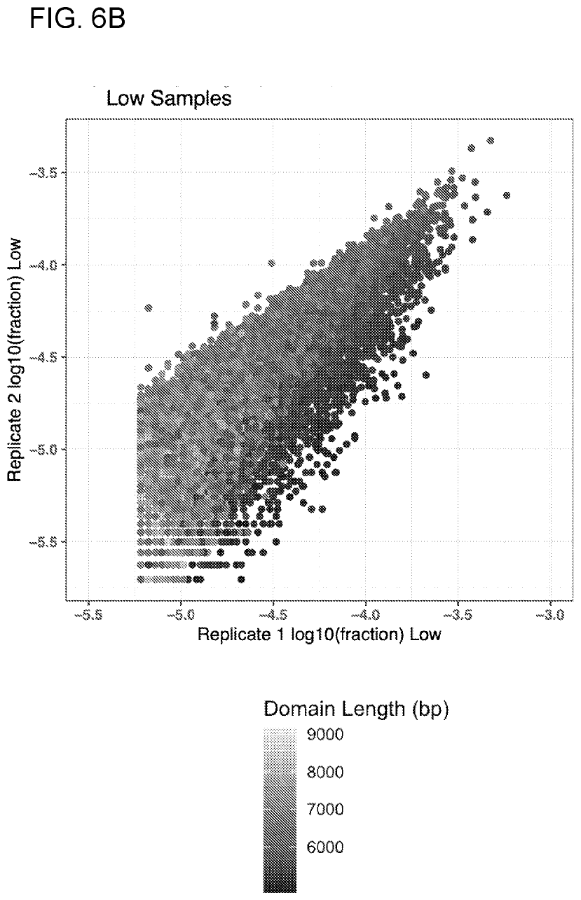

Large-scale combinatorial transcriptional effector discovery. Our large-scale library consists of combinations of 347 effector domains fused to the N or C-terminus of dCas9, resulting in 110,554 combinations of a possible 120,409 combinations after domain synthesis dropout, termed Library 2. Screens were carried out in K562 cells and the domain library was recruited to the target locus, CD55, via a single gRNA (as described in the library 1 screen). Sequencing depth was limited using 4 Sequel II lanes for two replicates of both high and low CD55-expressing cells, such that 72,547-77,472 of 110,554 domains were detected (65.6-70.1%, replicate and condition specific) prior to count threshold filtering. After filtering for fusion proteins with 10 counts per replicate and condition, counts were normalized (see methods) and Log₂(Fold Change) was calculated. Despite low library coverage in sequencing, Library 2 was highly correlated across replicates (FIG. 6) for both CD55^{HIGH} and CD55^{LOW} conditions [Pearson correlation coefficient (R): CD55^{high}=0.94, CD55^{low}=0.90].

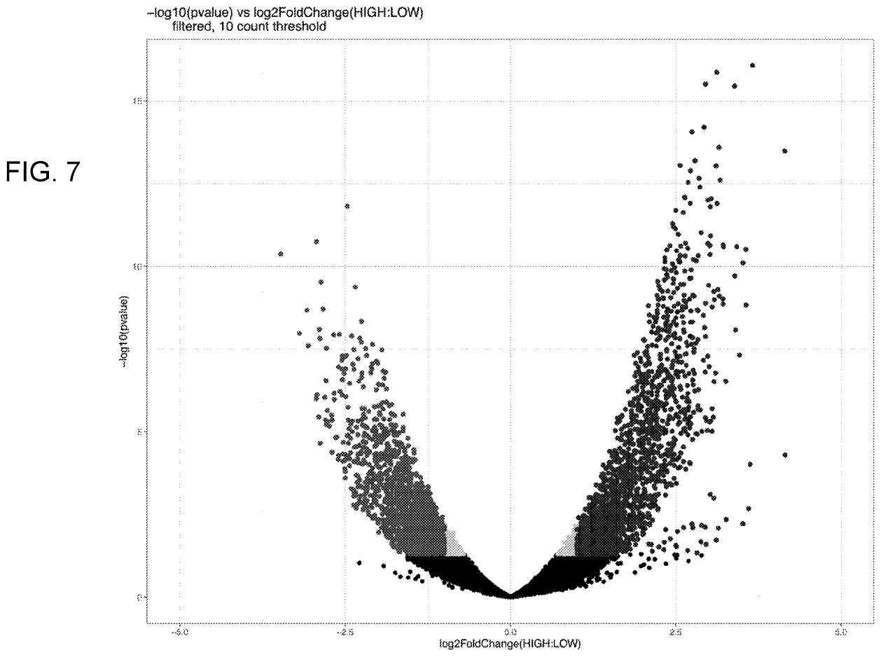

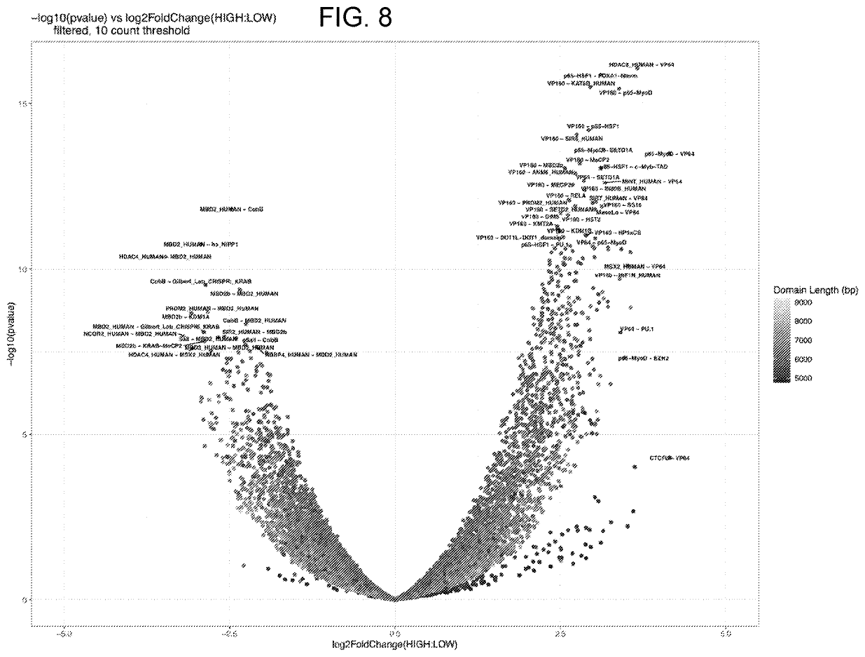

We observed 3709 significant hits (p value<=0.05); 1803 (48.6%) increased CD55 expression more than 2-fold, and 1602 (43.2%) repressed CD55 expression more than 2-fold (FIGS. 7 and 8). Notably, with the inclusion of additional domains in Library 2 relative to Library 1, we see an increase in the range of tunable CD55 expression, which was limited on the repression side in Library 1, emphasizing our system as a platform for epigenetic editor discovery and tunable locus-specific gene expression dependent on the domain pair combination.

Combinatorial effector domain screening enables domain epistasis analyses. Our library design of effectors fused to both termini of dCas9 enables analyses of position-specific effects when in either the N- or C-terminal position of a particular effector pair. Further, we can begin to uncover the transcriptional effects in the context of the same effector domains in different orientations with respect to dCas9, enabling future approaches in synthetic protein design for establishing what constitutes effective epigenetic editors.

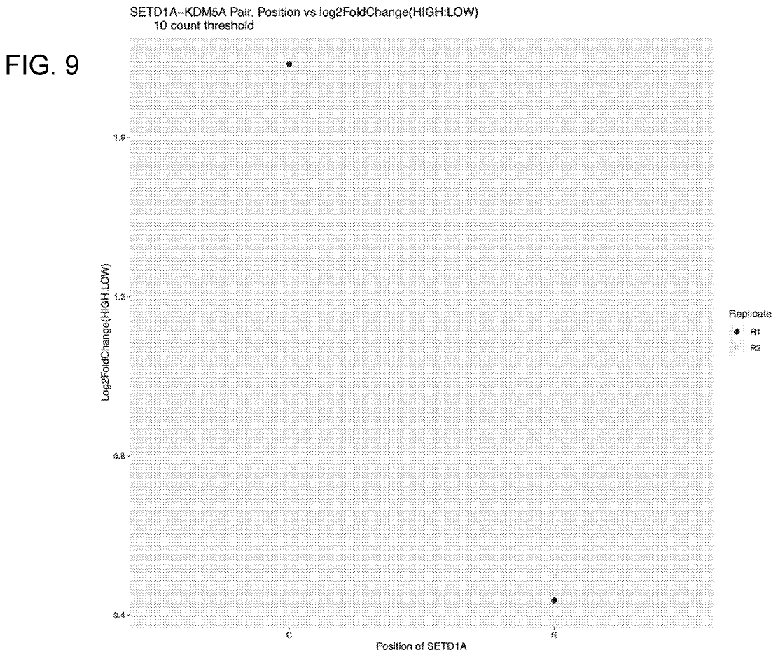

For example, including position for each domain (N- or C-terminal position of dCas9) as a factor in our analyses (using DESeq2, (6)) we can identify effector pairs that have significantly different expression profiles-enriched in activator or repressor profiles (or inactive)—suggesting that specific domains combinations in our library prefer specific conformations for effective activity. Specifically, we identify effectors SETD1A and KDM5A as one such pair in which N- vs C-terminal positions impact CD55 expression in the large-scale screen (FIG. 9). In this combination, when SETD1A is in the N-terminal position of the effector pair, it is not enriched in the CD55^{HIGH} population, compared to when SETD1A is in the C terminal position, where it is a strong transcriptional activator of CD55. Our screening platform enables future analyses like this one to identify both position-specific effects and synergy among domain combinations.

### Methods

### Library Design

Library 1 (37×37) Design. We designed two libraries that differ in size. The small-scale library is comprised of 37 domains; 32 domains were extracted in an unbiased fashion from 20 genes and 5 domains come from previously characterized gene modulators (1-4). The 20 genes represented in the small-scale library were selected because they have been thoroughly characterized as genes whose products modify gene expression via epigenetic or transcriptional modulation. The 37 domains were then synthesized by Twist Bioscience as DNA constructs expressed at the N- and C-termini of dCas9, resulting in 1,369 dual terminus fusion proteins. Library one ranges in size from 4896 base pairs (bp) in length to 11,346 bp in length, with the mean length equal to 7084 bp.

Library 2 (347×347) Design. The large-scale library is comprised of 347 domains. 266 of these domains were extracted from 194 genes obtained from dbEM, a database of epigenetic modifiers(7). 81 previously published domains were included as positive controls(8,9). The same domains of the small-scale library were included in the large-scale library to maximize the probability of finding highly effective pairs of domains. Individual domains in the large-scale library range between 135 and 3861 base pairs, with a median length of 1301 base pairs. The large-scale library was synthesized in the same manner as the small-scale library by Twist Bioscience. Of the 347 domains in the library, ˜95% were synthesized successfully, resulting in a total of 110,554 dual terminus dCas9 fusion proteins.

### Library Generation

Domain Synthesis. N and C terminal elements, along with the constant dCas9 fragment, were synthesized and assembled by Twist Biosciences. Our dCas9 fragment consists of: XTEN16 linker-2×NLS sequences-dCas9-XTEN80. The complete list of N-terminal and C-terminal domains can be found in Table 4.

### Library 1 Screens

Library 1 (37×37) Amplification. The library was diluted to 10 ng/uL. For the first transformation, 100 ng (10 uL of 10 ng/uL) were transformed into 75 uL competent Stellar E. coli cells (Takara) by heat shocking the cells at 42 C for 42s. Cells were allowed to recover on ice for 2 min then recovered in SOC medium (final volume of 1 mL) at 37 C and 300 rpm. After a one hour recovery, 250 μL of transformed cells were plated on each of two 24×24 cm (576 cm²) LB-agar plates supplemented with carbenicillin (100 μg/mL. The following day, colonies were counted and each plate contained 2.9e4 colonies, resulting in 20.5-fold coverage of the library per plate.

For the second transformation, 100 ng of the library were transformed into 75 uL competent Stellar E. coli cells (Takara) in each of two parallel transformation reactions. Cells were allowed to recover on ice for 2 min then recovered in pre-warmed SOC medium (final volume of 1 mL) at 37 C and 300 rpm. After a one hour recovery, the parallel transformations were combined and 350 μL of transformed cells were plated on each of five 24×24 cm (576 cm²) LB-agar plates supplemented with carbenicillin. The following day, the five plates were found to have a slightly larger number of colonies as the two plates from the first transformation by visual inspection, resulting in >150-fold coverage of the library.

All seven plates were scraped to collect the bacterial colonies using 20-25 mL LB with an additional 25 mL LB wash per plate. The library was isolated using the ZymoPure II Gigaprep kit (Zymo) following manufacturer's instructions. The yield was 1612 ng/uL in ˜4 mL, for a total amount of 6.5 mg library DNA.

Lentivirus preparation. On day −1 of lentiviral prep (D-1), 6.5e6 HEK293T cells were seeded in each of 14 15 cm dishes. Cells were grown in DMEM (Gibco) supplemented with 10% FBS (VWR) and 1% penicillin, streptomycin, and glutamine (Gibco), henceforth dubbed “complete DMEM”. The following day (DO), cells were transfected with the lentiviral packaging plasmids pGagPol, pTat, pRev, and VSVG and the library at a 1:1:1:2:15 ratio by mass using Opti-MEM (Gibco) and TransIT-LT1 reagent (Mirus Technologies). The following morning (D1), media was replaced with fresh DMEM-complete supplemented with 15 mM sterile HEPES and 1× ViralBoost reagent (ALSTEM Bio). In the afternoon of the following day (D2), viral supernatant was harvested and filtered on a 0.45 um PVDF filter bottle.

Lentiviral infection. The day before infection (D-1), 5e6 cells (K562 cells expressing endogenous CD55 and previously transduced with a single guide RNA vector targeting the CD55 promoter) were seeded in each of 14 15 cm dishes. On the day of infection (D0), harvested virus was supplemented with 8 mg/mL polybrene for a final concentration of ˜16 ug/mL polybrene. Reporter cells were 40-50% confluent when 24 mL viral supernatant was added per 15 cm dish, for a final volume of 44 mL media per dish and a final concentration of ˜8.7 ug/mL polybrene. Assuming a 24-hour cell division rate, ˜140e6 cells were present at infection. Assuming 2% infection rate (based on previous small-scale lentiviral titer assays), 2.8e6 cells were infected, resulting in ˜2000-fold coverage of the library at the time of infection.

Puromycin selection. 48 hours post infection (D2), media was aspirated, each 15 cm dish was gently washed with 5 mL PBS (Gibco), and cells were detached using 5 mL 0.25% trypsin-EDTA (Gibco). The trypsinization was quenched with 15 mL complete DMEM and cells were pooled then pelleted by centrifugation. After resuspension in 7 mL complete DMEM, cells were counted on the Countess II Automated Cell Counter (ThermoFisher) and 140e6 cells (representing 1,000-fold coverage of the library) were isolated and resuspended in complete DMEM supplemented with 2.5 ug/mL puromycin (Gibco). Two independent technical replicates were initiated at this step. 24 hours post initiation of puromycin treatment, cells were visually inspected by bright-field microscopy and appeared 20-30% live.

Four days post infection (D4) and 48 hours post initiation of puromycin treatment, media was replaced with fresh complete DMEM supplemented with 2.5 ug/mL puromycin (Gibco).

Recovery from puromycin treatment. Two days later (D6), media was aspirated, each 15 cm dish was gently washed with 5 mL PBS (Gibco), and cells were detached using 5 mL 0.25% trypsin-EDTA (Gibco). The trypsinization was quenched with 15 mL complete DMEM and cells within the same replicate were pooled then pelleted by centrifugation. Cells were counted three times per replicate and the average cell count was used to calculate an approximate infectivity rate by comparing the observed cell count to the expected cell count if 100% of cells had been infected, assuming cells divided every 24 hours. This produced approximate infection rates of 36% for replicate 1 and 28% for replicate 2, which were much higher than expected. Assuming 70% puro selection (i.e. only 70% of the cells at D6 were effector-positive), 2e3 cells would represent 1-fold coverage of the library. To maintain 10,000-fold coverage of the library, 20e6 cells would need to be plated. As such, 5e6 cells were seeded in each of 5 15 cm dishes per replicate, for a total of 25e6 cells per replicate and representing >10,000-fold coverage of the library.

FACS-based sort. On day 8 post infection, media was aspirated, each 15 cm dish was gently washed with 5 mL PBS (Gibco), and cells were detached using 5 mL 0.25% trypsin-EDTA (Gibco). The trypsinization was quenched with 15 mL complete DMEM and cells within the same replicate were pooled then pelleted by centrifugation. Each cell pellet was washed with FACS media, stained for CD55 expression (50 ul/mL CD55-APC, Biolegend) and sorted based on CD55 expression. Specifically, cells were stained at 10e6 cells/mL (in FACS buffer) with 50 ul/mL CD55-APC Ab for 25 min on ice, followed by FACS buffer addition and centrifugation. Cells were counted then pelleted by centrifugation and resuspended in FACS media-1 (1% FBS in PBS) for a final concentration of 10e6 cells/mL.

For each replicate, >20e6 cells were analyzed on a FACSAria Fusion (BD), maintaining >10,000-fold coverage of the library. 5e6 unsorted cells were pelleted by centrifugation at 1,000×g for 5 min, media was aspirated, and cells were placed at −80 C.

Harvesting samples, quartile bins. For quartile binning, 5e6 cells in the bottom 25% of CD55 expression and 5e6 cells in the top 25% of CD55 expression were sorted, corresponding to the “Q_low” and “Q_high” samples, respectively. Cells were collected in 15 mL conical tubes pre-coated with FBS. Cells were pelleted by centrifugation at 1,000×g for 5 min, media was aspirated, and cells were placed at −80 C.

Genomic DNA extraction. Genomic DNA was extracted using a NucleoSpin Tissue kit (Machery Nagel). Replicate 1 samples were eluted in 5e6 uL water and replicate 2 samples were eluted in 150 uL water. Sample concentrations were measured on a NanoDrop One (ThermoFisher). Expected yield for this genomic DNA extraction kit is 20-35 ug from up to e7 cells; however, all but one sample was outside that range (5 samples were below the range and 4 samples were above the range).

TABLE 3

| | | | | Total | | |
|---|---|---|---|---|---|---|
| | | Input | [gDNA] | DNA | Primer | # |
| Condition | Label | cells | (ng/uL) | (ug) | pair | PCRs |
| Replicate 1, | R1 uns | 5e6 | 774 | 77.4 | o180 | 16 |
| unsorted | | | | | o192 | |
| Replicate 1, high | R1 Qhi | 5e6 | 102 | 10.2 | o181 | 3 |
| expression quartile | | | | | o193 | |
| Replicate 1, low | R1 Qlo | 5e6 | 121 | 12.1 | o182 | 3 |
| expression quartile | | | | | o194 | |
| Replicate 1, high | R1 | 8e6 | 71 | 7.1 | o183 | 2 |
| expression grown out | high | | | | o195 | |
| Replicate 1, low | R1 off | 5e6 | 160 | 16.0 | o184 | 4 |
| expression grown out | | | | | o196 | |
| Replicate 2, | R2 uns | 5e6 | 124.5 | 18.7 | o185 | 4 |
| unsorted | | | | | o197 | |
| Replicate 2, high | R2 Qhi | 5e6 | 277.5 | 41.6 | o186 | 9 |
| expression quartile | | | | | o198 | |
| Replicate 2, low | R2 Qlo | 5e6 | 395.9 | 59.4 | o187 | 12 |
| expression quartile | | | | | o199 | |
| Replicate 2, high | R2 | 6e6 | 160.1 | 24.0 | o188 | 5 |
| expression grown out | high | | | | o200 | |
| Replicate 2, low | R2 off | 5e6 | 455.3 | 68.3 | o189 | 14 |
| expression grown out | | | | | o201 | |

PCRs. A first round of test PCRs was conducted with barcoded primers and 1 ug genomic DNA per sample per replicate. These test PCRs were analyzed by gel electrophoresis for visual confirmation of amplification before proceeding with the full set of genomic DNA PCRs. The test PCRs for replicate 1 included plasmid library amplification as a positive control and the test PCRs for replicate 2 included a no DNA amplification as a negative control. All PCRs worked and all controls validated.

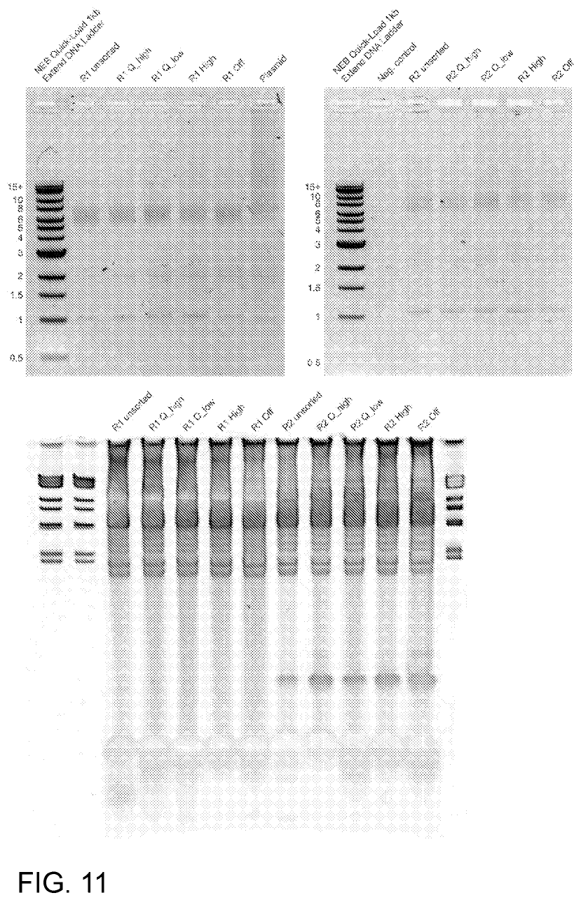

Genomic DNA PCRs for both replicates were conducted with ˜5 ug genomic DNA per 100 uL reaction. Each reaction consisted of 50 uL NEBNext Ultra II Q5 master mix, 1 uL 100 uM barcoded forward primer, 1 uL 100 uM barcoded reverse primer, ˜5 ug gDNA, water to 100 uL. Reactions were kept on ice until they were placed in a thermal cycler for the following PCR: 98° C. for 30s, 28×[98° C. for 10s, 71° C. for 30s, 72° C. for 11 min], 72° C. for 5 min, hold at 4° C. All gDNA was processed. PCRs for each sample of each replicate were pooled and analyzed by gel electrophoresis (FIG. 11).

AMPure PB magnetic bead PCR cleanup. Before conducting magnetic bead cleanup and concentration of the PCR products, an aliquot of each sample was diluted 1:10 and analyzed on a BioAnalyzer (Agilent).

Samples were then cleaned and concentrated using the AMPure PB bead protocol (Pacific Biosciences) with 0.45× bead to sample ratio to size select amplicons 3-15 kb in size. Magnetic bead purification was conducted according to “Procedure & Checklist—Preparing SMRTbell Libraries using Pacific Biosciences Barcoded Universal Primers for Multiplexing Amplicons” (Pacific Biosciences). Samples were eluted in 5 uL water per number of PCR reactions. An aliquot of each sample was diluted 1:10 and analyzed on a NanoDrop One (ThermoFisher), BioAnalyzer (Agilent), and Qubit (Invitrogen) for total nucleic acid concentration and quality, amplicon molarity, and total DNA concentration, respectively.

### Sequencing Read Processing and Analysis

Circular consensus sequence (CCS) generation. subreads.bam files were exported from Sequel IIe (Pacific Biosciences) and Circular Consensus Sequences (CCS) were generated using ccs (10) to produce a BAM file. Default parameters were used with the addition of the threads parameter (−j 40).

Demultiplexing of BAM files. BAM files of CCS were demultiplexed using lima (Pacific Biosciences) to produce BAM files for each sample. Default parameters were used with the addition of the threads parameter (−j 10).

Sequence processing. Briefly, individual BAM files were converted to fastq files using bam2fq (samtools¹¹) and trimmed using trimfq (https://github.com/lh3/seqtk) such that 16 nucleotides on either end were excluded. The parameters were as follows: seqtk trimfq -b 16 -e 16 sample.ccs.fq>sample.ccs.fq

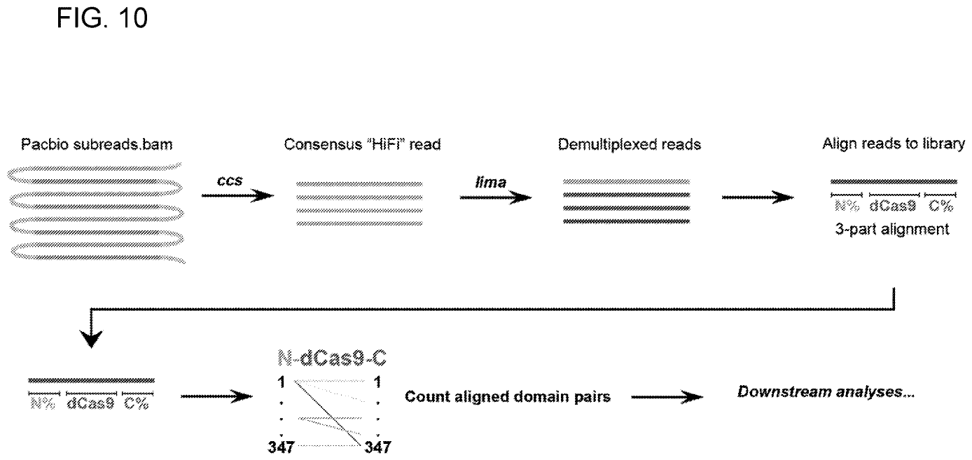

After trimming, samples were processed using scripts developed based on knock-knock(12) to generate three-part alignment counts (FIG. 10). A count threshold of 10 was set for all domain pairs per replicate. Following count filtering, DESeq2(6) was used for downstream analyses with condition (high vs low expression) used as the covariate(“Design”) unless otherwise noted.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### REFERENCES

1. Gilbert, L. A. et al. CRISPR-Mediated Modular RNA-Guided Regulation of Transcription in Eukaryotes. Cell 154, 442-451 (2013).
2. Chavez, A. et al. Highly efficient Cas9-mediated transcriptional programming. Nat Methods 12. 326-328 (2015).
3. Hilton, I. B. et al. Epigenome editing by a CRISPR-Cas9-based acetyltransferase activates genes from promoters and enhancers. Nat Biotechnol 33, 510-517 (2015).
4. Nuñez, J. K. et al. Genome-wide programmable transcriptional memory by CRISPR-based epigenome editing. Cell 184, 2503-2519.e17 (2021).
5. Gilbert, L. A. et al. Genome-Scale CRISPR-Mediated Control of Gene Repression and Activation. Cell 159, 647-661 (2014).
6. Love, M. I., Huber, W. & Anders, S. Moderated estimation of fold change and dispersion for RNA-seq data with DESeq2. Genome Biol 15, 550 (2014).
7. Nanda, J. S., Kumar, R. & Raghava, G. P. S. dbEM: A database of epigenetic modifiers curated from cancerous and normal genomes. Sci Rep-uk 6, 19340 (2016).
8. Konermann, S. et al. Optical control of mammalian endogenous transcription and epigenetic states. Nature 500, 472-476 (2013).
9. Yeo, N. C. et al. An enhanced CRISPR repressor for targeted mammalian gene regulation. Nat Methods 15, 611-616 (2018).
10. Travers, K. J., Chin, C.-S., Rank, D. R., Eid, J. S. & Turner, S. W. A flexible and efficient template format for circular consensus sequencing and SNP detection. Nucleic Acids Res 38, e159-e159 (2010).
11. Danecek, P. et al. Twelve years of SAMtools and BCFtools. Gigascience 10, giab008 (2021).
12. Canaj, H. et al. Deep profiling reveals substantial heterogeneity of integration outcomes in CRISPR knock-in experiments. Biorxiv 841098 (2019) doi:10.1101/841098.

TABLE 4
List of the N and C terminal domains of Library 1 and 2

| | | | Length | | | Length | |
|---|---|---|---|---|---|---|---|
| | | | of N | | | of C | |
| | N terminal | SEQ | terminal | C terminal | SEQ | terminal | |
| Library | Domain ID | ID NO: | domain | Domain ID | ID NO: | domain | Gene name |
| Lib1, Lib2 | N1 | 16 | 258 | C1 | 363 | 252 | SETD1A |
| Lib1, Lib2 | N2 | 17 | 1236 | C2 | 364 | 1230 | SETD1A |
| Lib1, Lib2 | N3 | 18 | 1290 | C3 | 365 | 1284 | HDAC3 |
| Lib1, Lib2 | N4 | 19 | 3291 | C4 | 366 | 3285 | KDM6A |
| Lib1, Lib2 | N5 | 20 | 519 | C5 | 367 | 513 | KDM1A |
| Lib1, Lib2 | N6 | 21 | 1728 | C6 | 368 | 1722 | KDM1A |
| Lib1, Lib2 | N7 | 22 | 624 | C7 | 369 | 618 | ZNF10 |
| Lib1, Lib2 | N8 | 23 | 225 | C8 | 370 | 219 | MECP2 |
| Lib1, Lib2 | N9 | 24 | 906 | C9 | 371 | 900 | MECP2 |
| Lib1, Lib2 | N10 | 25 | 1236 | C10 | 372 | 1230 | HSF1 |
| Lib1, Lib2 | N11 | 26 | 3447 | C11 | 373 | 3441 | KMT2A |
| Lib1, Lib2 | N12 | 27 | 714 | C12 | 374 | 708 | KMT2A |
| Lib1, Lib2 | N14 | 28 | 795 | C14 | 375 | 789 | RELA |
| Lib1, Lib2 | N15 | 29 | 975 | C15 | 376 | 969 | EP300 |
| Lib1, Lib2 | N16 | 30 | 1815 | C16 | 377 | 1809 | EP300 |
| Lib1, Lib2 | N17 | 31 | 483 | C17 | 378 | 477 | EZH2 |
| Lib1, Lib2 | N18 | 32 | 840 | C18 | 379 | 834 | EZH2 |
| Lib1, Lib2 | N20 | 33 | 822 | C20 | 380 | 816 | KDM1B |
| Lib1, Lib2 | N21 | 34 | 1341 | C21 | 381 | 1335 | KDM1B |
| Lib1, Lib2 | N22 | 35 | 1758 | C22 | 382 | 1752 | TET1 |
| Lib1, Lib2 | N23 | 36 | 2391 | C23 | 383 | 2385 | TET1 |
| Lib1, Lib2 | N24 | 37 | 774 | C24 | 384 | 768 | CTCFL |
| Lib1, Lib2 | N25 | 38 | 276 | C25 | 385 | 270 | CTCFL |
| Lib1, Lib2 | N26 | 39 | 411 | C26 | 386 | 405 | EZH1 |
| Lib1, Lib2 | N27 | 40 | 828 | C27 | 387 | 822 | EZH1 |
| Lib1, Lib2 | N28 | 41 | 2607 | C28 | 388 | 2601 | EHMT2 |
| Lib1, Lib2 | N29 | 42 | 1890 | C29 | 389 | 1884 | ASH2L |
| Lib1, Lib2 | N30 | 43 | 1989 | C30 | 390 | 1983 | KDM3A |
| Lib1, Lib2 | N31 | 44 | 2742 | C31 | 391 | 2736 | DNMT3A |
| Lib1, Lib2 | N32 | 45 | 222 | C32 | 392 | 216 | Gilbert_Lab CRISPRi_KRAB |
| Lib1, Lib2 | N33 | 46 | 1860 | C33 | 393 | 1854 | Hilton 2015 |
| Lib1, Lib2 | N34 | 47 | 912 | C34 | 394 | 906 | DNMT3A-3L_JKNp112 |
| Lib1, Lib2 | N35 | 48 | 1557 | C35 | 395 | 1551 | VPR_JKNp64 |
| Lib1, Lib2 | N36 | 49 | 2163 | C36 | 396 | 2157 | TET1CD_JKNp84 |
| Lib1, Lib2 | N37 | 50 | 927 | C37 | 397 | 921 | KDM6A |
| Lib1, Lib2 | N38 | 51 | 936 | C38 | 398 | 930 | EHMT2 |
| Lib1, Lib2 | N39 | 52 | 1911 | C39 | 399 | 1905 | KDM3A |
| Lib2 | N40 | 53 | 2754 | C40 | 400 | 2751 | OGA_HUMAN |
| Lib2 | N41 | 54 | 2523 | C41 | 401 | 2520 | SMCA5_HUMAN |
| Lib2 | N42 | 55 | 1347 | C42 | 402 | 1341 | KMT2E_HUMAN |
| Lib2 | N43 | 56 | 1326 | C43 | 403 | 1323 | SETD4_HUMAN |
| Lib2 | N44 | 57 | 3147 | C44 | 404 | 3141 | KMT2D_HUMAN |
| Lib2 | N45 | 58 | 657 | C45 | 405 | 651 | KMT2D_HUMAN |
| Lib2 | N46 | 59 | 1551 | C46 | 406 | 1548 | SMRD1_HUMAN |
| Lib2 | N47 | 60 | 1305 | C47 | 407 | 1302 | ANM2_HUMAN |
| Lib2 | N48 | 61 | 372 | C48 | 408 | 369 | SETD5_HUMAN |
| Lib2 | N49 | 62 | 2130 | C49 | 409 | 2127 | NCOA1_HUMAN |
| Lib2 | N50 | 63 | 2208 | C50 | 410 | 2202 | NCOA1_HUMAN |
| Lib2 | N51 | 64 | 993 | C51 | 411 | 990 | UCHL5_HUMAN |
| Lib2 | N52 | 65 | 2016 | C52 | 412 | 2013 | CBP_HUMAN |
| Lib2 | N53 | 66 | 1515 | C53 | 413 | 1509 | CBP_HUMAN |
| Lib2 | N54 | 67 | 2235 | C54 | 414 | 2229 | CBP_HUMAN |
| Lib2 | N55 | 68 | 1281 | C55 | 415 | 1278 | RBBP4_HUMAN |
| Lib2 | N56 | 69 | 924 | C56 | 416 | 918 | SUV92_HUMAN |
| Lib2 | N57 | 70 | 1188 | C57 | 417 | 1185 | ANM8_HUMAN |
| Lib2 | N58 | 71 | 2472 | C58 | 418 | 2469 | TF3C4_HUMAN |
| Lib2 | N59 | 72 | 843 | C59 | 419 | 840 | INO80_HUMAN |
| Lib2 | N60 | 73 | 3117 | C60 | 420 | 3111 | INO80_HUMAN |
| Lib2 | N61 | 74 | 1263 | C61 | 421 | 1260 | HAT1_HUMAN |
| Lib2 | N62 | 75 | 2661 | C62 | 422 | 2658 | KMT5B_HUMAN |
| Lib2 | N63 | 76 | 1260 | C63 | 423 | 1257 | SMYD5_HUMAN |
| Lib2 | N64 | 77 | 1470 | C64 | 424 | 1467 | HDAC2_HUMAN |
| Lib2 | N65 | 78 | 1392 | C65 | 425 | 1389 | MCRS1_HUMAN |
| Lib2 | N66 | 79 | 1176 | C66 | 426 | 1173 | DOT1L_HUMAN |
| Lib2 | N67 | 80 | 1167 | C67 | 427 | 1164 | PRDM9_HUMAN |
| Lib2 | N68 | 81 | 666 | C68 | 428 | 660 | MBD3_HUMAN |
| Lib2 | N69 | 82 | 474 | C69 | 429 | 471 | SETB2_HUMAN |
| Lib2 | N70 | 83 | 1479 | C70 | 430 | 1473 | SETB2_HUMAN |
| Lib2 | N71 | 84 | 1374 | C71 | 431 | 1371 | RUVB1_HUMAN |
| Lib2 | N72 | 85 | 2568 | C72 | 432 | 2562 | KDM7A_HUMAN |
| Lib2 | N73 | 86 | 240 | C73 | 433 | 237 | KDM5D_HUMAN |
| Lib2 | N74 | 87 | 447 | C74 | 434 | 441 | KDM5D_HUMAN |
| Lib2 | N75 | 88 | 1011 | C75 | 435 | 1005 | KDM5D_HUMAN |
| Lib2 | N76 | 89 | 1281 | C76 | 436 | 1275 | KDM5D_HUMAN |
| Lib2 | N77 | 90 | 921 | C77 | 437 | 915 | KDM5D_HUMAN |
| Lib2 | N78 | 91 | 621 | C78 | 438 | 618 | KAT6A_HUMAN |
| Lib2 | N79 | 92 | 1500 | C79 | 439 | 1494 | KAT6A_HUMAN |
| Lib2 | N80 | 93 | 1473 | C80 | 440 | 1467 | KAT6A_HUMAN |
| Lib2 | N81 | 94 | 2019 | C81 | 441 | 2016 | NCOA3_HUMAN |
| Lib2 | N82 | 95 | 1215 | C82 | 442 | 1209 | NCOA3_HUMAN |
| Lib2 | N83 | 96 | 1455 | C83 | 443 | 1452 | SMRD3_HUMAN |
| Lib2 | N84 | 97 | 3087 | C84 | 444 | 3084 | IN80D_HUMAN |
| Lib2 | N85 | 98 | 1290 | C85 | 445 | 1287 | SMYD3_HUMAN |
| Lib2 | N86 | 99 | 3129 | C86 | 446 | 3123 | PHF2_HUMAN |
| Lib2 | N87 | 100 | 1104 | C87 | 447 | 1101 | SETD7_HUMAN |
| Lib2 | N88 | 101 | 591 | C88 | 448 | 588 | ING4_HUMAN |
| Lib2 | N89 | 102 | 2646 | C89 | 449 | 2640 | CHD4_HUMAN |
| Lib2 | N90 | 103 | 1017 | C90 | 450 | 1011 | CHD4_HUMAN |
| Lib2 | N91 | 104 | 1917 | C91 | 451 | 1914 | ANM5_HUMAN |
| Lib2 | N92 | 105 | 1476 | C92 | 452 | 1473 | SMYD1_HUMAN |
| Lib2 | N93 | 106 | 1284 | C93 | 453 | 1281 | ACL6B_HUMAN |
| Lib2 | N94 | 107 | 2124 | C94 | 454 | 2118 | KDM3B_HUMAN |
| Lib2 | N95 | 108 | 1425 | C95 | 455 | 1422 | SETD6_HUMAN |
| Lib2 | N96 | 109 | 2463 | C96 | 456 | 2457 | CHD3_HUMAN |
| Lib2 | N97 | 110 | 1314 | C97 | 457 | 1308 | CHD3_HUMAN |
| Lib2 | N98 | 111 | 501 | C98 | 458 | 495 | A4FU79_HUMAN |
| Lib2 | N99 | 112 | 582 | C99 | 459 | 579 | IN80C_HUMAN |
| Lib2 | N100 | 113 | 1377 | C100 | 460 | 1374 | TAF1_HUMAN |
| Lib2 | N101 | 114 | 1695 | C101 | 461 | 1689 | TAF1_HUMAN |
| Lib2 | N102 | 115 | 1602 | C102 | 462 | 1596 | TAF1_HUMAN |
| Lib2 | N103 | 116 | 1431 | C103 | 463 | 1428 | B4DKA4_HUMAN |
| Lib2 | N104 | 117 | 438 | C104 | 464 | 435 | MBD2_HUMAN |
| Lib2 | N105 | 118 | 600 | C105 | 465 | 597 | MBD2_HUMAN |
| Lib2 | N106 | 119 | 642 | C106 | 466 | 639 | KAT6B_HUMAN |
| Lib2 | N107 | 120 | 1200 | C107 | 467 | 1194 | KAT6B_HUMAN |
| Lib2 | N108 | 121 | 1284 | C108 | 468 | 1278 | KAT6B_HUMAN |
| Lib2 | N109 | 122 | 2805 | C109 | 469 | 2799 | KAT6B_HUMAN |
| Lib2 | N110 | 123 | 1395 | C110 | 470 | 1392 | JMJD4_HUMAN |
| Lib2 | N111 | 124 | 255 | C111 | 471 | 252 | KDM5A_HUMAN |
| Lib2 | N112 | 125 | 1005 | C112 | 472 | 999 | KDM5A_HUMAN |
| Lib2 | N113 | 126 | 1305 | C113 | 473 | 1299 | KDM5A_HUMAN |
| Lib2 | N114 | 127 | 1173 | C114 | 474 | 1167 | KDM5A_HUMAN |
| Lib2 | N115 | 128 | 3090 | C115 | 475 | 3087 | HDAC5_HUMAN |
| Lib2 | N116 | 129 | 1131 | C116 | 476 | 1128 | ANM6_HUMAN |
| Lib2 | N117 | 130 | 2502 | C117 | 477 | 2499 | HDAC6_HUMAN |
| Lib2 | N118 | 131 | 1161 | C118 | 478 | 1155 | HDAC6_HUMAN |
| Lib2 | N119 | 132 | 1293 | C119 | 479 | 1290 | ACL6A_HUMAN |
| Lib2 | N120 | 133 | 1224 | C120 | 480 | 1221 | RING1_HUMAN |
| Lib2 | N121 | 134 | 3870 | C121 | 481 | 3864 | KMT2A_HUMAN |
| Lib2 | N122 | 135 | 3168 | C122 | 482 | 3162 | KMT2A_HUMAN |
| Lib2 | N123 | 136 | 657 | C123 | 483 | 651 | KMT2A_HUMAN |
| Lib2 | N124 | 137 | 3123 | C124 | 484 | 3120 | SET1B_HUMAN |
| Lib2 | N125 | 138 | 2790 | C125 | 485 | 2784 | SET1B_HUMAN |
| Lib2 | N126 | 139 | 1095 | C126 | 486 | 1092 | SETMR_HUMAN |
| Lib2 | N127 | 140 | 717 | C127 | 487 | 711 | SETMR_HUMAN |
| Lib2 | N128 | 141 | 1803 | C128 | 488 | 1800 | HAIR_HUMAN |
| Lib2 | N129 | 142 | 1701 | C129 | 489 | 1695 | HAIR_HUMAN |
| Lib2 | N130 | 143 | 1083 | C130 | 490 | 1080 | PRDM2_HUMAN |
| Lib2 | N131 | 144 | 1890 | C131 | 491 | 1884 | PRDM2_HUMAN |
| Lib2 | N132 | 145 | 1521 | C132 | 492 | 1515 | PRDM2_HUMAN |
| Lib2 | N133 | 146 | 1014 | C133 | 493 | 1008 | KAT8_HUMAN |
| Lib2 | N134 | 147 | 1695 | C134 | 494 | 1692 | KDM2A_HUMAN |
| Lib2 | N135 | 148 | 1461 | C135 | 495 | 1455 | KDM2A_HUMAN |
| Lib2 | N136 | 149 | 1215 | C136 | 496 | 1212 | JMJD6_HUMAN |
| Lib2 | N137 | 150 | 2565 | C137 | 497 | 2562 | DNM3B_HUMAN |
| Lib2 | N138 | 151 | 765 | C138 | 498 | 762 | TFPT_HUMAN |
| Lib2 | N139 | 152 | 2724 | C139 | 499 | 2721 | KDM6B_HUMAN |
| Lib2 | N140 | 153 | 2220 | C140 | 500 | 2214 | KDM6B_HUMAN |
| Lib2 | N141 | 154 | 2013 | C141 | 501 | 2010 | SMRC1_HUMAN |
| Lib2 | N142 | 155 | 1980 | C142 | 502 | 1974 | SMRC1_HUMAN |
| Lib2 | N143 | 156 | 2196 | C143 | 503 | 2193 | KDM4B_HUMAN |
| Lib2 | N144 | 157 | 1989 | C144 | 504 | 1986 | UTY_HUMAN |
| Lib2 | N145 | 158 | 2064 | C145 | 505 | 2061 | UTY_HUMAN |
| Lib2 | N146 | 159 | 2130 | C146 | 506 | 2127 | KDM4A_HUMAN |
| Lib2 | N147 | 160 | 2106 | C147 | 507 | 2103 | NSD3_HUMAN |
| Lib2 | N148 | 161 | 1455 | C148 | 508 | 1449 | NSD3_HUMAN |
| Lib2 | N149 | 162 | 678 | C149 | 509 | 672 | MBD1_HUMAN |
| Lib2 | N150 | 163 | 1047 | C150 | 510 | 1044 | HDA11_HUMAN |
| Lib2 | N151 | 164 | 1206 | C151 | 511 | 1203 | SIR7_HUMAN |
| Lib2 | N152 | 165 | 1626 | C152 | 512 | 1623 | CDY1_HUMAN |
| Lib2 | N153 | 166 | 1185 | C153 | 513 | 1179 | KAT7_HUMAN |
| Lib2 | N154 | 167 | 1362 | C154 | 514 | 1359 | NSD2_HUMAN |
| Lib2 | N155 | 168 | 1479 | C155 | 515 | 1473 | NSD2_HUMAN |
| Lib2 | N156 | 169 | 2250 | C156 | 516 | 2247 | A7E2E1_HUMAN |
| Lib2 | N157 | 170 | 2703 | C157 | 517 | 2700 | A7E2E1_HUMAN |
| Lib2 | N158 | 171 | 1785 | C158 | 518 | 1782 | SETB1_HUMAN |
| Lib2 | N159 | 172 | 1887 | C159 | 519 | 1881 | SETB1_HUMAN |
| Lib2 | N160 | 173 | 1185 | C160 | 520 | 1182 | KMT5A_HUMAN |
| Lib2 | N161 | 174 | 2841 | C161 | 521 | 2838 | TET2_HUMAN |
| Lib2 | N162 | 175 | 1071 | C162 | 522 | 1068 | SIR6_HUMAN |
| Lib2 | N163 | 176 | 2082 | C163 | 523 | 2079 | ANM7_HUMAN |
| Lib2 | N164 | 177 | 831 | C164 | 524 | 825 | SIR5_HUMAN |
| Lib2 | N165 | 178 | 2010 | C165 | 525 | 2007 | MTA2_HUMAN |
| Lib2 | N166 | 179 | 1935 | C166 | 526 | 1932 | EHMT1_HUMAN |
| Lib2 | N167 | 180 | 1974 | C167 | 527 | 1968 | EHMT1_HUMAN |
| Lib2 | N168 | 181 | 3162 | C168 | 528 | 3159 | ARI1B_HUMAN |
| Lib2 | N169 | 182 | 3285 | C169 | 529 | 3279 | ARI1B_HUMAN |
| Lib2 | N170 | 183 | 2844 | C170 | 530 | 2838 | NSD1_HUMAN |
| Lib2 | N171 | 184 | 1818 | C171 | 531 | 1812 | ASH1L_HUMAN |
| Lib2 | N172 | 185 | 1023 | C172 | 532 | 1017 | ASH1L_HUMAN |
| Lib2 | N173 | 186 | 867 | C173 | 533 | 861 | SIR4_HUMAN |
| Lib2 | N174 | 187 | 1452 | C174 | 534 | 1449 | HDAC1_HUMAN |
| Lib2 | N175 | 188 | 1395 | C175 | 535 | 1392 | RUVB2_HUMAN |
| Lib2 | N176 | 189 | 1878 | C176 | 536 | 1875 | ARP8_HUMAN |
| Lib2 | N177 | 190 | 1008 | C177 | 537 | 1005 | KDM5C_HUMAN |
| Lib2 | N178 | 191 | 1392 | C178 | 538 | 1389 | KMT5C_HUMAN |
| Lib2 | N179 | 192 | 1599 | C179 | 539 | 1596 | SMRD2_HUMAN |
| Lib2 | N180 | 193 | 2700 | C180 | 540 | 2697 | SET1A_HUMAN |
| Lib2 | N181 | 194 | 2436 | C181 | 541 | 2430 | SET1A_HUMAN |
| Lib2 | N182 | 195 | 1788 | C182 | 542 | 1785 | MTA3_HUMAN |
| Lib2 | N183 | 196 | 1221 | C183 | 543 | 1215 | KMT2C_HUMAN |
| Lib2 | N184 | 197 | 2913 | C184 | 544 | 2907 | PHF8_HUMAN |
| Lib2 | N185 | 198 | 1281 | C185 | 545 | 1278 | RBBP7_HUMAN |
| Lib2 | N186 | 199 | 1401 | C186 | 546 | 1398 | RIOX2_HUMAN |
| Lib2 | N187 | 200 | 1305 | C187 | 547 | 1299 | MBD4_HUMAN |
| Lib2 | N188 | 201 | 2418 | C188 | 548 | 2415 | SMYD4_HUMAN |
| Lib2 | N189 | 202 | 846 | C189 | 549 | 843 | ING2_HUMAN |
| Lib2 | N190 | 203 | 1119 | C190 | 550 | 1116 | ANM1_HUMAN |
| Lib2 | N191 | 204 | 1800 | C191 | 551 | 1797 | CDYL_HUMAN |
| Lib2 | N192 | 205 | 2016 | C192 | 552 | 2010 | JHD2C_HUMAN |
| Lib2 | N193 | 206 | 1575 | C193 | 553 | 1572 | KDM4D_HUMAN |
| Lib2 | N194 | 207 | 1272 | C194 | 554 | 1269 | ING1_HUMAN |
| Lib2 | N195 | 208 | 1788 | C195 | 555 | 1785 | SETD3_HUMAN |
| Lib2 | N196 | 209 | 2151 | C196 | 556 | 2148 | MTA1_HUMAN |
| Lib2 | N197 | 210 | 1242 | C197 | 557 | 1239 | SUV91_HUMAN |
| Lib2 | N198 | 211 | 744 | C198 | 558 | 738 | JMJD8_HUMAN |
| Lib2 | N199 | 212 | 1083 | C199 | 559 | 1080 | ING3_HUMAN |
| Lib2 | N200 | 213 | 1827 | C200 | 560 | 1824 | ARP5_HUMAN |
| Lib2 | N201 | 214 | 1821 | C201 | 561 | 1818 | KDM2B_HUMAN |
| Lib2 | N202 | 215 | 1842 | C202 | 562 | 1836 | KDM2B_HUMAN |
| Lib2 | N203 | 216 | 3168 | C203 | 563 | 3165 | SMCA1_HUMAN |
| Lib2 | N204 | 217 | 1053 | C204 | 564 | 1050 | HIF1N_HUMAN |
| Lib2 | N205 | 218 | 954 | C205 | 565 | 951 | JMJD7_HUMAN |
| Lib2 | N206 | 219 | 903 | C206 | 566 | 900 | SETD9_HUMAN |
| Lib2 | N207 | 220 | 2472 | C207 | 567 | 2469 | KDM1B_HUMAN |
| Lib2 | N208 | 221 | 2541 | C208 | 568 | 2538 | ANM9_HUMAN |
| Lib2 | N209 | 222 | 2013 | C209 | 569 | 2010 | HDA10_HUMAN |
| Lib2 | N210 | 223 | 1203 | C210 | 570 | 1200 | SIR3_HUMAN |
| Lib2 | N211 | 224 | 2976 | C211 | 571 | 2970 | KMT2B_HUMAN |
| Lib2 | N212 | 225 | 1137 | C212 | 572 | 1134 | HDAC8_HUMAN |
| Lib2 | N213 | 226 | 1470 | C213 | 573 | 1467 | HBAP1_HUMAN |
| Lib2 | N214 | 227 | 1515 | C214 | 574 | 1512 | HDAC4_HUMAN |
| Lib2 | N215 | 228 | 1749 | C215 | 575 | 1746 | HDAC4_HUMAN |
| Lib2 | N216 | 229 | 561 | C216 | 576 | 558 | ING5_HUMAN |
| Lib2 | N217 | 230 | 399 | C217 | 577 | 396 | CHRC1_HUMAN |
| Lib2 | N218 | 231 | 1074 | C218 | 578 | 1071 | IN80B_HUMAN |
| Lib2 | N219 | 232 | 1311 | C219 | 579 | 1305 | JARD2_HUMAN |
| Lib2 | N220 | 233 | 1608 | C220 | 580 | 1602 | JARD2_HUMAN |
| Lib2 | N221 | 234 | 1305 | C221 | 581 | 1302 | SMYD2_HUMAN |
| Lib2 | N222 | 235 | 738 | C222 | 582 | 735 | IN80E_HUMAN |
| Lib2 | N223 | 236 | 2874 | C223 | 583 | 2871 | FBX10_HUMAN |
| Lib2 | N224 | 237 | 2049 | C224 | 584 | 2043 | KDM4C_HUMAN |
| Lib2 | N225 | 238 | 2007 | C225 | 585 | 2001 | SETD2_HUMAN |
| Lib2 | N226 | 239 | 1473 | C226 | 586 | 1467 | SETD2_HUMAN |
| Lib2 | N227 | 240 | 2247 | C227 | 587 | 2244 | SIR1_HUMAN |
| Lib2 | N228 | 241 | 2757 | C228 | 588 | 2754 | HDAC7_HUMAN |
| Lib2 | N229 | 242 | 1113 | C229 | 589 | 1110 | NFRKB_HUMAN |
| Lib2 | N230 | 243 | 2421 | C230 | 590 | 2415 | NFRKB_HUMAN |
| Lib2 | N231 | 244 | 1005 | C231 | 591 | 999 | KDM5B_HUMAN |
| Lib2 | N232 | 245 | 3039 | C232 | 592 | 3036 | HDAC9_HUMAN |
| Lib2 | N233 | 246 | 444 | C233 | 593 | 438 | HINFP_HUMAN |
| Lib2 | N234 | 247 | 2931 | C234 | 594 | 2925 | SMRC2_HUMAN |
| Lib2 | N235 | 248 | 1830 | C235 | 595 | 1827 | CARM1_HUMAN |
| Lib2 | N236 | 249 | 2244 | C236 | 596 | 2241 | KAT2A_HUMAN |
| Lib2 | N237 | 250 | 1545 | C237 | 597 | 1542 | KAT5_HUMAN |
| Lib2 | N238 | 251 | 3081 | C238 | 598 | 3078 | NAT10_HUMAN |
| Lib2 | N239 | 252 | 1254 | C239 | 599 | 1251 | KDM8_HUMAN |
| Lib2 | N240 | 253 | 1389 | C240 | 600 | 1383 | ANM3_HUMAN |
| Lib2 | N241 | 254 | 1173 | C241 | 601 | 1170 | SIR2_HUMAN |
| Lib2 | N242 | 255 | 2223 | C242 | 602 | 2220 | KAT2B_HUMAN |
| Lib2 | N243 | 256 | 1941 | C243 | 603 | 1938 | DNMT1_HUMAN |
| Lib2 | N244 | 257 | 2781 | C244 | 604 | 2775 | DNMT1_HUMAN |
| Lib2 | N245 | 258 | 543 | C245 | 605 | 537 | TYY1_HUMAN |
| Lib2 | N246 | 259 | 423 | C246 | 606 | 417 | ATRX_HUMAN |
| Lib2 | N247 | 260 | 2745 | C247 | 607 | 2739 | ATRX_HUMAN |
| Lib2 | N248 | 261 | 1665 | C248 | 608 | 1662 | GLYR1_HUMAN |
| Lib2 | N249 | 262 | 879 | C264 | 609 | 873 | MTL5_HUMAN |
| Lib2 | N250 | 263 | 1095 | C250 | 610 | 1092 | HCFC1_HUMAN |
| Lib2 | N251 | 264 | 1971 | C251 | 611 | 1965 | HCFC1_HUMAN |
| Lib2 | N252 | 265 | 1845 | C252 | 612 | 1839 | HCFC1_HUMAN |
| Lib2 | N253 | 266 | 1620 | C253 | 613 | 1617 | RBBP5_HUMAN |
| Lib2 | N254 | 267 | 1851 | C254 | 614 | 1848 | MEN1_HUMAN |
| Lib2 | N255 | 268 | 1329 | C255 | 615 | 1326 | EED_HUMAN |
| Lib2 | N256 | 269 | 2562 | C256 | 616 | 2559 | LSD1-ORF |
| Lib2 | N257 | 270 | 168 | C257 | 617 | 162 | c-Myb-TAD |
| Lib2 | N258 | 271 | 1908 | C258 | 618 | 1902 | Nejire-HAT |
| Lib2 | N259 | 272 | 2661 | C259 | 619 | 2655 | ROS1-CD |
| Lib2 | N260 | 273 | 2700 | C260 | 620 | 2694 | Chd1-CD |
| Lib2 | N261 | 274 | 2286 | C261 | 621 | 2283 | EZH2_ORYLA |
| Lib2 | N262 | 275 | 2244 | C262 | 622 | 2241 | EZH2 |
| Lib2 | N263 | 276 | 1137 | C263 | 623 | 1134 | HDAC8 |
| Lib2 | N264 | 277 | 879 | C249 | 624 | 876 | HAC1-HAT |
| Lib2 | N265 | 278 | 2244 | C265 | 625 | 2241 | EZH2 |
| Lib2 | N266 | 279 | 501 | C266 | 626 | 498 | GADD45A |
| Lib2 | N267 | 280 | 510 | C267 | 627 | 507 | FOXA1-Nterm |
| Lib2 | N268 | 281 | 645 | C268 | 628 | 639 | FOXA1-Cterm |
| Lib2 | N269 | 282 | 513 | C269 | 629 | 510 | PU.1 |
| Lib2 | N270 | 283 | 1350 | C270 | 630 | 1344 | ENL |
| Lib2 | N271 | 284 | 666 | C271 | 631 | 663 | GCN5 |
| Lib2 | N272 | 285 | 825 | C272 | 632 | 822 | Sir2 |
| Lib2 | N273 | 286 | 1101 | C273 | 633 | 1098 | PRDM9-CD |
| Lib2 | N274 | 287 | 462 | C274 | 634 | 459 | UBE2A |
| Lib2 | N275 | 288 | 996 | C275 | 635 | 993 | DOT1L-DOT1 domain |
| Lib2 | N276 | 289 | 1155 | C276 | 636 | 1149 | EHMT2(G9a)-Cterm |
| Lib2 | N277 | 290 | 1242 | C277 | 637 | 1239 | SUV39H1 |
| Lib2 | N278 | 291 | 1941 | C278 | 638 | 1935 | TET2CD |
| Lib2 | N279 | 292 | 1362 | C279 | 639 | 1356 | 6TAL-VP128(TV) |
| Lib2 | N280 | 293 | 324 | C280 | 640 | 318 | CBF1-Cterm |
| Lib2 | N281 | 294 | 159 | C281 | 641 | 153 | VP64 |
| Lib2 | N282 | 295 | 393 | C282 | 642 | 387 | VP160 |
| Lib2 | N283 | 296 | 2847 | C283 | 643 | 2841 | TET3CD |
| Lib2 | N284 | 297 | 948 | C284 | 644 | 942 | p65-HSF1 |
| Lib2 | N285 | 298 | 765 | C285 | 645 | 759 | p65-MyoD |
| Lib2 | N286 | 299 | 393 | C286 | 646 | 387 | VP160 |
| Lib2 | N287 | 300 | 1167 | C287 | 647 | 1161 | G9A[SET] |
| Lib2 | N288 | 301 | 1032 | C288 | 648 | 1026 | SUV[SET] |
| Lib2 | N289 | 302 | 141 | C289 | 649 | 138 | FOG1 |
| Lib2 | N290 | 303 | 363 | C290 | 650 | 360 | HP1aCS |
| Lib2 | N291 | 304 | 1260 | C291 | 651 | 1257 | SS18 |
| Lib2 | N292 | 305 | 1179 | C292 | 652 | 1173 | MQ1(Q147L) |
| Lib2 | N293 | 306 | 1152 | C293 | 653 | 1149 | KRAB-MeCP2 |
| Lib2 | N294 | 307 | 1047 | C294 | 654 | 1044 | DIM5 |
| Lib2 | N295 | 308 | 921 | C295 | 655 | 918 | SET-TAF1B |
| Lib2 | N296 | 309 | 411 | C296 | 656 | 408 | HP1a |
| Lib2 | N297 | 310 | 954 | C297 | 657 | 951 | MesoLo |
| Lib2 | N298 | 311 | 708 | C298 | 658 | 705 | MBD2b |
| Lib2 | N299 | 312 | 927 | C299 | 659 | 924 | HST2 |
| Lib2 | N300 | 313 | 921 | C300 | 660 | 918 | SIRT6 |
| Lib2 | N301 | 314 | 825 | C301 | 661 | 822 | SIRT3 |
| Lib2 | N302 | 315 | 1095 | C302 | 662 | 1092 | HDAC11 |
| Lib2 | N303 | 316 | 2262 | C303 | 663 | 2259 | EZH2 |
| Lib2 | N304 | 317 | 2742 | C304 | 664 | 2739 | DNMT3a |
| Lib2 | N305 | 318 | 711 | C305 | 665 | 708 | NUE |
| Lib2 | N306 | 319 | 780 | C306 | 666 | 777 | CobB |
| Lib2 | N307 | 320 | 855 | C307 | 667 | 852 | KYP |
| Lib2 | N308 | 321 | 687 | C308 | 668 | 684 | HDT1 |
| Lib2 | N309 | 322 | 1029 | C309 | 669 | 1026 | HDAC8 |
| Lib2 | N310 | 323 | 966 | C310 | 670 | 963 | TgSET8 |
| Lib2 | N311 | 324 | 1038 | C311 | 671 | 1035 | Sin3a |
| Lib2 | N312 | 325 | 681 | C312 | 672 | 678 | SetD8 |
| Lib2 | N313 | 326 | 333 | C313 | 673 | 330 | Sall |
| Lib2 | N314 | 327 | 912 | C314 | 674 | 909 | MeCP2 |
| Lib2 | N315 | 328 | 261 | C315 | 675 | 258 | NCoR |
| Lib2 | N316 | 329 | 369 | C316 | 676 | 362 | HP1 |
| Lib2 | N317 | 330 | 1290 | C317 | 677 | 1287 | HDAC3 |
| Lib2 | N318 | 331 | 405 | C318 | 678 | 402 | 4xSID |
| Lib2 | N319 | 332 | 2304 | C319 | 679 | 2298 | mCBP-HAT |
| Lib2 | N320 | 333 | 570 | C320 | 680 | 567 | mMLL3-SET |
| Lib2 | N321 | 334 | 2877 | C321 | 681 | 2871 | MINT_HUMAN |
| Lib2 | N322 | 335 | 1743 | C322 | 682 | 1737 | MINT_HUMAN |
| Lib2 | N323 | 336 | 2325 | C323 | 683 | 2322 | HNRPU_HUMAN |
| Lib2 | N324 | 337 | 639 | C324 | 684 | 636 | LBR_HUMAN |
| Lib2 | N325 | 338 | 1851 | C325 | 685 | 1848 | LBR_HUMAN |
| Lib2 | N326 | 339 | 831 | C326 | 686 | 828 | pf_Sir2A |
| Lib2 | N327 | 340 | 999 | C327 | 687 | 996 | hs_NIPP1 |
| Lib2 | N328 | 341 | 1017 | C328 | 688 | 1014 | hs_PHF19 |
| Lib2 | N329 | 342 | 369 | C329 | 689 | 366 | pbcv1_vSET |
| Lib2 | N330 | 343 | 738 | C330 | 690 | 735 | ce_Set1 |
| Lib2 | N331 | 344 | 900 | C331 | 691 | 894 | mm_G9a |
| Lib2 | N332 | 345 | 837 | C332 | 692 | 831 | hs_SIRT5 |
| Lib2 | N333 | 346 | 981 | C333 | 693 | 975 | sc_RPD3 |
| Lib2 | N334 | 347 | 675 | C334 | 694 | 669 | hs_RCOR1 |
| Lib2 | N335 | 348 | 954 | C335 | 695 | 948 | at_SUVR4 |
| Lib2 | N336 | 349 | 876 | C336 | 696 | 873 | ce_Set4 |
| Lib2 | N337 | 350 | 3045 | C337 | 697 | 3039 | PHF3_HUMAN |
| Lib2 | N338 | 351 | 2937 | C338 | 698 | 2934 | RBM15_HUMAN |
| Lib2 | N339 | 352 | 1872 | C339 | 699 | 1866 | SPOC1_HUMAN |
| Lib2 | N340 | 353 | 2709 | C340 | 700 | 2706 | FPA_ARATH |
| Lib2 | N341 | 354 | 2466 | C341 | 701 | 2463 | NCOR2_HUMAN |
| Lib2 | N342 | 355 | 2655 | C342 | 702 | 2649 | NCOR2_HUMAN |
| Lib2 | N343 | 356 | 2442 | C343 | 703 | 2439 | NCOR2_HUMAN |
| Lib2 | N344 | 357 | 1290 | C344 | 704 | 1287 | HDAC3_HUMAN |
| Lib2 | N345 | 358 | 429 | C345 | 705 | 426 | MSX2_HUMAN |
| Lib2 | N346 | 359 | 207 | C346 | 706 | 201 | MSX2_HUMAN |
| Lib2 | N347 | 360 | 3168 | C347 | 707 | 3162 | DIDO1_HUMAN |
| Lib2 | N348 | 361 | 1767 | C348 | 708 | 1764 | BRDT_HUMAN |
| Lib2 | N349 | 362 | 840 | C349 | 709 | 837 | DAM_N132A |

TABLE 5
Exemplar Hits from Library 2

| Full_IDs | N | C | Full_Name | log2FoldChange | pvalue |
|---|---|---|---|---|---|
| 25 - 281 | N25 | C281 | CTCFL - VP64 | 4.15049289 | 5.08E−05 |
| 285 - 281 | N285 | C281 | p65-MyoD - VP64 | 4.14655304 | 3.24E−14 |
| 212 - 281 | N212 | C281 | HDAC8_HUMAN - VP64 | 3.65967799 | 8.46E−17 |
| 282 - 257 | N282 | C257 | VP160 - c-Myb-TAD | 3.62466825 | 9.65E−05 |
| 346 - 281 | N346 | C281 | MSX2_HUMAN - VP64 | 3.6004395 | 0.00211514 |
| 284 - 154 | N284 | C154 | p65-HSF1 - NSD2_HUMAN | 3.5618687 | 1.48E−09 |
| 190 - 281 | N190 | C281 | ANM1_HUMAN - VP64 | 3.5573888 | 3.07E−11 |
| 345 - 281 | N345 | C281 | MSX2_HUMAN - VP64 | 3.51582919 | 7.90E−11 |
| 281 - 315 | N281 | C315 | VP64 - NCoR | 3.51344384 | 0.00602017 |
| 285 - 18 | N285 | C18 | p65-MyoD - EZH2 | 3.46569257 | 4.81E−08 |
| 282 - 217 | N282 | C217 | VP160 - CHRC1_HUMAN | 3.41859537 | 2.51E−11 |
| 281 - 269 | N281 | C269 | VP64 - PU.1 | 3.40462175 | 8.32E−09 |
| 282 - 204 | N282 | C204 | VP160 - HIF1N_HUMAN | 3.39151178 | 1.96E−10 |
| 282 - 285 | N282 | C285 | VP160 - p65-MyoD | 3.38505548 | 3.54E−16 |
| 69 - 281 | N69 | C281 | SETB2_HUMAN - VP64 | 3.26337528 | 0.00454037 |
| 282 - 151 | N282 | C151 | VP160 - SIR7_HUMAN | 3.25619642 | 3.00E−07 |
| 317 - 281 | N317 | C281 | HDAC3 - VP64 | 3.22080428 | 2.52E−11 |
| 14 - 281 | N14 | C281 | RELA - VP64 | 3.2189722 | 2.26E−11 |
| 118 - 281 | N118 | C281 | HDAC6_HUMAN - VP64 | 3.21729179 | 1.38E−09 |
| 284 - 281 | N284 | C281 | p65-HSF1 - VP64 | 3.21294873 | 9.75E−10 |
| 322 - 281 | N322 | C281 | MINT_HUMAN - VP64 | 3.1696308 | 2.45E−13 |
| 91 - 281 | N91 | C281 | ANM5_HUMAN - VP64 | 3.15532258 | 7.96E−10 |
| 285 - 1 | N285 | C1 | p65-MyoD - SETD1A | 3.15078628 | 2.52E−14 |
| 281 - 280 | N281 | C280 | VP64 - CBF1-Cterm | 3.14508087 | 0.00829036 |
| 275 - 35 | N275 | C35 | DOT1L-DOT1_domain - | 3.13452827 | 5.02E−10 |
| | | | VPR_JKNp64 | | |
| 282 - 291 | N282 | C291 | VP160 - SS18 | 3.12357008 | 1.28E−12 |
| 284 - 267 | N284 | C267 | p65-HSF1 - FOXA1-Nterm | 3.11716631 | 1.36E−16 |
| 281 - 346 | N281 | C346 | VP64 - MSX2_HUMAN | 3.11054456 | 0.0189031 |
| 284 - 257 | N284 | C257 | p65-HSF1 - c-Myb-TAD | 3.10641044 | 9.21E−14 |
| 284 - 291 | N284 | C291 | p65-HSF1 - SS18 | 3.10151665 | 1.70E−07 |
| 306 - 281 | N306 | C281 | CobB - VP64 | 3.0838466 | 1.03E−09 |
| 339 - 281 | N339 | C281 | SPOC1_HUMAN - VP64 | 3.07365661 | 3.07E−07 |
| 222 - 281 | N222 | C281 | IN80E_HUMAN - VP64 | 3.07320703 | 5.81E−10 |
| 282 - 289 | N282 | C289 | VP160 - FOG1 | 3.0727682 | 0.00103697 |
| 284 - 280 | N284 | C280 | p65-HSF1 - CBF1-Cterm | 3.06468054 | 3.61E−06 |
| 162 - 35 | N162 | C35 | SIR6_HUMAN - | 3.04983874 | 2.02E−06 |
| | | | VPR_JKNp64 | | |
| 327 - 281 | N327 | C281 | hs_NIPP1 - VP64 | 3.04814332 | 1.71E−09 |
| 291 - 281 | N291 | C281 | SS18 - VP64 | 3.04720512 | 9.67E−06 |
| 151 - 281 | N151 | C281 | SIR7_HUMAN - VP64 | 3.03569397 | 9.15E−13 |
| 284 - 345 | N284 | C345 | p65-HSF1 - MSX2_HUMAN | 3.02853994 | 4.74E−06 |
| 281 - 285 | N281 | C285 | VP64 - p65-MyoD | 3.02395619 | 1.20E−11 |
| 297 - 281 | N297 | C281 | MesoLo - VP64 | 3.02283858 | 1.58E−12 |
| 282 - 315 | N282 | C315 | VP160 - NCoR | 3.01852669 | 7.92E−04 |
| 302 -281 | N302 | C281 | HDAC11 - VP64 | 3.01466293 | 2.36E−11 |
| 281 - 284 | N281 | C284 | VP64 - p65-HSF1 | 3.0143236 | 4.40E−11 |
| 154 - 281 | N154 | C281 | NSD2_HUMAN - VP64 | 3.01261589 | 5.12E−06 |
| 116 - 281 | N116 | C281 | ANM6_HUMAN - VP64 | 3.00494884 | 1.09E−07 |
| 285 - 99 | N285 | C99 | p65-MyoD - IN80C_HUMAN | 2.99379637 | 3.75E−10 |
| 164 - 281 | N164 | C281 | SIR5_HUMAN - VP64 | 2.98573061 | 9.96E−13 |
| 5 - 284 | N5 | C284 | KDM1A - p65-HSF1 | 2.9841417 | 2.03E−11 |
| 310 - 281 | N310 | C281 | TgSET8 - VP64 | 2.98174935 | 5.22E−08 |
| 233 - 281 | N233 | C281 | HINFP_HUMAN - VP64 | 2.97621976 | 9.21E−06 |
| 281 - 17 | N281 | C17 | VP64 - EZH2 | 2.96778242 | 4.71E−06 |
| 32 - 281 | N32 | C281 | Gilbert_Lab_CRISPRi_KRAB - | 2.95699143 | 0.0252263 |
| | | | VP64 | | |
| 281 - 278 | N281 | C278 | VP64 - TET2CD | 2.9520877 | 1.32E−09 |
| 282 -106 | N282 | C106 | VP160 - KAT6B_HUMAN | 2.95124009 | 3.14E−16 |
| 285 - 269 | N285 | C269 | p65-MyoD - PU.1 | 2.94786795 | 1.32E−08 |
| 281 - 5 | N281 | C5 | VP64 - KDM1A | 2.94706365 | 3.77E−06 |
| 300 - 281 | N300 | C281 | SIRT6 - VP64 | 2.94395234 | 1.56E−07 |
| 281 - 233 | N281 | C233 | VP64 - HINFP_HUMAN | 2.94137578 | 0.00765982 |
| 194 - 281 | N194 | C281 | ING1_HUMAN - VP64 | 2.93611071 | 7.15E−09 |
| 282 - 284 | N282 | C284 | VP160 - p65-HSF1 | 2.92713716 | 6.23E−15 |
| 284 - 314 | N284 | C314 | p65-HSF1 - MeCP2 | 2.91660605 | 6.91E−08 |
| 284 - 297 | N284 | C297 | p65-HSF1 - MesoLo | 2.91610449 | 3.02E−09 |
| 282 - 165 | N282 | C165 | VP160 - MTA2_HUMAN | 2.89247925 | 9.73E−06 |
| 282 - 346 | N282 | C346 | VP160 - MSX2_HUMAN | 2.88865492 | 0.00673929 |
| 282 - 290 | N282 | C290 | VP160 - HP1aCS | 2.88253485 | 9.58E−12 |
| 281 - 1 | N281 | C1 | VP64 - SETD1A | 2.87821341 | 0.02121545 |
| 73 - 281 | N73 | C281 | KDM5D_HUMAN - VP64 | 2.87005303 | 2.23E−06 |
| 282 - 218 | N282 | C218 | VP160 - IN80B_HUMAN | 2.86415696 | 4.05E−13 |
| 281 - 2 | N281 | C2 | VP64 - SETD1A | 2.85046697 | 2.16E−13 |
| 285 - 17 | N285 | C17 | p65-MyoD - EZH2 | 2.84367118 | 5.38E−07 |
| 197 - 281 | N197 | C281 | SUV91_HUMAN - VP64 | 2.83794687 | 4.76E−11 |
| 205 - 281 | N205 | C281 | JMJD7_HUMAN - VP64 | 2.8362683 | 2.18E−09 |
| 15 - 281 | N15 | C281 | EP300 - VP64 | 2.83532174 | 2.09E−06 |
| 282 - 345 | N282 | C345 | VP160 - MSX2_HUMAN | 2.83500973 | 1.65E−08 |
| 282 - 128 | N282 | C128 | VP160 - HAIR_HUMAN | 2.83414348 | 1.63E−06 |
| 284 - 310 | N284 | C310 | p65-HSF1 - TgSET8 | 2.8223996 | 2.07E−09 |
| 63 - 281 | N63 | C281 | SMYD5_HUMAN - VP64 | 2.82087595 | 1.19E−05 |
| 281 - 290 | N281 | C290 | VP64 - HP1aCS | 2.81778274 | 0.04643734 |
| 285 - 280 | N285 | C280 | p65-MyoD - CBF1-Cterm | 2.80684261 | 1.15E−05 |
| 282 - 269 | N282 | C269 | VP160 - PU.1 | 2.80606212 | 6.86E−11 |
| 282 - 280 | N282 | C280 | VP160 - CBF1-Cterm | 2.80282442 | 2.09E−08 |
| 282 - 314 | N282 | C314 | VP160 - MeCP2 | 2.79014817 | 6.40E−14 |
| 284 - 246 | N284 | C246 | p65-HSF1 - ATRX_HUMAN | 2.78917163 | 5.90E−07 |
| 284 - 313 | N284 | C313 | p65-HSF1 - SaII | 2.78308287 | 1.75E−07 |
| 285 - 291 | N285 | C291 | p65-MyoD - SS18 | 2.77720883 | 8.00E−10 |
| 123 - 35 | N123 | C35 | KMT2A_HUMAN - | 2.77668868 | 3.09E−08 |
| | | | VPR_JKNp64 | | |
| 285 - 233 | N285 | C233 | p65-MyoD - | 2.77392222 | 3.94E−08 |
| | | | HINFP_HUMAN | | |
| 281 - 149 | N281 | C149 | VP64 - MBD1_HUMAN | 2.76852205 | 2.59E−06 |
| 299 - 281 | N299 | C281 | HST2 - VP64 | 2.76763444 | 6.31E−11 |
| 78 - 285 | N78 | C285 | KAT6A_HUMAN - p65- | 2.76543284 | 3.86E−07 |
| | | | MyoD | | |
| 295 - 35 | N295 | C35 | SET-TAF1B - VPR_JKNp64 | 2.7629058 | 1.37E−07 |
| 59 - 281 | N59 | C281 | INO80_HUMAN - VP64 | 2.75885939 | 7.46E−10 |
| 281 - 345 | N281 | C345 | VP64 - MSX2_HUMAN | 2.75848131 | 0.0080578 |
| 135 - 281 | N135 | C281 | KDM2A_HUMAN - VP64 | 2.75679089 | 2.74E−07 |
| 284 - 239 | N284 | C239 | p65-HSF1 - KDM8_HUMAN | 2.75545412 | 1.45E−08 |
| 282 - 192 | N282 | C192 | VP160 - JHD2C_HUMAN | 2.75435431 | 1.72E−05 |
| 282 - 246 | N282 | C246 | VP160 - ATRX_HUMAN | 2.75164016 | 4.87E−09 |
| 284 - 63 | N284 | C63 | p65-HSF1 - | 2.74651564 | 1.41E−08 |
| | | | SMYD5_HUMAN | | |
| 282 - 111 | N282 | C111 | VP160 - KDM5A_HUMAN | 2.74546328 | 0.00619723 |
| 282 - 164 | N282 | C164 | VP160 - SIR5_HUMAN | 2.7443568 | 8.68E−15 |
| 93 - 281 | N93 | C281 | ACL6B_HUMAN - VP64 | 2.73873156 | 2.26E−08 |
| 282 - 78 | N282 | C78 | VP160 - KAT6A_HUMAN | 2.7332012 | 1.28E−10 |
| 27 - 281 | N27 | C281 | EZH1 - VP64 | 2.73046777 | 2.28E−10 |
| 281 - 116 | N281 | C116 | VP64 - ANM6_HUMAN | 2.72792321 | 2.21E−09 |
| 281 - 172 | N281 | C172 | VP64 - ASH1L_HUMAN | 2.72699841 | 5.70E−06 |
| 14 - 257 | N14 | C257 | RELA - c-Myb-TAD | 2.7253311 | 4.89E−09 |
| 61 - 281 | N61 | C281 | HAT1_HUMAN - VP64 | 2.72376845 | 3.56E−06 |
| 282 - 116 | N282 | C116 | VP160 - ANM6_HUMAN | 2.72281514 | 1.29E−13 |
| 282 - 226 | N282 | C226 | VP160 - SETD2_HUMAN | 2.72224372 | 1.25E−12 |
| 35 - 14 | N35 | C14 | VPR_JKNp64 - RELA | 2.72187198 | 9.16E−05 |
| 281 - 35 | N281 | C35 | VP64 - VPR_JKNp64 | 2.71628132 | 1.28E−08 |
| 282 - 227 | N282 | C227 | VP160 - SIR1_HUMAN | 2.71411227 | 6.74E−06 |
| 138 - 284 | N138 | C284 | TFPT_HUMAN - p65-HSF1 | 2.70984378 | 7.75E−06 |
| 282 - 198 | N282 | C198 | VP160 - JMJD8_HUMAN | 2.70959931 | 2.30E−10 |
| 99 - 35 | N99 | C35 | IN80C_HUMAN - | 2.70847321 | 8.64E−05 |
| | | | VPR_JKNp64 | | |
| 284 - 294 | N284 | C294 | p65-HSF1 - DIM5 | 2.70584304 | 9.73E−10 |
| 164 - 35 | N164 | C35 | SIR5_HUMAN - | 2.70304098 | 1.24E−06 |
| | | | VPR_JKNp64 | | |
| 284 - 99 | N284 | C99 | p65-HSF1 - IN80C_HUMAN | 2.70302654 | 5.13E−06 |
| 35 - 1 | N35 | C1 | VPR_JKNp64 - SETD1A | 2.69839543 | 1.76E−07 |
| 186 - 35 | N186 | C35 | RIOX2_HUMAN - | 2.69576566 | 1.78E−06 |
| | | | VPR_JKNp64 | | |
| 206 - 281 | N206 | C281 | SETD9_HUMAN - VP64 | 2.68722228 | 1.95E−06 |
| 245 - 281 | N245 | C281 | TYY1_HUMAN - VP64 | 2.68575964 | 2.25E−06 |
| 282 - 9 | N282 | C9 | VP160 - MECP2 | 2.68537809 | 2.89E−13 |
| 270 - 281 | N270 | C281 | ENL - VP64 | 2.68254141 | 1.25E−05 |
| 218 - 281 | N218 | C281 | IN80B_HUMAN - VP64 | 2.68150535 | 7.00E−07 |
| 281 - 218 | N281 | C218 | VP64 - IN80B_HUMAN | 2.68098205 | 2.84E−11 |
| 281 - 313 | N281 | C313 | VP64 - SaII | 2.6809743 | 0.03932104 |
| 284 - 135 | N284 | C135 | p65-HSF1 - | 2.68088458 | 4.21E−06 |
| | | | KDM2A_HUMAN | | |
| 285 - 331 | N285 | C331 | p65-MyoD - mm_G9a | 2.68045181 | 5.79E−06 |
| 282 - 176 | N282 | C176 | VP160 - ARP8_HUMAN | 2.67740061 | 4.05E−07 |
| 154 - 284 | N154 | C284 | NSD2_HUMAN - p65-HSF1 | 2.6772012 | 2.50E−08 |
| 285 - 8 | N285 | C8 | p65-MyoD - MECP2 | 2.67372976 | 9.98E−07 |
| 273 - 35 | N273 | C35 | PRDM9-CD - VPR_JKNp64 | 2.66787454 | 9.43E−07 |
| 216 - 284 | N216 | C284 | ING5_HUMAN - p65-HSF1 | 2.66597539 | 1.22E−06 |
| 282 - 24 | N282 | C24 | VP160 - CTCFL | 2.66025868 | 9.88E−08 |
| 162- 281 | N162 | C281 | SIR6_HUMAN - VP64 | 2.66001709 | 1.97E−08 |
| 345 - 284 | N345 | C284 | MSX2_HUMAN - p65-HSF1 | 2.65990945 | 4.41E−08 |
| 17 - 284 | N17 | C284 | EZH2 - p65-HSF1 | 2.65311997 | 1.73E−09 |
| 35 - 106 | N35 | C106 | VPR_JKNp64 - | 2.65008307 | 1.24E−09 |
| | | | KAT6B_HUMAN | | |
| 93 - 35 | N93 | C35 | ACL6B_HUMAN - | 2.64954943 | 1.35E−04 |
| | | | VPR_JKNp64 | | |
| 284 - 164 | N284 | C164 | p65-HSF1 - SIR5_HUMAN | 2.64708743 | 3.93E−06 |
| 281 - 66 | N281 | C66 | VP64 - DOT1L_HUMAN | 2.64586281 | 2.00E−11 |
| 266 - 281 | N266 | C281 | GADD45A - VP64 | 2.64044921 | 9.80E−06 |
| 18 - 281 | N18 | C281 | EZH2 - VP64 | 2.63981717 | 4.53E−08 |
| 281 - 291 | N281 | C291 | VP64 - SS18 | 2.63867198 | 4.90E−11 |
| 14 - 285 | N14 | C285 | RELA - p65-MyoD | 2.63768123 | 2.93E−06 |
| 112 - 281 | N112 | C281 | KDM5A_HUMAN - VP64 | 2.63553421 | 1.39E−10 |
| 282 - 212 | N282 | C212 | VP160 - HDAC8_HUMAN | 2.63519868 | 2.05E−07 |
| 315 - 35 | N315 | C35 | NCOR - VPR_JKNp64 | 2.63489284 | 2.12E−06 |
| 282 - 131 | N282 | C131 | VP160 - PRDM2_HUMAN | 2.63412752 | 8.42E−13 |
| 284 - 47 | N284 | C47 | p65-HSF1 - ANM2_HUMAN | 2.63355415 | 8.03E−06 |
| 166 - 284 | N166 | C284 | EHMT1_HUMAN - p65- | 2.63200795 | 4.33E−06 |
| | | | HSF1 | | |
| 282 - 14 | N282 | C14 | VP160 - RELA | 2.62789577 | 8.10E−13 |
| 282 - 52 | N282 | C52 | VP160 - CBP_HUMAN | 2.62436076 | 7.50E−10 |
| 313 - 35 | N313 | C35 | SaII - VPR_JKNp64 | 2.62254208 | 1.09E−09 |
| 282 - 38 | N282 | C38 | VP160 - EHMT2 | 2.62129505 | 9.77E−09 |
| 282 - 73 | N282 | C73 | VP160 - KDM5D_HUMAN | 2.61961629 | 8.70E−06 |
| 222 - 35 | N222 | C35 | IN80E_HUMAN - | 2.61840476 | 2.16E−06 |
| | | | VPR_JKNp64 | | |
| 284 - 106 | N284 | C106 | p65-HSF1 - | 2.61810906 | 2.54E−06 |
| | | | KAT6B_HUMAN | | |
| 35 - 63 | N35 | C63 | VPR_JKNp64 - | 2.61758698 | 8.36E−06 |
| | | | SMYD5_HUMAN | | |
| 282 - 123 | N282 | C123 | VP160 - KMT2A_HUMAN | 2.61646413 | 3.50E−10 |
| 280 - 35 | N280 | C35 | CBF1-Cterm - VPR_JKNp64 | 2.61619328 | 5.06E−09 |
| 282 - 50 | N282 | C50 | VP160 - NCOA1_HUMAN | 2.61560995 | 8.34E−09 |
| 282 - 313 | N282 | C313 | VP160 - SaII | 2.61503985 | 0.00788452 |
| 240 - 35 | N240 | C35 | ANM3_HUMAN - | 2.61404769 | 8.09E−05 |
| | | | VPR_JKNp64 | | |
| 49 - 281 | N49 | C281 | NCOA1_HUMAN - VP64 | 2.61101349 | 2.88E−05 |
| 282 - 299 | N282 | C299 | VP160 - HST2 | 2.60920978 | 2.33E−12 |
| 282 - 35 | N282 | C35 | VP160 - VPR_JKNp64 | 2.60336047 | 1.50E−07 |
| 186 - 284 | N186 | C284 | RIOX2_HUMAN - p65-HSF1 | 2.6008869 | 1.68E−09 |
| 281 - 268 | N281 | C268 | VP64 - FOXA1-Cterm | 2.59867323 | 9.04E−07 |
| 281 - 252 | N281 | C252 | VP64 - HCFC1_HUMAN | 2.59804204 | 3.28E−09 |
| 78 - 284 | N78 | C284 | KAT6A_HUMAN - p65- | 2.59515272 | 2.12E−09 |
| | | | HSF1 | | |
| 282 - 245 | N282 | C245 | VP160 - TYY1_HUMAN | 2.58769133 | 7.95E−08 |
| 114 - 284 | N114 | C284 | KDM5A_HUMAN - p65- | 2.57740362 | 5.26E−07 |
| | | | HSF1 | | |
| 281 - 15 | N281 | C15 | VP64 - EP300 | 2.57447676 | 1.12E−05 |
| 284 - 5 | N284 | C5 | p65-HSF1 - KDM1A | 2.57422956 | 3.53E−10 |
| 284 - 252 | N284 | C252 | p65-HSF1 - | 2.57314015 | 4.38E−05 |
| | | | HCFC1_HUMAN | | |
| 285 - 267 | N285 | C267 | p65-MyoD - FOXA1-Nterm | 2.57246203 | 2.45E−11 |
| 164 - 284 | N164 | C284 | SIR5_HUMAN - p65-HSF1 | 2.5704686 | 2.51E−06 |
| 281 - 10 | N281 | C10 | VP64 - HSF1 | 2.56634244 | 4.71E−11 |
| 144 - 281 | N144 | C281 | UTY_HUMAN - VP64 | 2.56629277 | 1.18E−04 |
| 282 - 298 | N282 | C298 | VP160 - MBD2b | 2.56585318 | 9.00E−14 |
| 282 - 233 | N282 | C233 | VP160 - HINFP_HUMAN | 2.56264986 | 5.28E−08 |
| 217 - 35 | N217 | C35 | CHRC1_HUMAN - | 2.56223041 | 4.31E−07 |
| | | | VPR_JKNp64 | | |
| 282 - 15 | N282 | C15 | VP160 - EP300 | 2.56179053 | 7.45E−11 |
| 282 - 17 | N282 | C17 | VP160 - EZH2 | 2.5589909 | 1.56E−08 |
| 273 - 281 | N273 | C281 | PRDM9-CD - VP64 | 2.55779896 | 5.21E−06 |
| 108 - 281 | N108 | C281 | KAT6B_HUMAN - VP64 | 2.55570931 | 1.50E−08 |
| 282 - 99 | N282 | C99 | VP160 - IN80C_HUMAN | 2.55184108 | 7.35E−11 |
| 282 - 239 | N282 | C239 | VP160 - KDM8_HUMAN | 2.54546274 | 6.43E−11 |
| 282 - 251 | N282 | C251 | VP160 - HCFC1_HUMAN | 2.54489169 | 6.03E−05 |
| 270 - 284 | N270 | C284 | ENL - p65-HSF1 | 2.54275435 | 1.76E−06 |
| 281 - 328 | N281 | C328 | VP64 - hs_PHF19 | 2.54245579 | 2.80E−08 |
| 284 - 332 | N284 | C332 | p65-HSF1 - hs_SIRT5 | 2.54128651 | 3.63E−06 |
| 106 - 281 | N106 | C281 | KAT6B_HUMAN - VP64 | 2.54107723 | 1.58E−07 |
| 282 - 281 | N282 | C281 | VP160 - VP64 | 2.54023976 | 5.94E−06 |
| 284 - 269 | N284 | C269 | p65-HSF1 - PU.1 | 2.53988937 | 1.08E−11 |
| 91 - 285 | N91 | C285 | ANM5_HUMAN - p65-MyoD | 2.53743604 | 1.25E−04 |
| 282 - 288 | N282 | C288 | VP160 - SUV[SET] | 2.53605349 | 1.37E−09 |
| 282 - 83 | N282 | C83 | VP160 - SMRD3_HUMAN | 2.53529775 | 2.19E−07 |
| 12 - 281 | N12 | C281 | KMT2A - VP64 | 2.53473909 | 7.77E−08 |
| 282 - 252 | N282 | C252 | VP160 - HCFC1_HUMAN | 2.52598846 | 4.68E−10 |
| 281 - 246 | N281 | C246 | VP64 - ATRX_HUMAN | 2.52568151 | 0.01052136 |
| 233 - 284 | N233 | C284 | HINFP_HUMAN - p65-HSF1 | 2.52252034 | 2.37E−09 |
| 282 - 214 | N282 | C214 | VP160 - HDAC4_HUMAN | 2.52063206 | 5.78E−08 |
| 285 - 63 | N285 | C63 | p65-MyoD - | 2.51910082 | 5.18E−08 |
| | | | SMYD5_HUMAN | | |
| 220 - 281 | N220 | C281 | JARD2_HUMAN - VP64 | 2.51496582 | 6.94E−05 |
| 280 - 281 | N280 | C281 | CBF1-Cterm - VP64 | 2.51015231 | 0.03015865 |
| 275 - 281 | N275 | C281 | DOT1L-DOT1_domain - | 2.51008485 | 1.48E−07 |
| | | | VP64 | | |
| 282 - 102 | N282 | C102 | VP160 - TAF1_HUMAN | 2.50934624 | 5.99E−09 |
| 282 - 295 | N282 | C295 | VP160 - SET-TAF1B | 2.50923113 | 3.38E−11 |
| 281 - 78 | N281 | C78 | VP64 - KAT6A_HUMAN | 2.50857066 | 4.96E−08 |
| 250 - 281 | N250 | C281 | HCFC1_HUMAN - VP64 | 2.50851306 | 3.65E−04 |
| 282 - 5 | N282 | C5 | VP160 - KDM1A | 2.50606406 | 1.25E−06 |
| 282 - 162 | N282 | C162 | VP160 - SIR6_HUMAN | 2.50553106 | 1.55E−10 |
| 281 - 47 | N281 | C47 | VP64 - ANM2_HUMAN | 2.50389568 | 1.53E−07 |
| 284 - 162 | N284 | C162 | p65-HSF1 - SIR6_HUMAN | 2.50286997 | 1.75E−07 |
| 77 - 284 | N77 | C284 | KDM5D_HUMAN - p65- | 2.50238752 | 3.01E−06 |
| | | | HSF1 | | |
| 282 - 294 | N282 | C294 | VP160 - DIM5 | 2.50143678 | 2.04E−12 |
| 284 - 249 | N284 | C249 | p65-HSF1 - HAC1-HAT | 2.50049495 | 3.57E−09 |
| 308 - 281 | N308 | C281 | HDT1 - VP64 | 2.49968864 | 3.29E−06 |
| 282 - 25 | N282 | C25 | VP160 - CTCFL | 2.49913717 | 0.02694462 |
| 194 - 284 | N194 | C284 | ING1_HUMAN - p65-HSF1 | 2.49845849 | 1.83E−07 |
| 282 - 275 | N282 | C275 | VP160 - DOT1L- | 2.49522308 | 7.32E−12 |
| | | | DOT1_domain | | |
| 285 - 218 | N285 | C218 | p65-MyoD - IN80B_HUMAN | 2.49481499 | 1.76E−05 |
| 284 - 69 | N284 | C69 | p65-HSF1 - SETB2_HUMAN | 2.49427424 | 1.15E−06 |
| 285 - 315 | N285 | C315 | p65-MyoD - NCOR | 2.4891583 | 3.82E−08 |
| 308 - 35 | N308 | C35 | HDT1 - VPR_JKNp64 | 2.48854565 | 1.46E−08 |
| 281 - 239 | N281 | C239 | VP64 - KDM8_HUMAN | 2.48500156 | 1.09E−10 |
| 284 - 78 | N284 | C78 | p65-HSF1 - | 2.48403777 | 1.07E−08 |
| | | | KAT6A_HUMAN | | |
| 285 - 45 | N285 | C45 | p65-MyoD - | 2.47975749 | 4.80E−05 |
| | | | KMT2D_HUMAN | | |
| 282 - 213 | N282 | C213 | VP160 - HBAP1_HUMAN | 2.47768866 | 5.04E−07 |
| 284 - 25 | N284 | C25 | p65-HSF1 - CTCFL | 2.47632 | 1.05E−04 |
| 282 - 156 | N282 | C156 | VP160 - A7E2E1_HUMAN | 2.47435824 | 2.42E−05 |
| 285 - 199 | N285 | C199 | p65-MyoD - ING3_HUMAN | 2.4741407 | 2.60E−04 |
| 282 - 173 | N282 | C173 | VP160 - SIR4_HUMAN | 2.47399655 | 1.62E−09 |
| 285 - 249 | N285 | C249 | p65-MyoD - HAC1-HAT | 2.47362395 | 8.57E−07 |
| 38 - 35 | N38 | C35 | EHMT2 - VPR_JKNp64 | 2.47117354 | 1.58E−05 |
| 282 - 68 | N282 | C68 | VP160 - MBD3_HUMAN | 2.47073731 | 6.24E−10 |
| 18 - 284 | N18 | C284 | EZH2 - p65-HSF1 | 2.46919218 | 4.68E−09 |
| 282 - 101 | N282 | C101 | VP160 - TAF1_HUMAN | 2.46741765 | 2.47E−10 |
| 284 - 315 | N284 | C315 | p65-HSF1 - NCoR | 2.46271116 | 2.39E−10 |
| 282 - 20 | N282 | C20 | VP160 - KDM1B | 2.46261072 | 6.31E−12 |
| 6 - 281 | N6 | C281 | KDM1A - VP64 | 2.46159087 | 7.32E−08 |
| 281 - 327 | N281 | C327 | VP64 - hs_NIPP1 | 2.46029115 | 3.28E−10 |
| 284 - 233 | N284 | C233 | p65-HSF1 - HINFP_HUMAN | 2.45654845 | 7.57E−07 |
| 216 - 35 | N216 | C35 | ING5_HUMAN - | 2.45541661 | 4.05E−06 |
| | | | VPR_JKNp64 | | |
| 246 - 239 | N246 | C239 | ATRX_HUMAN - | 2.45474969 | 7.18E−05 |
| | | | KDM8_HUMAN | | |
| 282 - 266 | N282 | C266 | VP160 - GADD45A | 2.45408854 | 1.22E−08 |
| 282 - 84 | N282 | C84 | VP160 - IN80D_HUMAN | 2.45310034 | 5.46E−05 |
| 282 - 12 | N282 | C12 | VP160 - KMT2A | 2.45167882 | 5.16E−12 |
| 282 - 250 | N282 | C250 | VP160 - HCFC1_HUMAN | 2.44960867 | 8.56E−11 |
| 285 - 257 | N285 | C257 | p65-MyoD - c-Myb-TAD | 2.44860557 | 2.47E−07 |
| 281 - 319 | N281 | C319 | VP64 - mCBP-HAT | 2.44839884 | 6.92E−05 |
| 136 - 281 | N136 | C281 | JMJD6_HUMAN - VP64 | 2.44792533 | 2.82E−05 |
| 134 - 281 | N134 | C281 | KDM2A_HUMAN - VP64 | 2.44760442 | 4.10E−07 |
| 281 - 26 | N281 | C26 | VP64 - EZH1 | 2.44602433 | 2.20E−05 |
| 282 - 148 | N282 | C148 | VP160 - NSD3_HUMAN | 2.4459255 | 9.59E−08 |
| 282 - 308 | N282 | C308 | VP160 - HDT1 | 2.44563357 | 2.74E−07 |
| 38 - 285 | N38 | C285 | EHMT2 - p65-MyoD | 2.44245026 | 9.61E−06 |
| 282 - 140 | N282 | C140 | VP160 - KDM6B_HUMAN | 2.44030819 | 5.98E−07 |
| 284 - 8 | N284 | C8 | p65-HSF1 - MECP2 | 2.43914721 | 1.16E−07 |
| 284 - 17 | N284 | C17 | p65-HSF1 - EZH2 | 2.43817423 | 2.96E−07 |
| 15 - 257 | N15 | C257 | EP300 - c-Myb-TAD | 2.43692842 | 4.10E−04 |
| 344 - 281 | N344 | C281 | HDAC3_HUMAN - VP64 | 2.43688415 | 2.18E−08 |
| 24 - 281 | N24 | C281 | CTCFL - VP64 | 2.43381249 | 5.58E−07 |
| 281 - 250 | N281 | C250 | VP64 - HCFC1_HUMAN | 2.43111827 | 1.34E−10 |
| 233 - 35 | N233 | C35 | HINFP_HUMAN - | 2.43092447 | 2.01E−06 |
| | | | VPR_JKNp64 | | |
| 296 - 35 | N296 | C35 | HP1a - VPR_JKNp64 | 2.43090457 | 2.27E−09 |
| 35 - 289 | N35 | C289 | VPR_JKNp64 - FOG1 | 2.43031245 | 4.94E−07 |
| 106 - 284 | N106 | C284 | KAT6B_HUMAN - p65- | 2.42932298 | 4.44E−07 |
| | | | HSF1 | | |
| 281 - 128 | N281 | C128 | VP64 - HAIR_HUMAN | 2.42723807 | 3.74E−05 |
| 281 - 266 | N281 | C266 | VP64 - GADD45A | 2.42686674 | 4.96E−05 |
| 282 - 301 | N282 | C301 | VP160 - SIRT3 | 2.42311735 | 8.89E−10 |
| 284 - 226 | N284 | C226 | p65-HSF1 - SETD2_HUMAN | 2.42051784 | 3.87E−04 |
| 282 - 268 | N282 | C268 | VP160 - FOXA1-Cterm | 2.42020028 | 6.16E−09 |
| 18 - 14 | N18 | C14 | EZH2 - RELA | 2.41997159 | 2.10E−06 |
| 282 - 8 | N282 | C8 | VP160 - MECP2 | 2.41888341 | 0.01725547 |
| 284 - 120 | N284 | C120 | p65-HSF1 - RING1_HUMAN | 2.41655813 | 4.78E−05 |
| 284 - 255 | N284 | C255 | p65-HSF1 - EED_HUMAN | 2.41629307 | 3.52E−05 |
| 339 - 284 | N339 | C284 | SPOC1_HUMAN - p65-HSF1 | 2.41419161 | 2.12E−07 |
| 284 - 222 | N284 | C222 | p65-HSF1 - IN80E_HUMAN | 2.41373102 | 9.15E−05 |
| 282 - 297 | N282 | C297 | VP160 - MesoLo | 2.41190571 | 2.38E−11 |
| 282 - 175 | N282 | C175 | VP160 - RUVB2_HUMAN | 2.40635756 | 4.18E−05 |
| 281 - 205 | N281 | C205 | VP64 - JMJD7_HUMAN | 2.40585323 | 4.54E−07 |
| 284 - 296 | N284 | C296 | p65-HSF1 - HP1a | 2.40571631 | 3.40E−06 |
| 281 - 63 | N281 | C63 | VP64 - SMYD5_HUMAN | 2.40151381 | 8.19E−09 |
| 285 - 108 | N285 | C108 | p65-MyoD - | 2.39830565 | 3.82E−04 |
| | | | KAT6B_HUMAN | | |
| 295 - 284 | N295 | C284 | SET-TAF1B - p65-HSF1 | 2.39737159 | 6.22E−07 |
| 281 - 162 | N281 | C162 | VP64 - SIR6_HUMAN | 2.39690963 | 9.43E−09 |
| 188 - 281 | N188 | C281 | SMYD4_HUMAN - VP64 | 2.3969055 | 1.89E−07 |
| 281 - 226 | N281 | C226 | VP64 - SETD2_HUMAN | 2.39244881 | 2.31E−05 |
| 282 - 267 | N282 | C267 | VP160 - FOXA1-Nterm | 2.39144748 | 4.32E−06 |
| 284 - 299 | N284 | C299 | p65-HSF1 - HST2 | 2.39013431 | 8.31E−07 |
| 282 - 69 | N282 | C69 | VP160 - SETB2_HUMAN | 2.38574983 | 1.89E−07 |
| 282 - 174 | N282 | C174 | VP160 - HDAC1_HUMAN | 2.3845532 | 5.48E−05 |
| 280 - 284 | N280 | C284 | CBF1-Cterm - p65-HSF1 | 2.38436452 | 2.31E−07 |
| 282 - 339 | N282 | C339 | VP160 - SPOC1_HUMAN | 2.38310497 | 1.36E−08 |
| 282 - 310 | N282 | C310 | VP160 - TgSET8 | 2.37863427 | 5.65E−10 |
| 106 - 285 | N106 | C285 | KAT6B_HUMAN - p65- | 2.37771894 | 1.94E−09 |
| | | | MyoD | | |
| 281 - 106 | N281 | C106 | VP64 - KAT6B_HUMAN | 2.37504576 | 5.83E−06 |
| 281 - 275 | N281 | C275 | VP64 - DOT1L- | 2.37437945 | 3.17E−09 |
| | | | DOT1_domain | | |
| 285 - 290 | N285 | C290 | p65-MyoD - HP1aCS | 2.37271098 | 2.14E−10 |
| 281 - 77 | N281 | C77 | VP64 - KDM5D_HUMAN | 2.37177561 | 6.37E−08 |
| 297 - 285 | N297 | C285 | MesoLo - p65-MyoD | 2.37086474 | 5.54E−06 |
| 282 - 95 | N282 | C95 | VP160 - SETD6_HUMAN | 2.36904949 | 1.61E−07 |
| 282 - 2 | N282 | C2 | VP160 - SETD1A | 2.36903449 | 4.43E−09 |
| 313 - 284 | N313 | C284 | SaII - p65-HSF1 | 2.3683689 | 3.53E−05 |
| 100 - 35 | N100 | C35 | TAF1_HUMAN - | 2.36781725 | 1.63E−04 |
| | | | VPR_JKNp64 | | |
| 35 - 313 | N35 | C313 | VPR_JKNp64 - SaII | 2.3654483 | 2.38E−09 |
| 282 - 154 | N282 | C154 | VP160 - NSD2_HUMAN | 2.36433715 | 3.15E−11 |
| 282 - 255 | N282 | C255 | VP160 - EED_HUMAN | 2.36389865 | 1.78E−10 |
| 281 - 200 | N281 | C200 | VP64 - ARP5_HUMAN | 2.3634855 | 3.69E−08 |
| 282 - 144 | N282 | C144 | VP160 - UTY_HUMAN | 2.36172966 | 1.48E−04 |
| 151 - 285 | N151 | C285 | SIR7_HUMAN - p65-MyoD | 2.36103329 | 1.47E−07 |
| 287 - 281 | N287 | C281 | G9A[SET] - VP64 | 2.3608579 | 3.26E−08 |
| 282 - 341 | N282 | C341 | VP160 - NCOR2_HUMAN | 2.35986047 | 7.97E−07 |
| 35 - 199 | N35 | C199 | VPR_JKNp64 - | 2.35882111 | 4.63E−04 |
| | | | ING3_HUMAN | | |
| 282 - 209 | N282 | C209 | VP160 - HDA10_HUMAN | 2.35789914 | 5.75E−09 |
| 312 - 281 | N312 | C281 | SetD8 - VP64 | 2.3572263 | 1.39E−06 |
| 35 - 269 | N35 | C269 | VPR_JKNp64 - PU.1 | 2.356532 | 9.78E−07 |
| 281 - 199 | N281 | C199 | VP64 - ING3_HUMAN | 2.35589673 | 8.76E−09 |
| 282 - 306 | N282 | C306 | VP160 - CobB | 2.35551229 | 1.25E−10 |
| 67 - 35 | N67 | C35 | PRDM9_HUMAN - | 2.35094186 | 3.87E−04 |
| | | | VPR_JKNp64 | | |
| 88 - 281 | N88 | C281 | ING4_HUMAN - VP64 | 2.35084197 | 8.29E−07 |
| 281 - 216 | N281 | C216 | VP64 - ING5_HUMAN | 2.3498918 | 8.95E−06 |
| 306 - 284 | N306 | C284 | CobB - p65-HSF1 | 2.34957576 | 1.03E−05 |
| 281 - 110 | N281 | C110 | VP64 - JMJD4_HUMAN | 2.34931303 | 6.36E−09 |
| 282 - 1 | N282 | C1 | VP160 - SETD1A | 2.34830052 | 0.00917087 |
| 281 - 201 | N281 | C201 | VP64 - KDM2B_HUMAN | 2.34758723 | 1.04E−04 |
| 282 - 10 | N282 | C10 | VP160 - HSF1 | 2.3460786 | 5.34E−09 |
| 346 - 35 | N346 | C35 | MSX2_HUMAN - | 2.34589432 | 1.70E−08 |
| | | | VPR_JKNp64 | | |
| 281 - 237 | N281 | C237 | VP64 - KAT5_HUMAN | 2.34572811 | 1.05E−05 |
| 284 - 10 | N284 | C10 | p65-HSF1 - HSF1 | 2.34448019 | 8.37E−04 |
| 35 - 218 | N35 | C218 | VPR_JKNp64 - | 2.34374481 | 4.75E−05 |
| | | | IN80B_HUMAN | | |
| 281 - 99 | N281 | C99 | VP64 - IN80C_HUMAN | 2.34205527 | 1.47E−06 |
| 42 - 281 | N42 | C281 | KMT2E_HUMAN - VP64 | 2.3389117 | 1.31E−07 |
| 282 - 27 | N282 | C27 | VP160 - EZH1 | 2.33807108 | 7.25E−11 |
| 174 - 281 | N174 | C281 | HDAC1_HUMAN - VP64 | 2.33597295 | 5.79E−07 |
| 282 - 56 | N282 | C56 | VP160 - SUV92_HUMAN | 2.33595238 | 5.58E−11 |
| 282 - 119 | N282 | C119 | VP160 - ACL6A_HUMAN | 2.33345055 | 2.21E−06 |
| 282 - 158 | N282 | C158 | VP160 - SETB1_HUMAN | 2.33150068 | 7.46E−05 |
| 281 - 190 | N281 | C190 | VP64 - ANM1_HUMAN | 2.32755942 | 2.59E−10 |
| 281 - 296 | N281 | C296 | VP64 - HP1a | 2.3264389 | 0.02195761 |
| 9 - 35 | N9 | C35 | MECP2 - VPR_JKNp64 | 2.32471582 | 2.73E−07 |
| 282 - 300 | N282 | C300 | VP160 - SIRT6 | 2.32436031 | 1.17E−10 |
| 2 - 281 | N2 | C281 | SETD1A - VP64 | 2.3234311 | 3.11E−04 |
| 99 - 281 | N99 | C281 | IN80C_HUMAN - VP64 | 2.32161407 | 1.12E−05 |
| 282 - 18 | N282 | C18 | VP160 - EZH2 | 2.3194372 | 1.32E−07 |
| 281 - 48 | N281 | C48 | VP64 - SETD5_HUMAN | 2.31730701 | 0.03666156 |
| 282 - 334 | N282 | C334 | VP160 - hs_RCOR1 | 2.31542994 | 2.95E−07 |
| 281 - 331 | N281 | C331 | VP64 - mm_G9a | 2.3145323 | 3.41E−09 |
| 284 - 98 | N284 | C98 | p65-HSF1 - | 2.31428435 | 1.19E−04 |
| | | | A4FU79_HUMAN | | |
| 200 - 99 | N200 | C99 | ARP5_HUMAN - | 2.31374165 | 2.11E−04 |
| | | | IN80C_HUMAN | | |
| 154 - 285 | N154 | C285 | NSD2_HUMAN - p65-MyoD | 2.31317635 | 1.43E−04 |
| 285 - 345 | N285 | C345 | p65-MyoD - MSX2_HUMAN | 2.3129054 | 9.41E−10 |
| 282 - 113 | N282 | C113 | VP160 - KDM5A_HUMAN | 2.3118538 | 7.92E−06 |
| 285 - 332 | N285 | C332 | p65-MyoD - hs_SIRT5 | 2.31144538 | 5.13E−05 |
| 284 - 288 | N284 | C288 | p65-HSF1 - SUV[SET] | 2.31002232 | 1.05E−04 |
| 282 - 45 | N282 | C45 | VP160 - KMT2D_HUMAN | 2.30870069 | 1.63E−09 |
| 282 - 349 | N282 | C349 | VP160 - DAM_N132A | 2.30786245 | 8.91E−10 |
| 281 - 221 | N281 | C221 | VP64 - SMYD2_HUMAN | 2.30127822 | 4.72E−06 |
| 281 - 129 | N281 | C129 | VP64 - HAIR_HUMAN | 2.30060611 | 1.71E−06 |
| 282 - 138 | N282 | C138 | VP160 - TFPT_HUMAN | 2.29824211 | 5.76E−10 |
| 282 - 215 | N282 | C215 | VP160 - HDAC4_HUMAN | 2.29452894 | 8.92E−06 |
| 281 - 309 | N281 | C309 | VP64 - HDAC8 | 2.29218206 | 8.26E−09 |
| 28 - 285 | N28 | C285 | EHMT2 - p65-MyoD | 2.28968877 | 1.56E−04 |
| 310 - 285 | N310 | C285 | TgSET8 - p65-MyoD | 2.2848314 | 3.98E−07 |
| 281 - 253 | N281 | C253 | VP64 - RBBP5_HUMAN | 2.28457316 | 8.40E−06 |
| 284 - 212 | N284 | C212 | p65-HSF1 - | 2.28442652 | 9.74E−08 |
| | | | HDAC8_HUMAN | | |
| 282 - 29 | N282 | C29 | VP160 - ASH2L | 2.28055267 | 0.00014668 |
| 14 - 308 | N14 | C308 | RELA - HDT1 | 2.2796922 | 1.52E−07 |
| 285 - 66 | N285 | C66 | p65-MyoD - | 2.27898531 | 2.76E−05 |
| | | | DOT1L_HUMAN | | |
| 304 - 281 | N304 | C281 | DNMT3a - VP64 | 2.27885885 | 1.07E−04 |
| 281 - 56 | N281 | C56 | VP64 - SUV92_HUMAN | 2.27489262 | 1.35E−06 |
| 222 - 284 | N222 | C284 | IN80E_HUMAN - p65-HSF1 | 2.2711804 | 5.58E−05 |
| 32 - 35 | N32 | C35 | Gilbert_Lab_CRISPRi_KRAB - | 2.27077555 | 3.17E−04 |
| | | | VPR_JKNp64 | | |
| 285 - 317 | N285 | C317 | p65-MyoD - HDAC3 | 2.26832612 | 6.41E−08 |
| 282 - 77 | N282 | C77 | VP160 - KDM5D_HUMAN | 2.26628276 | 4.60E−08 |
| 282 - 127 | N282 | C127 | VP160 - SETMR_HUMAN | 2.26450261 | 5.03E−10 |
| 282 - 278 | N282 | C278 | VP160 - TET2CD | 2.26449667 | 2.65E−05 |
| 212 - 285 | N212 | C285 | HDAC8_HUMAN - p65- | 2.2638459 | 4.88E−08 |
| | | | MyoD | | |
| 281 - 156 | N281 | C156 | VP64 - A7E2E1_HUMAN | 2.26241231 | 1.92E−04 |
| 282 - 150 | N282 | C150 | VP160 - HDA11_HUMAN | 2.26230285 | 1.29E−08 |
| 14 - 99 | N14 | C99 | RELA - IN80C_HUMAN | 2.26114765 | 1.37E−07 |
| 281 - 229 | N281 | C229 | VP64 - NFRKB_HUMAN | 2.25621282 | 3.82E−07 |
| 284 - 219 | N284 | C219 | p65-HSF1 - JARD2_HUMAN | 2.25610143 | 1.52E−08 |
| 281 - 100 | N281 | C100 | VP64 - TAF1_HUMAN | 2.2557008 | 7.36E−06 |
| 282 - 36 | N282 | C36 | VP160 - TET1CD_JKNp84 | 2.25299953 | 1.12E−07 |
| 5 - 285 | N5 | C285 | KDM1A - p65-MyoD | 2.24908377 | 1.22E−05 |
| 281 - 134 | N281 | C134 | VP64 - KDM2A_HUMAN | 2.24866378 | 1.25E−05 |
| 282 - 186 | N282 | C186 | VP160 - RIOX2_HUMAN | 2.24827011 | 1.01E−09 |
| 284 - 1 | N284 | C1 | p65-HSF1 - SETD1A | 2.24537787 | 3.08E−05 |
| 281 - 154 | N281 | C154 | VP64 - NSD2_HUMAN | 2.24533297 | 3.98E−10 |
| 173 - 281 | N173 | C281 | SIR4_HUMAN - VP64 | 2.24532671 | 1.95E−07 |
| 217 - 281 | N217 | C281 | CHRC1_HUMAN - VP64 | 2.24362966 | 0.03060538 |
| 282 - 293 | N282 | C293 | VP160 - KRAB-MeCP2 | 2.24242293 | 1.26E−06 |
| 173 - 285 | N173 | C285 | SIR4_HUMAN - p65-MyoD | 2.24234219 | 2.79E−05 |
| 313 - 285 | N313 | C285 | SaII - p65-MyoD | 2.24159283 | 4.75E−07 |
| 282 - 58 | N282 | C58 | VP160 - TF3C4_HUMAN | 2.24127139 | 1.64E−05 |
| 105 -281 | N105 | C281 | MBD2_HUMAN - VP64 | 2.23994084 | 1.62E−05 |
| 35 - 285 | N35 | C285 | VPR_JKNp64 - p65-MyoD | 2.23768968 | 7.77E−04 |
| 249 - 281 | N249 | C281 | MTL5_HUMAN - VP64 | 2.23767565 | 1.69E−04 |
| 35 - 290 | N35 | C290 | VPR_JKNp64 - HP1aCS | 2.23706773 | 7.45E−09 |
| 253 - 284 | N253 | C284 | RBBP5_HUMAN - p65-HSF1 | 2.23473704 | 0.00211784 |
| 282 - 55 | N282 | C55 | VP160 - RBBP4_HUMAN | 2.23199999 | 2.57E−04 |
| 281 - 34 | N281 | C34 | VP64 - DNMT3A- | 2.23144475 | 1.48E−09 |
| | | | 3L_JKNp112 | | |
| 281 - 332 | N281 | C332 | VP64 - hs_SIRT5 | 2.2308086 | 1.51E−07 |
| 282 - 42 | N282 | C42 | VP160 - KMT2E_HUMAN | 2.23043258 | 1.95E−07 |
| 280 - 285 | N280 | C285 | CBF1-Cterm - p65-MyoD | 2.23005976 | 4.59E−07 |
| 110- 281 | N110 | C281 | JMJD4_HUMAN - VP64 | 2.22992616 | 4.30E−05 |
| 282 - 237 | N282 | C237 | VP160 - KAT5_HUMAN | 2.22975962 | 1.91E−04 |
| 282 - 199 | N282 | C199 | VP160 - ING3_HUMAN | 2.22891547 | 9.45E−10 |
| 328 -281 | N328 | C281 | hs_PHF19 - VP64 | 2.2281139 | 2.06E−06 |
| 282 - 326 | N282 | C326 | VP160 - pf_Sir2A | 2.22660259 | 2.88E−10 |
| 253 - 14 | N253 | C14 | RBBP5_HUMAN - RELA | 2.2245583 | 0.00227472 |
| 284 - 9 | N284 | C9 | p65-HSF1 - MECP2 | 2.22382064 | 7.03E−06 |
| 282 - 236 | N282 | C236 | VP160 - KAT2A_HUMAN | 2.22201786 | 9.51E−09 |
| 281 - 83 | N281 | C83 | VP64 - SMRD3_HUMAN | 2.22192909 | 6.46E−06 |
| 281 - 87 | N281 | C87 | VP64 - SETD7_HUMAN | 2.22166893 | 1.66E−06 |
| 284 - 290 | N284 | C290 | p65-HSF1 - HP1aCS | 2.22138079 | 2.64E−09 |
| 282 - 179 | N282 | C179 | VP160 - SMRD2_HUMAN | 2.22122487 | 6.21E−07 |
| 284 - 190 | N284 | C190 | p65-HSF1 - ANM1_HUMAN | 2.21895399 | 3.89E−06 |
| 282 - 63 | N282 | C63 | VP160 - SMYD5_HUMAN | 2.2185528 | 7.61E−10 |
| 282 - 34 | N282 | C34 | VP160 - DNMT3A- | 2.21844748 | 1.67E−08 |
| | | | 3L_JKNp112 | | |
| 281 - 164 | N281 | C164 | VP64 - SIR5_HUMAN | 2.21820997 | 2.13E−09 |
| 282 - 193 | N282 | C193 | VP160 - KDM4D_HUMAN | 2.21817555 | 8.00E−07 |
| 281 - 324 | N281 | C324 | VP64 - LBR_HUMAN | 2.21765679 | 9.14E−06 |
| 281 - 133 | N281 | C133 | VP64 - KAT8_HUMAN | 2.2163792 | 7.08E−06 |
| 282 - 172 | N282 | C172 | VP160 - ASH1L_HUMAN | 2.21422087 | 3.60E−09 |
| 35 - 315 | N35 | C315 | VPR_JKNp64 - NCoR | 2.21347846 | 2.18E−07 |
| 343 - 281 | N343 | C281 | NCOR2_HUMAN - VP64 | 2.21324685 | 8.53E−04 |
| 281 - 24 | N281 | C24 | VP64 - CTCFL | 2.21216454 | 5.99E−06 |
| 282 - 216 | N282 | C216 | VP160 - ING5_HUMAN | 2.21198804 | 1.96E−07 |
| 187 - 281 | N187 | C281 | MBD4_HUMAN - VP64 | 2.2107555 | 1.64E−05 |
| 246 - 284 | N246 | C284 | ATRX_HUMAN - p65-HSF1 | 2.21072816 | 6.59E−06 |
| 212 - 14 | N212 | C14 | HDAC8_HUMAN - RELA | 2.20566605 | 1.54E−06 |
| 239 - 284 | N239 | C284 | KDM8_HUMAN - p65-HSF1 | 2.20562298 | 4.89E−06 |
| 281 - 320 | N281 | C320 | VP64 - mMLL3-SET | 2.20529114 | 4.59E−06 |
| 282 - 270 | N282 | C270 | VP160 - ENL | 2.20461588 | 2.54E−08 |
| 284 - 295 | N284 | C295 | p65-HSF1 - SET-TAF1B | 2.20276277 | 1.45E−05 |
| 64 - 281 | N64 | C281 | HDAC2_HUMAN - VP64 | 2.20275196 | 2.32E−04 |
| 282 - 92 | N282 | C92 | VP160 - SMYD1_HUMAN | 2.20210111 | 9.20E−05 |
| 282 - 211 | N282 | C211 | VP160 - KMT2B_HUMAN | 2.20116874 | 3.31E−04 |
| 281 - 305 | N281 | C305 | VP64 - NUE | 2.20083824 | 1.09E−06 |
| 35 - 288 | N35 | C288 | VPR_JKNp64 - SUV[SET] | 2.19996275 | 1.11E−04 |
| 281 - 138 | N281 | C138 | VP64 - TFPT_HUMAN | 2.19995535 | 8.38E−06 |
| 55 - 284 | N55 | C284 | RBBP4_HUMAN - p65-HSF1 | 2.19830129 | 2.99E−06 |
| 281 - 300 | N281 | C300 | VP64 - SIRT6 | 2.19734978 | 1.04E−08 |
| 151 - 284 | N151 | C284 | SIR7_HUMAN - p65-HSF1 | 2.19672988 | 0.00168931 |
| 17 - 285 | N17 | C285 | EZH2 - p65-MyoD | 2.19643707 | 4.59E−09 |
| 284 - 20 | N284 | C20 | p65-HSF1 - KDM1B | 2.19562478 | 8.14E−04 |
| 35 - 8 | N35 | C8 | VPR_JKNp64 - MECP2 | 2.1941769 | 2.31E−06 |
| 281 - 299 | N281 | C299 | VP64 - HST2 | 2.19313122 | 4.92E−09 |
| 112 - 35 | N112 | C35 | KDM5A_HUMAN - | 2.19307818 | 1.05E−05 |
| | | | VPR_JKNp64 | | |
| 282 - 149 | N282 | C149 | VP160 - MBD1_HUMAN | 2.18675073 | 1.71E−06 |
| 231 - 35 | N231 | C35 | KDM5B_HUMAN - | 2.18652808 | 5.11E−04 |
| | | | VPR_JKNp64 | | |
| 282 - 256 | N282 | C256 | VP160 - LSD1-ORF | 2.18641958 | 0.00122296 |
| 281 - 312 | N281 | C312 | VP64 - SetD8 | 2.18333825 | 1.59E−05 |
| 281 - 339 | N281 | C339 | VP64 - SPOC1_HUMAN | 2.18218474 | 4.34E−07 |
| 281 - 210 | N281 | C210 | VP64 - SIR3_HUMAN | 2.18034336 | 2.27E−07 |
| 313 - 10 | N313 | C10 | SaII - HSF1 | 2.17803452 | 1.26E−05 |
| 287 - 285 | N287 | C285 | G9A[SET] - p65-MyoD | 2.17783865 | 8.28E−06 |
| 281 - 119 | N281 | C119 | VP64 - ACL6A_HUMAN | 2.17733184 | 1.45E−06 |
| 282 - 328 | N282 | C328 | VP160 - hs_PHF19 | 2.17605254 | 1.71E−06 |
| 282 - 263 | N282 | C263 | VP160 - HDAC8 | 2.1751416 | 6.43E−05 |
| 275 - 285 | N275 | C285 | DOT1L-DOT1_domain - p65- | 2.17382721 | 4.10E−05 |
| | | | MyoD | | |
| 285 - 205 | N285 | C205 | p65-MyoD - | 2.17250078 | 0.00108665 |
| | | | JMJD7_HUMAN | | |
| 100 - 281 | N100 | C281 | TAF1_HUMAN - VP64 | 2.17210553 | 4.42E−04 |
| 281 - 14 | N281 | C14 | VP64 - RELA | 2.17047246 | 7.82E−06 |
| 87 - 281 | N87 | C281 | SETD7_HUMAN - VP64 | 2.16982206 | 2.49E−05 |
| 281 - 3 | N281 | C3 | VP64 - HDAC3 | 2.16915287 | 8.68E−04 |
| 281 - 348 | N281 | C348 | VP64 - BRDT_HUMAN | 2.16801834 | 3.45E−09 |
| 284 - 275 | N284 | C275 | p65-HSF1 - DOT1L- | 2.16603016 | 2.38E−04 |
| | | | DOT1_domain | | |
| 282 - 16 | N282 | C16 | VP160 - EP300 | 2.16322008 | 2.17E−04 |
| 14 - 15 | N14 | C15 | RELA - EP300 | 2.1627465 | 1.55E−06 |
| 284 - 133 | N284 | C133 | p65-HSF1 - KAT8_HUMAN | 2.16249123 | 2.76E−04 |
| 35 - 187 | N35 | C187 | VPR_JKNp64 - | 2.15803467 | 3.92E−06 |
| | | | MBD4_HUMAN | | |
| 282 - 97 | N282 | C97 | VP160 - CHD3_HUMAN | 2.15715565 | 6.65E−09 |
| 35 - 79 | N35 | C79 | VPR_JKNp64 - | 2.15586603 | 0.00166398 |
| | | | KAT6A_HUMAN | | |
| 282 - 57 | N282 | C57 | VP160 - ANM8_HUMAN | 2.15539535 | 4.48E−05 |
| 282 - 53 | N282 | C53 | VP160 - CBP_HUMAN | 2.15421419 | 4.59E−07 |
| 282 - 118 | N282 | C118 | VP160 - HDAC6_HUMAN | 2.15397819 | 9.87E−06 |
| 75 - 281 | N75 | C281 | KDM5D_HUMAN - VP64 | 2.15323922 | 1.26E−04 |
| 14 - 106 | N14 | C106 | RELA - KAT6B_HUMAN | 2.15100311 | 2.73E−05 |
| 268 - 35 | N268 | C35 | FOXA1-Cterm - | 2.15099614 | 0.00181817 |
| | | | VPR_JKNp64 | | |
| 285 - 116 | N285 | C116 | p65-MyoD - ANM6_HUMAN | 2.15044255 | 3.35E−06 |
| 284 - 191 | N284 | C191 | p65-HSF1 - CDYL_HUMAN | 2.14675114 | 0.00474092 |
| 65 - 281 | N65 | C281 | MCRS1_HUMAN - VP64 | 2.14578408 | 1.31E−05 |
| 285 - 77 | N285 | C77 | p65-MyoD - | 2.14518784 | 8.14E−05 |
| | | | KDM5D_HUMAN | | |
| 281 - 130 | N281 | C130 | VP64 - PRDM2_HUMAN | 2.14468024 | 3.08E−06 |
| 285 - 275 | N285 | C275 | p65-MyoD - DOT1L- | 2.14325576 | 2.89E−07 |
| | | | DOT1_domain | | |
| 280 - 257 | N280 | C257 | CBF1-Cterm - c-Myb-TAD | 2.14318851 | 0.0411472 |
| 281 - 95 | N281 | C95 | VP64 - SETD6_HUMAN | 2.14233763 | 4.61E−07 |
| 282 - 248 | N282 | C248 | VP160 - GLYR1_HUMAN | 2.14213909 | 1.31E−05 |
| 282 - 264 | N282 | C264 | VP160 - MTL5_HUMAN | 2.14094763 | 2.40E−08 |
| 282 - 93 | N282 | C93 | VP160 - ACL6B_HUMAN | 2.13863723 | 3.88E−07 |
| 1 - 284 | N1 | C284 | SETD1A - p65-HSF1 | 2.13846922 | 1.76E−06 |
| 285 - 289 | N285 | C289 | p65-MyoD - FOG1 | 2.13664779 | 2.32E−04 |
| 14 - 280 | N14 | C280 | RELA - CBF1-Cterm | 2.1365757 | 1.52E−07 |
| 281 - 97 | N281 | C97 | VP64 - CHD3_HUMAN | 2.13619432 | 7.76E−07 |
| 282 - 136 | N282 | C136 | VP160 - JMJD6_HUMAN | 2.13455071 | 6.47E−07 |
| 73 - 35 | N73 | C35 | KDM5D_HUMAN - | 2.1342701 | 0.00143559 |
| | | | VPR_JKNp64 | | |
| 285 - 190 | N285 | C190 | p65-MyoD - ANM1_HUMAN | 2.13262482 | 3.30E−05 |
| 282 - 302 | N282 | C302 | VP160 - HDAC11 | 2.131344 | 9.61E−09 |
| 25 - 284 | N25 | C284 | CTCFL - p65-HSF1 | 2.13070346 | 1.24E−06 |
| 281 - 297 | N281 | C297 | VP64 - MesoLo | 2.13065321 | 0.02591663 |
| 282 - 114 | N282 | C114 | VP160 - KDM5A_HUMAN | 2.12948495 | 7.00E−06 |
| 210 - 35 | N210 | C35 | SIR3_HUMAN - | 2.12665349 | 0.0010864 |
| | | | VPR_JKNp64 | | |
| 281 - 36 | N281 | C36 | VP64 - TET1CD_JKNp84 | 2.12657042 | 8.35E−06 |
| 281 - 45 | N281 | C45 | VP64 - KMT2D_HUMAN | 2.12509979 | 8.76E−05 |
| 47 - 281 | N47 | C281 | ANM2_HUMAN - VP64 | 2.12485226 | 5.20E−06 |
| 281 - 212 | N281 | C212 | VP64 - HDAC8_HUMAN | 2.11994934 | 2.85E−06 |
| 281 - 175 | N281 | C175 | VP64 - RUVB2_HUMAN | 2.11975956 | 3.37E−05 |
| 68 - 281 | N68 | C281 | MBD3_HUMAN - VP64 | 2.11935915 | 4.13E−06 |
| 281 - 144 | N281 | C144 | VP64 - UTY_HUMAN | 2.11870323 | 1.12E−05 |
| 190 - 284 | N190 | C284 | ANM1_HUMAN - p65-HSF1 | 2.11837628 | 2.68E−04 |
| 113 - 281 | N113 | C281 | KDM5A_HUMAN - VP64 | 2.11249937 | 1.79E−04 |
| 300 - 285 | N300 | C285 | SIRT6 - p65-MyoD | 2.11231506 | 2.09E−06 |
| 324 - 252 | N324 | C252 | LBR_HUMAN - | 2.1121918 | 0.00194586 |
| | | | HCFC1_HUMAN | | |
| 281 - 272 | N281 | C272 | VP64 - Sir2 | 2.11112776 | 6.28E−06 |
| 35 - 302 | N35 | C302 | VPR_JKNp64 - HDAC11 | 2.11061411 | 9.69E−04 |
| 284 - 131 | N284 | C131 | p65-HSF1 - | 2.11031994 | 8.72E−04 |
| | | | PRDM2_HUMAN | | |
| 282 - 189 | N282 | C189 | VP160 - ING2_HUMAN | 2.10888498 | 7.95E−05 |
| 35 - 272 | N35 | C272 | VPR_JKNp64 - Sir2 | 2.10760801 | 3.14E−04 |
| 88 - 35 | N88 | C35 | ING4_HUMAN - | 2.10668106 | 1.04E−05 |
| | | | VPR_JKNp64 | | |
| 255 - 35 | N255 | C35 | EED_HUMAN - | 2.1058882 | 0.0066058 |
| | | | VPR_JKNp64 | | |
| 282 - 107 | N282 | C107 | VP160 - KAT6B_HUMAN | 2.10291055 | 8.82E−08 |
| 285 - 250 | N285 | C250 | p65-MyoD - | 2.10186153 | 0.00608744 |
| | | | HCFC1_HUMAN | | |
| 35 - 185 | N35 | C185 | VPR_JKNp64 - | 2.09944774 | 0.00354754 |
| | | | RBBP7_HUMAN | | |
| 55 - 281 | N55 | C281 | RBBP4_HUMAN - VP64 | 2.09935178 | 4.81E−09 |
| 281 - 61 | N281 | C61 | VP64 - HAT1_HUMAN | 2.09777117 | 2.95E−09 |
| 284 - 306 | N284 | C306 | p65-HSF1 - CobB | 2.09575486 | 1.76E−05 |
| 285 - 55 | N285 | C55 | p65-MyoD - | 2.09522499 | 3.92E−05 |
| | | | RBBP4_HUMAN | | |
| 281 - 136 | N281 | C136 | VP64 - JMJD6_HUMAN | 2.09492147 | 4.80E−05 |
| 281 - 12 | N281 | C12 | VP64 - KMT2A | 2.09213372 | 2.24E−05 |
| 230 - 14 | N230 | C14 | NFRKB_HUMAN - RELA | 2.09156776 | 0.00171348 |
| 17 - 257 | N17 | C257 | EZH2 - c-Myb-TAD | 2.09096344 | 0.02495112 |
| 35 - 300 | N35 | C300 | VPR_JKNp64 - SIRT6 | 2.0908266 | 6.20E−05 |
| 282 - 167 | N282 | C167 | VP160 - EHMT1_HUMAN | 2.09050953 | 8.05E−06 |
| 281 - 219 | N281 | C219 | VP64 - JARD2_HUMAN | 2.09034567 | 7.80E−08 |
| 282 - 66 | N282 | C66 | VP160 - DOT1L_HUMAN | 2.09015107 | 7.71E−06 |
| 282 - 47 | N282 | C47 | VP160 - ANM2_HUMAN | 2.08995378 | 2.10E−07 |
| 282 - 222 | N282 | C222 | VP160 - IN80E_HUMAN | 2.08925667 | 1.50E−07 |
| 285 - 73 | N285 | C73 | p65-MyoD - | 2.08832051 | 1.38E−06 |
| | | | KDM5D_HUMAN | | |
| 79 - 281 | N79 | C281 | KAT6A_HUMAN - VP64 | 2.08796482 | 5.34E−05 |
| 219 - 284 | N219 | C284 | JARD2_HUMAN - p65-HSF1 | 2.08774676 | 0.00404254 |
| 281 - 183 | N281 | C183 | VP64 - KMT2C_HUMAN | 2.08423546 | 1.20E−04 |
| 284 - 2 | N284 | C2 | p65-HSF1 - SETD1A | 2.08294722 | 0.00297395 |
| 282 - 305 | N282 | C305 | VP160 - NUE | 2.08253006 | 5.87E−08 |
| 346 - 284 | N346 | C284 | MSX2_HUMAN - p65-HSF1 | 2.08221538 | 1.44E−06 |
| 282 - 311 | N282 | C311 | VP160 - Sin3a | 2.08211571 | 1.55E−09 |
| 250 - 296 | N250 | C296 | HCFC1_HUMAN - HP1a | 2.07899171 | 7.44E−05 |
| 74 - 285 | N74 | C285 | KDM5D_HUMAN - p65- | 2.07811607 | 0.00295941 |
| | | | MyoD | | |
| 285 - 130 | N285 | C130 | p65-MyoD - | 2.07806051 | 5.08E−06 |
| | | | PRDM2_HUMAN | | |
| 285 - 346 | N285 | C346 | p65-MyoD - MSX2_HUMAN | 2.0773162 | 6.57E−07 |
| 158 - 281 | N158 | C281 | SETB1_HUMAN - VP64 | 2.07689842 | 5.49E−05 |
| 281 - 18 | N281 | C18 | VP64 - EZH2 | 2.07369886 | 1.00E−06 |
| 173 - 284 | N173 | C284 | SIR4_HUMAN - p65-HSF1 | 2.07304839 | 2.24E−06 |
| 281 - 193 | N281 | C193 | VP64 - KDM4D_HUMAN | 2.07124304 | 2.62E−08 |
| 281 - 344 | N281 | C344 | VP64 - HDAC3_HUMAN | 2.07053932 | 3.37E−07 |
| 38 - 281 | N38 | C281 | EHMT2 - VP64 | 2.06912926 | 1.68E−07 |
| 284 - 205 | N284 | C205 | p65-HSF1 - JMJD7_HUMAN | 2.06883935 | 4.77E−05 |
| 245 - 252 | N245 | C252 | TYY1_HUMAN - | 2.06882456 | 0.00110138 |
| | | | HCFC1_HUMAN | | |
| 282 - 129 | N282 | C129 | VP160 - HAIR_HUMAN | 2.06872858 | 1.14E−05 |
| 35 - 293 | N35 | C293 | VPR_JKNp64 - KRAB- | 2.06754934 | 6.43E−05 |
| | | | MeCP2 | | |
| 281 - 159 | N281 | C159 | VP64 - SETB1_HUMAN | 2.06658828 | 1.61E−04 |
| 281 - 143 | N281 | C143 | VP64 - KDM4B_HUMAN | 2.06629216 | 7.66E−05 |
| 282 - 135 | N282 | C135 | VP160 - KDM2A_HUMAN | 2.0616614 | 3.00E−06 |
| 285 - 69 | N285 | C69 | p65-MyoD - | 2.06107531 | 4.75E−08 |
| | | | SETB2_HUMAN | | |
| 284 - 88 | N284 | C88 | p65-HSF1 - ING4_HUMAN | 2.05969798 | 2.82E−04 |
| 284 - 138 | N284 | C138 | p65-HSF1 - TFPT_HUMAN | 2.05968029 | 3.85E−06 |
| 282 - 133 | N282 | C133 | VP160 - KAT8_HUMAN | 2.05884521 | 2.23E−07 |
| 281 - 120 | N281 | C120 | VP64 - RING1_HUMAN | 2.05735372 | 3.03E−07 |
| 35 - 219 | N35 | C219 | VPR_JKNp64 - | 2.05724647 | 6.21E−05 |
| | | | JARD2_HUMAN | | |
| 239 - 281 | N239 | C281 | KDM8_HUMAN - VP64 | 2.05682256 | 6.23E−05 |
| 35 - 257 | N35 | C257 | VPR_JKNp64 - c-Myb-TAD | 2.05613909 | 3.69E−08 |
| 345 - 35 | N345 | C35 | MSX2_HUMAN - | 2.05493578 | 5.16E−05 |
| | | | VPR_JKNp64 | | |
| 284 - 308 | N284 | C308 | p65-HSF1 - HDT1 | 2.0516017 | 3.40E−06 |
| 282 - 85 | N282 | C85 | VP160 - SMYD3_HUMAN | 2.05024147 | 9.63E−08 |
| 282 - 146 | N282 | C146 | VP160 - KDM4A_HUMAN | 2.04938541 | 3.37E−04 |
| 284 - 15 | N284 | C15 | p65-HSF1 - EP300 | 2.04720103 | 1.67E−05 |
| 282 - 59 | N282 | C59 | VP160 - INO80_HUMAN | 2.0470798 | 4.79E−08 |
| 163 - 285 | N163 | C285 | ANM7_HUMAN - p65-MyoD | 2.04454795 | 0.00618745 |
| 257 - 35 | N257 | C35 | c-Myb-TAD - VPR_JKNp64 | 2.04447107 | 3.54E−07 |
| 285 - 78 | N285 | C78 | p65-MyoD - | 2.04282356 | 1.59E−05 |
| | | | KAT6A_HUMAN | | |
| 282 - 221 | N282 | C221 | VP160 - SMYD2_HUMAN | 2.04274035 | 9.40E−04 |
| 35 - 68 | N35 | C68 | VPR_JKNp64 - | 2.04263402 | 1.17E−06 |
| | | | MBD3_HUMAN | | |
| 222 - 285 | N222 | C285 | IN80E_HUMAN - p65-MyoD | 2.04185789 | 9.83E−08 |
| 282 - 276 | N282 | C276 | VP160 - EHMT2(G9a)-Cterm | 2.04064435 | 1.01E−04 |
| 281 - 186 | N281 | C186 | VP64 - RIOX2_HUMAN | 2.04015062 | 9.37E−08 |
| 136 - 285 | N136 | C285 | JMJD6_HUMAN - p65- | 2.04011751 | 3.54E−05 |
| | | | MyoD | | |
| 281 - 42 | N281 | C42 | VP64 - KMT2E_HUMAN | 2.03906919 | 1.84E−07 |
| 281 - 349 | N281 | C349 | VP64 - DAM_N132A | 2.03613229 | 3.21E−06 |
| 281 - 343 | N281 | C343 | VP64 - NCOR2_HUMAN | 2.03611409 | 0.00162656 |
| 281 - 310 | N281 | C310 | VP64 - TgSET8 | 2.0361093 | 1.06E−06 |
| 282 - 320 | N282 | C320 | VP160 - mMLL3-SET | 2.03583016 | 8.46E−06 |
| 35 - 296 | N35 | C296 | VPR_JKNp64 - HP1a | 2.03534409 | 2.82E−07 |
| 35 - 327 | N35 | C327 | VPR_JKNp64 - hs_NIPP1 | 2.03507915 | 6.77E−04 |
| 35 - 222 | N35 | C222 | VPR_JKNp64 - | 2.03467912 | 1.50E−05 |
| | | | IN80E_HUMAN | | |
| 51 - 281 | N51 | C281 | UCHL5_HUMAN - VP64 | 2.03449864 | 6.21E−05 |
| 2 - 285 | N2 | C285 | SETD1A - p65-MyoD | 2.03226426 | 7.89E−04 |
| 284 - 266 | N284 | C266 | p65-HSF1 - GADD45A | 2.0319566 | 5.01E−06 |
| 17 - 14 | N17 | C14 | EZH2 - RELA | 2.03155184 | 4.10E−08 |
| 285 - 129 | N285 | C129 | p65-MyoD - HAIR_HUMAN | 2.03104294 | 0.00869132 |
| 284 - 77 | N284 | C77 | p65-HSF1 - | 2.0305289 | 1.25E−04 |
| | | | KDM5D_HUMAN | | |
| 284 - 300 | N284 | C300 | p65-HSF1 - SIRT6 | 2.0302157 | 9.26E−05 |
| 35 - 308 | N35 | C308 | VPR_JKNp64 - HDT1 | 2.0285866 | 1.07E−04 |
| 165 - 281 | N165 | C281 | MTA2_HUMAN - VP64 | 2.02818101 | 0.00157059 |
| 281 - 151 | N281 | C151 | VP64 - SIR7_HUMAN | 2.0253243 | 0.00383897 |
| 281 - 173 | N281 | C173 | VP64 - SIR4_HUMAN | 2.02516507 | 2.10E−07 |
| 73 - 114 | N73 | C114 | KDM5D_HUMAN - | 2.0250142 | 0.00565123 |
| | | | KDM5A_HUMAN | | |
| 35 - 17 | N35 | C17 | VPR_JKNp64 - EZH2 | 2.02469703 | 0.00102692 |
| 282 - 287 | N282 | C287 | VP160 - G9A[SET] | 2.02419219 | 2.39E−07 |
| 282 - 327 | N282 | C327 | VP160 - hs_NIPP1 | 2.02390198 | 1.75E−08 |
| 282 - 336 | N282 | C336 | VP160 - ce_Set4 | 2.02382475 | 9.07E−07 |
| 284 - 176 | N284 | C176 | p65-HSF1 - ARP8_HUMAN | 2.02245899 | 5.40E−04 |
| 123 - 281 | N123 | C281 | KMT2A_HUMAN - VP64 | 2.02053994 | 7.04E−04 |
| 273 - 284 | N273 | C284 | PRDM9-CD - p65-HSF1 | 2.01903656 | 2.55E−04 |
| 284 - 264 | N284 | C264 | p65-HSF1 - MTL5_HUMAN | 2.01766977 | 1.20E−04 |
| 285 - 194 | N285 | C194 | p65-MyoD - ING1_HUMAN | 2.01465144 | 2.27E−05 |
| 282 - 332 | N282 | C332 | VP160 - hs_SIRT5 | 2.01333305 | 1.65E−08 |
| 285 - 265 | N285 | C265 | p65-MyoD - EZH2 | 2.01321769 | 0.01225195 |
| 33 - 290 | N33 | C290 | Hilton_2015 - HP1aCS | 2.01275581 | 3.33E−05 |
| 301 -281 | N301 | C281 | SIRT3 - VP64 | 2.01247983 | 4.38E−05 |
| 73 - 284 | N73 | C284 | KDM5D_HUMAN - p65- | 2.01019613 | 3.40E−07 |
| | | | HSF1 | | |
| 282 - 110 | N282 | C110 | VP160 - JMJD4_HUMAN | 2.0097467 | 6.28E−07 |
| 348 - 281 | N348 | C281 | BRDT_HUMAN - VP64 | 2.00642342 | 1.27E−04 |
| 284 - 312 | N284 | C312 | p65-HSF1 - SetD8 | 2.00542888 | 7.03E−06 |
| 153 - 5 | N153 | C5 | KAT7_HUMAN - KDM1A | 2.00426386 | 0.00803587 |
| 281 - 235 | N281 | C235 | VP64 - CARM1_HUMAN | 2.00354284 | 1.71E−05 |
| 35 - 5 | N35 | C5 | VPR_JKNp64 - KDM1A | 2.00296159 | 1.54E−05 |
| 334 - 166 | N334 | C166 | hs_RCOR1 - | 2.00286881 | 0.01289817 |
| | | | EHMT1_HUMAN | | |
| 35 - 345 | N35 | C345 | VPR_JKNp64 - | 2.00225994 | 3.81E−04 |
| | | | MSX2_HUMAN | | |
| 282 -201 | N282 | C201 | VP160 - KDM2B_HUMAN | 2.00155393 | 1.36E−04 |
| 35 - 317 | N35 | C317 | VPR_JKNp64 - HDAC3 | 2.00132914 | 0.01092336 |
| 284 - 142 | N284 | C142 | p65-HSF1 - | 2.00126164 | 0.00338073 |
| | | | SMRC1_HUMAN | | |
| 285 - 183 | N285 | C183 | p65-MyoD - | 2.00034505 | 0.00613708 |
| | | | KMT2C_HUMAN | | |
| 282 - 229 | N282 | C229 | VP160 - NFRKB_HUMAN | 1.99995616 | 1.89E−07 |
| 281 - 37 | N281 | C37 | VP64 - KDM6A | 1.99873976 | 4.59E−07 |
| 32 - 284 | N32 | C284 | Gilbert_Lab_CRISPRi_KRAB - | 1.99861414 | 6.57E−06 |
| | | | p65-HSF1 | | |
| 185 - 281 | N185 | C281 | RBBP7_HUMAN - VP64 | 1.99792689 | 3.05E−05 |
| 285 - 255 | N285 | C255 | p65-MyoD - EED_HUMAN | 1.99771158 | 0.00183136 |
| 14 - 267 | N14 | C267 | RELA - FOXA1-Nterm | 1.99704806 | 1.91E−07 |
| 274 - 284 | N274 | C284 | UBE2A - p65-HSF1 | 1.99538731 | 1.03E−05 |
| 285 - 266 | N285 | C266 | p65-MyoD - GADD45A | 1.99492751 | 2.23E−05 |
| 285 - 113 | N285 | C113 | p65-MyoD - | 1.99433294 | 8.65E−04 |
| | | | KDM5A_HUMAN | | |
| 281 - 336 | N281 | C336 | VP64 - ce_Set4 | 1.99427602 | 1.54E−05 |
| 285 - 314 | N285 | C314 | p65-MyoD - MeCP2 | 1.99261597 | 6.14E−06 |
| 323 - 281 | N323 | C281 | HNRPU_HUMAN - VP64 | 1.98630441 | 0.00310134 |
| 285 - 5 | N285 | C5 | p65-MyoD - KDM1A | 1.98614608 | 6.36E−05 |
| 281 - 295 | N281 | C295 | VP64 - SET-TAF1B | 1.98246657 | 1.70E−06 |
| 282 - 89 | N282 | C89 | VP160 - CHD4_HUMAN | 1.98181578 | 1.04E−04 |
| 285 - 312 | N285 | C312 | p65-MyoD - SetD8 | 1.98175316 | 7.57E−07 |
| 281 - 135 | N281 | C135 | VP64 - KDM2A_HUMAN | 1.98143357 | 2.67E−07 |
| 95 - 281 | N95 | C281 | SETD6_HUMAN - VP64 | 1.98013802 | 6.61E−05 |
| 56 - 281 | N56 | C281 | SUV92_HUMAN - VP64 | 1.97717575 | 2.43E−06 |
| 47 - 252 | N47 | C252 | ANM2_HUMAN - | 1.9770421 | 0.00183393 |
| | | | HCFC1_HUMAN | | |
| 284 - 186 | N284 | C186 | p65-HSF1 - RIOX2_HUMAN | 1.97668459 | 1.83E−04 |
| 233 - 285 | N233 | C285 | HINFP_HUMAN - p65- | 1.97470241 | 1.08E−04 |
| | | | MyoD | | |
| 285 - 179 | N285 | C179 | p65-MyoD - | 1.97456765 | 0.00119515 |
| | | | SMRD2_HUMAN | | |
| 285 - 246 | N285 | C246 | p65-MyoD - ATRX_HUMAN | 1.97404055 | 7.60E−08 |
| 281 - 204 | N281 | C204 | VP64 - HIF1N_HUMAN | 1.9730373 | 5.05E−06 |
| 282 -210 | N282 | C210 | VP160 - SIR3_HUMAN | 1.97299802 | 2.50E−07 |
| 281 - 16 | N281 | C16 | VP64 - EP300 | 1.97283717 | 1.77E−05 |
| 282 - 81 | N282 | C81 | VP160 - NCOA3_HUMAN | 1.97137444 | 0.00448076 |
| 281 - 249 | N281 | C249 | VP64 - HAC1-HAT | 1.97080605 | 2.54E−07 |
| 317 - 35 | N317 | C35 | HDAC3 - VPR_JKNp64 | 1.96994898 | 8.12E−05 |
| 285 - 135 | N285 | C135 | p65-MyoD - | 1.96978944 | 1.17E−04 |
| | | | KDM2A_HUMAN | | |
| 295 - 281 | N295 | C281 | SET-TAF1B - VP64 | 1.96842528 | 7.15E−05 |
| 14 - 204 | N14 | C204 | RELA - HIFIN_HUMAN | 1.96796472 | 1.74E−04 |
| 35 - 229 | N35 | C229 | VPR_JKNp64 - | 1.96589143 | 0.00605368 |
| | | | NFRKB_HUMAN | | |
| 284 - 73 | N284 | C73 | p65-HSF1 - | 1.9654438 | 1.92E−07 |
| | | | KDM5D_HUMAN | | |
| 33 - 314 | N33 | C314 | Hilton_2015 - MeCP2 | 1.96113448 | 0.00134362 |
| 284 - 27 | N284 | C27 | p65-HSF1 - EZH1 | 1.96096144 | 8.82E−06 |
| 35 - 346 | N35 | C346 | VPR_JKNp64 - | 1.96041127 | 6.00E−08 |
| | | | MSX2_HUMAN | | |
| 281 - 22 | N281 | C22 | VP64 - TET1 | 1.9599033 | 4.14E−05 |
| 35 - 38 | N35 | C38 | VPR_JKNp64 - EHMT2 | 1.95802492 | 9.33E−04 |
| 281 - 28 | N281 | C28 | VP64 - EHMT2 | 1.9576252 | 0.00733542 |
| 186 - 285 | N186 | C285 | RIOX2_HUMAN - p65- | 1.95703539 | 2.37E−04 |
| | | | MyoD | | |
| 35 - 77 | N35 | C77 | VPR_JKNp64 - | 1.95685339 | 1.05E−05 |
| | | | KDM5D_HUMAN | | |
| 21 - 281 | N21 | C281 | KDM1B - VP64 | 1.95457502 | 2.78E−04 |
| 66 - 281 | N66 | C281 | DOT1L_HUMAN - VP64 | 1.95360935 | 5.33E−06 |
| 201 - 281 | N201 | C281 | KDM2B_HUMAN - VP64 | 1.9534716 | 2.15E−05 |
| 45 - 35 | N45 | C35 | KMT2D_HUMAN - | 1.95017955 | 0.00474272 |
| | | | VPR_JKNp64 | | |
| 111 - 284 | N111 | C284 | KDM5A_HUMAN - p65- | 1.94990107 | 0.00102169 |
| | | | HSF1 | | |
| 200 - 24 | N200 | C24 | ARP5_HUMAN - CTCFL | 1.94966366 | 0.00277562 |
| 282 - 22 | N282 | C22 | VP160 - TET1 | 1.94683413 | 2.01E−06 |
| 285 - 9 | N285 | C9 | p65-MyoD - MECP2 | 1.94502328 | 5.29E−06 |
| 282 - 147 | N282 | C147 | VP160 - NSD3_HUMAN | 1.94421334 | 2.57E−05 |
| 281 - 123 | N281 | C123 | VP64 - KMT2A_HUMAN | 1.94340495 | 1.45E−04 |
| 284 - 150 | N284 | C150 | p65-HSF1 - | 1.94246633 | 1.16E−05 |
| | | | HDA11_HUMAN | | |
| 284 - 43 | N284 | C43 | p65-HSF1 - SETD4_HUMAN | 1.94176262 | 0.00182952 |
| 268 - 257 | N268 | C257 | FOXA1-Cterm - c-Myb-TAD | 1.94052674 | 2.35E−04 |
| 281 - 59 | N281 | C59 | VP64 - INO80_HUMAN | 1.94041681 | 1.96E−05 |
| 281 - 187 | N281 | C187 | VP64 - MBD4_HUMAN | 1.94007755 | 1.46E−07 |
| 296 - 284 | N296 | C284 | HP1a - p65-HSF1 | 1.93993584 | 3.30E−07 |
| 282 - 253 | N282 | C253 | VP160 - RBBP5_HUMAN | 1.93943511 | 5.23E−06 |
| 281 - 101 | N281 | C101 | VP64 - TAF1_HUMAN | 1.93812061 | 1.05E−06 |
| 114 - 14 | N114 | C14 | KDM5A_HUMAN - RELA | 1.93786076 | 1.07E−05 |
| 324 - 285 | N324 | C285 | LBR_HUMAN - p65-MyoD | 1.93753803 | 1.81E−05 |
| 35 - 266 | N35 | C266 | VPR_JKNp64 - GADD45A | 1.93645883 | 9.10E−04 |
| 284 - 305 | N284 | C305 | p65-HSF1 - NUE | 1.93457418 | 9.12E−07 |
| 282 - 271 | N282 | C271 | VP160 - GCN5 | 1.9344222 | 5.17E−06 |
| 49 - 284 | N49 | C284 | NCOA1_HUMAN - p65- | 1.93269272 | 0.00492592 |
| | | | HSF1 | | |
| 282 - 130 | N282 | C130 | VP160 - PRDM2_HUMAN | 1.93207524 | 1.10E−07 |
| 205 - 99 | N205 | C99 | JMJD7_HUMAN - | 1.93092621 | 0.00123955 |
| | | | IN80C_HUMAN | | |
| 284 - 216 | N284 | C216 | p65-HSF1 - ING5_HUMAN | 1.93086493 | 1.37E−06 |
| 285 - 127 | N285 | C127 | p65-MyoD - | 1.93063443 | 1.74E−06 |
| | | | SETMR_HUMAN | | |
| 26 - 281 | N26 | C281 | EZH1 - VP64 | 1.93024493 | 0.00582035 |
| 282 - 70 | N282 | C70 | VP160 - SETB2_HUMAN | 1.92912207 | 1.49E−06 |
| 35 - 132 | N35 | C132 | VPR_JKNp64 - | 1.92886237 | 0.00119139 |
| | | | PRDM2_HUMAN | | |
| 35 - 214 | N35 | C214 | VPR_JKNp64 - | 1.92868542 | 5.28E−04 |
| | | | HDAC4_HUMAN | | |
| 254 - 281 | N254 | C281 | MEN1_HUMAN - VP64 | 1.92754138 | 0.00368272 |
| 291 - 222 | N291 | C222 | SS18 - IN80E_HUMAN | 1.92736055 | 0.01785917 |
| 282 - 330 | N282 | C330 | VP160 - ce_Set1 | 1.92614015 | 2.20E−06 |
| 282 - 190 | N282 | C190 | VP160 - ANM1_HUMAN | 1.92478498 | 2.99E−07 |
| 285 - 268 | N285 | C268 | p65-MyoD - FOXA1-Cterm | 1.9222326 | 0.00213465 |
| 282 - 348 | N282 | C348 | VP160 - BRDT_HUMAN | 1.92197067 | 4.06E−08 |
| 267 - 284 | N267 | C284 | FOXA1-Nterm - p65-HSF1 | 1.9210844 | 1.32E−04 |
| 282 - 303 | N282 | C303 | VP160 - EZH2 | 1.91796494 | 8.41E−06 |
| 281 - 308 | N281 | C308 | VP64 - HDT1 | 1.91657265 | 6.82E−04 |
| 284 - 311 | N284 | C311 | p65-HSF1 - Sin3a | 1.91270844 | 5.73E−07 |
| 280 - 252 | N280 | C252 | CBF1-Cterm - | 1.91203499 | 4.91E−06 |
| | | | HCFC1_HUMAN | | |
| 65 - 285 | N65 | C285 | MCRS1_HUMAN - p65- | 1.91173345 | 0.01288536 |
| | | | MyoD | | |
| 282 - 112 | N282 | C112 | VP160 - KDMSA_HUMAN | 1.90975054 | 5.73E−07 |
| 282 - 324 | N282 | C324 | VP160 - LBR_HUMAN | 1.90933138 | 3.31E−06 |
| 292 - 91 | N292 | C91 | MQ1(Q147L) - | 1.90724795 | 0.01228607 |
| | | | ANM5_HUMAN | | |
| 282 - 143 | N282 | C143 | VP160 - KDM4B_HUMAN | 1.90679029 | 0.00198636 |
| 281 - 271 | N281 | C271 | VP64 - GCN5 | 1.90540686 | 6.48E−04 |
| 267 - 14 | N267 | C14 | FOXA1-Nterm - RELA | 1.90436128 | 0.012242 |
| 282 - 61 | N282 | C61 | VP160 - HAT1_HUMAN | 1.9041584 | 4.30E−07 |
| 182 - 281 | N182 | C281 | MTA3_HUMAN - VP64 | 1.90392918 | 8.67E−05 |
| 281 - 150 | N281 | C150 | VP64 - HDA11_HUMAN | 1.90332449 | 1.62E−07 |
| 272 - 281 | N272 | C281 | Sir2 - VP64 | 1.90290829 | 5.82E−04 |
| 219 - 281 | N219 | C281 | JARD2_HUMAN - VP64 | 1.90069476 | 0.00374917 |
| 14 - 296 | N14 | C296 | RELA - HP1a | 1.89892589 | 2.48E−04 |
| 35 - 205 | N35 | C205 | VPR_JKNp64 - | 1.89807771 | 0.00185102 |
| | | | JMJD7_HUMAN | | |
| 282 - 296 | N282 | C296 | VP160 - HP1a | 1.89704516 | 3.86E−05 |
| 143 - 281 | N143 | C281 | KDM4B_HUMAN - VP64 | 1.89704201 | 8.86E−04 |
| 282 - 132 | N282 | C132 | VP160 - PRDM2_HUMAN | 1.89457176 | 4.71E−07 |
| 282 - 307 | N282 | C307 | VP160 - KYP | 1.89413541 | 8.55E−08 |
| 230 - 332 | N230 | C332 | NFRKB_HUMAN - | 1.89300478 | 0.02578195 |
| | | | hs_SIRT5 | | |
| 25 - 252 | N25 | C252 | CTCFL - HCFC1_HUMAN | 1.89293832 | 2.15E−04 |
| 284 - 187 | N284 | C187 | p65-HSF1 - MBD4_HUMAN | 1.89176455 | 4.09E−05 |
| 199 - 284 | N199 | C284 | ING3_HUMAN - p65-HSF1 | 1.89081256 | 3.49E−04 |
| 307 - 281 | N307 | C281 | KYP - VP64 | 1.88786733 | 4.42E−05 |
| 35 - 217 | N35 | C217 | VPR_JKNp64 - | 1.88612749 | 2.27E−04 |
| | | | CHRC1_HUMAN | | |
| 240 - 281 | N240 | C281 | ANM3_HUMAN - VP64 | 1.88605515 | 0.00392448 |
| 35 - 216 | N35 | C216 | VPR_JKNp64 - | 1.88337149 | 8.10E−05 |
| | | | ING5_HUMAN | | |
| 281 - 317 | N281 | C317 | VP64 - HDAC3 | 1.88287303 | 2.08E−07 |
| 35 - 268 | N35 | C268 | VPR_JKNp64 - FOXA1- | 1.88043656 | 1.31E−04 |
| | | | Cterm | | |
| 285 - 326 | N285 | C326 | p65-MyoD - pf_Sir2A | 1.87990074 | 8.45E−06 |
| 295 - 285 | N295 | C285 | SET-TAF1B - p65-MyoD | 1.87814815 | 3.24E−06 |
| 35 - 108 | N35 | C108 | VPR_JKNp64 - | 1.87661628 | 0.00808688 |
| | | | KAT6B_HUMAN | | |
| 271 - 281 | N271 | C281 | GCN5 - VP64 | 1.8717501 | 0.00393244 |
| 57 - 284 | N57 | C284 | ANM8_HUMAN - p65-HSF1 | 1.87154695 | 1.40E−04 |
| 282 - 177 | N282 | C177 | VP160 - KDM5C_HUMAN | 1.87112709 | 1.04E−06 |
| 88 - 284 | N88 | C284 | ING4_HUMAN - p65-HSF1 | 1.87092393 | 3.78E−05 |
| 281 - 287 | N281 | C287 | VP64 - G9A[SET] | 1.87070852 | 1.67E−04 |
| 281 - 79 | N281 | C79 | VP64 - KAT6A_HUMAN | 1.87048484 | 4.92E−08 |
| 282 - 64 | N282 | C64 | VP160 - HDAC2_HUMAN | 1.86897441 | 0.00104748 |
| 18 - 252 | N18 | C252 | EZH2 - HCFC1_HUMAN | 1.86814975 | 0.00520327 |
| 252 - 186 | N252 | C186 | HCFC1_HUMAN - | 1.86708258 | 0.0182183 |
| | | | RIOX2_HUMAN | | |
| 35 - 56 | N35 | C56 | VPR_JKNp64 - | 1.8658229 | 0.00345381 |
| | | | SUV92_HUMAN | | |
| 282 - 76 | N282 | C76 | VP160 - KDM5D_HUMAN | 1.86568445 | 1.24E−04 |
| 14 - 214 | N14 | C214 | RELA - HDAC4_HUMAN | 1.86448598 | 2.46E−04 |
| 285 - 164 | N285 | C164 | p65-MyoD - SIR5_HUMAN | 1.86366354 | 0.00143534 |
| 285 - 88 | N285 | C88 | p65-MyoD - ING4_HUMAN | 1.86351717 | 1.62E−06 |
| 284 - 183 | N284 | C183 | p65-HSF1 - | 1.86097959 | 1.89E−04 |
| | | | KMT2C_HUMAN | | |
| 136 - 284 | N136 | C284 | JMJD6_HUMAN - p65-HSF1 | 1.85975617 | 4.14E−06 |
| 281 - 213 | N281 | C213 | VP64 - HBAP1_HUMAN | 1.85934508 | 2.35E−05 |
| 282 - 333 | N282 | C333 | VP160 - sc_RPD3 | 1.85934238 | 7.37E−05 |
| 146 - 281 | N146 | C281 | KDM4A_HUMAN - VP64 | 1.8581589 | 0.00618304 |
| 282 - 220 | N282 | C220 | VP160 - JARD2_HUMAN | 1.85745779 | 5.22E−06 |
| 87 - 285 | N87 | C285 | SETD7_HUMAN - p65- | 1.85643246 | 0.00281988 |
| | | | MyoD | | |
| 14 - 250 | N14 | C250 | RELA - HCFC1_HUMAN | 1.85562951 | 0.00282554 |
| 3 - 281 | N3 | C281 | HDAC3 - VP64 | 1.85548396 | 6.97E−05 |
| 282 - 331 | N282 | C331 | VP160 - mm_G9a | 1.85459834 | 6.87E−05 |
| 14 - 213 | N14 | C213 | RELA - HBAP1_HUMAN | 1.85272973 | 0.01756629 |
| 284 - 127 | N284 | C127 | p65-HSF1 - | 1.8520529 | 0.00159107 |
| | | | SETMR_HUMAN | | |
| 200 - 280 | N200 | C280 | ARP5_HUMAN - CBF1- | 1.85112699 | 0.00717696 |
| | | | Cterm | | |
| 227 - 281 | N227 | C281 | SIR1_HUMAN - VP64 | 1.85070357 | 0.00541639 |
| 281 - 127 | N281 | C127 | VP64 - SETMR_HUMAN | 1.84993394 | 1.65E−04 |
| 280 - 14 | N280 | C14 | CBF1-Cterm - RELA | 1.84964329 | 4.91E−07 |
| 281 - 147 | N281 | C147 | VP64 - NSD3_HUMAN | 1.84960728 | 0.00223977 |
| 14 - 130 | N14 | C130 | RELA - PRDM2_HUMAN | 1.84866998 | 1.91E−04 |
| 284 - 198 | N284 | C198 | p65-HSF1 - JMJD8_HUMAN | 1.84832111 | 2.40E−05 |
| 200 - 14 | N200 | C14 | ARP5_HUMAN - RELA | 1.84627457 | 0.00292666 |
| 282 - 329 | N282 | C329 | VP160 - pbcv1_vSET | 1.84570229 | 4.99E−05 |
| 252 - 46 | N252 | C46 | HCFC1_HUMAN - | 1.84557524 | 0.01401591 |
| | | | SMRD1_HUMAN | | |
| 282 - 317 | N282 | C317 | VP160 - HDAC3 | 1.8450851 | 1.33E−06 |
| 282 - 6 | N282 | C6 | VP160 - KDM1A | 1.84435642 | 2.48E−04 |
| 78 - 14 | N78 | C14 | KAT6A_HUMAN - RELA | 1.84412756 | 0.00826099 |
| 339 - 14 | N339 | C14 | SPOC1_HUMAN - RELA | 1.84216336 | 8.51E−04 |
| 277 - 284 | N277 | C284 | SUV39H1 - p65-HSF1 | 1.84198917 | 0.00791002 |
| 138 - 312 | N138 | C312 | TFPT_HUMAN - SetD8 | 1.84197551 | 0.00944124 |
| 5 - 10 | N5 | C10 | KDM1A - HSF1 | 1.8402101 | 3.51E−04 |
| 282 - 262 | N282 | C262 | VP160 - EZH2 | 1.84019137 | 4.63E−04 |
| 281 - 50 | N281 | C50 | VP64 - NCOA1_HUMAN | 1.83985376 | 5.34E−05 |
| 282 - 202 | N282 | C202 | VP160 - KDM2B_HUMAN | 1.8371192 | 2.33E−04 |
| 282 - 265 | N282 | C265 | VP160 - EZH2 | 1.83706036 | 0.01476984 |
| 14 - 348 | N14 | C348 | RELA - BRDT_HUMAN | 1.83634283 | 0.00206567 |
| 282 - 43 | N282 | C43 | VP160 - SETD4_HUMAN | 1.83588922 | 5.36E−06 |
| 160 - 281 | N160 | C281 | KMT5A_HUMAN - VP64 | 1.83549204 | 1.15E−04 |
| 282 - 7 | N282 | C7 | VP160 - ZNF10 | 1.83442866 | 3.32E−06 |
| 199 - 281 | N199 | C281 | ING3_HUMAN - VP64 | 1.83424721 | 3.83E−06 |
| 307 - 284 | N307 | C284 | KYP - p65-HSF1 | 1.83327206 | 1.66E−05 |
| 258 - 296 | N258 | C296 | Nejire-HAT - HP1a | 1.83286832 | 0.00808945 |
| 285 - 142 | N285 | C142 | p65-MyoD - | 1.83205599 | 0.00620332 |
| | | | SMRC1_HUMAN | | |
| 237 - 285 | N237 | C285 | KAT5_HUMAN - p65-MyoD | 1.83083781 | 4.91E−04 |
| 282 - 120 | N282 | C120 | VP160 - RING1_HUMAN | 1.83074624 | 2.62E−06 |
| 190 - 285 | N190 | C285 | ANM1_HUMAN - p65-MyoD | 1.82848444 | 1.18E−04 |
| 282 - 90 | N282 | C90 | VP160 - CHD4_HUMAN | 1.82839936 | 2.34E−06 |
| 281 - 69 | N281 | C69 | VP64 - SETB2_HUMAN | 1.82754346 | 3.84E−04 |
| 285 - 107 | N285 | C107 | p65-MyoD - | 1.82751287 | 0.00352436 |
| | | | KAT6B_HUMAN | | |
| 295 - 291 | N295 | C291 | SET-TAF1B - SS18 | 1.82724402 | 0.00403799 |
| 127 - 35 | N127 | C35 | SETMR_HUMAN - | 1.82711187 | 0.00766029 |
| | | | VPR_JKNp64 | | |
| 218 - 284 | N218 | C284 | IN80B_HUMAN - p65-HSF1 | 1.82548448 | 0.00741477 |
| 282 - 197 | N282 | C197 | VP160 - SUV91_HUMAN | 1.82374264 | 2.01E−04 |
| 9 - 281 | N9 | C281 | MECP2 - VP64 | 1.82363707 | 6.19E−06 |
| 35 - 164 | N35 | C164 | VPR_JKNp64 - | 1.8230199 | 2.51E−04 |
| | | | SIR5_HUMAN | | |
| 282 - 87 | N282 | C87 | VP160 - SETD7_HUMAN | 1.82208175 | 7.10E−05 |
| 294 - 14 | N294 | C14 | DIM5 - RELA | 1.82191351 | 0.0049491 |
| 281 - 326 | N281 | C326 | VP64 - pf_Sir2A | 1.82186314 | 1.15E−04 |
| 35 - 130 | N35 | C130 | VPR_JKNp64 - | 1.82181271 | 6.06E−05 |
| | | | PRDM2_HUMAN | | |
| 329 - 35 | N329 | C35 | pbcv1_vSET - VPR_JKNp64 | 1.82116633 | 4.58E−04 |
| 313 - 250 | N313 | C250 | SaII - HCFC1_HUMAN | 1.81967008 | 0.0091944 |
| 226 - 285 | N226 | C285 | SETD2_HUMAN - p65- | 1.81894825 | 2.53E−04 |
| | | | MyoD | | |
| 282 - 103 | N282 | C103 | VP160 - B4DKA4_HUMAN | 1.81770167 | 1.13E−05 |
| 90 - 150 | N90 | C150 | CHD4_HUMAN - | 1.81617181 | 0.00433672 |
| | | | HDA11_HUMAN | | |
| 24 - 14 | N24 | C14 | CTCFL - RELA | 1.81552218 | 0.00760944 |
| 281 - 306 | N281 | C306 | VP64 - CobB | 1.81543525 | 1.23E−04 |
| 281 - 220 | N281 | C220 | VP64 - JARD2_HUMAN | 1.81435484 | 1.56E−06 |
| 282 - 171 | N282 | C171 | VP160 - ASH1L_HUMAN | 1.81385056 | 4.85E−04 |
| 281 - 88 | N281 | C88 | VP64 - ING4_HUMAN | 1.81220459 | 5.89E−04 |
| 263 - 83 | N263 | C83 | HDAC8 - SMRD3_HUMAN | 1.81134846 | 0.03569605 |
| 106 - 35 | N106 | C35 | KAT6B_HUMAN - | 1.81125691 | 9.11E−05 |
| | | | VPR_JKNp64 | | |
| 282 - 219 | N282 | C219 | VP160 - JARD2_HUMAN | 1.81121055 | 1.35E−06 |
| 322 - 284 | N322 | C284 | MINT_HUMAN - p65-HSF1 | 1.80982867 | 0.0066497 |
| 296 - 281 | N296 | C281 | HP1a - VP64 | 1.8091595 | 6.27E−04 |
| 35 - 90 | N35 | C90 | VPR_JKNp64 - | 1.80897204 | 4.52E−04 |
| | | | CHD4_HUMAN | | |
| 281 - 113 | N281 | C113 | VP64 - KDM5A_HUMAN | 1.80861183 | 1.26E−04 |
| 189 - 285 | N189 | C285 | ING2_HUMAN - p65-MyoD | 1.8069013 | 1.48E−05 |
| 241 - 281 | N241 | C281 | SIR2_HUMAN - VP64 | 1.80645078 | 4.54E−04 |
| 281 - 277 | N281 | C277 | VP64 - SUV39H1 | 1.80614376 | 9.19E−06 |
| 281 - 118 | N281 | C118 | VP64 - HDAC6_HUMAN | 1.80600318 | 1.94E−05 |
| 14 - 1 | N14 | C1 | RELA - SETD1A | 1.80432207 | 2.29E−05 |
| 92 - 284 | N92 | C284 | SMYD1_HUMAN - p65- | 1.80432157 | 0.0017116 |
| | | | HSF1 | | |
| 282 - 48 | N282 | C48 | VP160 - SETD5_HUMAN | 1.80350248 | 3.04E−04 |
| 280 - 10 | N280 | C10 | CBF1-Cterm - HSF1 | 1.80208077 | 5.79E−05 |
| 20 - 284 | N20 | C284 | KDM1B - p65-HSF1 | 1.80185388 | 0.0033478 |
| 212 - 35 | N212 | C35 | HDAC8_HUMAN - | 1.80003583 | 0.00629441 |
| | | | VPR_JKNp64 | | |
| 277 - 281 | N277 | C281 | SUV39H1 - VP64 | 1.79885683 | 0.00483 |
| 35 - 332 | N35 | C332 | VPR_JKNp64 - hs_SIRT5 | 1.79876749 | 0.00536171 |
| 282 - 335 | N282 | C335 | VP160 - at_SUVR4 | 1.79740023 | 8.60E−05 |
| 284 - 45 | N284 | C45 | p65-HSF1 - | 1.7961639 | 7.92E−05 |
| | | | KMT2D_HUMAN | | |
| 281 - 71 | N281 | C71 | VP64 - RUVB1_HUMAN | 1.79600343 | 0.00672704 |
| 270 - 136 | N270 | C136 | ENL - JMJD6_HUMAN | 1.79527072 | 0.02511468 |
| 69 - 284 | N69 | C284 | SETB2_HUMAN - p65-HSF1 | 1.79460839 | 0.00030383 |
| 281 - 198 | N281 | C198 | VP64 - JMJD8_HUMAN | 1.79443956 | 8.90E−04 |
| 282 - 105 | N282 | C105 | VP160 - MBD2_HUMAN | 1.79267673 | 7.01E−05 |
| 285 - 306 | N285 | C306 | p65-MyoD - CobB | 1.79234691 | 0.00179337 |
| 200 - 106 | N200 | C106 | ARP5_HUMAN - | 1.79226015 | 0.0182875 |
| | | | KAT6B_HUMAN | | |
| 282 - 26 | N282 | C26 | VP160 - EZH1 | 1.79032041 | 1.97E−04 |
| 281 - 197 | N281 | C197 | VP64 - SUV91_HUMAN | 1.78975173 | 7.68E−04 |
| 285 - 186 | N285 | C186 | p65-MyoD - | 1.7882476 | 3.26E−05 |
| | | | RIOX2_HUMAN | | |
| 35 - 154 | N35 | C154 | VPR_JKNp64 - | 1.78715859 | 0.00574242 |
| | | | NSD2_HUMAN | | |
| 117 - 285 | N117 | C285 | HDAC6_HUMAN - p65- | 1.78674688 | 0.00549398 |
| | | | MyoD | | |
| 284 - 140 | N284 | C140 | p65-HSF1 - | 1.78622683 | 0.03080356 |
| | | | KDM6B_HUMAN | | |
| 14 - 199 | N14 | C199 | RELA - ING3_HUMAN | 1.78419314 | 0.001673 |
| 282 - 249 | N282 | C249 | VP160 - HAC1-HAT | 1.78363226 | 4.83E−05 |
| 284 - 231 | N284 | C231 | p65-HSF1 - | 1.78337713 | 0.01597079 |
| | | | KDM5B_HUMAN | | |
| 177 - 281 | N177 | C281 | KDM5C_HUMAN - VP64 | 1.78311927 | 6.12E−07 |
| 284 - 93 | N284 | C93 | p65-HSF1 - | 1.78310488 | 0.00158342 |
| | | | ACL6B_HUMAN | | |
| 285 - 134 | N285 | C134 | p65-MyoD - | 1.78208223 | 0.00174617 |
| | | | KDM2A_HUMAN | | |
| 35 - 294 | N35 | C294 | VPR_JKNp64 - DIM5 | 1.78075195 | 9.01E−05 |
| 281 - 102 | N281 | C102 | VP64 - TAF1_HUMAN | 1.78062433 | 0.00418935 |
| 76 - 281 | N76 | C281 | KDM5D_HUMAN - VP64 | 1.78051773 | 3.02E−04 |
| 282 - 241 | N282 | C241 | VP160 - SIR2_HUMAN | 1.77893025 | 1.11E−04 |
| 285 - 123 | N285 | C123 | p65-MyoD - | 1.77659817 | 7.27E−05 |
| | | | KMT2A_HUMAN | | |
| 230 - 257 | N230 | C257 | NFRKB_HUMAN - c-Myb- | 1.77643114 | 0.00903357 |
| | | | TAD | | |
| 282 - 37 | N282 | C37 | VP160 - KDM6A | 1.77529422 | 6.36E−06 |
| 281 - 264 | N281 | C264 | VP64 - MTL5_HUMAN | 1.77521046 | 1.98E−05 |
| 284 - 55 | N284 | C55 | p65-HSF1 - RBBP4_HUMAN | 1.77259613 | 8.94E−05 |
| 45 - 103 | N45 | C103 | KMT2D_HUMAN - | 1.772591 | 0.03442124 |
| | | | B4DKA4_HUMAN | | |
| 177 - 231 | N177 | C231 | KDM5C_HUMAN - | 1.77205077 | 0.02725873 |
| | | | KDM5B_HUMAN | | |
| 281 - 185 | N281 | C185 | VP64 - RBBP7_HUMAN | 1.77197486 | 9.37E−04 |
| 10 - 257 | N10 | C257 | HSF1 - c-Myb-TAD | 1.77131998 | 0.01556991 |
| 126 - 281 | N126 | C281 | SETMR_HUMAN - VP64 | 1.77074814 | 5.33E−05 |
| 285 - 305 | N285 | C305 | p65-MyoD - NUE | 1.77074429 | 4.98E−05 |
| 252 - 106 | N252 | C106 | HCFC1_HUMAN - | 1.76864917 | 0.03224572 |
| | | | KAT6B_HUMAN | | |
| 284 - 87 | N284 | C87 | p65-HSF1 - SETD7_HUMAN | 1.76768546 | 1.28E−05 |
| 327 - 284 | N327 | C284 | hs_NIPP1 - p65-HSF1 | 1.76709128 | 1.44E−04 |
| 14 - 212 | N14 | C212 | RELA - HDAC8_HUMAN | 1.76612365 | 0.00145428 |
| 284 - 209 | N284 | C209 | p65-HSF1 - | 1.76347539 | 0.0196543 |
| | | | HDA10_HUMAN | | |
| 35 - 267 | N35 | C267 | VPR_JKNp64 - FOXA1- | 1.7612904 | 4.46E−05 |
| | | | Nterm | | |
| 285 - 102 | N285 | C102 | p65-MyoD - TAF1_HUMAN | 1.7606933 | 3.47E−04 |
| 99 - 257 | N99 | C257 | IN80C_HUMAN - c-Myb- | 1.75958344 | 7.95E−04 |
| | | | TAD | | |
| 63 - 10 | N63 | C10 | SMYD5_HUMAN - HSF1 | 1.7595776 | 0.0189079 |
| 202 - 281 | N202 | C281 | KDM2B_HUMAN - VP64 | 1.75814293 | 0.00106836 |
| 282 - 191 | N282 | C191 | VP160 - CDYL_HUMAN | 1.75624831 | 1.11E−05 |
| 35 - 133 | N35 | C133 | VPR_JKNp64 - | 1.75530833 | 3.50E−04 |
| | | | KAT8_HUMAN | | |
| 231 - 281 | N231 | C281 | KDM5B_HUMAN - VP64 | 1.75374262 | 4.92E−04 |
| 285 - 213 | N285 | C213 | p65-MyoD - | 1.75359987 | 7.48E−04 |
| | | | HBAP1_HUMAN | | |
| 111 - 1 | N111 | C1 | KDM5A_HUMAN - SETD1A | 1.75325104 | 0.02000885 |
| 35 - 320 | N35 | C320 | VPR_JKNp64 - mMLL3-SET | 1.7529317 | 2.19E−04 |
| 5 - 14 | N5 | C14 | KDM1A - RELA | 1.74930934 | 1.78E−04 |
| 285 - 138 | N285 | C138 | p65-MyoD - TFPT_HUMAN | 1.74917564 | 0.00115623 |
| 291 - 257 | N291 | C257 | SS18 - c-Myb-TAD | 1.74807573 | 0.04095836 |
| 218 - 297 | N218 | C297 | IN80B_HUMAN - MesoLo | 1.74642431 | 0.01472868 |
| 281 - 155 | N281 | C155 | VP64 - NSD2_HUMAN | 1.74459017 | 2.68E−04 |
| 200 - 329 | N200 | C329 | ARP5_HUMAN - | 1.74307285 | 0.02540272 |
| | | | pbcv1_vSET | | |
| 281 - 330 | N281 | C330 | VP64 - ce_Set1 | 1.74212222 | 1.88E−04 |
| 285 - 56 | N285 | C56 | p65-MyoD - | 1.74186614 | 7.18E−04 |
| | | | SUV92_HUMAN | | |
| 289 - 284 | N289 | C284 | FOG1 - p65-HSF1 | 1.74168934 | 4.64E−05 |
| 282 - 49 | N282 | C49 | VP160 - NCOA1_HUMAN | 1.74144519 | 2.04E−05 |
| 37 - 281 | N37 | C281 | KDM6A - VP64 | 1.74124366 | 0.00547291 |
| 281 - 122 | N281 | C122 | VP64 - KMT2A_HUMAN | 1.74044238 | 0.02995729 |
| 88 - 285 | N88 | C285 | ING4_HUMAN - p65-MyoD | 1.73951218 | 2.10E−06 |
| 282 - 67 | N282 | C67 | VP160 - PRDM9_HUMAN | 1.73558727 | 4.09E−04 |
| 233 - 328 | N233 | C328 | HINFP_HUMAN - hs_PHF19 | 1.73341944 | 0.03861139 |
| 114 - 285 | N114 | C285 | KDM5A_HUMAN - p65- | 1.73255843 | 0.00275052 |
| | | | MyoD | | |
| 282 - 183 | N282 | C183 | VP160 - KMT2C_HUMAN | 1.73246931 | 5.30E−05 |
| 301 - 214 | N301 | C214 | SIRT3 - HDAC4_HUMAN | 1.73122289 | 0.03935844 |
| 285 - 328 | N285 | C328 | p65-MyoD - hs_PHF19 | 1.73010096 | 2.21E−04 |
| 289 - 35 | N289 | C35 | FOG1 - VPR_JKNp64 | 1.72927428 | 2.48E−05 |
| 24 - 285 | N24 | C285 | CTCFL - p65-MyoD | 1.72878617 | 0.00455254 |
| 237 - 222 | N237 | C222 | KAT5_HUMAN - | 1.72866686 | 0.0218706 |
| | | | IN80E_HUMAN | | |
| 273 - 252 | N273 | C252 | PRDM9-CD - | 1.72866686 | 0.0218706 |
| | | | HCFC1_HUMAN | | |
| 284 - 136 | N284 | C136 | p65-HSF1 - JMJD6_HUMAN | 1.7279361 | 5.71E−05 |
| 285 - 297 | N285 | C297 | p65-MyoD - MesoLo | 1.72775179 | 2.42E−04 |
| 285 - 2 | N285 | C2 | p65-MyoD - SETD1A | 1.72671653 | 6.17E−04 |
| 281 - 322 | N281 | C322 | VP64 - MINT_HUMAN | 1.72584207 | 9.34E−04 |
| 116 - 250 | N116 | C250 | ANM6_HUMAN - | 1.72562579 | 0.00170487 |
| | | | HCFC1_HUMAN | | |
| 284 - 307 | N284 | C307 | p65-HSF1 - KYP | 1.72499647 | 1.06E−04 |
| 295 - 14 | N295 | C14 | SET-TAF1B - RELA | 1.72333284 | 6.95E−06 |
| 35 - 172 | N35 | C172 | VPR_JKNp64 - | 1.72305875 | 0.00319592 |
| | | | ASH1L_HUMAN | | |
| 281 - 301 | N281 | C301 | VP64 - SIRT3 | 1.72116698 | 5.67E−04 |
| 205 - 50 | N205 | C50 | JMJD7_HUMAN - | 1.72035183 | 0.02801431 |
| | | | NCOA1_HUMAN | | |
| 35 - 61 | N35 | C61 | VPR_JKNp64 - | 1.71917802 | 0.00424423 |
| | | | HAT1_HUMAN | | |
| 78- 10 | N78 | C10 | KAT6A_HUMAN - HSF1 | 1.71889669 | 1.14E−04 |
| 200 - 257 | N200 | C257 | ARP5_HUMAN - c-Myb- | 1.71845457 | 2.27E−04 |
| | | | TAD | | |
| 35 - 275 | N35 | C275 | VPR_JKNp64 - DOT1L- | 1.71831898 | 0.00122792 |
| | | | DOT1_domain | | |
| 284 - 102 | N284 | C102 | p65-HSF1 - TAF1_HUMAN | 1.71757112 | 0.0030378 |
| 120 - 284 | N120 | C284 | RING1_HUMAN - p65-HSF1 | 1.71492643 | 0.00552937 |
| 281 - 338 | N281 | C338 | VP64 - RBM15_HUMAN | 1.71383592 | 0.01267639 |
| 21 - 14 | N21 | C14 | KDM1B - RELA | 1.71231662 | 0.00344785 |
| 111 - 10 | N111 | C10 | KDM5A_HUMAN - HSF1 | 1.71116012 | 0.00180243 |
| 253 - 278 | N253 | C278 | RBBP5_HUMAN - TET2CD | 1.7110399 | 0.01073171 |
| 91 - 14 | N91 | C14 | ANM5_HUMAN - RELA | 1.70867464 | 0.00931089 |
| 281 - 90 | N281 | C90 | VP64 - CHD4_HUMAN | 1.70787063 | 3.98E−05 |
| 282 - 134 | N282 | C134 | VP160 - KDM2A_HUMAN | 1.70773753 | 1.83E−04 |
| 284 - 134 | N284 | C134 | p65-HSF1 - | 1.70690765 | 0.0078851 |
| | | | KDM2A_HUMAN | | |
| 335 - 281 | N335 | C281 | at_SUVR4 - VP64 | 1.70639125 | 3.23E−04 |
| 14 - 149 | N14 | C149 | RELA - MBD1_HUMAN | 1.70516594 | 0.00797795 |
| 281 - 254 | N281 | C254 | VP64 - MEN1_HUMAN | 1.70497475 | 1.52E−04 |
| 285 - 87 | N285 | C87 | p65-MyoD - | 1.70497093 | 4.81E−04 |
| | | | SETD7_HUMAN | | |
| 281 - 176 | N281 | C176 | VP64 - ARP8_HUMAN | 1.70493717 | 0.00231242 |
| 269 - 35 | N269 | C35 | PU.1 - VPR_JKNp64 | 1.70323209 | 0.02694294 |
| 35 - 25 | N35 | C25 | VPR_JKNp64 - CTCFL | 1.7002713 | 0.02936786 |
| 282 - 235 | N282 | C235 | VP160 - CARM1_HUMAN | 1.6991373 | 0.04270499 |
| 217 - 285 | N217 | C285 | CHRC1_HUMAN - p65- | 1.69853428 | 6.62E−04 |
| | | | MyoD | | |
| 263 - 327 | N263 | C327 | HDAC8 - hs_NIPP1 | 1.69821826 | 0.02665714 |
| 285 - 24 | N285 | C24 | p65-MyoD - CTCFL | 1.69622818 | 1.13E−04 |
| 64 - 14 | N64 | C14 | HDAC2_HUMAN - RELA | 1.69492672 | 6.82E−04 |
| 79 - 284 | N79 | C284 | KAT6A_HUMAN - p65- | 1.692103 | 0.00525727 |
| | | | HSF1 | | |
| 285 - 79 | N285 | C79 | p65-MyoD - | 1.69158207 | 4.92E−04 |
| | | | KAT6A_HUMAN | | |
| 164 - 14 | N164 | C14 | SIR5_HUMAN - RELA | 1.69078588 | 2.05E−04 |
| 282 - 185 | N282 | C185 | VP160 - RBBP7_HUMAN | 1.69062218 | 0.00159386 |
| 310 - 257 | N310 | C257 | TgSET8 - c-Myb-TAD | 1.68990283 | 3.21E−06 |
| 281 - 307 | N281 | C307 | VP64 - KYP | 1.68855927 | 1.22E−05 |
| 14 - 339 | N14 | C339 | RELA - SPOC1_HUMAN | 1.68798305 | 0.00917492 |
| 114 - 99 | N114 | C99 | KDM5A_HUMAN - | 1.68716029 | 0.01099815 |
| | | | IN80C_HUMAN | | |
| 282 - 126 | N282 | C126 | VP160 - SETMR_HUMAN | 1.68708038 | 7.00E−05 |
| 285 - 299 | N285 | C299 | p65-MyoD - HST2 | 1.68672508 | 2.55E−04 |
| 138 - 14 | N138 | C14 | TFPT_HUMAN - RELA | 1.6847774 | 5.14E−04 |
| 285 - 25 | N285 | C25 | p65-MyoD - CTCFL | 1.68342718 | 2.34E−04 |
| 263 - 281 | N263 | C281 | HDAC8 - VP64 | 1.68231937 | 0.00263016 |
| 281 - 270 | N281 | C270 | VP64 - ENL | 1.68133686 | 4.25E−05 |
| 345 - 257 | N345 | C257 | MSX2_HUMAN - c-Myb- | 1.6811097 | 0.00605768 |
| | | | TAD | | |
| 282 - 312 | N282 | C312 | VP160 - SetD8 | 1.6803134 | 1.74E−05 |
| 281 - 188 | N281 | C188 | VP64 - SMYD4_HUMAN | 1.68003596 | 4.06E−04 |
| 150 - 10 | N150 | C10 | HDA11_HUMAN - HSF1 | 1.67909331 | 0.00970366 |
| 285 - 324 | N285 | C324 | p65-MyoD - LBR_HUMAN | 1.67874657 | 3.56E−06 |
| 126 - 14 | N126 | C14 | SETMR_HUMAN - RELA | 1.67776234 | 0.02697726 |
| 281 - 112 | N281 | C112 | VP64 - KDM5A_HUMAN | 1.67765822 | 8.48E−06 |
| 285 - 198 | N285 | C198 | p65-MyoD - | 1.6770391 | 2.30E−04 |
| | | | JMJD8_HUMAN | | |
| 284 - 95 | N284 | C95 | p65-HSF1 - SETD6_HUMAN | 1.67695456 | 0.00844105 |
| 14 - 66 | N14 | C66 | RELA - DOT1L_HUMAN | 1.67687566 | 0.0018241 |
| 112 - 284 | N112 | C284 | KDM5A_HUMAN - p65- | 1.67611741 | 0.00284385 |
| | | | HSF1 | | |
| 35 - 20 | N35 | C20 | VPR_JKNp64 - KDM1B | 1.67591494 | 0.00406447 |
| 150 - 284 | N150 | C284 | HDA11_HUMAN - p65- | 1.67487746 | 0.01251004 |
| | | | HSF1 | | |
| 277 - 100 | N277 | C100 | SUV39H1 - TAF1_HUMAN | 1.67353386 | 0.03406724 |
| 150 - 95 | N150 | C95 | HDA11_HUMAN - | 1.67294107 | 0.02557864 |
| | | | SETD6_HUMAN | | |
| 116 - 284 | N116 | C284 | ANM6_HUMAN - p65-HSF1 | 1.6714248 | 0.00140593 |
| 284 - 349 | N284 | C349 | p65-HSF1 - DAM_N132A | 1.66934662 | 0.00302589 |
| 284 - 143 | N284 | C143 | p65-HSF1 - | 1.66342822 | 0.0186432 |
| | | | KDM4B_HUMAN | | |
| 284 - 346 | N284 | C346 | p65-HSF1 - MSX2_HUMAN | 1.6633899 | 1.93E−04 |
| 285 - 243 | N285 | C243 | p65-MyoD - | 1.66271374 | 0.00263288 |
| | | | DNMT1_HUMAN | | |
| 257 - 285 | N257 | C285 | c-Myb-TAD - p65-MyoD | 1.66239091 | 6.54E−04 |
| 282 - 169 | N282 | C169 | VP160 - ARI1B_HUMAN | 1.66153886 | 0.0020561 |
| 35 - 336 | N35 | C336 | VPR_JKNp64 - ce_Set4 | 1.66132651 | 0.00219419 |
| 290 - 35 | N290 | C35 | HP1aCS - VPR_JKNp64 | 1.66130099 | 7.29E−05 |
| 185 - 284 | N185 | C284 | RBBP7_HUMAN - p65-HSF1 | 1.6601862 | 5.43E−04 |
| 35 - 297 | N35 | C297 | VPR_JKNp64 - MesoLo | 1.65932126 | 1.26E−04 |
| 66 - 280 | N66 | C280 | DOT1L_HUMAN - CBF1- | 1.65852972 | 0.01725435 |
| | | | Cterm | | |
| 281 - 228 | N281 | C228 | VP64 - HDAC7_HUMAN | 1.6584989 | 0.00803167 |
| 268 - 10 | N268 | C10 | FOXA1-Cterm - HSF1 | 1.65474043 | 1.68E−05 |
| 126 - 174 | N126 | C174 | SETMR_HUMAN - | 1.65457126 | 0.03241956 |
| | | | HDAC1_HUMAN | | |
| 281 - 167 | N281 | C167 | VP64 - EHMT1_HUMAN | 1.65387259 | 0.00456773 |
| 281 - 9 | N281 | C9 | VP64 - MECP2 | 1.65195698 | 9.23E−04 |
| 282 - 206 | N282 | C206 | VP160 - SETD9_HUMAN | 1.65176102 | 3.21E−05 |
| 14 - 313 | N14 | C313 | RELA - SaII | 1.64972155 | 3.71E−06 |
| 282 - 272 | N282 | C272 | VP160 - Sir2 | 1.64930571 | 4.87E−06 |
| 281 - 53 | N281 | C53 | VP64 - CBP_HUMAN | 1.64782686 | 8.44E−05 |
| 281 - 126 | N281 | C126 | VP64 - SETMR_HUMAN | 1.6476088 | 4.39E−04 |
| 285 - 176 | N285 | C176 | p65-MyoD - ARP8_HUMAN | 1.6467121 | 0.00355448 |
| 14 - 189 | N14 | C189 | RELA - ING2_HUMAN | 1.64567944 | 0.00395706 |
| 285 - 335 | N285 | C335 | p65-MyoD - at_SUVR4 | 1.64549256 | 1.63E−04 |
| 35 - 246 | N35 | C246 | VPR_JKNp64 - | 1.64520095 | 7.30E−04 |
| | | | ATRX_HUMAN | | |
| 282 - 194 | N282 | C194 | VP160 - ING1_HUMAN | 1.6443527 | 4.94E−04 |
| 70 - 281 | N70 | C281 | SETB2_HUMAN - VP64 | 1.64420223 | 0.03722299 |
| 99 - 291 | N99 | C291 | IN80C_HUMAN - SS18 | 1.64300505 | 7.19E−04 |
| 29 - 269 | N29 | C269 | ASH2L - PU.1 | 1.64284427 | 0.01112722 |
| 284 - 171 | N284 | C171 | p65-HSF1 - ASH1L_HUMAN | 1.64082993 | 0.00880446 |
| 348 - 285 | N348 | C285 | BRDT_HUMAN - p65-MyoD | 1.64022697 | 0.00186883 |
| 21 - 284 | N21 | C284 | KDM1B - p65-HSF1 | 1.63961504 | 4.29E−04 |
| 282 - 137 | N282 | C137 | VP160 - DNM3B_HUMAN | 1.63849846 | 0.00224433 |
| 14 - 145 | N14 | C145 | RELA - UTY_HUMAN | 1.63780832 | 0.01813702 |
| 281 - 67 | N281 | C67 | VP64 - PRDM9_HUMAN | 1.63775112 | 6.71E−04 |
| 281 - 20 | N281 | C20 | VP64 - KDM1B | 1.63569425 | 7.04E−04 |
| 208 - 257 | N208 | C257 | ANM9_HUMAN - c-Myb- | 1.63554573 | 0.01834321 |
| | | | TAD | | |
| 35 - 250 | N35 | C250 | VPR_JKNp64 - | 1.63427975 | 0.00125466 |
| | | | HCFC1_HUMAN | | |
| 149 - 257 | N149 | C257 | MBD1_HUMAN - c-Myb- | 1.63357808 | 1.31E−04 |
| | | | TAD | | |
| 273 - 249 | N273 | C249 | PRDM9-CD - HAC1-HAT | 1.63320251 | 0.01749932 |
| 281 - 30 | N281 | C30 | VP64 - KDM3A | 1.63229475 | 0.00193938 |
| 114 - 45 | N114 | C45 | KDM5A_HUMAN - | 1.63164306 | 0.00771937 |
| | | | KMT2D_HUMAN | | |
| 282 - 240 | N282 | C240 | VP160 - ANM3_HUMAN | 1.63061704 | 2.19E−04 |
| 284 - 18 | N284 | C18 | p65-HSF1 - EZH2 | 1.63049367 | 0.00204732 |
| 35 - 88 | N35 | C88 | VPR_JKNp64 - | 1.6300445 | 0.00308248 |
| | | | ING4_HUMAN | | |
| 35 - 191 | N35 | C191 | VPR_JKNp64 - | 1.62972537 | 0.02121395 |
| | | | CDYL_HUMAN | | |
| 281 - 206 | N281 | C206 | VP64 - SETD9_HUMAN | 1.62925733 | 2.35E−04 |
| 138 - 5 | N138 | C5 | TFPT_HUMAN - KDM1A | 1.62916174 | 0.01927479 |
| 281 - 114 | N281 | C114 | VP64 - KDM5A_HUMAN | 1.62816188 | 0.00171256 |
| 281 - 85 | N281 | C85 | VP64 - SMYD3_HUMAN | 1.6274716 | 0.00100379 |
| 175 - 281 | N175 | C281 | RUVB2_HUMAN - VP64 | 1.62664591 | 0.00327078 |
| 281 - 335 | N281 | C335 | VP64 - at_SUVR4 | 1.6265467 | 0.00166962 |
| 85 - 73 | N85 | C73 | SMYD3_HUMAN - | 1.62597032 | 0.01677691 |
| | | | KDM5D_HUMAN | | |
| 284 - 272 | N284 | C272 | p65-HSF1 - Sir2 | 1.62401765 | 0.00159268 |
| 310 - 252 | N310 | C252 | TgSET8 - HCFC1_HUMAN | 1.62283752 | 0.02662876 |
| 282 - 182 | N282 | C182 | VP160 - MTA3_HUMAN | 1.62170623 | 0.00139933 |
| 281 - 189 | N281 | C189 | VP64 - ING2_HUMAN | 1.62128539 | 0.00179222 |
| 14 - 26 | N14 | C26 | RELA - EZH1 | 1.61874497 | 1.55E−05 |
| 270 - 257 | N270 | C257 | ENL - c-Myb-TAD | 1.61871994 | 3.90E−06 |
| 69 - 285 | N69 | C285 | SETB2_HUMAN - p65- | 1.61861548 | 0.00761912 |
| | | | MyoD | | |
| 59 - 301 | N59 | C301 | INO80_HUMAN - SIRT3 | 1.61742639 | 0.0064373 |
| 281 - 334 | N281 | C334 | VP64 - hs_RCOR1 | 1.61421966 | 0.00519875 |
| 285 - 320 | N285 | C320 | p65-MyoD - mMLL3-SET | 1.61403082 | 1.40E−04 |
| 269 - 14 | N269 | C14 | PU.1 - RELA | 1.61356952 | 6.02E−05 |
| 147 - 281 | N147 | C281 | NSD3_HUMAN - VP64 | 1.61346295 | 0.00104764 |
| 10 - 154 | N10 | C154 | HSF1 - NSD2_HUMAN | 1.61302484 | 0.02444388 |
| 133 - 281 | N133 | C281 | KAT8_HUMAN - VP64 | 1.61205682 | 0.00666349 |
| 186 - 14 | N186 | C14 | RIOX2_HUMAN - RELA | 1.61018728 | 3.19E−04 |
| 78 - 257 | N78 | C257 | KAT6A_HUMAN - c-Myb- | 1.60911167 | 0.04382889 |
| | | | TAD | | |
| 266 - 10 | N266 | C10 | GADD45A - HSF1 | 1.60814225 | 0.00885879 |
| 35 - 59 | N35 | C59 | VPR_JKNp64 - | 1.60624155 | 0.00291917 |
| | | | INO80_HUMAN | | |
| 10 - 285 | N10 | C285 | HSF1 - p65-MyoD | 1.60623767 | 0.02738816 |
| 26 - 35 | N26 | C35 | EZH1 - VPR_JKNp64 | 1.60599679 | 0.0236144 |
| 282 - 75 | N282 | C75 | VP160 - KDM5D_HUMAN | 1.60527347 | 5.42E−06 |
| 140 - 190 | N140 | C190 | KDM6B_HUMAN - | 1.60464308 | 0.03636406 |
| | | | ANM1_HUMAN | | |
| 250 - 14 | N250 | C14 | HCFC1_HUMAN - RELA | 1.60453474 | 0.00954869 |
| 307 - 285 | N307 | C285 | KYP - p65-MyoD | 1.60411799 | 4.78E−05 |
| 114 - 15 | N114 | C15 | KDM5A_HUMAN - EP300 | 1.60320025 | 0.0045969 |
| 332 - 281 | N332 | C281 | hs_SIRT5 - VP64 | 1.60269229 | 0.00388825 |
| 87 - 10 | N87 | C10 | SETD7_HUMAN - HSF1 | 1.60237948 | 0.00422193 |
| 269 - 285 | N269 | C285 | PU.1 - p65-MyoD | 1.60227135 | 6.68E−04 |
| 14 - 327 | N14 | C327 | RELA - hs_NIPP1 | 1.60207765 | 9.24E−04 |
| 35 - 189 | N35 | C189 | VPR_JKNp64 - | 1.60160251 | 3.01E−04 |
| | | | ING2_HUMAN | | |
| 14 - 164 | N14 | C164 | RELA - SIR5_HUMAN | 1.60130597 | 4.31E−05 |
| 281 - 314 | N281 | C314 | VP64 - MeCP2 | 1.60118639 | 2.11E−04 |
| 284 - 189 | N284 | C189 | p65-HSF1 - ING2_HUMAN | 1.60056812 | 0.00519757 |
| 281 - 240 | N281 | C240 | VP64 - ANM3_HUMAN | 1.60033425 | 8.13E−05 |
| 332 - 285 | N332 | C285 | hs_SIRT5 - p65-MyoD | 1.60002691 | 6.35E−04 |
| 306 - 257 | N306 | C257 | CobB - c-Myb-TAD | 1.60001496 | 1.45E−04 |
| 14 - 252 | N14 | C252 | RELA - HCFC1_HUMAN | 1.59827988 | 0.02714981 |
| 253 - 10 | N253 | C10 | RBBP5_HUMAN - HSF1 | 1.59802152 | 0.01813257 |
| 281 - 27 | N281 | C27 | VP64 - EZH1 | 1.59700232 | 0.00152283 |
| 281 - 265 | N281 | C265 | VP64 - EZH2 | 1.59557677 | 0.02689313 |
| 59 - 284 | N59 | C284 | INO80_HUMAN - p65-HSF1 | 1.59512805 | 0.0026829 |
| 281 - 39 | N281 | C39 | VP64 - KDM3A | 1.59187816 | 4.19E−04 |
| 35 - 9 | N35 | C9 | VPR_JKNp64 - MECP2 | 1.59180824 | 0.02746355 |
| 14 - 269 | N14 | C269 | RELA - PU.1 | 1.59067831 | 4.48E−05 |
| 216 - 45 | N216 | C45 | ING5_HUMAN - | 1.59045251 | 0.01420661 |
| | | | KMT2D_HUMAN | | |
| 284 - 12 | N284 | C12 | p65-HSF1 - KMT2A | 1.58985245 | 0.01934908 |
| 252 - 326 | N252 | C326 | HCFC1_HUMAN - pf_Sir2A | 1.58972875 | 0.00468021 |
| 295 - 240 | N295 | C240 | SET-TAF1B - | 1.58967039 | 0.03447189 |
| | | | ANM3_HUMAN | | |
| 281 - 43 | N281 | C43 | VP64 - SETD4_HUMAN | 1.58842388 | 6.14E−05 |
| 33 - 105 | N33 | C105 | Hilton_2015 - | 1.58838078 | 0.00363636 |
| | | | MBD2_HUMAN | | |
| 46 - 14 | N46 | C14 | SMRD1_HUMAN - RELA | 1.58834631 | 0.02795695 |
| 162 - 285 | N162 | C285 | SIR6_HUMAN - p65-MyoD | 1.58800471 | 5.96E−04 |
| 35 - 299 | N35 | C299 | VPR_JKNp64 - HST2 | 1.58728818 | 0.00121837 |
| 284 - 235 | N284 | C235 | p65-HSF1 - | 1.58696951 | 0.04380174 |
| | | | CARM1_HUMAN | | |
| 281 - 236 | N281 | C236 | VP64 - KAT2A_HUMAN | 1.58663744 | 0.04328424 |
| 285 - 122 | N285 | C122 | p65-MyoD - | 1.58653083 | 0.02013649 |
| | | | KMT2A_HUMAN | | |
| 17 - 171 | N17 | C171 | EZH2 - ASH1L_HUMAN | 1.58588401 | 0.04869337 |
| 257 - 10 | N257 | C10 | c-Myb-TAD - HSF1 | 1.58578646 | 7.52E−05 |
| 282 - 325 | N282 | C325 | VP160 - LBR_HUMAN | 1.58509327 | 0.01361873 |
| 38 - 284 | N38 | C284 | EHMT2 - p65-HSF1 | 1.58478541 | 4.26E−04 |
| 284 - 56 | N284 | C56 | p65-HSF1 - SUV92_HUMAN | 1.5844657 | 0.01585973 |
| 281 - 214 | N281 | C214 | VP64 - HDAC4_HUMAN | 1.58394929 | 9.34E−06 |
| 282 - 274 | N282 | C274 | VP160 - UBE2A | 1.58377603 | 7.22E−05 |
| 281 - 255 | N281 | C255 | VP64 - EED_HUMAN | 1.58333623 | 2.06E−04 |
| 14 - 249 | N14 | C249 | RELA - HAC1-HAT | 1.58055623 | 3.78E−05 |
| 349 - 35 | N349 | C35 | DAM_N132A - VPR_JKNp64 | 1.57969914 | 0.00114422 |
| 294 - 281 | N294 | C281 | DIM5 - VP64 | 1.57961703 | 4.22E−04 |
| 280 - 102 | N280 | C102 | CBF1-Cterm - | 1.57944154 | 0.03878189 |
| | | | TAF1_HUMAN | | |
| 71 -281 | N71 | C281 | RUVB1_HUMAN - VP64 | 1.57920896 | 0.0014375 |
| 282 - 231 | N282 | C231 | VP160 - KDM5B_HUMAN | 1.57741171 | 3.06E−04 |
| 149 - 285 | N149 | C285 | MBD1_HUMAN - p65-MyoD | 1.57713849 | 0.00307848 |
| 218 - 54 | N218 | C54 | IN80B_HUMAN - | 1.576874 | 0.01850924 |
| | | | CBP_HUMAN | | |
| 285 - 111 | N285 | C111 | p65-MyoD - | 1.57672231 | 3.70E−05 |
| | | | KDM5A_HUMAN | | |
| 138 - 267 | N138 | C267 | TFPT_HUMAN - FOXA1- | 1.57407283 | 0.02474032 |
| | | | Nterm | | |
| 284 - 320 | N284 | C320 | p65-HSF1 - mMLL3-SET | 1.57377752 | 7.18E−05 |
| 189 - 14 | N189 | C14 | ING2_HUMAN - RELA | 1.57139325 | 0.00805675 |
| 285 - 131 | N285 | C131 | p65-MyoD - | 1.57027792 | 0.04492886 |
| | | | PRDM2_HUMAN | | |
| 284 - 326 | N284 | C326 | p65-HSF1 - pf_Sir2A | 1.56954065 | 0.00312923 |
| 88 - 265 | N88 | C265 | ING4_HUMAN - EZH2 | 1.56946612 | 0.03766659 |
| 306 - 285 | N306 | C285 | CobB - p65-MyoD | 1.5685524 | 5.45E−04 |
| 281 - 192 | N281 | C192 | VP64 - JHD2C_HUMAN | 1.56764892 | 0.00232387 |
| 257 - 14 | N257 | C14 | c-Myb-TAD - RELA | 1.56552121 | 0.00321302 |
| 313 - 54 | N313 | C54 | SaII - CBP_HUMAN | 1.56258862 | 0.04634094 |
| 285 - 222 | N285 | C222 | p65-MyoD - IN80E_HUMAN | 1.56074994 | 1.53E−04 |
| 25 - 285 | N25 | C285 | CTCFL - p65-MyoD | 1.55999856 | 7.76E−04 |
| 282 - 277 | N282 | C277 | VP160 - SUV39H1 | 1.55988342 | 0.00768639 |
| 14 - 332 | N14 | C332 | RELA - hs_SIRT5 | 1.55968372 | 0.00402291 |
| 110 - 285 | N110 | C285 | JMJD4_HUMAN - p65- | 1.55953256 | 0.01422648 |
| | | | MyoD | | |
| 14 - 288 | N14 | C288 | RELA - SUV[SET] | 1.5569087 | 6.42E−04 |
| 216 - 250 | N216 | C250 | ING5_HUMAN - | 1.55592826 | 0.01303981 |
| | | | HCFC1_HUMAN | | |
| 27 - 252 | N27 | C252 | EZH1 - HCFC1_HUMAN | 1.55531432 | 0.01050977 |
| 285 - 294 | N285 | C294 | p65-MyoD - DIM5 | 1.55523886 | 4.76E−04 |
| 59 - 63 | N59 | C63 | INO80_HUMAN - | 1.55404628 | 0.01110993 |
| | | | SMYD5_HUMAN | | |
| 253 - 136 | N253 | C136 | RBBP5_HUMAN - | 1.55311702 | 0.03878908 |
| | | | JMJD6_HUMAN | | |
| 328 - 284 | N328 | C284 | hs_PHF19 - p65-HSF1 | 1.55222662 | 0.00107071 |
| 35 - 301 | N35 | C301 | VPR_JKNp64 - SIRT3 | 1.55168331 | 0.00536336 |
| 284 - 301 | N284 | C301 | p65-HSF1 - SIRT3 | 1.55134381 | 7.91E−04 |
| 285 - 34 | N285 | C34 | p65-MyoD - DNMT3A- | 1.55122556 | 0.00513144 |
| | | | 3L_JKNp112 | | |
| 255 - 281 | N255 | C281 | EED_HUMAN - VP64 | 1.55112044 | 0.00140195 |
| 35 - 7 | N35 | C7 | VPR_JKNp64 - ZNF10 | 1.55056823 | 0.01193903 |
| 284 - 273 | N284 | C273 | p65-HSF1 - PRDM9-CD | 1.54956461 | 0.00225928 |
| 269 - 257 | N269 | C257 | PU.1 - c-Myb-TAD | 1.5494266 | 0.00145299 |
| 284 - 26 | N284 | C26 | p65-HSF1 - EZH1 | 1.5493312 | 8.44E−04 |
| 252 - 85 | N252 | C85 | HCFC1_HUMAN - | 1.5490757 | 0.02633885 |
| | | | SMYD3_HUMAN | | |
| 144 - 14 | N144 | C14 | UTY_HUMAN - RELA | 1.54711201 | 0.00105905 |
| 281 - 276 | N281 | C276 | VP64 - EHMT2(G9a)-Cterm | 1.54500102 | 4.44E−05 |
| 35 - 127 | N35 | C127 | VPR_JKNp64 - | 1.5446824 | 0.00116845 |
| | | | SETMR_HUMAN | | |
| 257 - 284 | N257 | C284 | c-Myb-TAD - p65-HSF1 | 1.54374851 | 0.00251784 |
| 280 - 291 | N280 | C291 | CBF1-Cterm - SS18 | 1.54354187 | 1.90E−04 |
| 14 - 25 | N14 | C25 | RELA - CTCFL | 1.54255671 | 0.01494911 |
| 171 - 281 | N171 | C281 | ASH1L_HUMAN - VP64 | 1.54248753 | 0.00188905 |
| 226 - 257 | N226 | C257 | SETD2_HUMAN - c-Myb- | 1.5417356 | 4.92E−04 |
| | | | TAD | | |
| 269 - 284 | N269 | C284 | PU.1 - p65-HSF1 | 1.54139364 | 4.95E−05 |
| 268 - 14 | N268 | C14 | FOXA1-Cterm - RELA | 1.5378363 | 5.62E−05 |
| 14 - 275 | N14 | C275 | RELA - DOT1L- | 1.53705987 | 8.84E−04 |
| | | | DOT1_domain | | |
| 281 - 137 | N281 | C137 | VP64 - DNM3B_HUMAN | 1.53669325 | 0.00558619 |
| 212 - 263 | N212 | C263 | HDAC8_HUMAN - HDAC8 | 1.53647717 | 0.04662314 |
| 34 - 281 | N34 | C281 | DNMT3A-3L_JKNp112 - | 1.53590499 | 0.02534546 |
| | | | VP64 | | |
| 63 - 284 | N63 | C284 | SMYD5_HUMAN - p65- | 1.53583403 | 0.00135382 |
| | | | HSF1 | | |
| 14 - 305 | N14 | C305 | RELA - NUE | 1.53528625 | 1.34E−04 |
| 285 - 300 | N285 | C300 | p65-MyoD - SIRT6 | 1.53345889 | 8.19E−04 |
| 105 - 35 | N105 | C35 | MBD2_HUMAN - | 1.53326515 | 0.01880264 |
| | | | VPR_JKNp64 | | |
| 252 - 250 | N252 | C250 | HCFC1_HUMAN - | 1.53174409 | 0.00906374 |
| | | | HCFC1_HUMAN | | |
| 123 - 285 | N123 | C285 | KMT2A_HUMAN - p65- | 1.53169304 | 0.0030248 |
| | | | MyoD | | |
| 15 - 290 | N15 | C290 | EP300 - HP1aCS | 1.53017234 | 0.00131846 |
| 14 - 176 | N14 | C176 | RELA - ARP8_HUMAN | 1.52907944 | 0.00464082 |
| 282 - 60 | N282 | C60 | VP160 - INO80_HUMAN | 1.52603533 | 0.00860228 |
| 285 - 149 | N285 | C149 | p65-MyoD - MBD1_HUMAN | 1.5255623 | 0.00300666 |
| 306 - 10 | N306 | C10 | CobB - HSF1 | 1.52515453 | 0.00420098 |
| 344 - 83 | N344 | C83 | HDAC3_HUMAN - | 1.52404303 | 0.0458854 |
| | | | SMRD3_HUMAN | | |
| 14 - 294 | N14 | C294 | RELA - DIM5 | 1.52301593 | 3.71E−04 |
| 14 - 14 | N14 | C14 | RELA - RELA | 1.52216375 | 8.45E−05 |
| 1 - 285 | N1 | C285 | SETD1A - p65-MyoD | 1.52179859 | 0.01203858 |
| 14 - 198 | N14 | C198 | RELA - JMJD8_HUMAN | 1.52092846 | 2.67E−04 |
| 285 - 162 | N285 | C162 | p65-MyoD - SIR6_HUMAN | 1.52055289 | 5.05E−04 |
| 285 - 349 | N285 | C349 | p65-MyoD - DAM_N132A | 1.52039546 | 5.03E−04 |
| 85 - 10 | N85 | C10 | SMYD3_HUMAN - HSF1 | 1.52037052 | 0.00431254 |
| 35 - 97 | N35 | C97 | VPR_JKNp64 - | 1.51840767 | 0.01215036 |
| | | | CHD3_HUMAN | | |
| 74 - 20 | N74 | C20 | KDM5D_HUMAN - KDM1B | 1.51801672 | 0.0202217 |
| 257 - 291 | N257 | C291 | c-Myb-TAD - SS18 | 1.51616676 | 0.00808347 |
| 281 - 170 | N281 | C170 | VP64 - NSD1_HUMAN | 1.51577299 | 0.03237031 |
| 114 - 257 | N114 | C257 | KDM5A_HUMAN - c-Myb- | 1.51523787 | 6.79E−04 |
| | | | TAD | | |
| 282 - 159 | N282 | C159 | VP160 - SETB1_HUMAN | 1.51464827 | 2.71E−04 |
| 298 - 281 | N298 | C281 | MBD2b - VP64 | 1.51282906 | 0.00399473 |
| 25 - 250 | N25 | C250 | CTCFL - HCFC1_HUMAN | 1.51000871 | 0.00305979 |
| 35 - 73 | N35 | C73 | VPR_JKNp64 - | 1.50955712 | 1.15E−04 |
| | | | KDM5D_HUMAN | | |
| 18 - 257 | N18 | C257 | EZH2 - c-Myb-TAD | 1.50753455 | 7.55E−04 |
| 285 - 216 | N285 | C216 | p65-MyoD - ING5_HUMAN | 1.50730534 | 0.01900918 |
| 285 - 118 | N285 | C118 | p65-MyoD - | 1.5063705 | 0.00159123 |
| | | | HDAC6_HUMAN | | |
| 35 - 120 | N35 | C120 | VPR_JKNp64 - | 1.50545472 | 0.01242782 |
| | | | RING1_HUMAN | | |
| 285 - 330 | N285 | C330 | p65-MyoD - ce_Set1 | 1.50336663 | 0.00183188 |
| 5 - 280 | N5 | C280 | KDM1A - CBF1-Cterm | 1.50319364 | 0.03392263 |
| 239 - 176 | N239 | C176 | KDM8_HUMAN - | 1.50216757 | 0.04233754 |
| | | | ARP8_HUMAN | | |
| 273 - 107 | N273 | C107 | PRDM9-CD - | 1.50047056 | 0.03529449 |
| | | | KAT6B_HUMAN | | |
| 14 - 290 | N14 | C290 | RELA - HP1aCS | 1.50012614 | 3.95E−05 |
| 330 - 281 | N330 | C281 | ce_Set1 - VP64 | 1.49938209 | 0.01044499 |
| 245 - 285 | N245 | C285 | TYY1_HUMAN - p65-MyoD | 1.49934092 | 0.00179762 |
| 83 - 273 | N83 | C273 | SMRD3_HUMAN - PRDM9- | 1.49932717 | 0.03694386 |
| | | | CD | | |
| 194 - 14 | N194 | C14 | ING1_HUMAN - RELA | 1.49923731 | 0.00300279 |
| 119 - 281 | N119 | C281 | ACL6A_HUMAN - VP64 | 1.49855159 | 0.00672128 |
| 85 - 284 | N85 | C284 | SMYD3_HUMAN - p65- | 1.49751144 | 0.02060346 |
| | | | HSF1 | | |
| 73 - 14 | N73 | C14 | KDM5D_HUMAN - RELA | 1.49498269 | 8.40E−05 |
| 135 - 14 | N135 | C14 | KDM2A_HUMAN - RELA | 1.49454647 | 0.01537235 |
| 285 - 240 | N285 | C240 | p65-MyoD - ANM3_HUMAN | 1.49407286 | 0.0424921 |
| 285 - 329 | N285 | C329 | p65-MyoD - pbcv1_vSET | 1.49299934 | 8.44E−04 |
| 281 - 132 | N281 | C132 | VP64 - PRDM2_HUMAN | 1.49224771 | 0.00176866 |
| 118 - 285 | N118 | C285 | HDAC6_HUMAN - p65- | 1.49204918 | 0.02441575 |
| | | | MyoD | | |
| 99 - 285 | N99 | C285 | IN80C_HUMAN - p65-MyoD | 1.49201866 | 0.00427285 |
| 237 - 162 | N237 | C162 | KAT5_HUMAN - | 1.49166806 | 0.01728096 |
| | | | SIR6_HUMAN | | |
| 164 - 285 | N164 | C285 | SIR5_HUMAN - p65-MyoD | 1.49054212 | 0.03914761 |
| 258 - 250 | N258 | C250 | Nejire-HAT - | 1.49037671 | 0.02344268 |
| | | | HCFC1_HUMAN | | |
| 300 - 10 | N300 | C10 | SIRT6 - HSF1 | 1.48888622 | 0.00383335 |
| 271 - 35 | N271 | C35 | GCN5 - VPR_JKNp64 | 1.48801463 | 0.03083084 |
| 218 - 118 | N218 | C118 | IN80B_HUMAN - | 1.48795715 | 0.01848906 |
| | | | HDAC6_HUMAN | | |
| 53 -281 | N53 | C281 | CBP_HUMAN - VP64 | 1.4876408 | 1.09E−04 |
| 5 - 257 | N5 | C257 | KDM1A - c-Myb-TAD | 1.48682905 | 0.01417685 |
| 285 - 271 | N285 | C271 | p65-MyoD - GCN5 | 1.48662533 | 0.00219323 |
| 134 - 250 | N134 | C250 | KDM2A_HUMAN - | 1.48443 | 0.04987611 |
| | | | HCFC1_HUMAN | | |
| 35 - 26 | N35 | C26 | VPR_JKNp64 - EZH1 | 1.48420774 | 8.90E−04 |
| 250 - 212 | N250 | C212 | HCFC1_HUMAN - | 1.48255878 | 0.01980637 |
| | | | HDAC8_HUMAN | | |
| 14 - 333 | N14 | C333 | RELA - sc_RPD3 | 1.48077513 | 0.01110163 |
| 48 - 35 | N48 | C35 | SETD5_HUMAN - | 1.48053238 | 1.79E−04 |
| | | | VPR_JKNp64 | | |
| 282 - 344 | N282 | C344 | VP160 - HDAC3_HUMAN | 1.47956399 | 2.97E−04 |
| 284 - 79 | N284 | C79 | p65-HSF1 - | 1.47937717 | 0.00564387 |
| | | | KAT6A_HUMAN | | |
| 26 - 284 | N26 | C284 | EZH1 - p65-HSF1 | 1.47819365 | 0.002487 |
| 63 - 14 | N63 | C14 | SMYD5_HUMAN - RELA | 1.47708024 | 0.00154289 |
| 300 - 284 | N300 | C284 | SIRT6 - p65-HSF1 | 1.47256103 | 0.02141407 |
| 14 - 8 | N14 | C8 | RELA - MECP2 | 1.46994754 | 4.55E−04 |
| 277 - 280 | N277 | C280 | SUV39H1 - CBF1-Cterm | 1.46986191 | 0.01338292 |
| 275 - 14 | N275 | C14 | DOT1L-DOT1_domain - | 1.46927788 | 0.03054786 |
| | | | RELA | | |
| 207 - 257 | N207 | C257 | KDM1B_HUMAN - c-Myb- | 1.46823706 | 0.00217306 |
| | | | TAD | | |
| 18 - 285 | N18 | C285 | EZH2 - p65-MyoD | 1.46769001 | 0.04023973 |
| 285 - 100 | N285 | C100 | p65-MyoD - TAF1_HUMAN | 1.46688171 | 0.00234777 |
| 83 - 2 | N83 | C2 | SMRD3_HUMAN - SETD1A | 1.46554009 | 0.03043132 |
| 284 - 271 | N284 | C271 | p65-HSF1 - GCN5 | 1.46534595 | 0.00282377 |
| 285 - 26 | N285 | C26 | p65-MyoD - EZH1 | 1.46424914 | 1.19E−04 |
| 178 - 285 | N178 | C285 | KMT5C_HUMAN - p65- | 1.46291428 | 0.01978442 |
| | | | MyoD | | |
| 282 - 322 | N282 | C322 | VP160 - MINT_HUMAN | 1.46274344 | 0.00266918 |
| 285 - 229 | N285 | C229 | p65-MyoD - | 1.46204123 | 9.21E−04 |
| | | | NFRKB_HUMAN | | |
| 66 - 145 | N66 | C145 | DOT1L_HUMAN - | 1.4616684 | 0.04025443 |
| | | | UTY_HUMAN | | |
| 285 - 22 | N285 | C22 | p65-MyoD - TET1 | 1.45967829 | 0.00768301 |
| 327 - 132 | N327 | C132 | hs_NIPP1 - | 1.45925219 | 0.01893385 |
| | | | PRDM2_HUMAN | | |
| 281 - 6 | N281 | C6 | VP64 - KDM1A | 1.45886179 | 0.01642142 |
| 113 - 285 | N113 | C285 | KDM5A_HUMAN - p65- | 1.45704928 | 0.00570373 |
| | | | MyoD | | |
| 88 - 14 | N88 | C14 | ING4_HUMAN - RELA | 1.45640559 | 7.67E−04 |
| 1 - 291 | N1 | C291 | SETD1A - SS18 | 1.4562791 | 0.00110255 |
| 285 - 313 | N285 | C313 | p65-MyoD - SaII | 1.45519801 | 0.00333286 |
| 335 - 284 | N335 | C284 | at_SUVR4 - p65-HSF1 | 1.45508492 | 0.02096935 |
| 282 - 230 | N282 | C230 | VP160 - NFRKB_HUMAN | 1.454641 | 0.01585528 |
| 285 - 272 | N285 | C272 | p65-MyoD - Sir2 | 1.45382272 | 3.01E−04 |
| 285 - 143 | N285 | C143 | p65-MyoD - | 1.45265198 | 0.02493775 |
| | | | KDM4B_HUMAN | | |
| 282 - 200 | N282 | C200 | VP160 - ARP5_HUMAN | 1.45189008 | 0.00695816 |
| 348 - 42 | N348 | C42 | BRDT_HUMAN - | 1.45059008 | 0.0400898 |
| | | | KMT2E_HUMAN | | |
| 12 - 35 | N12 | C35 | KMT2A - VPR_JKNp64 | 1.44922331 | 0.01668561 |
| 76 - 285 | N76 | C285 | KDM5D_HUMAN - p65- | 1.44696202 | 0.04459597 |
| | | | MyoD | | |
| 149 - 284 | N149 | C284 | MBD1_HUMAN - p65-HSF1 | 1.44609667 | 0.01069147 |
| 281 - 23 | N281 | C23 | VP64 - TET1 | 1.44572924 | 0.01909298 |
| 14 - 113 | N14 | C113 | RELA - KDM5A_HUMAN | 1.44530943 | 0.0250035 |
| 35 - 326 | N35 | C326 | VPR_JKNp64 - pf_Sir2A | 1.44489311 | 0.01429182 |
| 200 - 136 | N200 | C136 | ARP5_HUMAN - | 1.44465797 | 0.01290881 |
| | | | JMJD6_HUMAN | | |
| 245 - 14 | N245 | C14 | TYY1_HUMAN - RELA | 1.44446967 | 0.04963582 |
| 297 - 214 | N297 | C214 | MesoLo - HDAC4_HUMAN | 1.4433783 | 0.04085094 |
| 332 - 10 | N332 | C10 | hs_SIRT5 - HSF1 | 1.44308899 | 0.00106633 |
| 38 - 266 | N38 | C266 | EHMT2 - GADD45A | 1.44300684 | 0.00939172 |
| 294 - 35 | N294 | C35 | DIM5 - VPR_JKNp64 | 1.44231491 | 0.04680469 |
| 253 - 281 | N253 | C281 | RBBP5_HUMAN - VP64 | 1.44224082 | 0.02492022 |
| 281 - 70 | N281 | C70 | VP64 - SETB2_HUMAN | 1.44221894 | 0.01381088 |
| 14 - 266 | N14 | C266 | RELA - GADD45A | 1.44214823 | 1.30E−04 |
| 25 - 14 | N25 | C14 | CTCFL - RELA | 1.442076 | 0.00314028 |
| 281 - 182 | N281 | C182 | VP64 - MTA3_HUMAN | 1.44152218 | 0.00623305 |
| 258 - 106 | N258 | C106 | Nejire-HAT - | 1.44140429 | 0.00871414 |
| | | | KAT6B_HUMAN | | |
| 285 - 20 | N285 | C20 | p65-MyoD - KDM1B | 1.44117144 | 0.00457481 |
| 284 - 107 | N284 | C107 | p65-HSF1 - | 1.44053915 | 0.00968076 |
| | | | KAT6B_HUMAN | | |
| 301 - 285 | N301 | C285 | SIRT3 - p65-MyoD | 1.43992607 | 0.00308223 |
| 252 - 233 | N252 | C233 | HCFC1_HUMAN - | 1.43887499 | 0.00519119 |
| | | | HINFP_HUMAN | | |
| 336 - 35 | N336 | C35 | ce_Set4 - VPR_JKNp64 | 1.4384217 | 0.00994079 |
| 309 - 35 | N309 | C35 | HDAC8 - VPR_JKNp64 | 1.43711775 | 0.00573444 |
| 66 - 99 | N66 | C99 | DOT1L_HUMAN - | 1.43296008 | 0.02522957 |
| | | | IN80C_HUMAN | | |
| 284 - 324 | N284 | C324 | p65-HSF1 - LBR_HUMAN | 1.43272147 | 5.99E−04 |
| 35 - 212 | N35 | C212 | VPR_JKNp64 - | 1.43132191 | 0.02016517 |
| | | | HDAC8_HUMAN | | |
| 281 - 93 | N281 | C93 | VP64 - ACL6B_HUMAN | 1.43124774 | 0.02165089 |
| 190 - 252 | N190 | C252 | ANM1_HUMAN - | 1.43024434 | 0.03972577 |
| | | | HCFC1_HUMAN | | |
| 296 - 285 | N296 | C285 | HP1a - p65-MyoD | 1.42970221 | 0.0050608 |
| 285 - 155 | N285 | C155 | p65-MyoD - NSD2_HUMAN | 1.42948205 | 0.01651686 |
| 281 - 302 | N281 | C302 | VP64 - HDAC11 | 1.4286805 | 2.50E−04 |
| 69 - 14 | N69 | C14 | SETB2_HUMAN - RELA | 1.42828357 | 2.65E−04 |
| 346 - 1 | N346 | C1 | MSX2_HUMAN - SETD1A | 1.4282463 | 0.03474915 |
| 281 - 231 | N281 | C231 | VP64 - KDM5B_HUMAN | 1.42700995 | 8.84E−04 |
| 345 - 14 | N345 | C14 | MSX2_HUMAN - RELA | 1.42683357 | 0.02857723 |
| 285 - 53 | N285 | C53 | p65-MyoD - CBP_HUMAN | 1.42676556 | 0.00464202 |
| 275 - 252 | N275 | C252 | DOT1L-DOT1_domain - | 1.42674789 | 0.01867902 |
| | | | HCFC1_HUMAN | | |
| 14 - 50 | N14 | C50 | RELA - NCOA1_HUMAN | 1.42634512 | 0.00642213 |
| 14 - 95 | N14 | C95 | RELA - SETD6_HUMAN | 1.42542863 | 0.02881434 |
| 299 - 285 | N299 | C285 | HST2 - p65-MyoD | 1.42541864 | 0.00313632 |
| 138 - 285 | N138 | C285 | TFPT_HUMAN - p65-MyoD | 1.42524662 | 0.00687258 |
| 284 - 182 | N284 | C182 | p65-HSF1 - MTA3_HUMAN | 1.42413613 | 0.04848941 |
| 242 - 267 | N242 | C267 | KAT2B_HUMAN - FOXA1- | 1.42374531 | 0.04466929 |
| | | | Nterm | | |
| 287 - 217 | N287 | C217 | G9A[SET] - | 1.42358584 | 0.03920295 |
| | | | CHRC1_HUMAN | | |
| 285 - 308 | N285 | C308 | p65-MyoD - HDT1 | 1.42289852 | 0.04394723 |
| 346 - 291 | N346 | C291 | MSX2_HUMAN - SS18 | 1.42011109 | 0.00171065 |
| 212 - 199 | N212 | C199 | HDAC8_HUMAN - | 1.41971467 | 0.02221756 |
| | | | ING3_HUMAN | | |
| 134 - 257 | N134 | C257 | KDM2A_HUMAN - c-Myb- | 1.41820141 | 0.00698328 |
| | | | TAD | | |
| 154 - 186 | N154 | C186 | NSD2_HUMAN - | 1.41781723 | 0.03855592 |
| | | | RIOX2_HUMAN | | |
| 78 - 335 | N78 | C335 | KAT6A_HUMAN - | 1.41711367 | 0.00855432 |
| | | | at_SUVR4 | | |
| 59 - 10 | N59 | C10 | INO80_HUMAN - HSF1 | 1.41666427 | 0.00628699 |
| 35 - 295 | N35 | C295 | VPR_JKNp64 - SET-TAF1B | 1.41665484 | 0.00393259 |
| 272 - 284 | N272 | C284 | Sir2 - p65-HSF1 | 1.41529334 | 0.024521 |
| 282 - 254 | N282 | C254 | VP160 - MEN1_HUMAN | 1.41474093 | 0.00452283 |
| 252 - 327 | N252 | C327 | HCFC1_HUMAN - hs_NIPP1 | 1.41395112 | 0.00902051 |
| 258 - 306 | N258 | C306 | Nejire-HAT - CobB | 1.4136598 | 0.01694461 |
| 91 - 326 | N91 | C326 | ANM5_HUMAN - pf_Sir2A | 1.41245654 | 0.01907974 |
| 7 - 281 | N7 | C281 | ZNF10 - VP64 | 1.41224932 | 0.03207402 |
| 282 - 72 | N282 | C72 | VP160 - KDM7A_HUMAN | 1.41200951 | 0.00128603 |
| 14 - 78 | N14 | C78 | RELA - KAT6A_HUMAN | 1.41189021 | 0.00443422 |
| 218 - 251 | N218 | C251 | IN80B_HUMAN - | 1.41188433 | 0.03120802 |
| | | | HCFC1_HUMAN | | |
| 20 - 281 | N20 | C281 | KDM1B - VP64 | 1.41161308 | 3.60E−04 |
| 281 - 238 | N281 | C238 | VP64 - NAT10_HUMAN | 1.41141674 | 0.01838922 |
| 154 - 333 | N154 | C333 | NSD2_HUMAN - sc_RPD3 | 1.41094782 | 0.01783571 |
| 189 - 257 | N189 | C257 | ING2_HUMAN - c-Myb- | 1.41046766 | 9.51E−04 |
| | | | TAD | | |
| 252 - 25 | N252 | C25 | HCFC1_HUMAN - CTCFL | 1.4104196 | 0.01246308 |
| 14 - 270 | N14 | C270 | RELA - ENL | 1.41000357 | 0.00222564 |
| 154 - 257 | N154 | C257 | NSD2_HUMAN - c-Myb- | 1.4096785 | 0.00312973 |
| | | | TAD | | |
| 282 - 196 | N282 | C196 | VP160 - MTA1_HUMAN | 1.40935538 | 0.02898047 |
| 282 - 79 | N282 | C79 | VP160 - KAT6A_HUMAN | 1.40899064 | 2.70E−04 |
| 233 - 14 | N233 | C14 | HINFP_HUMAN - RELA | 1.40789714 | 0.00233503 |
| 282 - 170 | N282 | C170 | VP160 - NSD1_HUMAN | 1.40723635 | 0.01399653 |
| 14 - 229 | N14 | C229 | RELA - NFRKB_HUMAN | 1.40697348 | 1.89E−04 |
| 14 - 98 | N14 | C98 | RELA - A4FU79_HUMAN | 1.40644841 | 2.16E−04 |
| 25 - 10 | N25 | C10 | CTCFL - HSF1 | 1.40430235 | 0.01648079 |
| 281 - 55 | N281 | C55 | VP64 - RBBP4_HUMAN | 1.40186512 | 0.00045126 |
| 14 - 63 | N14 | C63 | RELA - SMYD5_HUMAN | 1.40184944 | 0.00213421 |
| 151 - 257 | N151 | C257 | SIR7_HUMAN - c-Myb-TAD | 1.40110474 | 1.02E−04 |
| 35 - 348 | N35 | C348 | VPR_JKNp64 - | 1.40080706 | 0.04945669 |
| | | | BRDT_HUMAN | | |
| 313 - 291 | N313 | C291 | SaII - SS18 | 1.40063462 | 0.00701428 |
| 130 - 284 | N130 | C284 | PRDM2_HUMAN - p65- | 1.39945461 | 0.01980963 |
| | | | HSF1 | | |
| 97 - 281 | N97 | C281 | CHD3_HUMAN - VP64 | 1.39894767 | 0.01516921 |
| 205 - 14 | N205 | C14 | JMJD7_HUMAN - RELA | 1.39780826 | 5.94E−04 |
| 281 - 202 | N281 | C202 | VP64 - KDM2B_HUMAN | 1.39751189 | 5.80E−04 |
| 346 - 285 | N346 | C285 | MSX2_HUMAN - p65-MyoD | 1.39711038 | 0.00533335 |
| 284 - 145 | N284 | C145 | p65-HSF1 - UTY_HUMAN | 1.39683044 | 0.03449043 |
| 35 - 136 | N35 | C136 | VPR_JKNp64 - | 1.39629679 | 0.02506347 |
| | | | JMJD6_HUMAN | | |
| 77 - 257 | N77 | C257 | KDM5D_HUMAN - c-Myb- | 1.39537976 | 0.00240864 |
| | | | TAD | | |
| 114 - 10 | N114 | C10 | KDM5A_HUMAN - HSF1 | 1.39494976 | 0.01541978 |
| 271 - 38 | N271 | C38 | GCN5 - EHMT2 | 1.39396707 | 0.04155336 |
| 291 - 14 | N291 | C14 | SS18 - RELA | 1.39304089 | 0.03829349 |
| 285 - 21 | N285 | C21 | p65-MyoD - KDM1B | 1.39202055 | 0.02780167 |
| 285 - 76 | N285 | C76 | p65-MyoD - | 1.39198467 | 0.01278481 |
| | | | KDM5D_HUMAN | | |
| 285 - 296 | N285 | C296 | p65-MyoD - HP1a | 1.39191763 | 0.00188028 |
| 188 - 199 | N188 | C199 | SMYD4_HUMAN - | 1.39129137 | 0.04663814 |
| | | | ING3_HUMAN | | |
| 239 - 200 | N239 | C200 | KDM8_HUMAN - | 1.39109327 | 0.04797224 |
| | | | ARP5_HUMAN | | |
| 252 - 269 | N252 | C269 | HCFC1_HUMAN - PU.1 | 1.38981561 | 0.00406851 |
| 174 - 218 | N174 | C218 | HDAC1_HUMAN - | 1.38957713 | 0.02038141 |
| | | | IN80B_HUMAN | | |
| 281 - 191 | N281 | C191 | VP64 - CDYL_HUMAN | 1.38928955 | 0.00406054 |
| 315 - 285 | N315 | C285 | NCoR - p65-MyoD | 1.38918722 | 9.02E−04 |
| 281 - 109 | N281 | C109 | VP64 - KAT6B_HUMAN | 1.3887011 | 0.02813421 |
| 284 - 330 | N284 | C330 | p65-HSF1 - ce_Set1 | 1.38813758 | 0.00156605 |
| 61 - 14 | N61 | C14 | HAT1_HUMAN - RELA | 1.38769422 | 0.02523724 |
| 297 - 284 | N297 | C284 | MesoLo - p65-HSF1 | 1.38717861 | 0.02432778 |
| 217 - 339 | N217 | C339 | CHRC1_HUMAN - | 1.38712554 | 0.02129277 |
| | | | SPOC1_HUMAN | | |
| 282 - 155 | N282 | C155 | VP160 - NSD2_HUMAN | 1.38640868 | 0.00774988 |
| 83 - 257 | N83 | C257 | SMRD3_HUMAN - c-Myb- | 1.38458952 | 1.91E−04 |
| | | | TAD | | |
| 217 - 284 | N217 | C284 | CHRC1_HUMAN - p65- | 1.3844553 | 0.00314841 |
| | | | HSF1 | | |
| 14 - 301 | N14 | C301 | RELA - SIRT3 | 1.38368007 | 9.94E−04 |
| 14 - 319 | N14 | C319 | RELA - mCBP-HAT | 1.38357966 | 0.03282103 |
| 285 - 187 | N285 | C187 | p65-MyoD - MBD4_HUMAN | 1.38326982 | 0.00178139 |
| 32 - 33 | N32 | C33 | Gilbert_Lab_CRISPRi_KRAB - | 1.38303773 | 0.02861312 |
| | | | Hilton_2015 | | |
| 285 - 37 | N285 | C37 | p65-MyoD - KDM6A | 1.38186644 | 0.00814501 |
| 250 - 77 | N250 | C77 | HCFC1_HUMAN - | 1.38121585 | 0.03022882 |
| | | | KDM5D_HUMAN | | |
| 285 - 307 | N285 | C307 | p65-MyoD - KYP | 1.37978947 | 2.30E−04 |
| 14 - 12 | N14 | C12 | RELA - KMT2A | 1.37946107 | 0.00105643 |
| 285 - 214 | N285 | C214 | p65-MyoD - | 1.37921509 | 0.0103919 |
| | | | HDAC4_HUMAN | | |
| 107 - 35 | N107 | C35 | KAT6B_HUMAN - | 1.37805554 | 0.0294667 |
| | | | VPR_JKNp64 | | |
| 8 - 35 | N8 | C35 | MECP2 - VPR_JKNp64 | 1.37756205 | 0.01619403 |
| 15 - 250 | N15 | C250 | EP300 - HCFC1_HUMAN | 1.37746649 | 0.01259343 |
| 116 - 78 | N116 | C78 | ANM6_HUMAN - | 1.37735124 | 0.01968566 |
| | | | KAT6A_HUMAN | | |
| 38 - 333 | N38 | C333 | EHMT2 - sc_RPD3 | 1.37581811 | 0.01794391 |
| 114 - 269 | N114 | C269 | KDM5A_HUMAN - PU.1 | 1.37565995 | 0.04677597 |
| 212 - 257 | N212 | C257 | HDAC8_HUMAN - c-Myb- | 1.37435417 | 2.24E−04 |
| | | | TAD | | |
| 327 - 77 | N327 | C77 | hs_NIPP1 - | 1.37405394 | 0.02842579 |
| | | | KDM5D_HUMAN | | |
| 281 - 75 | N281 | C75 | VP64 - KDM5D_HUMAN | 1.37358689 | 0.00230994 |
| 282 - 309 | N282 | C309 | VP160 - HDAC8 | 1.37259268 | 0.01095896 |
| 297 - 14 | N297 | C14 | MesoLo - RELA | 1.37001376 | 0.00124942 |
| 35 - 111 | N35 | C111 | VPR_JKNp64 - | 1.36994891 | 0.00121177 |
| | | | KDM5A_HUMAN | | |
| 35 - 305 | N35 | C305 | VPR_JKNp64 - NUE | 1.36904442 | 4.30E−04 |
| 65 - 226 | N65 | C226 | MCRS1_HUMAN - | 1.36884593 | 0.04303744 |
| | | | SETD2_HUMAN | | |
| 143 - 257 | N143 | C257 | KDM4B_HUMAN - c-Myb- | 1.36846084 | 0.01145761 |
| | | | TAD | | |
| 231 - 280 | N231 | C280 | KDM5B_HUMAN - CBF1- | 1.36842317 | 0.0195752 |
| | | | Cterm | | |
| 212 - 317 | N212 | C317 | HDAC8_HUMAN - HDAC3 | 1.3672855 | 0.02391772 |
| 189 - 281 | N189 | C281 | ING2_HUMAN - VP64 | 1.36574136 | 0.0025137 |
| 282 - 152 | N282 | C152 | VP160 - CDY1_HUMAN | 1.36405631 | 0.0022828 |
| 285 - 114 | N285 | C114 | p65-MyoD - | 1.36392555 | 0.04485262 |
| | | | KDM5A_HUMAN | | |
| 88 -200 | N88 | C200 | ING4_HUMAN - | 1.36369069 | 0.01821144 |
| | | | ARP5_HUMAN | | |
| 189 - 284 | N189 | C284 | ING2_HUMAN - p65-HSF1 | 1.36362736 | 0.02166561 |
| 233 - 280 | N233 | C280 | HINFP_HUMAN - CBF1- | 1.3636016 | 0.00812802 |
| | | | Cterm | | |
| 218 - 14 | N218 | C14 | IN80B_HUMAN - RELA | 1.36192882 | 0.04737697 |
| 99 - 264 | N99 | C264 | IN80C_HUMAN - | 1.36145584 | 0.04693328 |
| | | | MTL5_HUMAN | | |
| 120 - 281 | N120 | C281 | RING1_HUMAN - VP64 | 1.36063433 | 0.00313648 |
| 285 - 173 | N285 | C173 | p65-MyoD - SIR4_HUMAN | 1.35742821 | 0.00563096 |
| 311 - 281 | N311 | C281 | Sin3a - VP64 | 1.35714977 | 0.00605773 |
| 220 - 285 | N220 | C285 | JARD2_HUMAN - p65- | 1.35714124 | 5.21E−04 |
| | | | MyoD | | |
| 295 - 1 | N295 | C1 | SET-TAF1B - SETD1A | 1.35675388 | 0.00381267 |
| 35 - 75 | N35 | C75 | VPR_JKNp64 - | 1.35461193 | 0.0282864 |
| | | | KDM5D_HUMAN | | |
| 282 - 91 | N282 | C91 | VP160 - ANM5_HUMAN | 1.35277754 | 0.00871209 |
| 174 - 14 | N174 | C14 | HDAC1_HUMAN - RELA | 1.35254333 | 0.03665257 |
| 204 - 284 | N204 | C284 | HIFIN_HUMAN - p65-HSF1 | 1.35168551 | 0.0297856 |
| 99 - 14 | N99 | C14 | IN80C_HUMAN - RELA | 1.35074263 | 0.00133935 |
| 285 - 311 | N285 | C311 | p65-MyoD - Sin3a | 1.34569137 | 0.00198258 |
| 284 - 287 | N284 | C287 | p65-HSF1 - G9A[SET] | 1.34534782 | 0.00292155 |
| 235 - 284 | N235 | C284 | CARM1_HUMAN - p65- | 1.34458057 | 0.0311949 |
| | | | HSF1 | | |
| 111 - 14 | N111 | C14 | KDM5A_HUMAN - RELA | 1.34347686 | 0.01283457 |
| 281 - 163 | N281 | C163 | VP64 - ANM7_HUMAN | 1.34344502 | 0.04561601 |
| 285 - 274 | N285 | C274 | p65-MyoD - UBE2A | 1.34156977 | 0.01985893 |
| 42 - 285 | N42 | C285 | KMT2E_HUMAN - p65- | 1.34089592 | 0.00682479 |
| | | | MyoD | | |
| 273 - 285 | N273 | C285 | PRDM9-CD - p65-MyoD | 1.34082318 | 0.00195313 |
| 35 - 307 | N35 | C307 | VPR_JKNp64 - KYP | 1.34061311 | 0.01442868 |
| 281 - 131 | N281 | C131 | VP64 - PRDM2_HUMAN | 1.33880279 | 0.04839608 |
| 14- 216 | N14 | C216 | RELA - ING5_HUMAN | 1.33855532 | 0.01280726 |
| 284 - 220 | N284 | C220 | p65-HSF1 - JARD2_HUMAN | 1.33838326 | 0.01030078 |
| 134 - 14 | N134 | C14 | KDM2A_HUMAN - RELA | 1.33761352 | 0.03706062 |
| 253 - 280 | N253 | C280 | RBBP5_HUMAN - CBF1- | 1.3351087 | 0.02102931 |
| | | | Cterm | | |
| 252 - 313 | N252 | C313 | HCFC1_HUMAN - SaII | 1.33484703 | 0.04896993 |
| 257 - 250 | N257 | C250 | c-Myb-TAD - | 1.33421431 | 0.00131015 |
| | | | HCFC1_HUMAN | | |
| 218 - 17 | N218 | C17 | IN80B_HUMAN - EZH2 | 1.33399396 | 0.00945789 |
| 42 - 14 | N42 | C14 | KMT2E_HUMAN - RELA | 1.33350405 | 0.0287274 |
| 10 - 218 | N10 | C218 | HSF1 - IN80B_HUMAN | 1.33333729 | 0.0448695 |
| 63 - 250 | N63 | C250 | SMYD5_HUMAN - | 1.33217239 | 0.00771666 |
| | | | HCFC1_HUMAN | | |
| 285 - 273 | N285 | C273 | p65-MyoD - PRDM9-CD | 1.33144881 | 0.00367688 |
| 267 - 257 | N267 | C257 | FOXA1-Nterm - c-Myb-TAD | 1.33120191 | 0.04485757 |
| 197 - 135 | N197 | C135 | SUV91_HUMAN - | 1.32917393 | 0.04680826 |
| | | | KDM2A_HUMAN | | |
| 246 - 285 | N246 | C285 | ATRX_HUMAN - p65-MyoD | 1.32861373 | 8.50E−04 |
| 71 - 35 | N71 | C35 | RUVB1_HUMAN - | 1.32859677 | 0.03312713 |
| | | | VPR_JKNp64 | | |
| 14 - 47 | N14 | C47 | RELA - ANM2_HUMAN | 1.32812364 | 0.01351396 |
| 35 - 194 | N35 | C194 | VPR_JKNp64 - | 1.32785877 | 0.01532409 |
| | | | ING1_HUMAN | | |
| 218 - 328 | N218 | C328 | IN80B_HUMAN - hs_PHF19 | 1.32657605 | 0.04507431 |
| 35 - 152 | N35 | C152 | VPR_JKNp64 - | 1.32516777 | 0.04517351 |
| | | | CDY1_HUMAN | | |
| 197 - 14 | N197 | C14 | SUV91_HUMAN - RELA | 1.3251165 | 0.01493781 |
| 200 - 87 | N200 | C87 | ARP5_HUMAN - | 1.32447818 | 0.03311752 |
| | | | SETD7_HUMAN | | |
| 14 - 324 | N14 | C324 | RELA - LBR_HUMAN | 1.32341807 | 8.86E−04 |
| 35 - 76 | N35 | C76 | VPR_JKNp64 - | 1.32321989 | 0.00699858 |
| | | | KDM5D_HUMAN | | |
| 348 - 204 | N348 | C204 | BRDT_HUMAN - | 1.32297897 | 0.01718 |
| | | | HIFIN_HUMAN | | |
| 284 - 118 | N284 | C118 | p65-HSF1 - | 1.3222233 | 0.0393834 |
| | | | HDAC6_HUMAN | | |
| 281 - 41 | N281 | C41 | VP64 - SMCA5_HUMAN | 1.32009467 | 0.02906756 |
| 217 - 1 | N217 | C1 | CHRC1_HUMAN - SETD1A | 1.31798982 | 0.01486025 |
| 200 - 69 | N200 | C69 | ARP5_HUMAN - | 1.31783112 | 0.02366878 |
| | | | SETB2_HUMAN | | |
| 111 - 285 | N111 | C285 | KDM5A_HUMAN - p65- | 1.31750008 | 0.0468475 |
| | | | MyoD | | |
| 51 - 35 | N51 | C35 | UCHL5_HUMAN - | 1.31642291 | 0.01577542 |
| | | | VPR_JKNp64 | | |
| 285 - 106 | N285 | C106 | p65-MyoD - | 1.31521237 | 0.00469907 |
| | | | KAT6B_HUMAN | | |
| 151 - 15 | N151 | C15 | SIR7_HUMAN - EP300 | 1.3151133 | 0.02524305 |
| 106 - 257 | N106 | C257 | KAT6B_HUMAN - c-Myb- | 1.31487801 | 0.00530516 |
| | | | TAD | | |
| 246 - 14 | N246 | C14 | ATRX_HUMAN - RELA | 1.31486324 | 0.00455976 |
| 233 - 10 | N233 | C10 | HINFP_HUMAN - HSF1 | 1.31470417 | 0.01675853 |
| 118 - 14 | N118 | C14 | HDAC6_HUMAN - RELA | 1.31054309 | 8.86E−04 |
| 28 - 257 | N28 | C257 | EHMT2 - c-Myb-TAD | 1.30999861 | 0.00794745 |
| 332 - 257 | N332 | C257 | hs_SIRT5 - c-Myb-TAD | 1.30996794 | 0.00785043 |
| 174 - 285 | N174 | C285 | HDAC1_HUMAN - p65- | 1.30830943 | 0.00733429 |
| | | | MyoD | | |
| 246 - 10 | N246 | C10 | ATRX_HUMAN - HSF1 | 1.30736949 | 0.02905818 |
| 98 - 249 | N98 | C249 | A4FU79_HUMAN - HAC1- | 1.30715087 | 0.03504789 |
| | | | HAT | | |
| 136 - 257 | N136 | C257 | JMJD6_HUMAN - c-Myb- | 1.30628371 | 0.00237972 |
| | | | TAD | | |
| 284 - 339 | N284 | C339 | p65-HSF1 - SPOC1_HUMAN | 1.30620557 | 0.02786141 |
| 271 - 87 | N271 | C87 | GCN5 - SETD7_HUMAN | 1.30571613 | 0.04629136 |
| 281 - 108 | N281 | C108 | VP64 - KAT6B_HUMAN | 1.30527161 | 0.02776681 |
| 136 - 14 | N136 | C14 | JMJD6_HUMAN - RELA | 1.30447635 | 0.00655957 |
| 118 - 10 | N118 | C10 | HDAC6_HUMAN - HSF1 | 1.30298197 | 0.02534365 |
| 127 - 281 | N127 | C281 | SETMR_HUMAN - VP64 | 1.30087027 | 0.02166445 |
| 35 - 280 | N35 | C280 | VPR_JKNp64 - CBF1-Cterm | 1.299785 | 0.00881533 |
| 282 - 41 | N282 | C41 | VP160 - SMCA5_HUMAN | 1.29972568 | 0.01206102 |
| 33 - 327 | N33 | C327 | Hilton_2015 - hs_NIPP1 | 1.29963449 | 0.00621345 |
| 284 - 34 | N284 | C34 | p65-HSF1 - DNMT3A- | 1.29943636 | 0.00105472 |
| | | | 3L_JKNp112 | | |
| 204 - 35 | N204 | C35 | HIFIN_HUMAN - | 1.29904482 | 0.03238975 |
| | | | VPR_JKNp64 | | |
| 73 - 10 | N73 | C10 | KDM5D_HUMAN - HSF1 | 1.29773752 | 0.00252424 |
| 14 - 34 | N14 | C34 | RELA - DNMT3A- | 1.29678105 | 4.16E−04 |
| | | | 3L_JKNp112 | | |
| 285 - 336 | N285 | C336 | p65-MyoD - ce_Set4 | 1.29654094 | 0.01762316 |
| 296 - 54 | N296 | C54 | HP1a - CBP_HUMAN | 1.29528981 | 0.00763034 |
| 282 - 51 | N282 | C51 | VP160 - UCHL5_HUMAN | 1.2947998 | 0.00114236 |
| 61 - 284 | N61 | C284 | HAT1_HUMAN - p65-HSF1 | 1.29425781 | 0.02774423 |
| 148 - 284 | N148 | C284 | NSD3_HUMAN - p65-HSF1 | 1.29396738 | 0.01457633 |
| 150 - 99 | N150 | C99 | HDA11_HUMAN - | 1.2933064 | 0.00226719 |
| | | | IN80C_HUMAN | | |
| 67 - 281 | N67 | C281 | PRDM9_HUMAN - VP64 | 1.29260042 | 7.91E−04 |
| 282 - 96 | N282 | C96 | VP160 - CHD3_HUMAN | 1.29193963 | 0.04359672 |
| 15 - 240 | N15 | C240 | EP300 - ANM3_HUMAN | 1.29113884 | 0.02557326 |
| 20 - 35 | N20 | C35 | KDM1B - VPR_JKNp64 | 1.29093048 | 0.00905715 |
| 118 - 15 | N118 | C15 | HDAC6_HUMAN - EP300 | 1.29080922 | 0.00848423 |
| 285 - 85 | N285 | C85 | p65-MyoD - | 1.28921612 | 0.01653297 |
| | | | SMYD3_HUMAN | | |
| 284 - 126 | N284 | C126 | p65-HSF1 - | 1.28882589 | 0.00386316 |
| | | | SETMR_HUMAN | | |
| 149 - 14 | N149 | C14 | MBD1_HUMAN - RELA | 1.28741891 | 0.00103181 |
| 14 - 210 | N14 | C210 | RELA - SIR3_HUMAN | 1.28653601 | 0.03904283 |
| 284 - 329 | N284 | C329 | p65-HSF1 - pbcv1_vSET | 1.28588539 | 0.02816396 |
| 97 - 305 | N97 | C305 | CHD3_HUMAN - NUE | 1.28580367 | 0.04570827 |
| 59 - 99 | N59 | C99 | INO80_HUMAN - | 1.28569053 | 0.00300938 |
| | | | IN80C_HUMAN | | |
| 222 - 173 | N222 | C173 | IN80E_HUMAN - | 1.28498426 | 0.02496508 |
| | | | SIR4_HUMAN | | |
| 281 - 333 | N281 | C333 | VP64 - sc_RPD3 | 1.28454344 | 0.01491404 |
| 141 - 284 | N141 | C284 | SMRC1_HUMAN - p65- | 1.28430122 | 0.02370791 |
| | | | HSF1 | | |
| 107 - 281 | N107 | C281 | KAT6B_HUMAN - VP64 | 1.27988453 | 0.00269248 |
| 285 - 120 | N285 | C120 | p65-MyoD - | 1.27828564 | 0.0125886 |
| | | | RING1_HUMAN | | |
| 14 - 38 | N14 | C38 | RELA - EHMT2 | 1.27820268 | 0.0180353 |
| 285 - 75 | N285 | C75 | p65-MyoD - | 1.27809448 | 0.00120087 |
| | | | KDM5D_HUMAN | | |
| 281 - 103 | N281 | C103 | VP64 - B4DKA4_HUMAN | 1.27796152 | 0.01586955 |
| 14 - 154 | N14 | C154 | RELA - NSD2_HUMAN | 1.27791369 | 0.04588529 |
| 222 - 257 | N222 | C257 | IN80E_HUMAN - c-Myb- | 1.27785729 | 0.00704491 |
| | | | TAD | | |
| 284 - 90 | N284 | C90 | p65-HSF1 - CHD4_HUMAN | 1.277606 | 0.00275486 |
| 88 - 257 | N88 | C257 | ING4_HUMAN - c-Myb- | 1.27743141 | 0.01608693 |
| | | | TAD | | |
| 282 - 243 | N282 | C243 | VP160 - DNMT1_HUMAN | 1.277358 | 0.01057679 |
| 74 - 99 | N74 | C99 | KDM5D_HUMAN - | 1.27599983 | 0.01765566 |
| | | | IN80C_HUMAN | | |
| 281 - 273 | N281 | C273 | VP64 - PRDM9-CD | 1.27555364 | 0.00160741 |
| 154 - 296 | N154 | C296 | NSD2_HUMAN - HP1a | 1.27525309 | 0.04698377 |
| 48 - 284 | N48 | C284 | SETD5_HUMAN - p65-HSF1 | 1.27294037 | 0.03728062 |
| 313 - 14 | N313 | C14 | SaII - RELA | 1.27276221 | 0.04766708 |
| 297 - 257 | N297 | C257 | MesoLo - c-Myb-TAD | 1.27232189 | 0.00202764 |
| 212 - 245 | N212 | C245 | HDAC8_HUMAN - | 1.27144282 | 0.02697181 |
| | | | TYY1_HUMAN | | |
| 24 - 284 | N24 | C284 | CTCFL - p65-HSF1 | 1.26847633 | 0.03835803 |
| 14 - 297 | N14 | C297 | RELA - MesoLo | 1.26843928 | 0.00256499 |
| 255 - 99 | N255 | C99 | EED_HUMAN - | 1.26751318 | 0.01812762 |
| | | | IN80C_HUMAN | | |
| 300 - 252 | N300 | C252 | SIRT6 - HCFC1_HUMAN | 1.26443344 | 0.00818746 |
| 285 - 270 | N285 | C270 | p65-MyoD - ENL | 1.26189925 | 0.02072998 |
| 285 - 302 | N285 | C302 | p65-MyoD - HDAC11 | 1.2617895 | 0.00468276 |
| 218 - 257 | N218 | C257 | IN80B_HUMAN - c-Myb- | 1.26136866 | 0.00543591 |
| | | | TAD | | |
| 14 - 101 | N14 | C101 | RELA - TAF1_HUMAN | 1.26079684 | 0.04633285 |
| 281 - 311 | N281 | C311 | VP64 - Sin3a | 1.26042564 | 0.00101697 |
| 162 - 257 | N162 | C257 | SIR6_HUMAN - c-Myb-TAD | 1.25959501 | 0.00890762 |
| 299 - 14 | N299 | C14 | HST2 - RELA | 1.25721055 | 0.00428442 |
| 162 - 14 | N162 | C14 | SIR6_HUMAN - RELA | 1.25603575 | 0.04226079 |
| 308 - 14 | N308 | C14 | HDT1 - RELA | 1.25538493 | 0.00105626 |
| 173 - 252 | N173 | C252 | SIR4_HUMAN - | 1.25538486 | 0.02641928 |
| | | | HCFC1_HUMAN | | |
| 285 - 262 | N285 | C262 | p65-MyoD - EZH2 | 1.25509465 | 0.04272417 |
| 285 - 48 | N285 | C48 | p65-MyoD - | 1.25503052 | 0.00140344 |
| | | | SETD5_HUMAN | | |
| 275 - 10 | N275 | C10 | DOT1L-DOT1_domain - | 1.25454207 | 0.04547223 |
| | | | HSF1 | | |
| 14 - 218 | N14 | C218 | RELA - IN80B_HUMAN | 1.25381147 | 0.00590569 |
| 250 - 267 | N250 | C267 | HCFC1_HUMAN - FOXA1- | 1.25229756 | 0.02892508 |
| | | | Nterm | | |
| 252 - 1 | N252 | C1 | HCFC1_HUMAN - SETD1A | 1.25164779 | 0.00676375 |
| 69 - 114 | N69 | C114 | SETB2_HUMAN - | 1.25157027 | 0.00999102 |
| | | | KDM5A_HUMAN | | |
| 281 - 107 | N281 | C107 | VP64 - KAT6B_HUMAN | 1.25093404 | 0.00931099 |
| 14 - 59 | N14 | C59 | RELA - INO80_HUMAN | 1.25077395 | 0.00115173 |
| 210 - 281 | N210 | C281 | SIR3_HUMAN - VP64 | 1.24906588 | 0.02576719 |
| 331 - 281 | N331 | C281 | mm_G9a - VP64 | 1.24851424 | 0.00150538 |
| 173 - 17 | N173 | C17 | SIR4_HUMAN - EZH2 | 1.24700213 | 0.01458457 |
| 205 - 257 | N205 | C257 | JMJD7_HUMAN - c-Myb- | 1.24563043 | 0.0056145 |
| | | | TAD | | |
| 14 - 306 | N14 | C306 | RELA - CobB | 1.24542427 | 0.00234543 |
| 73 - 291 | N73 | C291 | KDM5D_HUMAN - SS18 | 1.24443468 | 0.00461808 |
| 162 - 345 | N162 | C345 | SIR6_HUMAN - | 1.24440333 | 0.03531449 |
| | | | MSX2_HUMAN | | |
| 270 - 187 | N270 | C187 | ENL - MBD4_HUMAN | 1.24304035 | 0.01629168 |
| 27 - 284 | N27 | C284 | EZH1 - p65-HSF1 | 1.24202732 | 0.02343438 |
| 199 - 133 | N199 | C133 | ING3_HUMAN - | 1.24142431 | 0.02001021 |
| | | | KAT8_HUMAN | | |
| 305 - 281 | N305 | C281 | NUE - VP64 | 1.24139616 | 0.04333359 |
| 285 - 12 | N285 | C12 | p65-MyoD - KMT2A | 1.23994609 | 0.01232446 |
| 14 - 27 | N14 | C27 | RELA - EZH1 | 1.23864752 | 0.00361728 |
| 123 - 12 | N123 | C12 | KMT2A_HUMAN - KMT2A | 1.23823007 | 0.04705236 |
| 322 - 257 | N322 | C257 | MINT_HUMAN - c-Myb- | 1.23749328 | 0.01303805 |
| | | | TAD | | |
| 14 - 346 | N14 | C346 | RELA - MSX2_HUMAN | 1.23741423 | 0.01797255 |
| 284 - 111 | N284 | C111 | p65-HSF1 - | 1.23721114 | 0.00185564 |
| | | | KDM5A_HUMAN | | |
| 252 - 98 | N252 | C98 | HCFC1_HUMAN - | 1.23638298 | 0.04060605 |
| | | | A4FU79_HUMAN | | |
| 285 - 212 | N285 | C212 | p65-MyoD - | 1.23547827 | 0.01181548 |
| | | | HDAC8_HUMAN | | |
| 200 - 294 | N200 | C294 | ARP5_HUMAN - DIM5 | 1.23441894 | 0.01469099 |
| 285 - 338 | N285 | C338 | p65-MyoD - | 1.23070662 | 0.03569942 |
| | | | RBM15_HUMAN | | |
| 281 - 76 | N281 | C76 | VP64 - KDM5D_HUMAN | 1.23057128 | 0.02024573 |
| 118 - 8 | N118 | C8 | HDAC6_HUMAN - MECP2 | 1.22884746 | 0.02147749 |
| 345 - 118 | N345 | C118 | MSX2_HUMAN | 1.22794318 | 0.04283543 |
| | | | HDAC6_HUMAN | | |
| 73 - 285 | N73 | C285 | KDM5D_HUMAN - p65- | 1.22699008 | 0.00829886 |
| | | | MyoD | | |
| 14 - 37 | N14 | C37 | RELA - KDM6A | 1.22694249 | 0.00233962 |
| 284 - 317 | N284 | C317 | p65-HSF1 - HDAC3 | 1.2261329 | 0.0115686 |
| 312 - 284 | N312 | C284 | SetD8 - p65-HSF1 | 1.22409631 | 0.00230538 |
| 281 - 294 | N281 | C294 | VP64 - DIM5 | 1.22360837 | 0.00168143 |
| 252 - 290 | N252 | C290 | HCFC1_HUMAN - HP1aCS | 1.22300506 | 0.00315879 |
| 17 - 280 | N17 | C280 | EZH2 - CBF1-Cterm | 1.22248939 | 0.01624525 |
| 309 - 281 | N309 | C281 | HDAC8 - VP64 | 1.22075595 | 0.00364504 |
| 339 - 257 | N339 | C257 | SPOC1_HUMAN - c-Myb- | 1.21944739 | 0.01767294 |
| | | | TAD | | |
| 277 - 285 | N277 | C285 | SUV39H1 - p65-MyoD | 1.21908613 | 0.00538528 |
| 220 - 148 | N220 | C148 | JARD2_HUMAN - | 1.21852351 | 0.04485188 |
| | | | NSD3_HUMAN | | |
| 25 - 99 | N25 | C99 | CTCFL - IN80C_HUMAN | 1.21814514 | 0.03793855 |
| 200 - 116 | N200 | C116 | ARP5_HUMAN - | 1.21805121 | 0.03558566 |
| | | | ANM6_HUMAN | | |
| 8 - 284 | N8 | C284 | MECP2 - p65-HSF1 | 1.2178612 | 9.99E−04 |
| 14 - 295 | N14 | C295 | RELA - SET-TAF1B | 1.21782074 | 0.00689414 |
| 118 - 284 | N118 | C284 | HDAC6_HUMAN - p65- | 1.21723985 | 0.02053728 |
| | | | HSF1 | | |
| 324 - 14 | N324 | C14 | LBR_HUMAN - RELA | 1.21708084 | 0.00262102 |
| 285 - 172 | N285 | C172 | p65-MyoD - | 1.21649096 | 0.01412798 |
| | | | ASH1L_HUMAN | | |
| 200 - 313 | N200 | C313 | ARP5_HUMAN - SaII | 1.21355577 | 0.03269682 |
| 1 - 218 | N1 | C218 | SETD1A - IN80B_HUMAN | 1.21287417 | 0.03497552 |
| 285 - 177 | N285 | C177 | p65-MyoD - | 1.2126358 | 0.00702533 |
| | | | KDM5C_HUMAN | | |
| 26 - 138 | N26 | C138 | EZH1 - TFPT_HUMAN | 1.21176152 | 0.03936804 |
| 200 - 214 | N200 | C214 | ARP5_HUMAN - | 1.21080692 | 0.04074797 |
| | | | HDAC4_HUMAN | | |
| 267 - 285 | N267 | C285 | FOXA1-Nterm - p65-MyoD | 1.20729689 | 0.03185364 |
| 10 - 78 | N10 | C78 | HSF1 - KAT6A_HUMAN | 1.20463101 | 0.02364018 |
| 136 - 237 | N136 | C237 | JMJD6_HUMAN - | 1.20353362 | 0.04303552 |
| | | | KAT5_HUMAN | | |
| 294 - 15 | N294 | C15 | DIM5 - EP300 | 1.20314549 | 0.01245629 |
| 315 - 29 | N315 | C29 | NCoR - ASH2L | 1.20195337 | 0.01688794 |
| 75 - 14 | N75 | C14 | KDM5D_HUMAN - RELA | 1.20067552 | 0.03037766 |
| 63 - 285 | N63 | C285 | SMYD5_HUMAN - p65- | 1.20002823 | 0.04483307 |
| | | | MyoD | | |
| 14 - 289 | N14 | C289 | RELA - FOG1 | 1.19905603 | 0.03922023 |
| 1 - 14 | N1 | C14 | SETD1A - RELA | 1.19735734 | 0.00160344 |
| 327 - 285 | N327 | C285 | hs_NIPP1 - p65-MyoD | 1.19719099 | 0.03805002 |
| 103 - 281 | N103 | C281 | B4DKA4_HUMAN - VP64 | 1.19547925 | 0.0270257 |
| 213 - 281 | N213 | C281 | HBAP1_HUMAN - VP64 | 1.19519759 | 0.00271487 |
| 284 - 302 | N284 | C302 | p65-HSF1 - HDAC11 | 1.19455265 | 0.02191715 |
| 129 - 257 | N129 | C257 | HAIR_HUMAN - c-Myb- | 1.19268356 | 0.01692662 |
| | | | TAD | | |
| 14 - 17 | N14 | C17 | RELA - EZH2 | 1.19149308 | 0.00523058 |
| 148 - 280 | N148 | C280 | NSD3_HUMAN - CBF1- | 1.19120475 | 0.02123235 |
| | | | Cterm | | |
| 326 - 15 | N326 | C15 | pf_Sir2A - EP300 | 1.19093602 | 0.04640505 |
| 14 - 326 | N14 | C326 | RELA - pf_Sir2A | 1.18966201 | 0.00707283 |
| 231 - 190 | N231 | C190 | KDM5B_HUMAN - | 1.18935173 | 0.04327003 |
| | | | ANM1_HUMAN | | |
| 135 - 284 | N135 | C284 | KDM2A_HUMAN - p65- | 1.18643895 | 0.04051119 |
| | | | HSF1 | | |
| 280 - 250 | N280 | C250 | CBF1-Cterm - | 1.18643531 | 9.88E−04 |
| | | | HCFC1_HUMAN | | |
| 284 - 48 | N284 | C48 | p65-HSF1 - SETD5_HUMAN | 1.1863083 | 0.00203831 |
| 149 - 250 | N149 | C250 | MBD1_HUMAN - | 1.18630155 | 0.03665783 |
| | | | HCFC1_HUMAN | | |
| 47 - 10 | N47 | C10 | ANM2_HUMAN - HSF1 | 1.18567017 | 0.03303675 |
| 14 - 302 | N14 | C302 | RELA - HDAC11 | 1.18420986 | 0.0171533 |
| 200 - 250 | N200 | C250 | ARP5_HUMAN - | 1.18382014 | 0.04444352 |
| | | | HCFC1_HUMAN | | |
| 138 - 257 | N138 | C257 | TFPT_HUMAN - c-Myb- | 1.1834844 | 0.00569724 |
| | | | TAD | | |
| 66 - 10 | N66 | C10 | DOT1L_HUMAN - HSF1 | 1.18340257 | 0.01764687 |
| 280 - 267 | N280 | C267 | CBF1-Cterm - FOXA1-Nterm | 1.18303592 | 0.04722155 |
| 282 - 98 | N282 | C98 | VP160 - A4FU79_HUMAN | 1.18284676 | 0.00717264 |
| 200 - 233 | N200 | C233 | ARP5_HUMAN - | 1.18279256 | 0.00492612 |
| | | | HINFP_HUMAN | | |
| 14 - 205 | N14 | C205 | RELA - JMJD7_HUMAN | 1.18163765 | 0.00737654 |
| 317 - 284 | N317 | C284 | HDAC3 - p65-HSF1 | 1.18119845 | 0.01266285 |
| 14 - 226 | N14 | C226 | RELA - SETD2_HUMAN | 1.18038903 | 0.03022385 |
| 35 - 329 | N35 | C329 | VPR_JKNp64 - pbcv1_vSET | 1.17930846 | 0.04943535 |
| 138 - 252 | N138 | C252 | TFPT_HUMAN - | 1.17897562 | 0.01635413 |
| | | | HCFC1_HUMAN | | |
| 14 - 309 | N14 | C309 | RELA - HDAC8 | 1.17605585 | 0.01230685 |
| 173 - 257 | N173 | C257 | SIR4_HUMAN - c-Myb-TAD | 1.17586959 | 0.01285672 |
| 300 - 257 | N300 | C257 | SIRT6 - c-Myb-TAD | 1.17498871 | 0.00415894 |
| 12 - 252 | N12 | C252 | KMT2A - HCFC1_HUMAN | 1.17466407 | 0.03012158 |
| 329 - 285 | N329 | C285 | pbcv1_vSET - p65-MyoD | 1.17344779 | 0.01077113 |
| 282 - 100 | N282 | C100 | VP160 - TAF1_HUMAN | 1.17085754 | 0.00658288 |
| 14 - 271 | N14 | C271 | RELA - GCN5 | 1.17084937 | 0.00225809 |
| 17 - 310 | N17 | C310 | EZH2 - TgSET8 | 1.17076872 | 0.0329971 |
| 197 - 257 | N197 | C257 | SUV91_HUMAN - c-Myb- | 1.16738757 | 0.00211464 |
| | | | TAD | | |
| 250 - 246 | N250 | C246 | HCFC1_HUMAN - | 1.16724347 | 0.04056063 |
| | | | ATRX_HUMAN | | |
| 250 - 233 | N250 | C233 | HCFC1_HUMAN - | 1.16605358 | 0.00447943 |
| | | | HINFP_HUMAN | | |
| 252 - 239 | N252 | C239 | HCFC1_HUMAN - | 1.16551227 | 0.02433917 |
| | | | KDM8_HUMAN | | |
| 282 - 30 | N282 | C30 | VP160 - KDM3A | 1.16408259 | 0.01795862 |
| 35 - 32 | N35 | C32 | VPR_JKNp64 - | 1.16364503 | 0.01461138 |
| | | | Gilbert_Lab_CRISPRi_KRAB | | |
| 327 - 291 | N327 | C291 | hs_NIPP1 - SS18 | 1.16077983 | 0.03312439 |
| 166 - 257 | N166 | C257 | EHMT1_HUMAN - c-Myb- | 1.16073488 | 0.0251635 |
| | | | TAD | | |
| 255 -20 | N255 | C20 | EED_HUMAN - KDM1B | 1.16067055 | 0.02860744 |
| 87 - 257 | N87 | C257 | SETD7_HUMAN - c-Myb- | 1.16036174 | 0.00733947 |
| | | | TAD | | |
| 79 - 285 | N79 | C285 | KAT6A_HUMAN - p65- | 1.15980803 | 0.02098242 |
| | | | MyoD | | |
| 284 - 53 | N284 | C53 | p65-HSF1 - CBP_HUMAN | 1.15934091 | 0.01725859 |
| 250 - 327 | N250 | C327 | HCFC1_HUMAN - hs_NIPP1 | 1.15807599 | 0.02685557 |
| 59 - 102 | N59 | C102 | INO80_HUMAN - | 1.15310073 | 0.03471362 |
| | | | TAF1_HUMAN | | |
| 112 - 10 | N112 | C10 | KDM5A_HUMAN - HSF1 | 1.15260331 | 0.03319136 |
| 257 - 99 | N257 | C99 | c-Myb-TAD - | 1.15022566 | 0.01712779 |
| | | | IN80C_HUMAN | | |
| 59 - 285 | N59 | C285 | INO80_HUMAN - p65-MyoD | 1.14963845 | 0.02255725 |
| 285 - 309 | N285 | C309 | p65-MyoD - HDAC8 | 1.14909558 | 0.04356006 |
| 281 - 241 | N281 | C241 | VP64 - SIR2_HUMAN | 1.14904638 | 0.00394925 |
| 14 - 9 | N14 | C9 | RELA - MECP2 | 1.14901459 | 0.04223749 |
| 14 - 69 | N14 | C69 | RELA - SETB2_HUMAN | 1.14761442 | 0.00733734 |
| 280 - 99 | N280 | C99 | CBF1-Cterm - | 1.14715006 | 0.00632289 |
| | | | IN80C_HUMAN | | |
| 200 - 266 | N200 | C266 | ARP5_HUMAN - GADD45A | 1.14702003 | 0.03718701 |
| 284 - 348 | N284 | C348 | p65-HSF1 - BRDT_HUMAN | 1.146704 | 0.03575883 |
| 14 - 136 | N14 | C136 | RELA - JMJD6_HUMAN | 1.14663395 | 0.02792436 |
| 14 - 20 | N14 | C20 | RELA - KDM1B | 1.14565466 | 0.02093099 |
| 25 - 119 | N25 | C119 | CTCFL - ACL6A_HUMAN | 1.1452643 | 0.01691316 |
| 73 - 218 | N73 | C218 | KDM5D_HUMAN - | 1.14426942 | 0.02501967 |
| | | | IN80B_HUMAN | | |
| 14 - 77 | N14 | C77 | RELA - KDM5D_HUMAN | 1.14363619 | 0.02664823 |
| 15 - 1 | N15 | C1 | EP300 - SETD1A | 1.14191575 | 0.00393777 |
| 282 - 273 | N282 | C273 | VP160 - PRDM9-CD | 1.14176406 | 0.00707942 |
| 282 - 187 | N282 | C187 | VP160 - MBD4_HUMAN | 1.14118589 | 0.00215247 |
| 327 - 280 | N327 | C280 | hs_NIPP1 - CBF1-Cterm | 1.1408709 | 0.00443957 |
| 32 - 14 | N32 | C14 | Gilbert_Lab_CRISPRi_KRAB - | 1.13962232 | 0.00860258 |
| | | | RELA | | |
| 14 - 312 | N14 | C312 | RELA - SetD8 | 1.13961277 | 0.00491463 |
| 14 - 268 | N14 | C268 | RELA - FOXA1-Cterm | 1.13938881 | 0.01184859 |
| 285 - 301 | N285 | C301 | p65-MyoD - SIRT3 | 1.13891315 | 0.01597431 |
| 322 - 266 | N322 | C266 | MINT_HUMAN - GADD45A | 1.13880772 | 0.03924703 |
| 83 - 111 | N83 | C111 | SMRD3_HUMAN - | 1.1383584 | 0.01757555 |
| | | | KDM5A_HUMAN | | |
| 33 - 315 | N33 | C315 | Hilton_2015 - NCoR | 1.1383347 | 0.00721579 |
| 14 - 90 | N14 | C90 | RELA - CHD4_HUMAN | 1.13809204 | 0.02966496 |
| 14 - 73 | N14 | C73 | RELA - KDM5D_HUMAN | 1.13658617 | 0.01179978 |
| 150 - 14 | N150 | C14 | HDA11_HUMAN - RELA | 1.13511203 | 0.01212077 |
| 14 - 112 | N14 | C112 | RELA - KDM5A_HUMAN | 1.13486584 | 0.01647148 |
| 38 - 14 | N38 | C14 | EHMT2 - RELA | 1.13433608 | 0.00654559 |
| 268 -266 | N268 | C266 | FOXA1-Cterm - GADD45A | 1.13368964 | 0.03510333 |
| 293 - 281 | N293 | C281 | KRAB-MeCP2 - VP64 | 1.13333888 | 0.01658656 |
| 319 - 314 | N319 | C314 | mCBP-HAT - MeCP2 | 1.13214042 | 0.04755309 |
| 281 - 288 | N281 | C288 | VP64 - SUV[SET] | 1.13206951 | 0.0105383 |
| 310 - 14 | N310 | C14 | TgSET8 - RELA | 1.13205687 | 0.01446142 |
| 281 - 51 | N281 | C51 | VP64 - UCHL5_HUMAN | 1.13155493 | 0.01155956 |
| 285 - 59 | N285 | C59 | p65-MyoD - INO80_HUMAN | 1.13151285 | 0.00661797 |
| 14 - 88 | N14 | C88 | RELA - ING4_HUMAN | 1.13064324 | 0.00223551 |
| 315 - 10 | N315 | C10 | NCoR - HSF1 | 1.12986904 | 0.02617633 |
| 285 - 90 | N285 | C90 | p65-MyoD - CHD4_HUMAN | 1.1297965 | 0.00866607 |
| 241 - 14 | N241 | C14 | SIR2_HUMAN - RELA | 1.12948219 | 0.02256749 |
| 281 - 222 | N281 | C222 | VP64 - IN80E_HUMAN | 1.12893299 | 0.02391655 |
| 266 - 14 | N266 | C14 | GADD45A - RELA | 1.12717093 | 0.00507326 |
| 48 - 10 | N48 | C10 | SETD5_HUMAN - HSF1 | 1.12575539 | 0.02621402 |
| 281 - 243 | N281 | C243 | VP64 - DNMT1_HUMAN | 1.12530222 | 0.03213164 |
| 266 - 284 | N266 | C284 | GADD45A - p65-HSF1 | 1.12450186 | 0.01985237 |
| 252 - 267 | N252 | C267 | HCFC1_HUMAN - FOXA1- | 1.12447912 | 0.0497102 |
| | | | Nterm | | |
| 35 - 98 | N35 | C98 | VPR_JKNp64 - | 1.12421824 | 0.02376499 |
| | | | A4FU79_HUMAN | | |
| 219 - 257 | N219 | C257 | JARD2_HUMAN - c-Myb- | 1.11994248 | 0.04222132 |
| | | | TAD | | |
| 306 - 14 | N306 | C14 | CobB - RELA | 1.1192136 | 0.01079551 |
| 285 - 191 | N285 | C191 | p65-MyoD - CDYL_HUMAN | 1.11864951 | 0.03803893 |
| 189 - 291 | N189 | C291 | ING2_HUMAN - SS18 | 1.11729471 | 0.02556547 |
| 14 - 87 | N14 | C87 | RELA - SETD7_HUMAN | 1.11726953 | 0.0269548 |
| 14 - 18 | N14 | C18 | RELA - EZH2 | 1.11642954 | 0.00564617 |
| 281 - 215 | N281 | C215 | VP64 - HDAC4_HUMAN | 1.11613755 | 0.005461 |
| 346 - 10 | N346 | C10 | MSX2_HUMAN - HSF1 | 1.11558973 | 0.0039591 |
| 78 -218 | N78 | C218 | KAT6A_HUMAN - | 1.11296987 | 0.04854407 |
| | | | IN80B_HUMAN | | |
| 35 - 69 | N35 | C69 | VPR_JKNp64 - | 1.11277135 | 0.0128902 |
| | | | SETB2_HUMAN | | |
| 274 - 285 | N274 | C285 | UBE2A - p65-MyoD | 1.1108325 | 0.02199268 |
| 14 - 24 | N14 | C24 | RELA - CTCFL | 1.10991226 | 0.00515855 |
| 14 - 219 | N14 | C219 | RELA - JARD2_HUMAN | 1.10939798 | 0.00338032 |
| 270 - 106 | N270 | C106 | ENL - KAT6B_HUMAN | 1.10937097 | 0.02433989 |
| 26 - 285 | N26 | C285 | EZH1 - p65-MyoD | 1.10788874 | 0.01819086 |
| 136 - 266 | N136 | C266 | JMJD6_HUMAN - | 1.10573172 | 0.01460198 |
| | | | GADD45A | | |
| 287 - 257 | N287 | C257 | G9A[SET] - c-Myb-TAD | 1.10263155 | 0.01003452 |
| 281 - 152 | N281 | C152 | VP64 - CDY1_HUMAN | 1.10246536 | 0.01500151 |
| 296 - 14 | N296 | C14 | HP1a - RELA | 1.09872393 | 0.00352055 |
| 112 - 14 | N112 | C14 | KDM5A_HUMAN - RELA | 1.09858358 | 0.03660809 |
| 14 - 177 | N14 | C177 | RELA - KDM5C_HUMAN | 1.09787204 | 0.03781477 |
| 1 - 10 | N1 | C10 | SETD1A - HSF1 | 1.09654 | 0.01846073 |
| 24 - 328 | N24 | C328 | CTCFL - hs_PHF19 | 1.09227553 | 0.01598669 |
| 34 - 249 | N34 | C249 | DNMT3A-3L_JKNp112 - | 1.09203444 | 0.04315488 |
| | | | HAC1-HAT | | |
| 14 - 287 | N14 | C287 | RELA - G9A[SET] | 1.08942129 | 0.0125785 |
| 111 - 99 | N111 | C99 | KDM5A_HUMAN - | 1.08878362 | 0.01662696 |
| | | | IN80C_HUMAN | | |
| 239 - 257 | N239 | C257 | KDM8_HUMAN - c-Myb- | 1.0851327 | 0.00503321 |
| | | | TAD | | |
| 2 - 257 | N2 | C257 | SETD1A - c-Myb-TAD | 1.08337102 | 0.00809951 |
| 250 - 269 | N250 | C269 | HCFC1_HUMAN - PU.1 | 1.08292585 | 0.00565562 |
| 285 - 264 | N285 | C264 | p65-MyoD - MTL5_HUMAN | 1.08178566 | 0.00966733 |
| 14 - 107 | N14 | C107 | RELA - KAT6B_HUMAN | 1.07953117 | 0.02529427 |
| 295 - 218 | N295 | C218 | SET-TAF1B - | 1.07837581 | 0.03229402 |
| | | | IN80B_HUMAN | | |
| 148 - 257 | N148 | C257 | NSD3_HUMAN - c-Myb- | 1.07654246 | 0.00566102 |
| | | | TAD | | |
| 155 - 281 | N155 | C281 | NSD2_HUMAN - VP64 | 1.07349354 | 0.0122466 |
| 10 - 290 | N10 | C290 | HSF1 - HP1aCS | 1.0734928 | 0.01621253 |
| 284 - 298 | N284 | C298 | p65-HSF1 - MBD2b | 1.07326315 | 0.00680382 |
| 138 - 269 | N138 | C269 | TFPT_HUMAN - PU.1 | 1.07267711 | 0.02235393 |
| 328 - 267 | N328 | C267 | hs_PHF19 - FOXA1-Nterm | 1.07264166 | 0.04978377 |
| 200 - 346 | N200 | C346 | ARP5_HUMAN - | 1.07193488 | 0.01089144 |
| | | | MSX2_HUMAN | | |
| 315 - 284 | N315 | C284 | NCoR - p65-HSF1 | 1.06974047 | 0.03516013 |
| 77 - 290 | N77 | C290 | KDM5D_HUMAN - HP1aCS | 1.0695219 | 0.01453364 |
| 270 - 69 | N270 | C69 | ENL - SETB2_HUMAN | 1.06907556 | 0.02698443 |
| 263 - 285 | N263 | C285 | HDAC8 - p65-MyoD | 1.06605014 | 0.03966031 |
| 6 - 280 | N6 | C280 | KDM1A - CBF1-Cterm | 1.06512222 | 0.01826827 |
| 18 - 99 | N18 | C99 | EZH2 - IN80C_HUMAN | 1.06501131 | 0.01003563 |
| 14 - 48 | N14 | C48 | RELA - SETD5_HUMAN | 1.06501078 | 0.00558666 |
| 130 - 99 | N130 | C99 | PRDM2_HUMAN - | 1.06358797 | 0.016521 |
| | | | IN80C_HUMAN | | |
| 253 - 246 | N253 | C246 | RBBP5_HUMAN - | 1.05820379 | 0.02247181 |
| | | | ATRX_HUMAN | | |
| 78 - 162 | N78 | C162 | KAT6A_HUMAN - | 1.05812536 | 0.0408961 |
| | | | SIR6_HUMAN | | |
| 212 - 269 | N212 | C269 | HDAC8_HUMAN - PU.1 | 1.05767437 | 0.01101144 |
| 14 - 263 | N14 | C263 | RELA - HDAC8 | 1.05600064 | 0.03581272 |
| 324 - 281 | N324 | C281 | LBR_HUMAN - VP64 | 1.05282922 | 0.03366386 |
| 14 - 264 | N14 | C264 | RELA - MTL5_HUMAN | 1.05262503 | 0.0131832 |
| 190 - 269 | N190 | C269 | ANM1_HUMAN - PU.1 | 1.05130914 | 0.04947621 |
| 166 - 99 | N166 | C99 | EHMT1_HUMAN - | 1.05122615 | 0.04300957 |
| | | | IN80C_HUMAN | | |
| 17 - 10 | N17 | C10 | EZH2 - HSF1 | 1.04953351 | 0.01880229 |
| 188 - 257 | N188 | C257 | SMYD4_HUMAN - c-Myb- | 1.04934261 | 0.02085587 |
| | | | TAD | | |
| 26 - 99 | N26 | C99 | EZH1 - IN80C_HUMAN | 1.04841583 | 0.03770376 |
| 200 - 320 | N200 | C320 | ARP5_HUMAN - mMLL3- | 1.04795271 | 0.03265444 |
| | | | SET | | |
| 293 - 285 | N293 | C285 | KRAB-MeCP2 - p65-MyoD | 1.04662582 | 0.04520191 |
| 14 - 222 | N14 | C222 | RELA - IN80E_HUMAN | 1.04615471 | 0.01319151 |
| 218 - 288 | N218 | C288 | IN80B_HUMAN - SUV[SET] | 1.04387908 | 0.01403782 |
| 218 - 233 | N218 | C233 | IN80B_HUMAN - | 1.04387026 | 0.04937932 |
| | | | HINFP_HUMAN | | |
| 274 - 99 | N274 | C99 | UBE2A - IN80C_HUMAN | 1.04110336 | 0.02160648 |
| 38 - 250 | N38 | C250 | EHMT2 - HCFC1_HUMAN | 1.04092092 | 0.04387085 |
| 322 - 14 | N322 | C14 | MINT_HUMAN - RELA | 1.03976038 | 0.04986186 |
| 212 - 15 | N212 | C15 | HDAC8_HUMAN - EP300 | 1.03646773 | 0.02729509 |
| 149 - 99 | N149 | C99 | MBD1_HUMAN - | 1.03615769 | 0.00878247 |
| | | | IN80C_HUMAN | | |
| 78 - 288 | N78 | C288 | KAT6A_HUMAN - | 1.0360823 | 0.04889088 |
| | | | SUV[SET] | | |
| 28 - 290 | N28 | C290 | EHMT2 - HP1aCS | 1.03482047 | 0.0456725 |
| 218 - 99 | N218 | C99 | IN80B_HUMAN - | 1.03360118 | 0.02600464 |
| | | | IN80C_HUMAN | | |
| 100 - 285 | N100 | C285 | TAF1_HUMAN - p65-MyoD | 1.03334644 | 0.01742126 |
| 15 - 313 | N15 | C313 | EP300 - SaII | 1.03232303 | 0.02082581 |
| 27 - 14 | N27 | C14 | EZH1 - RELA | 1.03187224 | 0.02298941 |
| 285 - 7 | N285 | C7 | p65-MyoD - ZNF10 | 1.03086356 | 0.01694813 |
| 282 - 205 | N282 | C205 | VP160 - JMJD7_HUMAN | 1.03043851 | 0.00400922 |
| 346 - 14 | N346 | C14 | MSX2_HUMAN - RELA | 1.03038739 | 0.0300438 |
| 252 - 246 | N252 | C246 | HCFC1_HUMAN - | 1.02997078 | 0.03767331 |
| | | | ATRX_HUMAN | | |
| 153 - 281 | N153 | C281 | KAT7_HUMAN - VP64 | 1.02948056 | 0.04584821 |
| 294 - 291 | N294 | C291 | DIM5 - SS18 | 1.02947101 | 0.0389922 |
| 123 - 99 | N123 | C99 | KMT2A_HUMAN - | 1.02945396 | 0.01473389 |
| | | | IN80C_HUMAN | | |
| 295 - 250 | N295 | C250 | SET-TAF1B - | 1.02901382 | 0.0236325 |
| | | | HCFC1_HUMAN | | |
| 63 - 1 | N63 | C1 | SMYD5_HUMAN - SETD1A | 1.02778287 | 0.02627526 |
| 183 - 281 | N183 | C281 | KMT2C_HUMAN - VP64 | 1.02758921 | 0.0135268 |
| 25 - 12 | N25 | C12 | CTCFL - KMT2A | 1.02726157 | 0.0358928 |
| 73 - 99 | N73 | C99 | KDM5D_HUMAN - | 1.02430177 | 0.02536517 |
| | | | IN80C_HUMAN | | |
| 14 - 108 | N14 | C108 | RELA - KAT6B_HUMAN | 1.02415713 | 0.01884049 |
| 263 - 290 | N263 | C290 | HDAC8 - HP1aCS | 1.02162365 | 0.0182147 |
| 266 - 285 | N266 | C285 | GADD45A - p65-MyoD | 1.01983897 | 0.04469583 |
| 280 - 239 | N280 | C239 | CBF1-Cterm - | 1.01979531 | 0.03497921 |
| | | | KDM8_HUMAN | | |
| 56 - 35 | N56 | C35 | SUV92_HUMAN - | 1.01676902 | 0.04009927 |
| | | | VPR_JKNp64 | | |
| 35 - 324 | N35 | C324 | VPR_JKNp64 - | 1.01478733 | 0.01586394 |
| | | | LBR_HUMAN | | |
| 258 - 311 | N258 | C311 | Nejire-HAT - Sin3a | 1.01440308 | 0.03359209 |
| 3 - 285 | N3 | C285 | HDAC3 - p65-MyoD | 1.01422945 | 0.02760447 |
| 284 - 113 | N284 | C113 | p65-HSF1 - | 1.01361455 | 0.04309891 |
| | | | KDM5A_HUMAN | | |
| 250 - 73 | N250 | C73 | HCFC1_HUMAN - | 1.00934197 | 0.03097212 |
| | | | KDM5D_HUMAN | | |
| 257 - 58 | N257 | C58 | c-Myb-TAD - | 1.00894618 | 0.03462541 |
| | | | TF3C4_HUMAN | | |
| 210 - 14 | N210 | C14 | SIR3_HUMAN - RELA | 1.00887845 | 0.04950121 |
| 73 - 102 | N73 | C102 | KDM5D_HUMAN - | 1.00762846 | 0.03717307 |
| | | | TAF1_HUMAN | | |
| 284 - 173 | N284 | C173 | p65-HSF1 - SIR4_HUMAN | 1.00716464 | 0.03509227 |
| 17 - 29 | N17 | C291 | EZH2 - SS18 | 1.00633001 | 0.01615274 |
| 284 - 274 | N284 | C274 | p65-HSF1 - UBE2A | 1.00410496 | 0.00924136 |
| 63 - 17 | N63 | C17 | SMYD5_HUMAN - EZH2 | 1.00409871 | 0.03543477 |
| 268 - 280 | N268 | C280 | FOXA1-Cterm - CBF1-Cterm | 1.00226306 | 0.01746321 |
| 14 - 123 | N14 | C123 | RELA - KMT2A_HUMAN | 1.00156509 | 0.02522992 |
| 269 - 99 | N269 | C99 | PU.1 - IN80C_HUMAN | 1.00053884 | 0.02908537 |
| 136 - 1 | N136 | C1 | JMJD6_HUMAN - SETD1A | 0.99886188 | 0.01974094 |
| 218 - 313 | N218 | C313 | IN80B_HUMAN - SaII | 0.99875561 | 0.02849551 |
| 197 - 1 | N197 | C1 | SUV91_HUMAN - SETD1A | 0.99821636 | 0.04458237 |
| 295 - 257 | N295 | C257 | SET-TAF1B - c-Myb-TAD | 0.99761616 | 0.00737257 |
| 14 - 135 | N14 | C135 | RELA - KDM2A_HUMAN | 0.99692147 | 0.02179975 |
| 10 - 312 | N10 | C312 | HSF1 - SetD8 | 0.99307389 | 0.02813546 |
| 65 - 138 | N65 | C138 | MCRS1_HUMAN - | 0.99115754 | 0.03522588 |
| | | | TFPT_HUMAN | | |
| 335 - 285 | N335 | C285 | at_SUVR4 - p65-MyoD | 0.9897436 | 0.04514325 |
| 54 - 14 | N54 | C14 | CBP_HUMAN - RELA | 0.98640239 | 0.04570452 |
| 276 - 281 | N276 | C281 | EHMT2(G9a)-Cterm - VP64 | 0.98632034 | 0.00922971 |
| 106 - 99 | N106 | C99 | KAT6B_HUMAN - | 0.98594559 | 0.0073533 |
| | | | IN80C_HUMAN | | |
| 218 - 266 | N218 | C266 | IN80B_HUMAN - | 0.98525265 | 0.01743483 |
| | | | GADD45A | | |
| 281 - 177 | N281 | C177 | VP64 - KDM5C_HUMAN | 0.98381561 | 0.01533711 |
| 243 - 257 | N243 | C257 | DNMT1_HUMAN - c-Myb- | 0.98271225 | 0.04173652 |
| | | | TAD | | |
| 186 - 257 | N186 | C257 | RIOX2_HUMAN - c-Myb- | 0.98090591 | 0.00884522 |
| | | | TAD | | |
| 160 - 285 | N160 | C285 | KMT5A_HUMAN - p65- | 0.98041675 | 0.02626092 |
| | | | MyoD | | |
| 1 - 106 | N1 | C106 | SETD1A - KAT6B_HUMAN | 0.97738308 | 0.02082689 |
| 280 - 348 | N280 | C348 | CBF1-Cterm - | 0.97531875 | 0.02905937 |
| | | | BRDT_HUMAN | | |
| 274 - 291 | N274 | C291 | UBE2A - SS18 | 0.97088459 | 0.03631894 |
| 285 - 159 | N285 | C159 | p65-MyoD - | 0.97055193 | 0.03545823 |
| | | | SETB1_HUMAN | | |
| 324 - 257 | N324 | C257 | LBR_HUMAN - c-Myb-TAD | 0.9701607 | 0.0490446 |
| 229 - 281 | N229 | C281 | NFRKB_HUMAN - VP64 | 0.96902502 | 0.02766495 |
| 190 - 257 | N190 | C257 | ANM1_HUMAN - c-Myb- | 0.96660368 | 0.01269822 |
| | | | TAD | | |
| 329 - 284 | N329 | C284 | pbcv1_vSET - p65-HSF1 | 0.96382764 | 0.01806067 |
| 20 - 285 | N20 | C285 | KDM1B - p65-MyoD | 0.96243553 | 0.04196771 |
| 245 - 99 | N245 | C99 | TYY1_HUMAN - | 0.96199967 | 0.04472133 |
| | | | IN80C_HUMAN | | |
| 92 - 257 | N92 | C257 | SMYD1_HUMAN - c-Myb- | 0.9619325 | 0.01659486 |
| | | | TAD | | |
| 149 - 237 | N149 | C237 | MBD1_HUMAN - | 0.96138642 | 0.04860564 |
| | | | KAT5_HUMAN | | |
| 116 - 257 | N116 | C257 | ANM6_HUMAN - c-Myb- | 0.96096033 | 0.01414271 |
| | | | TAD | | |
| 263 - 257 | N263 | C257 | HDAC8 - c-Myb-TAD | 0.95772603 | 0.01000992 |
| 67 - 14 | N67 | C14 | PRDM9_HUMAN - RELA | 0.95671565 | 0.03431252 |
| 300 - 250 | N300 | C250 | SIRT6 - HCFC1_HUMAN | 0.95577046 | 0.03745966 |
| 212 - 138 | N212 | C138 | HDAC8_HUMAN - | 0.95319755 | 0.02445342 |
| | | | TFPT_HUMAN | | |
| 284 - 101 | N284 | C101 | p65-HSF1 - TAF1_HUMAN | 0.95219367 | 0.04281041 |
| 14 - 233 | N14 | C233 | RELA - HINFP_HUMAN | 0.9468737 | 0.04744352 |
| 275 - 17 | N275 | C17 | DOT1L-DOT1_domain - | 0.94683703 | 0.04738474 |
| | | | EZH2 | | |
| 280 - 173 | N280 | C173 | CBF1-Cterm - SIR4_HUMAN | 0.9448661 | 0.02896694 |
| 246 - 291 | N246 | C291 | ATRX_HUMAN - SS18 | 0.94378829 | 0.04174082 |
| 59 - 257 | N59 | C257 | INO80_HUMAN - c-Myb- | 0.94327685 | 0.0425688 |
| | | | TAD | | |
| 311 - 284 | N311 | C284 | Sin3a - p65-HSF1 | 0.94206185 | 0.02002131 |
| 14 - 331 | N14 | C331 | RELA - mm_G9a | 0.94200612 | 0.04138447 |
| 257 - 239 | N257 | C239 | c-Myb-TAD - | 0.94143201 | 0.02054801 |
| | | | KDM8_HUMAN | | |
| 327 - 257 | N327 | C257 | hs_NIPP1 - c-Myb-TAD | 0.94138298 | 0.02509911 |
| 295 - 280 | N295 | C280 | SET-TAF1B - CBF1-Cterm | 0.94049727 | 0.02013853 |
| 88 - 250 | N88 | C250 | ING4_HUMAN - | 0.93877685 | 0.04614257 |
| | | | HCFC1_HUMAN | | |
| 45 - 59 | N45 | C59 | KMT2D_HUMAN - | 0.93666925 | 0.044932 |
| | | | INO80_HUMAN | | |
| 35 - 312 | N35 | C312 | VPR_JKNp64 - SetD8 | 0.93495936 | 0.04209803 |
| 185 - 257 | N185 | C257 | RBBP7_HUMAN - c-Myb- | 0.93418701 | 0.04346015 |
| | | | TAD | | |
| 281 - 248 | N281 | C248 | VP64 - GLYR1_HUMAN | 0.93333171 | 0.03122294 |
| 106 - 200 | N106 | C200 | KAT6B_HUMAN - | 0.92752963 | 0.03974384 |
| | | | ARP5_HUMAN | | |
| 271 - 312 | N271 | C312 | GCN5 - SetD8 | 0.9264463 | 0.04583436 |
| 14 - 246 | N14 | C246 | RELA - ATRX_HUMAN | 0.925397 | 0.03251741 |
| 35 - 298 | N35 | C298 | VPR_JKNp64 - MBD2b | 0.92392242 | 0.02557586 |
| 305 - 284 | N305 | C284 | NUE - p65-HSF1 | 0.92341942 | 0.04372243 |
| 119 - 257 | N119 | C257 | ACL6A_HUMAN - c-Myb- | 0.92190457 | 0.01655259 |
| | | | TAD | | |
| 324 - 138 | N324 | C138 | LBR_HUMAN - | 0.92086817 | 0.03452895 |
| | | | TFPT_HUMAN | | |
| 20 - 14 | N20 | C14 | KDM1B - RELA | 0.91973814 | 0.04730788 |
| 250 - 290 | N250 | C290 | HCFC1_HUMAN - HP1aCS | 0.91915087 | 0.01546859 |
| 14 - 349 | N14 | C349 | RELA - DAM_N132A | 0.91836608 | 0.02015866 |
| 332 - 99 | N332 | C99 | hs_SIRT5 - IN80C_HUMAN | 0.91325631 | 0.04176604 |
| 285 - 126 | N285 | C126 | p65-MyoD - | 0.9131949 | 0.0240351 |
| | | | SETMR_HUMAN | | |
| 14 - 118 | N14 | C118 | RELA - HDAC6_HUMAN | 0.91190701 | 0.02969381 |
| 114 - 326 | N114 | C326 | KDM5A_HUMAN - pf_Sir2A | 0.90874231 | 0.0495106 |
| 310 - 99 | N310 | C99 | TgSET8 - IN80C_HUMAN | 0.90691429 | 0.03456457 |
| 12 - 285 | N12 | C285 | KMT2A - p65-MyoD | 0.90678049 | 0.02830441 |
| 295 - 245 | N295 | C245 | SET-TAF1B - | 0.90645733 | 0.0359991 |
| | | | TYY1_HUMAN | | |
| 186 - 290 | N186 | C290 | RIOX2_HUMAN - HP1aCS | 0.90402174 | 0.03598545 |
| 42 - 257 | N42 | C257 | KMT2E_HUMAN - c-Myb- | 0.90389605 | 0.02167323 |
| | | | TAD | | |
| 212 - 99 | N212 | C99 | HDAC8_HUMAN - | 0.90370205 | 0.02270657 |
| | | | IN80C_HUMAN | | |
| 14 - 272 | N14 | C272 | RELA - Sir2 | 0.8955985 | 0.0369525 |
| 5 - 66 | N5 | C66 | KDM1A - DOT1L_HUMAN | 0.89551079 | 0.04644566 |
| 296 - 214 | N296 | C214 | HP1a - HDAC4_HUMAN | 0.89239517 | 0.02667198 |
| 266 - 266 | N266 | C266 | GADD45A - GADD45A | 0.89183361 | 0.02379984 |
| 218 - 8 | N218 | C8 | IN80B_HUMAN - MECP2 | 0.89029861 | 0.04595099 |
| 218 - 290 | N218 | C290 | IN80B_HUMAN - HP1aCS | 0.88975429 | 0.01673461 |
| 32 - 285 | N32 | C285 | Gilbert_Lab_CRISPRi_KRAB - | 0.88872125 | 0.03012568 |
| | | | p65-MyoD | | |
| 246 - 59 | N246 | C59 | ATRX_HUMAN - | 0.88685519 | 0.03235477 |
| | | | INO80_HUMAN | | |
| 106 - 280 | N106 | C280 | KAT6B_HUMAN - CBF1 - | 0.88667192 | 0.02656272 |
| | | | Cterm | | |
| 14 - 310 | N14 | C310 | RELA - TgSET8 | 0.8843018 | 0.04743979 |
| 200 - 246 | N200 | C246 | ARP5_HUMAN - | 0.87738088 | 0.04443306 |
| | | | ATRX_HUMAN | | |
| 14 - 314 | N14 | C314 | RELA - MeCP2 | 0.87726172 | 0.03234951 |
| 48 - 285 | N48 | C285 | SETD5_HUMAN - p65- | 0.87283661 | 0.0449286 |
| | | | MyoD | | |
| 69 - 99 | N69 | C99 | SETB2_HUMAN - | 0.87174006 | 0.01554285 |
| | | | IN80C_HUMAN | | |
| 14 - 111 | N14 | C111 | RELA - KDM5A_HUMAN | 0.87159841 | 0.02269282 |
| 189 - 280 | N189 | C280 | ING2_HUMAN - CBF1- | 0.87109579 | 0.04880136 |
| | | | Cterm | | |
| 222 - 10 | N222 | C10 | IN80E_HUMAN - HSF1 | 0.8694092 | 0.03976155 |
| 14 - 320 | N14 | C320 | RELA - mMLL3-SET | 0.8693318 | 0.01608695 |
| 35 - 18 | N35 | C18 | VPR_JKNp64 - EZH2 | 0.86873738 | 0.04149399 |
| 150 - 257 | N150 | C257 | HDA11_HUMAN - c-Myb- | 0.86711792 | 0.02984073 |
| | | | TAD | | |
| 222 - 14 | N222 | C14 | IN80E_HUMAN - RELA | 0.86461968 | 0.0471924 |
| 174 - 99 | N174 | C99 | HDAC1_HUMAN - | 0.86366643 | 0.04609655 |
| | | | IN80C_HUMAN | | |
| 280 - 269 | N280 | C269 | CBF1-Cterm - PU.1 | 0.86242107 | 0.04696826 |
| 190 - 99 | N190 | C99 | ANM1_HUMAN - | 0.86038068 | 0.03947845 |
| | | | IN80C_HUMAN | | |
| 290 - 284 | N290 | C284 | HP1aCS - p65-HSF1 | 0.85515213 | 0.02260029 |
| 231 - 285 | N231 | C285 | KDM5B_HUMAN - p65- | 0.84615891 | 0.04190879 |
| | | | MyoD | | |
| 280 - 229 | N280 | C229 | CBF1-Cterm - | 0.837022 | 0.03379074 |
| | | | NFRKB_HUMAN | | |
| 61 - 99 | N61 | C99 | HAT1_HUMAN - | 0.8355363 | 0.04832549 |
| | | | IN80C_HUMAN | | |
| 151 - 14 | N151 | C14 | SIR7_HUMAN - RELA | 0.83467648 | 0.04268233 |
| 218 - 26 | N218 | C26 | IN80B_HUMAN - EZH1 | 0.8269143 | 0.04293645 |
| 253 - 257 | N253 | C257 | RBBP5_HUMAN - c-Myb- | 0.8269122 | 0.04785235 |
| | | | TAD | | |
| 282 - 88 | N282 | C88 | VP160 - ING4_HUMAN | 0.82293155 | 0.03972225 |
| 285 - 98 | N285 | C98 | p65-MyoD - | 0.82044313 | 0.02269891 |
| | | | A4FU79_HUMAN | | |
| 55 - 285 | N55 | C285 | RBBP4_HUMAN - p65- | 0.81982179 | 0.04886549 |
| | | | MyoD | | |
| 9 - 284 | N9 | C284 | MECP2 - p65-HSF1 | 0.8080385 | 0.03517797 |
| 253 - 269 | N253 | C269 | RBBP5_HUMAN - PU.1 | 0.80534336 | 0.04164577 |
| 105 - 284 | N105 | C284 | MBD2_HUMAN - p65-HSF1 | 0.80483477 | 0.04956215 |
| 328 - 257 | N328 | C257 | hs_PHF19 - c-Myb-TAD | 0.80294725 | 0.04287015 |
| 99 - 99 | N99 | C99 | IN80C_HUMAN - | 0.79630111 | 0.04566009 |
| | | | IN80C_HUMAN | | |
| 194 - 1 | N194 | C1 | ING1_HUMAN - SETD1A | 0.78982555 | 0.03754344 |
| 216 - 99 | N216 | C99 | ING5_HUMAN - | 0.78196225 | 0.03883945 |
| | | | IN80C_HUMAN | | |
| 88 - 269 | N88 | C269 | ING4_HUMAN - PU.1 | 0.77792268 | 0.03634269 |
| 212 - 233 | N212 | C233 | HDAC8_HUMAN - | 0.77670792 | 0.04425386 |
| | | | HINFP_HUMAN | | |
| 299 - 257 | N299 | C257 | HST2 - c-Myb-TAD | 0.77661449 | 0.04418636 |
| 15 - 106 | N15 | C106 | EP300 - KAT6B_HUMAN | 0.77447499 | 0.04507055 |
| 1 - 250 | N1 | C250 | SETD1A - HCFC1_HUMAN | 0.77323995 | 0.03659671 |
| 30 - 257 | N30 | C257 | KDM3A - c-Myb-TAD | 0.76793443 | 0.04381739 |
| 194 - 290 | N194 | C290 | ING1_HUMAN - HP1aCS | 0.76268834 | 0.04784584 |
| 280 - 154 | N280 | C154 | CBF1-Cterm - | 0.74507343 | 0.04830198 |
| | | | NSD2_HUMAN | | |
| 310 - 280 | N310 | C280 | TgSET8 - CBF1-Cterm | 0.74269989 | 0.04307829 |
| 294 - 257 | N294 | C257 | DIM5 - c-Myb-TAD | 0.71706963 | 0.04889726 |
| 305 - 105 | N305 | C105 | NUE - MBD2_HUMAN | −0.7096653 | 0.04890039 |
| 3- 26 | N3 | C26 | HDAC3 - EZH1 | −0.7280986 | 0.04472263 |
| 73 - 299 | N73 | C299 | KDM5D_HUMAN - HST2 | −0.7506284 | 0.04769153 |
| 105 - 59 | N105 | C59 | MBD2_HUMAN - | −0.7607073 | 0.0372834 |
| | | | INO80_HUMAN | | |
| 306 - 349 | N306 | C349 | CobB - DAM_N132A | −0.7611696 | 0.04946855 |
| 328 - 68 | N328 | C68 | hs_PHF19 - MBD3_HUMAN | −0.7668205 | 0.03863585 |
| 32 - 314 | N32 | C314 | Gilbert_Lab_CRISPRi_KRAB - | −0.7727348 | 0.04980644 |
| | | | MeCP2 | | |
| 105 - 330 | N105 | C330 | MBD2_HUMAN - ce_Set1 | −0.7732729 | 0.03148844 |
| 3 - 267 | N3 | C267 | HDAC3 - FOXA1-Nterm | −0.7818816 | 0.04538894 |
| 206 - 68 | N206 | C68 | SETD9_HUMAN - | −0.8019932 | 0.03645094 |
| | | | MBD3_HUMAN | | |
| 315 - 306 | N315 | C306 | NCoR - CobB | −0.8021611 | 0.0424883 |
| 249 - 105 | N249 | C105 | MTL5_HUMAN - | −0.8037262 | 0.03938878 |
| | | | MBD2_HUMAN | | |
| 346 - 9 | N346 | C9 | MSX2_HUMAN - MECP2 | −0.8037972 | 0.04314084 |
| 296 - 306 | N296 | C306 | HP1a - CobB | −0.807908 | 0.04798527 |
| 55 - 246 | N55 | C246 | RBBP4_HUMAN - | −0.8134488 | 0.02866591 |
| | | | ATRX_HUMAN | | |
| 198 - 306 | N198 | C306 | JMJD8_HUMAN - CobB | −0.8141974 | 0.04841622 |
| 55 - 305 | N55 | C305 | RBBP4_HUMAN - NUE | −0.8143865 | 0.03744856 |
| 331 - 299 | N331 | C299 | mm_G9a - HST2 | −0.816602 | 0.04339977 |
| 3 - 273 | N3 | C273 | HDAC3 - PRDM9-CD | −0.8170625 | 0.04851448 |
| 68 - 326 | N68 | C326 | MBD3_HUMAN - pf_Sir2A | −0.8174698 | 0.04014204 |
| 274 - 105 | N274 | C105 | UBE2A - MBD2_HUMAN | −0.8184059 | 0.03477752 |
| 9 - 217 | N9 | C217 | MECP2 - CHRC1_HUMAN | −0.8217543 | 0.04945116 |
| 3 - 205 | N3 | C205 | HDAC3 - JMJD7_HUMAN | −0.8268833 | 0.04448428 |
| 329 - 68 | N329 | C68 | pbcv1_vSET - | −0.827644 | 0.04879093 |
| | | | MBD3_HUMAN | | |
| 334 - 68 | N334 | C68 | hs_RCOR1 - | −0.8288154 | 0.02648879 |
| | | | MBD3_HUMAN | | |
| 271 - 105 | N271 | C105 | GCN5 - MBD2_HUMAN | −0.8302323 | 0.04540624 |
| 346 - 299 | N346 | C299 | MSX2_HUMAN - HST2 | −0.8321898 | 0.03194122 |
| 306 - 112 | N306 | C112 | CobB - KDM5A_HUMAN | −0.8332674 | 0.02212851 |
| 182 - 305 | N182 | C305 | MTA3_HUMAN - NUE | −0.833335 | 0.03446563 |
| 106 - 314 | N106 | C314 | KAT6B_HUMAN - MeCP2 | −0.8356044 | 0.04564956 |
| 88 - 191 | N88 | C191 | ING4_HUMAN - | −0.836365 | 0.04514178 |
| | | | CDYL_HUMAN | | |
| 55 - 18 | N55 | C18 | RBBP4_HUMAN - EZH2 | −0.837695 | 0.0351463 |
| 150 - 68 | N150 | C68 | HDA11_HUMAN - | −0.8383311 | 0.03239411 |
| | | | MBD3_HUMAN | | |
| 349 - 105 | N349 | C105 | DAM_N132A - | −0.8423693 | 0.02115676 |
| | | | MBD2_HUMAN | | |
| 9 - 32 | N9 | C32 | MECP2 - | −0.8430384 | 0.04090687 |
| | | | Gilbert_Lab_CRISPRi_KRAB | | |
| 324 - 68 | N324 | C68 | LBR_HUMAN - | −0.8469402 | 0.02458677 |
| | | | MBD3_HUMAN | | |
| 301 - 68 | N301 | C68 | SIRT3 - MBD3_HUMAN | −0.849936 | 0.02630305 |
| 280 - 191 | N280 | C191 | CBF1-Cterm - | −0.852393 | 0.045446 |
| | | | CDYL_HUMAN | | |
| 1 - 306 | N1 | C306 | SETD1A - CobB | −0.8528454 | 0.03349326 |
| 257 - 202 | N257 | C202 | c-Myb-TAD - | −0.8536751 | 0.02420565 |
| | | | KDM2B_HUMAN | | |
| 12 - 334 | N12 | C334 | KMT2A - hs_RCOR1 | −0.855811 | 0.0487242 |
| 306 - 98 | N306 | C98 | CobB - A4FU79_HUMAN | −0.8558462 | 0.02313278 |
| 99 - 334 | N99 | C334 | IN80C_HUMAN - | −0.8591778 | 0.04022742 |
| | | | hs RCOR1 | | |
| 257 - 306 | N257 | C306 | c-Myb-TAD - CobB | −0.8619406 | 0.04949716 |
| 64 - 8 | N64 | C8 | HDAC2_HUMAN - MECP2 | −0.861961 | 0.0357532 |
| 334 - 69 | N334 | C69 | hs_RCOR1 - | −0.8623571 | 0.03861085 |
| | | | SETB2_HUMAN | | |
| 106 - 306 | N106 | C306 | KAT6B_HUMAN - CobB | −0.8634006 | 0.03275628 |
| 306 - 73 | N306 | C73 | CobB - KDM5D_HUMAN | −0.8640893 | 0.02714014 |
| 331 - 293 | N331 | C293 | mm_G9a - KRAB-MeCP2 | −0.8660201 | 0.04798021 |
| 315 - 299 | N315 | C299 | NCoR - HST2 | −0.8666148 | 0.03328493 |
| 206 - 334 | N206 | C334 | SETD9_HUMAN - | −0.8667561 | 0.0306304 |
| | | | hs_RCOR1 | | |
| 3 - 274 | N3 | C274 | HDAC3 - UBE2A | −0.8689051 | 0.03155761 |
| 7 - 105 | N7 | C105 | ZNF10 - MBD2_HUMAN | −0.8703929 | 0.01206163 |
| 296 - 68 | N296 | C68 | HP1a - MBD3_HUMAN | −0.8748848 | 0.04379107 |
| 299 - 69 | N299 | C69 | HST2 - SETB2_HUMAN | −0.8794144 | 0.01603984 |
| 178 - 298 | N178 | C298 | KMT5C_HUMAN - MBD2b | −0.8804525 | 0.03691214 |
| 3 - 272 | N3 | C272 | HDAC3 - Sir2 | −0.8813947 | 0.04327273 |
| 3 - 302 | N3 | C302 | HDAC3 - HDAC11 | −0.8825378 | 0.03230839 |
| 295 - 55 | N295 | C55 | SET-TAF1B - | −0.8861273 | 0.02423242 |
| | | | RBBP4_HUMAN | | |
| 149 - 299 | N149 | C299 | MBD1_HUMAN - HST2 | −0.8862234 | 0.02155704 |
| 9 - 306 | N9 | C306 | MECP2 - CobB | −0.889198 | 0.02866609 |
| 116 - 306 | N116 | C306 | ANM6_HUMAN - CobB | −0.8913183 | 0.03528675 |
| 191 - 313 | N191 | C313 | CDYL_HUMAN - SaII | −0.8922318 | 0.03021425 |
| 14 - 105 | N14 | C105 | RELA - MBD2_HUMAN | −0.8927757 | 0.0128138 |
| 269 - 68 | N269 | C68 | PU.1 - MBD3_HUMAN | −0.8928238 | 0.04894686 |
| 93 - 105 | N93 | C105 | ACL6B_HUMAN - | −0.8944015 | 0.02505972 |
| | | | MBD2_HUMAN | | |
| 12 - 55 | N12 | C55 | KMT2A - RBBP4_HUMAN | −0.8960045 | 0.02749937 |
| 110 - 68 | N110 | C68 | JMJD4_HUMAN - | −0.8965348 | 0.02581839 |
| | | | MBD3_HUMAN | | |
| 55 - 320 | N55 | C320 | RBBP4_HUMAN - mMLL3- | −0.8978107 | 0.015056 |
| | | | SET | | |
| 293 - 313 | N293 | C313 | KRAB-MeCP2 - SaII | −0.898571 | 0.02459207 |
| 68 - 294 | N68 | C294 | MBD3_HUMAN - DIM5 | −0.8987594 | 0.04039954 |
| 344 - 32 | N344 | C32 | HDAC3_HUMAN - | −0.9005727 | 0.04177548 |
| | | | Gilbert_Lab_CRISPRi_KRAB | | |
| 317 - 105 | N317 | C105 | HDAC3 - MBD2_HUMAN | −0.9015505 | 0.0327984 |
| 165 - 257 | N165 | C257 | MTA2_HUMAN - c-Myb- | −0.9022729 | 0.04822938 |
| | | | TAD | | |
| 105 - 240 | N105 | C240 | MBD2_HUMAN - | −0.9030649 | 0.0429935 |
| | | | ANM3_HUMAN | | |
| 111 - 306 | N111 | C306 | KDM5A_HUMAN - CobB | −0.9031689 | 0.04109673 |
| 306 - 48 | N306 | C48 | CobB - SETD5_HUMAN | −0.9037086 | 0.0196052 |
| 306 - 173 | N306 | C173 | CobB - SIR4_HUMAN | −0.9040912 | 0.03412106 |
| 9 - 105 | N9 | C105 | MECP2 - MBD2_HUMAN | −0.9065253 | 0.01740606 |
| 293 - 8 | N293 | C8 | KRAB-MeCP2 - MECP2 | −0.912982 | 0.02534426 |
| 306 - 12 | N306 | C12 | CobB - KMT2A | −0.9148071 | 0.03554585 |
| 3 - 264 | N3 | C264 | HDAC3 - MTL5_HUMAN | −0.9148416 | 0.03063826 |
| 24 - 68 | N24 | C68 | CTCFL - MBD3_HUMAN | −0.9149332 | 0.02531457 |
| 298 - 59 | N298 | C59 | MBD2b - INO80_HUMAN | −0.9165078 | 0.02324262 |
| 64 - 68 | N64 | C68 | HDAC2_HUMAN - | −0.9181411 | 0.02030319 |
| | | | MBD3_HUMAN | | |
| 55 - 311 | N55 | C311 | RBBP4_HUMAN - Sin3a | −0.9205268 | 0.04200011 |
| 105 - 311 | N105 | C311 | MBD2_HUMAN - Sin3a | −0.9205612 | 0.01590709 |
| 17 - 306 | N17 | C306 | EZH2 - CobB | −0.9206735 | 0.02079487 |
| 299 - 70 | N299 | C70 | HST2 - SETB2_HUMAN | −0.920761 | 0.03547361 |
| 55 - 328 | N55 | C328 | RBBP4_HUMAN - | −0.9220582 | 0.04318922 |
| | | | hs_PHF19 | | |
| 196 - 111 | N196 | C111 | MTA1_HUMAN - | −0.923157 | 0.04443009 |
| | | | KDM5A_HUMAN | | |
| 3 - 187 | N3 | C187 | HDAC3 - MBD4_HUMAN | −0.9273379 | 0.03961541 |
| 152 - 314 | N152 | C314 | CDY1_HUMAN - MeCP2 | −0.9273586 | 0.02189626 |
| 3 - 90 | N3 | C90 | HDAC3 - CHD4_HUMAN | −0.9282359 | 0.01887574 |
| 298 - 222 | N298 | C222 | MBD2b - IN80E_HUMAN | −0.928589 | 0.04599651 |
| 345 - 306 | N345 | C306 | MSX2_HUMAN - CobB | −0.9288748 | 0.02803579 |
| 3 - 162 | N3 | C162 | HDAC3 - SIR6_HUMAN | −0.9301049 | 0.0376456 |
| 216 - 68 | N216 | C68 | ING5_HUMAN - | −0.9316134 | 0.04587804 |
| | | | MBD3_HUMAN | | |
| 148 - 32 | N148 | C32 | NSD3_HUMAN - | −0.9318579 | 0.01817869 |
| | | | Gilbert_Lab_CRISPRi_KRAB | | |
| 293 - 308 | N293 | C308 | KRAB-MeCP2 - HDT1 | −0.9320302 | 0.04996968 |
| 306 - 194 | N306 | C194 | CobB - ING1_HUMAN | −0.9321735 | 0.03835802 |
| 105 - 326 | N105 | C326 | MBD2_HUMAN - pf_Sir2A | −0.9323 | 0.01276008 |
| 217 - 306 | N217 | C306 | CHRC1_HUMAN - CobB | −0.9333309 | 0.01838788 |
| 55 - 294 | N55 | C294 | RBBP4_HUMAN - DIM5 | −0.9342276 | 0.01985834 |
| 313 - 185 | N313 | C185 | SaII - RBBP7_HUMAN | −0.9347245 | 0.02487677 |
| 105 - 67 | N105 | C67 | MBD2_HUMAN - | −0.934855 | 0.0376503 |
| | | | PRDM9_HUMAN | | |
| 317 - 77 | N317 | C77 | HDAC3 - KDM5D_HUMAN | −0.9349179 | 0.04230552 |
| 344 - 105 | N344 | C105 | HDAC3_HUMAN - | −0.9363938 | 0.04004722 |
| | | | MBD2_HUMAN | | |
| 240 - 306 | N240 | C306 | ANM3_HUMAN - CobB | −0.9373057 | 0.04617757 |
| 110 - 306 | N110 | C306 | JMJD4_HUMAN - CobB | −0.9424261 | 0.03735208 |
| 34 - 293 | N34 | C293 | DNMT3A-3L_JKNp112 - | −0.9426812 | 0.04476823 |
| | | | KRAB-MeCP2 | | |
| 344 - 68 | N344 | C68 | HDAC3_HUMAN - | −0.9428188 | 0.03167669 |
| | | | MBD3_HUMAN | | |
| 95 - 298 | N95 | C298 | SETD6_HUMAN - MBD2b | −0.943388 | 0.03196042 |
| 55 - 307 | N55 | C307 | RBBP4_HUMAN - KYP | −0.9443581 | 0.01114836 |
| 17 - 68 | N17 | C68 | EZH2 - MBD3_HUMAN | −0.9445766 | 0.02529228 |
| 105 - 198 | N105 | C198 | MBD2_HUMAN - | −0.9471001 | 0.01598412 |
| | | | JMJD8_HUMAN | | |
| 306 - 198 | N306 | C198 | CobB - JMJD8_HUMAN | −0.9481221 | 0.01793147 |
| 293 - 249 | N293 | C249 | KRAB-MeCP2 - HAC1-HAT | −0.9481891 | 0.04203393 |
| 136 - 334 | N136 | C334 | JMJD6_HUMAN - | −0.9486035 | 0.04148121 |
| | | | hs_RCOR1 | | |
| 106 - 191 | N106 | C191 | KAT6B_HUMAN - | −0.9493616 | 0.03925581 |
| | | | CDYL_HUMAN | | |
| 116 - 32 | N116 | C32 | ANM6_HUMAN - | −0.9493879 | 0.01467407 |
| | | | Gilbert_Lab_CRISPRi_KRAB | | |
| 182 - 266 | N182 | C266 | MTA3_HUMAN - | −0.9504726 | 0.04236039 |
| | | | GADD45A | | |
| 293 - 332 | N293 | C332 | KRAB-MeCP2 - hs_SIRT5 | −0.9510193 | 0.02525373 |
| 97 - 105 | N97 | C105 | CHD3_HUMAN - | −0.9510415 | 0.01450643 |
| | | | MBD2_HUMAN | | |
| 190 - 306 | N190 | C306 | ANM1_HUMAN - CobB | −0.9515736 | 0.02024869 |
| 107 - 98 | N107 | C98 | KAT6B_HUMAN - | −0.9518743 | 0.0393631 |
| | | | A4FU79_HUMAN | | |
| 123 - 293 | N123 | C293 | KMT2A_HUMAN - KRAB- | −0.9520689 | 0.01601515 |
| | | | MeCP2 | | |
| 105 - 113 | N105 | C113 | MBD2_HUMAN - | −0.9545914 | 0.0373083 |
| | | | KDM5A_HUMAN | | |
| 344 - 315 | N344 | C315 | HDAC3_HUMAN - NCoR | −0.9546251 | 0.0462916 |
| 55 - 346 | N55 | C346 | RBBP4_HUMAN - | −0.9547442 | 0.02125452 |
| | | | MSX2_HUMAN | | |
| 105 - 75 | N105 | C75 | MBD2_HUMAN - | −0.9563708 | 0.02831842 |
| | | | KDM5D_HUMAN | | |
| 182 - 17 | N182 | C17 | MTA3_HUMAN - EZH2 | −0.9567312 | 0.04275125 |
| 68 - 307 | N68 | C307 | MBD3_HUMAN - KYP | −0.957024 | 0.03190693 |
| 133 - 68 | N133 | C68 | KAT8_HUMAN - | −0.9588149 | 0.04965198 |
| | | | MBD3_HUMAN | | |
| 306 - 38 | N306 | C38 | CobB - EHMT2 | −0.9594752 | 0.01464886 |
| 114 - 312 | N114 | C312 | KDM5A_HUMAN - SetD8 | −0.9605539 | 0.04762153 |
| 68 - 205 | N68 | C205 | MBD3_HUMAN - | −0.9606339 | 0.01121701 |
| | | | JMJD7_HUMAN | | |
| 160 - 105 | N160 | C105 | KMT5A_HUMAN - | −0.9606821 | 0.04823924 |
| | | | MBD2_HUMAN | | |
| 7 - 314 | N7 | C314 | ZNF10 - MeCP2 | −0.9614913 | 0.03196762 |
| 182 - 329 | N182 | C329 | MTA3_HUMAN - | −0.9616121 | 0.03013193 |
| | | | pbcv1_vSET | | |
| 111 - 182 | N111 | C182 | KDM5A_HUMAN - | −0.9620861 | 0.03900995 |
| | | | MTA3_HUMAN | | |
| 182 - 127 | N182 | C127 | MTA3_HUMAN - | −0.9626564 | 0.04934178 |
| | | | SETMR_HUMAN | | |
| 182 - 98 | N182 | C98 | MTA3_HUMAN - | −0.9632167 | 0.03574115 |
| | | | A4FU79_HUMAN | | |
| 55 - 326 | N55 | C326 | RBBP4_HUMAN - pf_Sir2A | −0.9636666 | 0.03000368 |
| 314 - 56 | N314 | C56 | MeCP2 - SUV92_HUMAN | −0.9645615 | 0.02158173 |
| 344 - 241 | N344 | C241 | HDAC3_HUMAN - | −0.9651722 | 0.04465046 |
| | | | SIR2_HUMAN | | |
| 246 - 55 | N246 | C55 | ATRX_HUMAN - | −0.9677047 | 0.03384891 |
| | | | RBBP4_HUMAN | | |
| 17 - 159 | N17 | C159 | EZH2 - SETB1_HUMAN | −0.9679473 | 0.03800782 |
| 48 - 306 | N48 | C306 | SETD5_HUMAN - CobB | −0.9696999 | 0.01512652 |
| 68 - 213 | N68 | C213 | MBD3_HUMAN - | −0.9703471 | 0.02548835 |
| | | | HBAP1_HUMAN | | |
| 64 - 306 | N64 | C306 | HDAC2_HUMAN - CobB | −0.9704444 | 0.03382249 |
| 3 - 5 | N3 | C5 | HDAC3 - KDM1A | −0.9704956 | 0.04752246 |
| 152 - 306 | N152 | C306 | CDY1_HUMAN - CobB | −0.9705761 | 0.03678002 |
| 105 - 307 | N105 | C307 | MBD2_HUMAN - KYP | −0.9758561 | 0.00991856 |
| 70 - 272 | N70 | C272 | SETB2_HUMAN - Sir2 | −0.9759851 | 0.03902477 |
| 299 - 306 | N299 | C306 | HST2 - CobB | −0.9773907 | 0.0114648 |
| 334 - 306 | N334 | C306 | hs_RCOR1 - CobB | −0.9774973 | 0.01524066 |
| 222 - 120 | N222 | C120 | IN80E_HUMAN - | −0.9784421 | 0.04291692 |
| | | | RING1_HUMAN | | |
| 68 - 123 | N68 | C123 | MBD3_HUMAN - | −0.9792208 | 0.01066874 |
| | | | KMT2A_HUMAN | | |
| 306 - 214 | N306 | C214 | CobB - HDAC4_HUMAN | −0.9796415 | 0.01739203 |
| 55 - 130 | N55 | C130 | RBBP4_HUMAN - | −0.9796604 | 0.03691028 |
| | | | PRDM2_HUMAN | | |
| 68 - 249 | N68 | C249 | MBD3_HUMAN - HAC1- | −0.9800445 | 0.03910064 |
| | | | HAT | | |
| 306 - 315 | N306 | C315 | CobB - NCoR | −0.9814031 | 0.02475436 |
| 105 - 274 | N105 | C274 | MBD2_HUMAN - UBE2A | −0.9814465 | 0.01120869 |
| 18 - 334 | N18 | C334 | EZH2 - hs_RCOR1 | −0.9816402 | 0.03436169 |
| 51 - 298 | N51 | C298 | UCHL5_HUMAN - MBD2b | −0.9831036 | 0.00782736 |
| 306 - 132 | N306 | C132 | CobB - PRDM2_HUMAN | −0.9857575 | 0.04542866 |
| 299 - 118 | N299 | C118 | HST2 - HDAC6_HUMAN | −0.9862852 | 0.0425698 |
| 301 - 298 | N301 | C298 | SIRT3 - MBD2b | −0.9871333 | 0.0304159 |
| 317 - 271 | N317 | C271 | HDAC3 - GCN5 | −0.9872627 | 0.02946941 |
| 317 - 7 | N317 | C7 | HDAC3 - ZNF10 | −0.9876734 | 0.03968013 |
| 108 - 241 | N108 | C241 | KAT6B_HUMAN - | −0.9887059 | 0.03349353 |
| | | | SIR2_HUMAN | | |
| 105 - 305 | N105 | C305 | MBD2_HUMAN - NUE | −0.9895004 | 0.00665336 |
| 324 - 272 | N324 | C272 | LBR_HUMAN - Sir2 | −0.9921594 | 0.02832094 |
| 268 - 241 | N268 | C241 | FOXA1-Cterm - | −0.9927853 | 0.02868779 |
| | | | SIR2_HUMAN | | |
| 105 - 317 | N105 | C317 | MBD2_HUMAN - HDAC3 | −0.9934778 | 0.02458553 |
| 173 - 68 | N173 | C68 | SIR4_HUMAN - | −0.9946145 | 0.01087598 |
| | | | MBD3_HUMAN | | |
| 222 - 9 | N222 | C9 | IN80E_HUMAN - MECP2 | −0.9948162 | 0.01560088 |
| 315 - 293 | N315 | C293 | NCoR - KRAB-MeCP2 | −0.9969902 | 0.01413661 |
| 334 - 314 | N334 | C314 | hs_RCOR1 - MeCP2 | −0.9974173 | 0.01654964 |
| 216 - 215 | N216 | C215 | ING5_HUMAN - | −0.997547 | 0.02084241 |
| | | | HDAC4_HUMAN | | |
| 218 - 32 | N218 | C32 | IN80B_HUMAN - | −0.9976831 | 0.018373 |
| | | | Gilbert_Lab_CRISPRi_KRAB | | |
| 275 - 68 | N275 | C68 | DOT1L-DOT1_domain - | −0.9977186 | 0.00818521 |
| | | | MBD3_HUMAN | | |
| 298 - 302 | N298 | C302 | MBD2b - HDAC11 | −0.9980778 | 0.01509792 |
| 3 - 329 | N3 | C329 | HDAC3 - pbcv1_vSET | −1.0004442 | 0.03371301 |
| 324 - 101 | N324 | C101 | LBR_HUMAN - | −1.0006384 | 0.03727279 |
| | | | TAF1_HUMAN | | |
| 299 - 315 | N299 | C315 | HST2 - NCoR | −1.0008074 | 0.00769792 |
| 130 - 105 | N130 | C105 | PRDM2_HUMAN - | −1.0009196 | 0.00905205 |
| | | | MBD2_HUMAN | | |
| 241 - 313 | N241 | C313 | SIR2_HUMAN - SaII | −1.0009662 | 0.04141994 |
| 299 - 314 | N299 | C314 | HST2 - MeCP2 | −1.0021141 | 0.01681023 |
| 26 - 191 | N26 | C191 | EZH1 - CDYL_HUMAN | −1.0021514 | 0.03385219 |
| 105 - 253 | N105 | C253 | MBD2_HUMAN - | −1.0022448 | 0.02858658 |
| | | | RBBP5_HUMAN | | |
| 55 - 271 | N55 | C271 | RBBP4_HUMAN - GCN5 | −1.0027822 | 0.00876914 |
| 48 - 105 | N48 | C105 | SETD5_HUMAN - | −1.0030502 | 0.03034523 |
| | | | MBD2_HUMAN | | |
| 105 -205 | N105 | C205 | MBD2_HUMAN - | −1.003914 | 0.02100605 |
| | | | JMJD7_HUMAN | | |
| 68 - 12 | N68 | C12 | MBD3_HUMAN - KMT2A | −1.0051479 | 0.02385168 |
| 246 - 191 | N246 | C191 | ATRX_HUMAN - | −1.0056981 | 0.04822208 |
| | | | CDYL_HUMAN | | |
| 112 - 299 | N112 | C299 | KDM5A_HUMAN - HST2 | −1.0066206 | 0.02799353 |
| 334 - 24 | N334 | C24 | hs_RCOR1 - CTCFL | −1.0086335 | 0.04752628 |
| 55 - 218 | N55 | C218 | RBBP4_HUMAN - | −1.0107754 | 0.03250857 |
| | | | IN80B_HUMAN | | |
| 105 - 199 | N105 | C199 | MBD2_HUMAN - | −1.0110029 | 0.02683139 |
| | | | ING3_HUMAN | | |
| 127 - 55 | N127 | C55 | SETMR_HUMAN - | −1.0119695 | 0.02153403 |
| | | | RBBP4_HUMAN | | |
| 307 - 32 | N307 | C32 | KYP - | −1.011974 | 0.00745792 |
| | | | Gilbert_Lab_CRISPRi_KRAB | | |
| 341 - 257 | N341 | C257 | NCOR2_HUMAN - c-Myb- | −1.0141537 | 0.03607145 |
| | | | TAD | | |
| 344 - 187 | N344 | C187 | HDAC3_HUMAN - | −1.0143205 | 0.03945905 |
| | | | MBD4_HUMAN | | |
| 275 - 306 | N275 | C306 | DOT1L-DOT1_domain - | −1.014421 | 0.01379276 |
| | | | CobB | | |
| 312 - 32 | N312 | C32 | SetD8 - | −1.0168854 | 0.02452912 |
| | | | Gilbert_Lab_CRISPRi_KRAB | | |
| 68 - 63 | N68 | C63 | MBD3_HUMAN - | −1.0169899 | 0.04052079 |
| | | | SMYD5_HUMAN | | |
| 21 - 293 | N21 | C293 | KDM1B - KRAB-MeCP2 | −1.0199815 | 0.04617611 |
| 151 - 299 | N151 | C299 | SIR7_HUMAN - HST2 | −1.02137 | 0.03740468 |
| 257 - 334 | N257 | C334 | c-Myb-TAD - hs_RCOR1 | −1.0230369 | 0.04536257 |
| 297 - 306 | N297 | C306 | MesoLo - CobB | −1.02341 | 0.03193605 |
| 3 - 123 | N3 | C123 | HDAC3 - KMT2A_HUMAN | −1.02393 | 0.03003599 |
| 68 - 190 | N68 | C190 | MBD3_HUMAN - | −1.0240703 | 0.03888487 |
| | | | ANM1_HUMAN | | |
| 216 - 191 | N216 | C191 | ING5_HUMAN - | −1.0249298 | 0.02154985 |
| | | | CDYL_HUMAN | | |
| 306 - 346 | N306 | C346 | CobB - MSX2_HUMAN | −1.0258624 | 0.01337699 |
| 266 - 105 | N266 | C105 | GADD45A - | −1.0260416 | 0.01802933 |
| | | | MBD2_HUMAN | | |
| 205 - 68 | N205 | C68 | JMJD7_HUMAN - | −1.0262792 | 0.00861769 |
| | | | MBD3_HUMAN | | |
| 112 - 306 | N112 | C306 | KDM5A_HUMAN - CobB | −1.0268359 | 0.01298875 |
| 299 - 255 | N299 | C255 | HST2 - EED_HUMAN | −1.0270233 | 0.01984675 |
| 37 - 105 | N37 | C105 | KDM6A - MBD2_HUMAN | −1.0273041 | 0.04264092 |
| 55- 301 | N55 | C301 | RBBP4_HUMAN - SIRT3 | −1.028757 | 0.01989918 |
| 345 - 191 | N345 | C191 | MSX2_HUMAN - | −1.0292576 | 0.03045319 |
| | | | CDYL_HUMAN | | |
| 219 - 32 | N219 | C32 | JARD2_HUMAN - | −1.0296542 | 0.0333184 |
| | | | Gilbert_Lab_CRISPRi_KRAB | | |
| 1 - 68 | N1 | C68 | SETD1A - MBD3_HUMAN | −1.0302828 | 0.03628168 |
| 334 - 255 | N334 | C255 | hs_RCOR1 - EED_HUMAN | −1.0303542 | 0.02091723 |
| 55 - 308 | N55 | C308 | RBBP4_HUMAN - HDT1 | −1.031211 | 0.00627947 |
| 112 - 215 | N112 | C215 | KDM5A_HUMAN - | −1.0312786 | 0.0314425 |
| | | | HDAC4_HUMAN | | |
| 68 - 26 | N68 | C26 | MBD3_HUMAN - EZH1 | −1.03205 | 0.04652736 |
| 3 - 248 | N3 | C248 | HDAC3 - GLYR1_HUMAN | −1.0320535 | 0.04209586 |
| 202 - 246 | N202 | C246 | KDM2B_HUMAN - | −1.0321336 | 0.03609716 |
| | | | ATRX_HUMAN | | |
| 153 - 105 | N153 | C105 | KAT7_HUMAN - | −1.0321752 | 0.03474105 |
| | | | MBD2_HUMAN | | |
| 55 - 26 | N55 | C26 | RBBP4_HUMAN - EZH1 | −1.0325618 | 0.00382336 |
| 75 - 179 | N75 | C179 | KDM5D_HUMAN - | −1.0332611 | 0.04263479 |
| | | | SMRD2_HUMAN | | |
| 306 - 334 | N306 | C334 | CobB - hs_RCOR1 | −1.0337931 | 0.00749476 |
| 331 - 306 | N331 | C306 | mm_G9a - CobB | −1.0343478 | 0.02671083 |
| 131 - 346 | N131 | C346 | PRDM2_HUMAN - | −1.0348068 | 0.0322101 |
| | | | MSX2_HUMAN | | |
| 17 - 55 | N17 | C55 | EZH2 - RBBP4_HUMAN | −1.0350938 | 0.03747672 |
| 3 - 116 | N3 | C116 | HDAC3 - ANM6_HUMAN | −1.0352093 | 0.03325349 |
| 3 - 301 | N3 | C301 | HDAC3 - SIRT3 | −1.0391541 | 0.03204255 |
| 315 - 191 | N315 | C191 | NCoR - CDYL_HUMAN | −1.039578 | 0.01975919 |
| 55 -1 | N55 | C1 | RBBP4_HUMAN - SETD1A | −1.0397781 | 0.00530034 |
| 198 - 105 | N198 | C105 | JMJD8_HUMAN - | −1.0412798 | 0.00423853 |
| | | | MBD2_HUMAN | | |
| 55 - 233 | N55 | C233 | RBBP4_HUMAN - | −1.0421154 | 0.00651682 |
| | | | HINFP_HUMAN | | |
| 105 - 277 | N105 | C277 | MBD2_HUMAN - SUV39H1 | −1.0424544 | 0.04269971 |
| 344 - 348 | N344 | C348 | HDAC3_HUMAN - | −1.0426226 | 0.04060724 |
| | | | BRDT_HUMAN | | |
| 45 - 131 | N45 | C131 | KMT2D_HUMAN - | −1.0433583 | 0.04672602 |
| | | | PRDM2_HUMAN | | |
| 61 - 189 | N61 | C189 | HAT1_HUMAN - | −1.0447386 | 0.04556948 |
| | | | ING2_HUMAN | | |
| 206 - 299 | N206 | C299 | SETD9_HUMAN - HST2 | −1.0456466 | 0.01341223 |
| 312 - 191 | N312 | C191 | SetD8 - CDYL_HUMAN | −1.0458998 | 0.02742139 |
| 8 - 306 | N8 | C306 | MECP2 - CobB | −1.0463324 | 0.01851408 |
| 198 - 298 | N198 | C298 | JMJD8_HUMAN - MBD2b | −1.0465953 | 0.04175433 |
| 317 - 133 | N317 | C133 | HDAC3 - KAT8_HUMAN | −1.0471015 | 0.0189839 |
| 114 - 105 | N114 | C105 | KDM5A_HUMAN - | −1.0474753 | 0.00833394 |
| | | | MBD2_HUMAN | | |
| 280 - 68 | N280 | C68 | CBF1-Cterm - | −1.0477046 | 0.02004965 |
| | | | MBD3_HUMAN | | |
| 68 - 85 | N68 | C85 | MBD3_HUMAN - | −1.0496158 | 0.01143337 |
| | | | SMYD3_HUMAN | | |
| 336 - 332 | N336 | C332 | ce_Set4 - hs_SIRT5 | −1.050722 | 0.04477041 |
| 3 - 349 | N3 | C349 | HDAC3 - DAM_N132A | −1.0512051 | 0.00473302 |
| 293 - 216 | N293 | C216 | KRAB-MeCP2 - | −1.0517161 | 0.02351314 |
| | | | ING5_HUMAN | | |
| 314 - 328 | N314 | C328 | MeCP2 - hs_PHF19 | −1.0517575 | 0.04588606 |
| 306 - 56 | N306 | C56 | CobB - SUV92_HUMAN | −1.0522014 | 0.00489633 |
| 306 - 327 | N306 | C327 | CobB - hs_NIPP1 | −1.0529254 | 0.02130192 |
| 306 - 213 | N306 | C213 | CobB - HBAP1_HUMAN | −1.0537058 | 0.02395416 |
| 174 - 55 | N174 | C55 | HDAC1_HUMAN - | −1.0539197 | 0.02328572 |
| | | | RBBP4_HUMAN | | |
| 3 - 113 | N3 | C113 | HDAC3 - KDM5A_HUMAN | −1.0548659 | 0.04255794 |
| 68 - 99 | N68 | C99 | MBD3_HUMAN - | −1.0552102 | 0.02292798 |
| | | | IN80C_HUMAN | | |
| 298 - 98 | N298 | C98 | MBD2b - A4FU79_HUMAN | −1.0559273 | 0.04032337 |
| 99 - 105 | N99 | C105 | IN80C_HUMAN - | −1.0569275 | 0.01439029 |
| | | | MBD2_HUMAN | | |
| 306 - 339 | N306 | C339 | CobB - SPOC1_HUMAN | −1.0585621 | 0.02610275 |
| 306 - 165 | N306 | C165 | CobB - MTA2_HUMAN | −1.0598681 | 0.02006791 |
| 346 - 306 | N346 | C306 | MSX2_HUMAN - CobB | −1.0609136 | 0.00555032 |
| 317 - 130 | N317 | C130 | HDAC3 - PRDM2_HUMAN | −1.0615276 | 0.0246484 |
| 55 - 312 | N55 | C312 | RBBP4_HUMAN - SetD8 | −1.0620524 | 0.03251547 |
| 246 - 152 | N246 | C152 | ATRX_HUMAN - | −1.0625222 | 0.01554699 |
| | | | CDY1_HUMAN | | |
| 53 - 272 | N53 | C272 | CBP_HUMAN - Sir2 | −1.0625649 | 0.02332174 |
| 222 - 255 | N222 | C255 | IN80E_HUMAN - | −1.0626487 | 0.02513504 |
| | | | EED_HUMAN | | |
| 42 - 105 | N42 | C105 | KMT2E_HUMAN - | −1.0628727 | 0.01940013 |
| | | | MBD2_HUMAN | | |
| 185 - 32 | N185 | C32 | RBBP7_HUMAN - | −1.0663611 | 0.01023072 |
| | | | Gilbert_Lab_CRISPRi_KRAB | | |
| 298 - 123 | N298 | C123 | MBD2b - KMT2A_HUMAN | −1.0663868 | 0.01047282 |
| 298 - 127 | N298 | C127 | MBD2b - SETMR_HUMAN | −1.0673426 | 0.00661031 |
| 68 - 47 | N68 | C47 | MBD3_HUMAN - | −1.0684175 | 0.03477035 |
| | | | ANM2_HUMAN | | |
| 313 - 182 | N313 | C182 | SaII - MTA3_HUMAN | −1.068855 | 0.04365798 |
| 298 - 183 | N298 | C183 | MBD2b - KMT2C_HUMAN | −1.0691228 | 0.0196351 |
| 68 - 73 | N68 | C73 | MBD3_HUMAN - | −1.0692642 | 0.0196972 |
| | | | KDM5D_HUMAN | | |
| 105 - 301 | N105 | C301 | MBD2_HUMAN - SIRT3 | −1.0701441 | 0.00486377 |
| 334 - 299 | N334 | C299 | hs_RCOR1 - HST2 | −1.0720622 | 0.00925233 |
| 256 - 349 | N256 | C349 | LSD1-ORF - DAM_N132A | −1.0720979 | 0.0381458 |
| 306 - 27 | N306 | C27 | CobB - EZH1 | −1.0734454 | 0.0105935 |
| 329 - 293 | N329 | C293 | pbcv1_vSET - KRAB-MeCP2 | −1.0744457 | 0.02274611 |
| 306 - 212 | N306 | C212 | CobB - HDAC8_HUMAN | −1.0748079 | 0.01609368 |
| 8 - 105 | N8 | C105 | MECP2 - MBD2_HUMAN | −1.0756978 | 0.04662386 |
| 76 - 105 | N76 | C105 | KDM5D_HUMAN - | −1.077082 | 0.01579199 |
| | | | MBD2_HUMAN | | |
| 9 - 298 | N9 | C298 | MECP2 - MBD2b | −1.0772667 | 0.0109274 |
| 273 - 334 | N273 | C334 | PRDM9-CD - hs_RCOR1 | −1.0773677 | 0.03734943 |
| 309 - 189 | N309 | C189 | HDAC8 - ING2_HUMAN | −1.0791442 | 0.02960011 |
| 313 - 210 | N313 | C210 | SaII - SIR3_HUMAN | −1.0797043 | 0.02118921 |
| 263 - 105 | N263 | C105 | HDAC8 - MBD2_HUMAN | −1.0812784 | 0.00895141 |
| 345 - 120 | N345 | C120 | MSX2_HUMAN - | −1.0819609 | 0.01629167 |
| | | | RING1_HUMAN | | |
| 246 - 215 | N246 | C215 | ATRX_HUMAN - | −1.0826768 | 0.01786685 |
| | | | HDAC4_HUMAN | | |
| 119 - 105 | N119 | C105 | ACL6A_HUMAN - | −1.0853299 | 0.03819898 |
| | | | MBD2_HUMAN | | |
| 105 - 95 | N105 | C95 | MBD2_HUMAN - | −1.0853915 | 0.01019326 |
| | | | SETD6_HUMAN | | |
| 182 - 51 | N182 | C51 | MTA3_HUMAN - | −1.0854769 | 0.04632882 |
| | | | UCHL5_HUMAN | | |
| 269 - 306 | N269 | C306 | PU.1 - CobB | −1.0896083 | 0.01396673 |
| 297 - 299 | N297 | C299 | MesoLo - HST2 | −1.0900788 | 0.03427989 |
| 227 - 299 | N227 | C299 | SIR1_HUMAN - HST2 | −1.0914553 | 0.0444958 |
| 345 - 68 | N345 | C68 | MSX2_HUMAN - | −1.0921822 | 0.02653435 |
| | | | MBD3_HUMAN | | |
| 67 - 298 | N67 | C298 | PRDM9_HUMAN - MBD2b | −1.0927784 | 0.00608492 |
| 324 - 105 | N324 | C105 | LBR_HUMAN - | −1.0933781 | 0.00415363 |
| | | | MBD2_HUMAN | | |
| 204 - 32 | N204 | C32 | HIFIN_HUMAN - | −1.0951782 | 0.02901006 |
| | | | Gilbert_Lab_CRISPRi_KRAB | | |
| 306 - 305 | N306 | C305 | CobB - NUE | −1.0951921 | 0.01311964 |
| 216 - 7 | N216 | C7 | ING5_HUMAN - ZNF10 | −1.0960796 | 0.02381687 |
| 257 - 165 | N257 | C165 | c-Myb-TAD - | −1.0961791 | 0.02207992 |
| | | | MTA2_HUMAN | | |
| 106 - 182 | N106 | C182 | KAT6B_HUMAN - | −1.0963274 | 0.03748168 |
| | | | MTA3_HUMAN | | |
| 302 - 306 | N302 | C306 | HDAC11 - CobB | −1.0968145 | 0.03436083 |
| 306 - 310 | N306 | C310 | CobB - TgSET8 | −1.0974434 | 0.00531421 |
| 45 - 298 | N45 | C298 | KMT2D_HUMAN - MBD2b | −1.100898 | 0.00419112 |
| 299 - 298 | N299 | C298 | HST2 - MBD2b | −1.1021451 | 0.00780196 |
| 55 - 110 | N55 | C110 | RBBP4_HUMAN - | −1.1028697 | 0.03485299 |
| | | | JMJD4_HUMAN | | |
| 299 - 226 | N299 | C226 | HST2 - SETD2_HUMAN | −1.10321 | 0.0062239 |
| 105 - 349 | N105 | C349 | MBD2_HUMAN - | −1.1045283 | 0.00773046 |
| | | | DAM_N132A | | |
| 306 - 217 | N306 | C217 | CobB - CHRC1_HUMAN | −1.1060173 | 0.00741513 |
| 344 - 313 | N344 | C313 | HDAC3_HUMAN - SaII | −1.1072537 | 0.00410744 |
| 38 - 32 | N38 | C32 | EHMT2 - | −1.1089348 | 0.0053771 |
| | | | Gilbert_Lab_CRISPRi_KRAB | | |
| 55 - 187 | N55 | C187 | RBBP4_HUMAN - | −1.1096985 | 0.01470158 |
| | | | MBD4_HUMAN | | |
| 182 - 312 | N182 | C312 | MTA3_HUMAN - SetD8 | −1.1107623 | 0.0301305 |
| 154 - 306 | N154 | C306 | NSD2_HUMAN - CobB | −1.1107746 | 0.02936919 |
| 298 - 173 | N298 | C173 | MBD2b - SIR4_HUMAN | −1.1108274 | 0.01039517 |
| 296 - 206 | N296 | C206 | HP1a - SETD9_HUMAN | −1.1118594 | 0.0150508 |
| 7 - 334 | N7 | C334 | ZNF10 - hs_RCOR1 | −1.1122925 | 0.01654218 |
| 3 - 77 | N3 | C77 | HDAC3 - KDM5D_HUMAN | −1.1129116 | 0.01768058 |
| 3 - 56 | N3 | C56 | HDAC3 - SUV92_HUMAN | −1.1129828 | 0.00512942 |
| 167 - 315 | N167 | C315 | EHMT1_HUMAN - NCoR | −1.1133318 | 0.01265285 |
| 69 - 344 | N69 | C344 | SETB2_HUMAN - | −1.1136108 | 0.02906772 |
| | | | HDAC3_HUMAN | | |
| 231 - 298 | N231 | C298 | KDM5B_HUMAN - MBD2b | −1.1141531 | 0.02679908 |
| 18 - 299 | N18 | C299 | EZH2 - HST2 | −1.114752 | 0.01760116 |
| 199 - 105 | N199 | C105 | ING3_HUMAN - | −1.1151373 | 0.01571416 |
| | | | MBD2_HUMAN | | |
| 73 - 334 | N73 | C334 | KDM5D_HUMAN - | −1.1152733 | 0.01368043 |
| | | | hs RCOR1 | | |
| 107 - 334 | N107 | C334 | KAT6B_HUMAN - | −1.1153376 | 0.03956565 |
| | | | hs_RCOR1 | | |
| 165 - 269 | N165 | C269 | MTA2_HUMAN - PU.1 | −1.1166466 | 0.01598225 |
| 306 - 150 | N306 | C150 | CobB - HDA11_HUMAN | −1.116721 | 0.01084533 |
| 334 - 328 | N334 | C328 | hs_RCOR1 - hs_PHF19 | −1.1167772 | 0.02960846 |
| 68 - 297 | N68 | C297 | MBD3_HUMAN - MesoLo | −1.1168196 | 0.00304578 |
| 68 - 56 | N68 | C56 | MBD3_HUMAN - | −1.1173499 | 0.00658753 |
| | | | SUV92_HUMAN | | |
| 306 - 274 | N306 | C274 | CobB - UBE2A | −1.1174172 | 0.01252272 |
| 68 - 346 | N68 | C346 | MBD3_HUMAN - | −1.117736 | 0.01061328 |
| | | | MSX2_HUMAN | | |
| 215 - 326 | N215 | C326 | HDAC4_HUMAN - pf_Sir2A | −1.1179218 | 0.03662769 |
| 68 - 287 | N68 | C287 | MBD3_HUMAN - G9A[SET] | −1.1181832 | 0.00642735 |
| 112 - 68 | N112 | C68 | KDM5A_HUMAN - | −1.1183068 | 0.03925258 |
| | | | MBD3_HUMAN | | |
| 105 - 213 | N105 | C213 | MBD2_HUMAN - | −1.1184602 | 0.01341649 |
| | | | HBAP1_HUMAN | | |
| 220 - 68 | N220 | C68 | JARD2_HUMAN - | −1.1187564 | 0.01678901 |
| | | | MBD3_HUMAN | | |
| 105 -250 | N105 | C250 | MBD2_HUMAN - | −1.1192258 | 0.00772996 |
| | | | HCFC1_HUMAN | | |
| 164 - 334 | N164 | C334 | SIR5_HUMAN - hs_RCOR1 | −1.1193037 | 0.02821446 |
| 119 - 298 | N119 | C298 | ACL6A_HUMAN - MBD2b | −1.1196597 | 0.03617778 |
| 298 - 158 | N298 | C158 | MBD2b - SETB1_HUMAN | −1.1205985 | 0.03801515 |
| 3 - 271 | N3 | C271 | HDAC3 - GCN5 | −1.1214741 | 0.01383531 |
| 315 - 215 | N315 | C215 | NCoR - HDAC4_HUMAN | −1.1219799 | 0.0174359 |
| 334 - 107 | N334 | C107 | hs_RCOR1 - | −1.1226131 | 0.02028194 |
| | | | KAT6B_HUMAN | | |
| 298 - 339 | N298 | C339 | MBD2b - SPOC1_HUMAN | −1.1230257 | 0.04903756 |
| 57 - 306 | N57 | C306 | ANM8_HUMAN - CobB | −1.1230637 | 0.01005067 |
| 68 - 77 | N68 | C77 | MBD3_HUMAN - | −1.1230837 | 0.01501509 |
| | | | KDM5D_HUMAN | | |
| 100 - 105 | N100 | C105 | TAF1_HUMAN - | −1.1232314 | 0.0091678 |
| | | | MBD2_HUMAN | | |
| 334 - 105 | N334 | C105 | hs_RCOR1 - | −1.1232924 | 0.00202709 |
| | | | MBD2_HUMAN | | |
| 277 - 68 | N277 | C68 | SUV39H1 - MBD3_HUMAN | −1.1238133 | 0.02717255 |
| 345 - 131 | N345 | C131 | MSX2_HUMAN - | −1.1250176 | 0.0479504 |
| | | | PRDM2_HUMAN | | |
| 313 - 189 | N313 | C189 | SaII - ING2_HUMAN | −1.1255031 | 0.01270411 |
| 293 - 288 | N293 | C288 | KRAB-MeCP2 - SUV[SET] | −1.1269544 | 0.04504326 |
| 306 - 276 | N306 | C276 | CobB - EHMT2(G9a)-Cterm | −1.1276173 | 0.04340083 |
| 266 - 293 | N266 | C293 | GADD45A - KRAB-MeCP2 | −1.1281033 | 0.02028235 |
| 105 - 177 | N105 | C177 | MBD2_HUMAN - | −1.1281501 | 0.01443018 |
| | | | KDM5C_HUMAN | | |
| 345 - 186 | N345 | C186 | MSX2_HUMAN - | −1.1287238 | 0.03444423 |
| | | | RIOX2_HUMAN | | |
| 68 - 296 | N68 | C296 | MBD3_HUMAN - HP1a | −1.129694 | 0.02562654 |
| 105 - 98 | N105 | C98 | MBD2_HUMAN - | −1.1301046 | 0.00639036 |
| | | | A4FU79_HUMAN | | |
| 3 - 9 | N3 | C9 | HDAC3 - MECP2 | −1.1303388 | 0.0186794 |
| 105 - 221 | N105 | C221 | MBD2_HUMAN - | −1.1309518 | 0.01244784 |
| | | | SMYD2_HUMAN | | |
| 105 - 136 | N105 | C136 | MBD2_HUMAN - | −1.1314019 | 0.0255724 |
| | | | JMJD6_HUMAN | | |
| 306 - 302 | N306 | C302 | CobB - HDAC11 | −1.1316662 | 0.00518394 |
| 133 - 241 | N133 | C241 | KAT8_HUMAN - | −1.1317431 | 0.02850798 |
| | | | SIR2_HUMAN | | |
| 105 - 130 | N105 | C130 | MBD2_HUMAN - | −1.132387 | 0.00775862 |
| | | | PRDM2_HUMAN | | |
| 95 - 68 | N95 | C68 | SETD6_HUMAN - | −1.1328969 | 0.02467867 |
| | | | MBD3_HUMAN | | |
| 255 - 32 | N255 | C32 | EED_HUMAN - | −1.1331292 | 0.00827365 |
| | | | Gilbert_Lab_CRISPRi_KRAB | | |
| 61 - 241 | N61 | C241 | HAT1_HUMAN - | −1.1331982 | 0.03062288 |
| | | | SIR2_HUMAN | | |
| 196 - 233 | N196 | C233 | MTA1_HUMAN - | −1.1340928 | 0.03716628 |
| | | | HINFP_HUMAN | | |
| 306 - 34 | N306 | C34 | CobB - DNMT3A- | −1.1343713 | 0.00779404 |
| | | | 3L_JKNp112 | | |
| 306 - 175 | N306 | C175 | CobB - RUVB2_HUMAN | −1.1354303 | 0.03126122 |
| 3 - 69 | N3 | C69 | HDAC3 - SETB2_HUMAN | −1.1355944 | 0.00723298 |
| 117 - 32 | N117 | C32 | HDAC6_HUMAN - | −1.1363265 | 0.04353037 |
| | | | Gilbert_Lab_CRISPRi_KRAB | | |
| 212 - 334 | N212 | C334 | HDAC8_HUMAN - | −1.1366467 | 0.0132466 |
| | | | hs RCOR1 | | |
| 67 - 68 | N67 | C68 | PRDM9_HUMAN - | −1.1369054 | 0.01583047 |
| | | | MBD3_HUMAN | | |
| 195 - 306 | N195 | C306 | SETD3_HUMAN - CobB | −1.1372276 | 0.04166453 |
| 105 - 273 | N105 | C273 | MBD2_HUMAN - PRDM9- | −1.1401715 | 0.01451992 |
| | | | CD | | |
| 315 - 55 | N315 | C55 | NCoR - RBBP4_HUMAN | −1.1402967 | 0.0367029 |
| 171 - 349 | N171 | C349 | ASH1L_HUMAN - | −1.1418241 | 0.04533101 |
| | | | DAM_N132A | | |
| 212 - 248 | N212 | C248 | HDAC8_HUMAN - | −1.1420659 | 0.0497809 |
| | | | GLYR1_HUMAN | | |
| 69 - 306 | N69 | C306 | SETB2_HUMAN - CobB | −1.1432647 | 0.00224514 |
| 68 - 288 | N68 | C288 | MBD3_HUMAN - SUV[SET] | −1.1436134 | 0.0027344 |
| 298 - 95 | N298 | C95 | MBD2b - SETD6_HUMAN | −1.1440139 | 0.04731002 |
| 68 - 59 | N68 | C59 | MBD3_HUMAN - | −1.1444215 | 0.0051794 |
| | | | INO80_HUMAN | | |
| 250 - 68 | N250 | C68 | HCFC1_HUMAN - | −1.1445334 | 0.01581748 |
| | | | MBD3_HUMAN | | |
| 68 - 277 | N68 | C277 | MBD3_HUMAN - SUV39H1 | −1.1449319 | 0.0246287 |
| 8 - 298 | N8 | C298 | MECP2 - MBD2b | −1.1449788 | 0.01946759 |
| 88 - 215 | N88 | C215 | ING4_HUMAN - | −1.1453784 | 0.04493502 |
| | | | HDAC4_HUMAN | | |
| 313 - 152 | N313 | C152 | SaII - CDY1_HUMAN | −1.1456486 | 0.04685083 |
| 233 - 306 | N233 | C306 | HINFP_HUMAN - CobB | −1.1458054 | 0.01780628 |
| 3 - 277 | N3 | C277 | HDAC3 - SUV39H1 | −1.1458999 | 0.03576155 |
| 64 - 69 | N64 | C69 | HDAC2_HUMAN - | −1.1463461 | 0.03458111 |
| | | | SETB2_HUMAN | | |
| 290 - 298 | N290 | C298 | HP1aCS - MBD2b | −1.1470507 | 0.01432165 |
| 105 - 249 | N105 | C249 | MBD2_HUMAN - HAC1- | −1.1494202 | 0.0085069 |
| | | | HAT | | |
| 293 - 212 | N293 | C212 | KRAB-MeCP2 - | −1.1498539 | 0.04461968 |
| | | | HDAC8_HUMAN | | |
| 3 - 112 | N3 | C112 | HDAC3 - KDM5A_HUMAN | −1.1514925 | 0.04276474 |
| 222 - 105 | N222 | C105 | IN80E_HUMAN - | −1.1515105 | 0.0033915 |
| | | | MBD2_HUMAN | | |
| 105 - 26 | N105 | C26 | MBD2_HUMAN - EZH1 | −1.1522228 | 0.01022857 |
| 105 - 266 | N105 | C266 | MBD2_HUMAN - | −1.1529236 | 0.00211512 |
| | | | GADD45A | | |
| 73 - 152 | N73 | C152 | KDM5D_HUMAN - | −1.153 | 0.00840693 |
| | | | CDY1_HUMAN | | |
| 306 - 190 | N306 | C190 | CobB - ANM1_HUMAN | −1.1530614 | 0.00627117 |
| 55 - 213 | N55 | C213 | RBBP4_HUMAN - | −1.153206 | 0.02826899 |
| | | | HBAP1_HUMAN | | |
| 324 - 293 | N324 | C293 | LBR_HUMAN - KRAB- | −1.1538979 | 0.0299308 |
| | | | MeCP2 | | |
| 112 - 191 | N112 | C191 | KDM5A_HUMAN - | −1.1541945 | 0.04157354 |
| | | | CDYL_HUMAN | | |
| 344 - 280 | N344 | C280 | HDAC3_HUMAN - CBF1- | −1.1542003 | 0.04008525 |
| | | | Cterm | | |
| 306 - 95 | N306 | C95 | CobB - SETD6_HUMAN | −1.1546868 | 0.00694617 |
| 68 - 310 | N68 | C310 | MBD3_HUMAN - TgSET8 | −1.1549558 | 0.02077691 |
| 105 - 24 | N105 | C24 | MBD2_HUMAN - CTCFL | −1.1559424 | 0.0072152 |
| 105 - 345 | N105 | C345 | MBD2_HUMAN - | −1.1570526 | 0.00464171 |
| | | | MSX2_HUMAN | | |
| 14 - 32 | N14 | C32 | RELA - | −1.1584566 | 0.03588684 |
| | | | Gilbert_Lab_CRISPRi_KRAB | | |
| 298 - 271 | N298 | C271 | MBD2b - GCN5 | −1.158682 | 0.00661271 |
| 182 - 257 | N182 | C257 | MTA3_HUMAN - c-Myb- | −1.1607249 | 0.01697763 |
| | | | TAD | | |
| 130 - 298 | N130 | C298 | PRDM2_HUMAN - MBD2b | −1.1627471 | 0.00627609 |
| 287 - 105 | N287 | C105 | G9A[SET] - MBD2_HUMAN | −1.1630279 | 0.00155449 |
| 131 - 302 | N131 | C302 | PRDM2_HUMAN - HDAC11 | −1.1635788 | 0.04747865 |
| 158 - 105 | N158 | C105 | SETB1_HUMAN - | −1.1640895 | 0.04980888 |
| | | | MBD2_HUMAN | | |
| 105 - 271 | N105 | C271 | MBD2_HUMAN - GCN5 | −1.1644723 | 0.01041076 |
| 106 - 55 | N106 | C55 | KAT6B_HUMAN - | −1.1650799 | 0.01435098 |
| | | | RBBP4_HUMAN | | |
| 55 - 113 | N55 | C113 | RBBP4_HUMAN - | −1.1653266 | 0.02288629 |
| | | | KDM5A_HUMAN | | |
| 173 - 314 | N173 | C314 | SIR4_HUMAN - MeCP2 | −1.1662494 | 0.0489906 |
| 298 - 272 | N298 | C272 | MBD2b - Sir2 | −1.1664163 | 0.00539393 |
| 68 - 226 | N68 | C226 | MBD3_HUMAN - | −1.1668142 | 0.02803102 |
| | | | SETD2_HUMAN | | |
| 3 - 320 | N3 | C320 | HDAC3 - mMLL3-SET | −1.1677596 | 0.00269038 |
| 153 - 320 | N153 | C320 | KAT7_HUMAN - mMLL3- | −1.1684234 | 0.03912455 |
| | | | SET | | |
| 201 - 299 | N201 | C299 | KDM2B_HUMAN - HST2 | −1.1685123 | 0.03368301 |
| 110 - 32 | N110 | C32 | JMJD4_HUMAN - | −1.1689725 | 0.00164422 |
| | | | Gilbert_Lab_CRISPRi_KRAB | | |
| 66 - 105 | N66 | C105 | DOT1L_HUMAN - | −1.1694558 | 0.00844392 |
| | | | MBD2_HUMAN | | |
| 280 - 215 | N280 | C215 | CBF1-Cterm - | −1.1695092 | 0.00507853 |
| | | | HDAC4_HUMAN | | |
| 306 - 219 | N306 | C219 | CobB - JARD2_HUMAN | −1.1712699 | 0.00175023 |
| 334 - 8 | N334 | C8 | hs_RCOR1 - MECP2 | −1.1722383 | 0.01037453 |
| 112 - 165 | N112 | C165 | KDM5A_HUMAN - | −1.173361 | 0.02723483 |
| | | | MTA2_HUMAN | | |
| 174 - 68 | N174 | C68 | HDAC1_HUMAN - | −1.1746845 | 0.03082822 |
| | | | MBD3_HUMAN | | |
| 267 - 272 | N267 | C272 | FOXA1-Nterm - Sir2 | −1.1764656 | 0.04759792 |
| 306 - 300 | N306 | C300 | CobB - SIRT6 | −1.1772638 | 0.00561799 |
| 273 - 298 | N273 | C298 | PRDM9-CD - MBD2b | −1.1774178 | 0.03124595 |
| 149 - 7 | N149 | C7 | MBD1_HUMAN - ZNF10 | −1.177978 | 0.00726658 |
| 68 - 267 | N68 | C267 | MBD3_HUMAN - FOXA1- | −1.17816 | 0.00651457 |
| | | | Nterm | | |
| 9 - 293 | N9 | C293 | MECP2 - KRAB-MeCP2 | −1.1784267 | 0.01268893 |
| 68 - 27 | N68 | C27 | MBD3_HUMAN - EZH1 | −1.1792375 | 0.0076529 |
| 273 - 191 | N273 | C191 | PRDM9-CD - | −1.1798788 | 0.02425262 |
| | | | CDYL_HUMAN | | |
| 255 - 68 | N255 | C68 | EED_HUMAN - | −1.1799264 | 0.01738823 |
| | | | MBD3_HUMAN | | |
| 68 - 233 | N68 | C233 | MBD3_HUMAN - | −1.180471 | 0.00492287 |
| | | | HINFP_HUMAN | | |
| 202 - 290 | N202 | C290 | KDM2B_HUMAN - HP1aCS | −1.1808414 | 0.01880286 |
| 66 - 162 | N66 | C162 | DOT1L_HUMAN - | −1.1826074 | 0.02168409 |
| | | | SIR6_HUMAN | | |
| 105 - 263 | N105 | C263 | MBD2_HUMAN - HDAC8 | −1.1830346 | 0.04227082 |
| 17 - 334 | N17 | C334 | EZH2 - hs_RCOR1 | −1.1831717 | 0.00611871 |
| 25 - 147 | N25 | C147 | CTCFL - NSD3_HUMAN | −1.1843459 | 0.01682866 |
| 298 - 329 | N298 | C329 | MBD2b - pbcv1_vSET | −1.1843982 | 0.0252023 |
| 267 - 293 | N267 | C293 | FOXA1-Nterm - KRAB- | −1.185559 | 0.04396317 |
| | | | MeCP2 | | |
| 75 - 68 | N75 | C68 | KDM5D_HUMAN - | −1.1865453 | 0.0075903 |
| | | | MBD3_HUMAN | | |
| 306 - 20 | N306 | C20 | CobB - KDM1B | −1.1868919 | 0.00754871 |
| 273 - 68 | N273 | C68 | PRDM9-CD - | −1.187848 | 0.00585447 |
| | | | MBD3_HUMAN | | |
| 310 - 334 | N310 | C334 | TgSET8 - hs_RCOR1 | −1.1880287 | 0.01892282 |
| 280 - 293 | N280 | C293 | CBF1-Cterm - KRAB-MeCP2 | −1.1880902 | 0.01961471 |
| 83 - 255 | N83 | C255 | SMRD3_HUMAN - | −1.1881856 | 0.04419152 |
| | | | EED_HUMAN | | |
| 311 - 202 | N311 | C202 | Sin3a - KDM2B_HUMAN | −1.1885474 | 0.02903824 |
| 5 - 152 | N5 | C152 | KDM1A - CDY1_HUMAN | −1.1885733 | 0.01267099 |
| 25 - 334 | N25 | C334 | CTCFL - hs_RCOR1 | −1.189583 | 0.04192416 |
| 25 - 295 | N25 | C295 | CTCFL - SET-TAF1B | −1.189664 | 0.04224211 |
| 3 - 275 | N3 | C275 | HDAC3 - DOT1L- | −1.189942 | 0.01687497 |
| | | | DOT1_domain | | |
| 220 - 131 | N220 | C131 | JARD2_HUMAN - | −1.1901779 | 0.04022975 |
| | | | PRDM2_HUMAN | | |
| 106 - 68 | N106 | C68 | KAT6B_HUMAN - | −1.1901825 | 0.00257116 |
| | | | MBD3_HUMAN | | |
| 47 - 298 | N47 | C298 | ANM2_HUMAN - MBD2b | −1.1902207 | 0.03226335 |
| 68 - 150 | N68 | C150 | MBD3_HUMAN - | −1.190486 | 0.04113157 |
| | | | HDA11_HUMAN | | |
| 345 - 105 | N345 | C105 | MSX2_HUMAN - | −1.1909137 | 0.00720547 |
| | | | MBD2_HUMAN | | |
| 257 - 215 | N257 | C215 | c-Myb-TAD - | −1.1910864 | 0.03139481 |
| | | | HDAC4_HUMAN | | |
| 297 - 68 | N297 | C68 | MesoLo - MBD3_HUMAN | −1.191593 | 0.00826629 |
| 68 - 130 | N68 | C130 | MBD3_HUMAN - | −1.1920593 | 0.00730459 |
| | | | PRDM2_HUMAN | | |
| 306 - 297 | N306 | C297 | CobB - MesoLo | −1.1922855 | 0.00148208 |
| 135 - 272 | N135 | C272 | KDM2A_HUMAN - Sir2 | −1.1928632 | 0.04648234 |
| 78 - 131 | N78 | C131 | KAT6A_HUMAN - | −1.1935132 | 0.02645971 |
| | | | PRDM2_HUMAN | | |
| 105 - 123 | N105 | C123 | MBD2_HUMAN - | −1.193592 | 0.03442472 |
| | | | KMT2A_HUMAN | | |
| 205 - 55 | N205 | C55 | JMJD7_HUMAN - | −1.1942332 | 0.03144964 |
| | | | RBBP4_HUMAN | | |
| 3 - 218 | N3 | C218 | HDAC3 - IN80B_HUMAN | −1.1942826 | 0.00962538 |
| 1 - 7 | N1 | C7 | SETD1A - ZNF10 | −1.194756 | 0.04646763 |
| 293 - 315 | N293 | C315 | KRAB-MeCP2 - NCoR | −1.1950921 | 0.00544617 |
| 196 - 330 | N196 | C330 | MTA1_HUMAN - ce_Set1 | −1.1952458 | 0.01038501 |
| 298 - 255 | N298 | C255 | MBD2b - EED_HUMAN | −1.1954883 | 0.00158966 |
| 3 - 306 | N3 | C306 | HDAC3 - CobB | −1.1955032 | 0.00582815 |
| 296 - 105 | N296 | C105 | HP1a - MBD2_HUMAN | −1.1958539 | 0.00202929 |
| 12 - 341 | N12 | C341 | KMT2A - NCOR2_HUMAN | −1.1964881 | 0.02458941 |
| 130 - 68 | N130 | C68 | PRDM2_HUMAN - | −1.1974138 | 0.02254673 |
| | | | MBD3_HUMAN | | |
| 312 - 293 | N312 | C293 | SetD8 - KRAB-MeCP2 | −1.1980458 | 0.0106824 |
| 63 - 105 | N63 | C105 | SMYD5_HUMAN - | −1.1983103 | 0.01389487 |
| | | | MBD2_HUMAN | | |
| 255 - 299 | N255 | C299 | EED_HUMAN - HST2 | −1.199794 | 0.02577271 |
| 166 - 7 | N166 | C7 | EHMT1_HUMAN - ZNF10 | −1.2001654 | 0.03827989 |
| 126 - 298 | N126 | C298 | SETMR_HUMAN - MBD2b | −1.2008579 | 0.0080318 |
| 37 - 55 | N37 | C55 | KDM6A - RBBP4_HUMAN | −1.2010894 | 0.03908863 |
| 69 - 108 | N69 | C108 | SETB2_HUMAN - | −1.2021008 | 0.00893935 |
| | | | KAT6B_HUMAN | | |
| 3 - 34 | N3 | C34 | HDAC3 - DNMT3A- | −1.2023238 | 0.00392255 |
| | | | 3L_JKNp112 | | |
| 269 - 215 | N269 | C215 | PU.1 - HDAC4_HUMAN | −1.2029817 | 0.01362601 |
| 158 - 306 | N158 | C306 | SETB1_HUMAN - CobB | −1.2033338 | 0.04343765 |
| 17 - 191 | N17 | C191 | EZH2 - CDYL_HUMAN | −1.2038811 | 0.00555868 |
| 25 - 68 | N25 | C68 | CTCFL - MBD3_HUMAN | −1.2041685 | 0.00580924 |
| 68 - 251 | N68 | C251 | MBD3_HUMAN - | −1.2042853 | 0.02003576 |
| | | | HCFC1_HUMAN | | |
| 34 - 105 | N34 | C105 | DNMT3A-3L_JKNp112 - | −1.2043135 | 0.02206037 |
| | | | MBD2_HUMAN | | |
| 257 - 131 | N257 | C131 | c-Myb-TAD - | −1.2048637 | 0.00394994 |
| | | | PRDM2_HUMAN | | |
| 289 - 105 | N289 | C105 | FOG1 - MBD2_HUMAN | −1.2050654 | 0.01635753 |
| 344 - 246 | N344 | C246 | HDAC3_HUMAN - | −1.2059461 | 0.00117435 |
| | | | ATRX_HUMAN | | |
| 138 - 105 | N138 | C105 | TFPT_HUMAN - | −1.2062056 | 0.00332475 |
| | | | MBD2_HUMAN | | |
| 344 - 69 | N344 | C69 | HDAC3_HUMAN - | −1.206282 | 0.04026467 |
| | | | SETB2_HUMAN | | |
| 300 - 306 | N300 | C306 | SIRT6 - CobB | −1.2071562 | 0.00439927 |
| 105 - 27 | N105 | C27 | MBD2_HUMAN - EZH1 | −1.2074474 | 0.00122129 |
| 151 - 191 | N151 | C191 | SIR7_HUMAN - | −1.209277 | 0.02344826 |
| | | | CDYL_HUMAN | | |
| 105 - 149 | N105 | C149 | MBD2_HUMAN - | −1.209505 | 0.00540665 |
| | | | MBD1_HUMAN | | |
| 293 - 130 | N293 | C130 | KRAB-MeCP2 - | −1.2095667 | 0.02818996 |
| | | | PRDM2_HUMAN | | |
| 134 - 68 | N134 | C68 | KDM2A_HUMAN - | −1.2096081 | 0.01677297 |
| | | | MBD3_HUMAN | | |
| 189 - 191 | N189 | C191 | ING2_HUMAN - | −1.2107728 | 0.03604838 |
| | | | CDYL_HUMAN | | |
| 293 - 149 | N293 | C149 | KRAB-MeCP2 - | −1.2108382 | 0.0347253 |
| | | | MBD1_HUMAN | | |
| 68 - 120 | N68 | C120 | MBD3_HUMAN - | −1.2121826 | 0.00840935 |
| | | | RING1_HUMAN | | |
| 105 - 269 | N105 | C269 | MBD2_HUMAN - PU.1 | −1.212999 | 0.00907613 |
| 216 - 306 | N216 | C306 | ING5_HUMAN - CobB | −1.2132132 | 7.42E−04 |
| 298 - 305 | N298 | C305 | MBD2b - NUE | −1.2137614 | 0.00603407 |
| 105 - 289 | N105 | C289 | MBD2_HUMAN - FOG1 | −1.2138124 | 0.0421484 |
| 273 - 293 | N273 | C293 | PRDM9-CD - KRAB-MeCP2 | −1.2140649 | 0.01923716 |
| 5 - 68 | N5 | C68 | KDM1A - MBD3_HUMAN | −1.2140748 | 0.01904977 |
| 108 - 32 | N108 | C32 | KAT6B_HUMAN - | −1.2162406 | 0.00174654 |
| | | | Gilbert_Lab_CRISPRi_KRAB | | |
| 306 - 69 | N306 | C69 | CobB - SETB2_HUMAN | −1.2162866 | 0.0039222 |
| 269 - 55 | N269 | C55 | PU.1 - RBBP4_HUMAN | −1.2163234 | 0.02249287 |
| 306 - 314 | N306 | C314 | CobB - MeCP2 | −1.2171074 | 0.01944157 |
| 187 - 293 | N187 | C293 | MBD4_HUMAN - KRAB- | −1.217522 | 0.01803806 |
| | | | MeCP2 | | |
| 55 - 267 | N55 | C267 | RBBP4_HUMAN - FOXA1- | −1.2194119 | 0.00380561 |
| | | | Nterm | | |
| 106 - 7 | N106 | C7 | KAT6B_HUMAN - ZNF10 | −1.219444 | 0.00458683 |
| 165 - 264 | N165 | C264 | MTA2_HUMAN - | −1.2207026 | 0.0265596 |
| | | | MTL5_HUMAN | | |
| 75 -105 | N75 | C105 | KDM5D_HUMAN - | −1.2207895 | 0.00618035 |
| | | | MBD2_HUMAN | | |
| 344 - 51 | N344 | C51 | HDAC3_HUMAN - | −1.2208833 | 0.04979993 |
| | | | UCHLS_HUMAN | | |
| 306 - 309 | N306 | C309 | CobB - HDAC8 | −1.2212759 | 0.01003049 |
| 246 - 306 | N246 | C306 | ATRX_HUMAN - CobB | −1.2215584 | 0.00396427 |
| 215 - 38 | N215 | C38 | HDAC4_HUMAN - EHMT2 | −1.2222827 | 0.03549515 |
| 298 - 221 | N298 | C221 | MBD2b - SMYD2_HUMAN | −1.2236058 | 0.02054816 |
| 3 - 164 | N3 | C164 | HDAC3 - SIR5_HUMAN | −1.2252469 | 0.00249696 |
| 226 - 68 | N226 | C68 | SETD2_HUMAN - | −1.2257255 | 0.00390166 |
| | | | MBD3_HUMAN | | |
| 55 - 164 | N55 | C164 | RBBP4_HUMAN - | −1.2258207 | 0.00106263 |
| | | | SIR5_HUMAN | | |
| 182 - 45 | N182 | C45 | MTA3_HUMAN - | −1.2262605 | 0.0397372 |
| | | | KMT2D_HUMAN | | |
| 182 - 320 | N182 | C320 | MTA3_HUMAN - mMLL3- | −1.226788 | 0.00221939 |
| | | | SET | | |
| 26 - 306 | N26 | C306 | EZH1 - CobB | −1.2269183 | 0.00644796 |
| 346 - 68 | N346 | C68 | MSX2_HUMAN - | −1.2275914 | 0.01074495 |
| | | | MBD3_HUMAN | | |
| 341 - 296 | N341 | C296 | NCOR2_HUMAN - HP1a | −1.2276746 | 0.03233805 |
| 296 - 298 | N296 | C298 | HP1a - MBD2b | −1.2280604 | 0.00112753 |
| 97 - 298 | N97 | C298 | CHD3_HUMAN - MBD2b | −1.2282415 | 0.01174699 |
| 68 - 280 | N68 | C280 | MBD3_HUMAN - CBF1- | −1.2301122 | 0.01480597 |
| | | | Cterm | | |
| 55 - 334 | N55 | C334 | RBBP4_HUMAN - | −1.230145 | 0.02360917 |
| | | | hs_RCOR1 | | |
| 228 - 26 | N228 | C26 | HDAC7_HUMAN - EZH1 | −1.2304769 | 0.03364942 |
| 32 - 306 | N32 | C306 | Gilbert_Lab_CRISPRi_KRAB - | −1.2304885 | 0.01055678 |
| | | | CobB | | |
| 185 - 105 | N185 | C105 | RBBP7_HUMAN - | −1.2315946 | 0.01952822 |
| | | | MBD2_HUMAN | | |
| 202 - 105 | N202 | C105 | KDM2B_HUMAN - | −1.2317344 | 0.01018781 |
| | | | MBD2_HUMAN | | |
| 271 - 272 | N271 | C272 | GCN5 - Sir2 | −1.2320744 | 0.0173872 |
| 201 - 298 | N201 | C298 | KDM2B_HUMAN - MBD2b | −1.2320797 | 0.00870992 |
| 68 - 138 | N68 | C138 | MBD3_HUMAN - | −1.2323302 | 0.00527849 |
| | | | TFPT_HUMAN | | |
| 204 - 299 | N204 | C299 | HIF1N_HUMAN - HST2 | −1.2332619 | 0.031855 |
| 306 - 320 | N306 | C320 | CobB - mMLL3-SET | −1.2335597 | 9.40E−04 |
| 237 - 105 | N237 | C105 | KAT5_HUMAN - | −1.2339212 | 0.00265871 |
| | | | MBD2_HUMAN | | |
| 328 - 105 | N328 | C105 | hs_PHF19 - MBD2_HUMAN | −1.2345866 | 0.00298394 |
| 105 - 162 | N105 | C162 | MBD2_HUMAN - | −1.2389014 | 0.00267179 |
| | | | SIR6_HUMAN | | |
| 18 - 68 | N18 | C68 | EZH2 - MBD3_HUMAN | −1.239342 | 0.04203516 |
| 68 - 68 | N68 | C68 | MBD3_HUMAN - | −1.2394888 | 0.01204173 |
| | | | MBD3_HUMAN | | |
| 306 - 204 | N306 | C204 | CobB - HIF1N_HUMAN | −1.2397432 | 0.01259062 |
| 311 - 298 | N311 | C298 | Sin3a - MBD2b | −1.2401202 | 0.02517373 |
| 135 - 299 | N135 | C299 | KDM2A_HUMAN - HST2 | −1.2408065 | 0.04181804 |
| 212 - 68 | N212 | C68 | HDAC8_HUMAN - | −1.2418579 | 0.01786708 |
| | | | MBD3_HUMAN | | |
| 148 - 191 | N148 | C191 | NSD3_HUMAN - | −1.2419943 | 0.03833889 |
| | | | CDYL_HUMAN | | |
| 55 - 263 | N55 | C263 | RBBP4_HUMAN - HDAC8 | −1.2423145 | 0.03204441 |
| 320 - 328 | N320 | C328 | mMLL3-SET - hs_PHF19 | −1.2424058 | 0.02824765 |
| 105 - 18 | N105 | C18 | MBD2_HUMAN - EZH2 | −1.2433939 | 0.02108537 |
| 302 - 293 | N302 | C293 | HDAC11 - KRAB-MeCP2 | −1.2448775 | 0.01454248 |
| 1 - 191 | N1 | C191 | SETD1A - CDYL_HUMAN | −1.2453092 | 0.01417863 |
| 177 - 171 | N177 | C171 | KDM5C_HUMAN - | −1.2458118 | 0.03495492 |
| | | | ASH1L_HUMAN | | |
| 218 - 55 | N218 | C55 | IN80B_HUMAN - | −1.245886 | 0.01038774 |
| | | | RBBP4_HUMAN | | |
| 310 - 298 | N310 | C298 | TgSET8 - MBD2b | −1.2459458 | 0.00891549 |
| 105 - 107 | N105 | C107 | MBD2_HUMAN - | −1.2472551 | 6.20E−04 |
| | | | KAT6B_HUMAN | | |
| 245 - 32 | N245 | C32 | TYY1_HUMAN - | −1.2474757 | 0.01582482 |
| | | | Gilbert_Lab_CRISPRi_KRAB | | |
| 112 - 105 | N112 | C105 | KDM5A_HUMAN - | −1.2486112 | 4.67E−04 |
| | | | MBD2_HUMAN | | |
| 105 - 15 | N105 | C15 | MBD2_HUMAN - EP300 | −1.2488214 | 0.00282733 |
| 332 - 7 | N332 | C7 | hs_SIRT5 - ZNF10 | −1.2490729 | 0.03782275 |
| 3 - 299 | N3 | C299 | HDAC3 - HST2 | −1.2492137 | 0.02470862 |
| 253 - 32 | N253 | C32 | RBBP5_HUMAN - | −1.2499792 | 0.00834305 |
| | | | Gilbert_Lab_CRISPRi_KRAB | | |
| 136 - 105 | N136 | C105 | JMJD6_HUMAN - | −1.2502292 | 6.08E−04 |
| | | | MBD2_HUMAN | | |
| 306 - 273 | N306 | C273 | CobB - PRDM9-CD | −1.2505526 | 0.01385185 |
| 73 - 306 | N73 | C306 | KDM5D_HUMAN - CobB | −1.2507223 | 0.002031 |
| 68 - 221 | N68 | C221 | MBD3_HUMAN - | −1.2507467 | 0.0265672 |
| | | | SMYD2_HUMAN | | |
| 296 - 293 | N296 | C293 | HP1a - KRAB-MeCP2 | −1.2511653 | 0.0146328 |
| 174 - 306 | N174 | C306 | HDAC1_HUMAN - CobB | −1.2512962 | 0.04307571 |
| 42 - 293 | N42 | C293 | KMT2E_HUMAN - KRAB- | −1.2515425 | 0.02859632 |
| | | | MeCP2 | | |
| 334 - 293 | N334 | C293 | hs_RCOR1 - KRAB-MeCP2 | −1.2515709 | 0.00328435 |
| 299 - 5 | N299 | C5 | HST2 - KDM1A | −1.2521314 | 0.03644005 |
| 298 - 20 | N298 | C20 | MBD2b - KDM1B | −1.2522706 | 0.04425689 |
| 68 - 272 | N68 | C272 | MBD3_HUMAN - Sir2 | −1.2526891 | 0.00315072 |
| 105 - 87 | N105 | C87 | MBD2_HUMAN - | −1.2534152 | 0.00575384 |
| | | | SETD7_HUMAN | | |
| 329 - 334 | N329 | C334 | pbcv1_vSET - hs_RCOR1 | −1.2538074 | 0.04326601 |
| 105 - 296 | N105 | C296 | MBD2_HUMAN - HP1a | −1.2542018 | 0.0196984 |
| 3 - 100 | N3 | C100 | HDAC3 - TAF1_HUMAN | −1.2542167 | 0.01589577 |
| 341 - 271 | N341 | C271 | NCOR2_HUMAN - GCN5 | −1.2550285 | 0.04807251 |
| 165 - 330 | N165 | C330 | MTA2_HUMAN - ce_Set1 | −1.2552944 | 0.03634341 |
| 105 - 43 | N105 | C43 | MBD2_HUMAN - | −1.2562639 | 0.01304264 |
| | | | SETD4_HUMAN | | |
| 315 - 165 | N315 | C165 | NCoR - MTA2_HUMAN | −1.2567213 | 0.00512499 |
| 105 - 288 | N105 | C288 | MBD2_HUMAN - SUV[SET] | −1.2573625 | 0.04850555 |
| 212 - 131 | N212 | C131 | HDAC8_HUMAN - | −1.2576288 | 0.03242931 |
| | | | PRDM2_HUMAN | | |
| 197 - 314 | N197 | C314 | SUV91_HUMAN - MeCP2 | −1.2578936 | 0.0289431 |
| 268 - 306 | N268 | C306 | FOXA1-Cterm - CobB | −1.258186 | 0.00928236 |
| 55 - 34 | N55 | C34 | RBBP4_HUMAN - | −1.2586099 | 0.00332953 |
| | | | DNMT3A-3L_JKNp112 | | |
| 1 - 165 | N1 | C165 | SETD1A - MTA2_HUMAN | −1.2587681 | 0.02361206 |
| 306 - 43 | N306 | C43 | CobB - SETD4_HUMAN | −1.2589326 | 0.01300731 |
| 165 - 280 | N165 | C280 | MTA2_HUMAN - CBF1- | −1.259486 | 0.01477668 |
| | | | Cterm | | |
| 165 - 59 | N165 | C59 | MTA2_HUMAN - | −1.2595972 | 0.02871112 |
| | | | INO80_HUMAN | | |
| 298 - 70 | N298 | C70 | MBD2b - SETB2_HUMAN | −1.2596314 | 0.01276072 |
| 95 - 213 | N95 | C213 | SETD6_HUMAN - | −1.2605223 | 0.03010746 |
| | | | HBAP1_HUMAN | | |
| 105 - 111 | N105 | C111 | MBD2_HUMAN - | −1.261235 | 0.00556345 |
| | | | KDM5A_HUMAN | | |
| 55 - 313 | N55 | C313 | RBBP4_HUMAN - SaII | −1.261631 | 7.17E−04 |
| 190 - 105 | N190 | C105 | ANM1_HUMAN - | −1.2621063 | 0.01131789 |
| | | | MBD2_HUMAN | | |
| 3 - 127 | N3 | C127 | HDAC3 - SETMR_HUMAN | −1.2622793 | 0.00337701 |
| 105 - 344 | N105 | C344 | MBD2_HUMAN - | −1.2626101 | 0.00430186 |
| | | | HDAC3_HUMAN | | |
| 3 - 98 | N3 | C98 | HDAC3 - A4FU79_HUMAN | −1.2627924 | 0.00155766 |
| 79 - 191 | N79 | C191 | KAT6A_HUMAN - | −1.2628452 | 0.03724013 |
| | | | CDYL_HUMAN | | |
| 341 - 346 | N341 | C346 | NCOR2_HUMAN - | −1.2632024 | 0.00559746 |
| | | | MSX2_HUMAN | | |
| 165 - 298 | N165 | C298 | MTA2_HUMAN - MBD2b | −1.2632706 | 0.04190117 |
| 110 - 75 | N110 | C75 | JMJD4_HUMAN - | −1.2639866 | 0.03460628 |
| | | | KDM5D_HUMAN | | |
| 105 -260 | N105 | C260 | MBD2_HUMAN - Chd1-CD | −1.2647116 | 0.04529498 |
| 226 - 298 | N226 | C298 | SETD2_HUMAN - MBD2b | −1.2652683 | 0.03217587 |
| 55 - 275 | N55 | C275 | RBBP4_HUMAN - DOT1L- | −1.2656394 | 0.0392043 |
| | | | DOT1_domain | | |
| 3 - 107 | N3 | C107 | HDAC3 - KAT6B_HUMAN | −1.2667905 | 0.01660274 |
| 105 - 132 | N105 | C132 | MBD2_HUMAN - | −1.2681857 | 0.04509242 |
| | | | PRDM2_HUMAN | | |
| 111 - 68 | N111 | C68 | KDM5A_HUMAN - | −1.2686683 | 0.01951547 |
| | | | MBD3_HUMAN | | |
| 298 - 32 | N298 | C32 | MBD2b - | −1.2697078 | 0.01560616 |
| | | | Gilbert_Lab_CRISPRi_KRAB | | |
| 68 - 299 | N68 | C299 | MBD3_HUMAN - HST2 | −1.2699826 | 9.74E−04 |
| 68 - 164 | N68 | C164 | MBD3_HUMAN - | −1.2701674 | 0.00340881 |
| | | | SIR5_HUMAN | | |
| 306 - 189 | N306 | C189 | CobB - ING2_HUMAN | −1.2702123 | 0.00983567 |
| 55 - 289 | N55 | C289 | RBBP4_HUMAN - FOG1 | −1.2706306 | 0.00103536 |
| 313 - 241 | N313 | C241 | SaII - SIR2_HUMAN | −1.2706717 | 0.03232708 |
| 88 - 68 | N88 | C68 | ING4_HUMAN - | −1.2712167 | 9.65E−04 |
| | | | MBD3_HUMAN | | |
| 267 - 299 | N267 | C299 | FOXA1-Nterm - HST2 | −1.2720677 | 0.01825752 |
| 131 - 290 | N131 | C290 | PRDM2_HUMAN - HP1aCS | −1.2722904 | 0.02340748 |
| 47 - 105 | N47 | C105 | ANM2_HUMAN - | −1.2733446 | 0.03200674 |
| | | | MBD2_HUMAN | | |
| 3 - 324 | N3 | C324 | HDAC3 - LBR_HUMAN | −1.2737251 | 0.00177021 |
| 165 - 150 | N165 | C150 | MTA2_HUMAN - | −1.2739414 | 0.04388519 |
| | | | HDA11_HUMAN | | |
| 68 - 290 | N68 | C290 | MBD3_HUMAN - HP1aCS | −1.274449 | 0.00126717 |
| 131 - 135 | N131 | C135 | PRDM2_HUMAN - | −1.2746128 | 0.03876748 |
| | | | KDM2A_HUMAN | | |
| 226 - 32 | N226 | C32 | SETD2_HUMAN - | −1.2751918 | 0.02084131 |
| | | | Gilbert_Lab_CRISPRi_KRAB | | |
| 55 - 310 | N55 | C310 | RBBP4_HUMAN - TgSET8 | −1.2756356 | 0.02983284 |
| 222 - 306 | N222 | C306 | IN80E_HUMAN - CobB | −1.2762761 | 6.28E−04 |
| 189 - 306 | N189 | C306 | ING2_HUMAN - CobB | −1.2767206 | 0.0231859 |
| 108 - 105 | N108 | C105 | KAT6B_HUMAN - | −1.2784026 | 0.00116178 |
| | | | MBD2_HUMAN | | |
| 329 - 231 | N329 | C231 | pbcv1_vSET - | −1.2790812 | 0.03222191 |
| | | | KDM5B_HUMAN | | |
| 105 - 47 | N105 | C47 | MBD2_HUMAN - | −1.2791281 | 0.00686429 |
| | | | ANM2_HUMAN | | |
| 3 - 25 | N3 | C25 | HDAC3 - CTCFL | −1.2793688 | 0.00368417 |
| 332 - 9 | N332 | C9 | hs_SIRT5 - MECP2 | −1.2793958 | 0.02971773 |
| 55 - 333 | N55 | C333 | RBBP4_HUMAN - sc_RPD3 | −1.2801274 | 0.0169562 |
| 110 - 293 | N110 | C293 | JMJD4_HUMAN - KRAB- | −1.2810374 | 0.02345322 |
| | | | MeCP2 | | |
| 344 - 306 | N344 | C306 | HDAC3_HUMAN - CobB | −1.2817938 | 0.00994917 |
| 344 - 5 | N344 | C5 | HDAC3_HUMAN - KDM1A | −1.2818127 | 0.02933174 |
| 307 - 105 | N307 | C105 | KYP - MBD2_HUMAN | −1.2826327 | 0.00118362 |
| 327 - 9 | N327 | C9 | hs_NIPP1 - MECP2 | −1.2830803 | 0.02797928 |
| 182 - 273 | N182 | C273 | MTA3_HUMAN - PRDM9- | −1.2838056 | 0.0180128 |
| | | | CD | | |
| 185 - 298 | N185 | C298 | RBBP7_HUMAN - MBD2b | −1.2841132 | 0.00401221 |
| 3 - 346 | N3 | C346 | HDAC3 - MSX2_HUMAN | −1.2846673 | 0.00183264 |
| 120 - 105 | N120 | C105 | RING1_HUMAN - | −1.2851436 | 0.01782829 |
| | | | MBD2_HUMAN | | |
| 187 - 298 | N187 | C298 | MBD4_HUMAN - MBD2b | −1.2860175 | 0.01006653 |
| 64 - 221 | N64 | C221 | HDAC2_HUMAN - | −1.2866228 | 0.04909983 |
| | | | SMYD2_HUMAN | | |
| 120 - 32 | N120 | C32 | RING1_HUMAN - | −1.2867747 | 0.00245384 |
| | | | Gilbert_Lab_CRISPRi_KRAB | | |
| 51 - 97 | N51 | C97 | UCHL5_HUMAN - | −1.2873841 | 0.04118227 |
| | | | CHD3_HUMAN | | |
| 3 - 194 | N3 | C194 | HDAC3 - ING1_HUMAN | −1.2909422 | 0.02747323 |
| 148 - 105 | N148 | C105 | NSD3_HUMAN - | −1.2909764 | 0.00174073 |
| | | | MBD2_HUMAN | | |
| 196 - 246 | N196 | C246 | MTA1_HUMAN - | −1.2911916 | 0.00117573 |
| | | | ATRX_HUMAN | | |
| 131 - 216 | N131 | C216 | PRDM2_HUMAN - | −1.2914974 | 0.01851684 |
| | | | ING5_HUMAN | | |
| 202 - 73 | N202 | C73 | KDM2B_HUMAN - | −1.2914985 | 0.0079028 |
| | | | KDM5D_HUMAN | | |
| 302 - 105 | N302 | C105 | HDAC11 - MBD2_HUMAN | −1.2924162 | 9.43E−04 |
| 307 - 68 | N307 | C68 | KYP - MBD3_HUMAN | −1.2928288 | 0.02038705 |
| 59 - 32 | N59 | C32 | INO80_HUMAN - | −1.2937103 | 0.03677024 |
| | | | Gilbert_Lab_CRISPRi_KRAB | | |
| 68 - 165 | N68 | C165 | MBD3_HUMAN - | −1.2945457 | 0.02461479 |
| | | | MTA2_HUMAN | | |
| 182 - 315 | N182 | C315 | MTA3_HUMAN - NCoR | −1.2957782 | 0.00185704 |
| 315 - 341 | N315 | C341 | NCoR - NCOR2_HUMAN | −1.2958573 | 0.01641226 |
| 305 - 67 | N305 | C67 | NUE - PRDM9_HUMAN | −1.2959426 | 0.01193027 |
| 42 - 191 | N42 | C191 | KMT2E_HUMAN - | −1.2959953 | 0.02575776 |
| | | | CDYL_HUMAN | | |
| 87 - 32 | N87 | C32 | SETD7_HUMAN - | −1.2964482 | 0.00685437 |
| | | | Gilbert_Lab_CRISPRi_KRAB | | |
| 165 - 349 | N165 | C349 | MTA2_HUMAN - | −1.2972928 | 0.00146293 |
| | | | DAM_N132A | | |
| 295 - 32 | N295 | C32 | SET-TAF1B - | −1.2973976 | 0.0014858 |
| | | | Gilbert_Lab_CRISPRi_KRAB | | |
| 269 - 131 | N269 | C131 | PU.1 - PRDM2_HUMAN | −1.2992151 | 0.00182267 |
| 175 - 298 | N175 | C298 | RUVB2_HUMAN - MBD2b | −1.3000711 | 0.03959747 |
| 182 - 154 | N182 | C154 | MTA3_HUMAN - | −1.3004823 | 0.02127899 |
| | | | NSD2_HUMAN | | |
| 61 - 191 | N61 | C191 | HAT1_HUMAN - | −1.3010876 | 0.01941164 |
| | | | CDYL_HUMAN | | |
| 105 - 218 | N105 | C218 | MBD2_HUMAN - | −1.3012229 | 0.00182478 |
| | | | IN80B_HUMAN | | |
| 197 - 334 | N197 | C334 | SUV91_HUMAN - | −1.3012438 | 0.04194521 |
| | | | hs_RCOR1 | | |
| 165 -210 | N165 | C210 | MTA2_HUMAN - | −1.3017269 | 0.02970764 |
| | | | SIR3_HUMAN | | |
| 297 - 32 | N297 | C32 | MesoLo - | −1.3018007 | 0.02463452 |
| | | | Gilbert_Lab_CRISPRi_KRAB | | |
| 331 - 105 | N331 | C105 | mm_G9a - MBD2_HUMAN | −1.3020073 | 0.00510701 |
| 341 - 300 | N341 | C300 | NCOR2_HUMAN - SIRT6 | −1.3026291 | 0.01457083 |
| 195 - 298 | N195 | C298 | SETD3_HUMAN - MBD2b | −1.3029051 | 0.03569851 |
| 61 - 293 | N61 | C293 | HAT1_HUMAN - KRAB- | −1.3036137 | 0.02836508 |
| | | | MeCP2 | | |
| 256 - 257 | N256 | C257 | LSD1-ORF - c-Myb-TAD | −1.3037812 | 0.00796565 |
| 293 - 255 | N293 | C255 | KRAB-MeCP2 - | −1.3038579 | 0.00884744 |
| | | | EED_HUMAN | | |
| 298 - 297 | N298 | C297 | MBD2b - MesoLo | −1.3053527 | 0.01095851 |
| 298 - 50 | N298 | C50 | MBD2b - NCOA1_HUMAN | −1.3065578 | 0.03065957 |
| 182 - 334 | N182 | C334 | MTA3_HUMAN - | −1.3066042 | 0.00777007 |
| | | | hs_RCOR1 | | |
| 308 - 68 | N308 | C68 | HDT1 - MBD3_HUMAN | −1.3066883 | 0.02091254 |
| 8 - 334 | N8 | C334 | MECP2 - hs_RCOR1 | −1.3073012 | 0.04807322 |
| 245 - 215 | N245 | C215 | TYY1_HUMAN - | −1.3082651 | 0.00800201 |
| | | | HDAC4_HUMAN | | |
| 239 - 32 | N239 | C32 | KDM8_HUMAN - | −1.3104587 | 0.01701347 |
| | | | Gilbert_Lab_CRISPRi_KRAB | | |
| 331 - 201 | N331 | C201 | mm_G9a - KDM2B_HUMAN | −1.3105324 | 0.04114378 |
| 185 - 332 | N185 | C332 | RBBP7_HUMAN - hs_SIRT5 | −1.3105776 | 0.02720184 |
| 105 - 77 | N105 | C77 | MBD2_HUMAN - | −1.3108287 | 0.00125837 |
| | | | KDM5D_HUMAN | | |
| 215 - 346 | N215 | C346 | HDAC4_HUMAN - | −1.3121046 | 0.02454284 |
| | | | MSX2_HUMAN | | |
| 298 - 314 | N298 | C314 | MBD2b - MeCP2 | −1.3122596 | 0.00359052 |
| 17 - 131 | N17 | C131 | EZH2 - PRDM2_HUMAN | −1.312473 | 0.02365943 |
| 216 - 32 | N216 | C32 | ING5_HUMAN - | −1.3143587 | 0.01693839 |
| | | | Gilbert_Lab_CRISPRi_KRAB | | |
| 263 - 191 | N263 | C191 | HDAC8 - CDYL_HUMAN | −1.3151021 | 0.04419684 |
| 7 - 123 | N7 | C123 | ZNF10 - KMT2A_HUMAN | −1.315188 | 0.0229147 |
| 289 - 191 | N289 | C191 | FOG1 - CDYL_HUMAN | −1.3167432 | 0.0045072 |
| 3 - 1 | N3 | C1 | HDAC3 - SETD1A | −1.3168151 | 0.00754908 |
| 55 - 297 | N55 | C297 | RBBP4_HUMAN - MesoLo | −1.3169849 | 0.017109 |
| 68 - 198 | N68 | C198 | MBD3_HUMAN - | −1.3178023 | 0.00557427 |
| | | | JMJD8_HUMAN | | |
| 182 - 111 | N182 | C111 | MTA3_HUMAN - | −1.3179629 | 7.86E−04 |
| | | | KDM5A_HUMAN | | |
| 306 - 172 | N306 | C172 | CobB - ASH1L_HUMAN | −1.3186284 | 0.00821866 |
| 3 - 221 | N3 | C221 | HDAC3 - SMYD2_HUMAN | −1.3200234 | 0.01778023 |
| 105 - 272 | N105 | C272 | MBD2_HUMAN - Sir2 | −1.3200334 | 0.00336617 |
| 245 - 68 | N245 | C68 | TYY1_HUMAN - | −1.3204912 | 0.00206419 |
| | | | MBD3_HUMAN | | |
| 131 - 308 | N131 | C308 | PRDM2_HUMAN - HDT1 | −1.3206402 | 0.04207069 |
| 317 - 315 | N317 | C315 | HDAC3 - NCoR | −1.3208018 | 6.97E−04 |
| 298 - 326 | N298 | C326 | MBD2b - pf_Sir2A | −1.3208303 | 0.00327666 |
| 317 - 249 | N317 | C249 | HDAC3 - HAC1-HAT | −1.3212584 | 0.04482727 |
| 268 - 7 | N268 | C7 | FOXA1-Cterm - ZNF10 | −1.3213509 | 0.03007043 |
| 185 - 68 | N185 | C68 | RBBP7_HUMAN - | −1.321652 | 0.02060359 |
| | | | MBD3_HUMAN | | |
| 306 - 135 | N306 | C135 | CobB - KDM2A_HUMAN | −1.3216545 | 0.0113458 |
| 233 - 298 | N233 | C298 | HINFP_HUMAN - MBD2b | −1.3220991 | 0.01280417 |
| 105 - 135 | N105 | C135 | MBD2_HUMAN - | −1.3229215 | 0.0016407 |
| | | | KDM2A_HUMAN | | |
| 55 - 17 | N55 | C17 | RBBP4_HUMAN - EZH2 | −1.3244575 | 0.03499289 |
| 63 - 32 | N63 | C32 | SMYD5_HUMAN - | −1.3276783 | 4.59E−04 |
| | | | Gilbert_Lab_CRISPRi_KRAB | | |
| 219 - 191 | N219 | C191 | JARD2_HUMAN - | −1.3282201 | 0.03169401 |
| | | | CDYL_HUMAN | | |
| 55 - 241 | N55 | C241 | RBBP4_HUMAN - | −1.3286652 | 0.00211317 |
| | | | SIR2_HUMAN | | |
| 166 - 68 | N166 | C68 | EHMT1_HUMAN - | −1.3308428 | 0.01048376 |
| | | | MBD3_HUMAN | | |
| 91 - 105 | N91 | C105 | ANM5_HUMAN - | −1.3309922 | 0.0030836 |
| | | | MBD2_HUMAN | | |
| 32 - 293 | N32 | C293 | Gilbert_Lab_CRISPRi_KRAB - | −1.3310517 | 0.00268475 |
| | | | KRAB-MeCP2 | | |
| 3 - 110 | N3 | C110 | HDAC3 - JMJD4_HUMAN | −1.3311765 | 0.04871336 |
| 6 - 32 | N6 | C32 | KDM1A - | −1.331333 | 0.04007449 |
| | | | Gilbert_Lab_CRISPRi_KRAB | | |
| 272 - 306 | N272 | C306 | Sir2 - CobB | −1.3314335 | 0.01689697 |
| 105 - 302 | N105 | C302 | MBD2_HUMAN - HDAC11 | −1.3325723 | 0.00134318 |
| 222 - 341 | N222 | C341 | IN80E_HUMAN - | −1.3334864 | 0.04384401 |
| | | | NCOR2_HUMAN | | |
| 131 - 327 | N131 | C327 | PRDM2_HUMAN - | −1.3337444 | 0.04445744 |
| | | | hs_NIPP1 | | |
| 252 - 32 | N252 | C32 | HCFC1_HUMAN - | −1.3338692 | 0.0103823 |
| | | | Gilbert_Lab_CRISPRi_KRAB | | |
| 152 - 280 | N152 | C280 | CDY1_HUMAN - CBF1- | −1.3339884 | 0.01666105 |
| | | | Cterm | | |
| 68 - 330 | N68 | C330 | MBD3_HUMAN - ce_Set1 | −1.3357006 | 0.00836193 |
| 306 - 210 | N306 | C210 | CobB - SIR3_HUMAN | −1.3363326 | 0.03911843 |
| 5 - 105 | N5 | C105 | KDM1A - MBD2_HUMAN | −1.3375134 | 7.15E−04 |
| 239 - 191 | N239 | C191 | KDM8_HUMAN - | −1.3385342 | 0.0246406 |
| | | | CDYL_HUMAN | | |
| 68 - 334 | N68 | C334 | MBD3_HUMAN - | −1.3391011 | 0.02362537 |
| | | | hs_RCOR1 | | |
| 273 - 306 | N273 | C306 | PRDM9-CD - CobB | −1.3399371 | 0.00137256 |
| 298 - 88 | N298 | C88 | MBD2b - ING4_HUMAN | −1.3406365 | 0.02235093 |
| 68 - 8 | N68 | C8 | MBD3_HUMAN - MECP2 | −1.3408919 | 0.02036753 |
| 300 - 105 | N300 | C105 | SIRT6 - MBD2_HUMAN | −1.3409066 | 0.00635382 |
| 17 - 293 | N17 | C293 | EZH2 - KRAB-MeCP2 | −1.3413378 | 0.0066314 |
| 275 - 215 | N275 | C215 | DOT1L-DOT1_domain - | −1.3417102 | 0.00350227 |
| | | | HDAC4_HUMAN | | |
| 182 - 219 | N182 | C219 | MTA3_HUMAN - | −1.342263 | 0.01673852 |
| | | | JARD2_HUMAN | | |
| 83 - 191 | N83 | C191 | SMRD3_HUMAN - | −1.3426999 | 0.03937916 |
| | | | CDYL_HUMAN | | |
| 257 - 68 | N257 | C68 | c-Myb-TAD - | −1.343505 | 0.00342522 |
| | | | MBD3_HUMAN | | |
| 55 - 68 | N55 | C68 | RBBP4_HUMAN - | −1.3437344 | 0.00426615 |
| | | | MBD3_HUMAN | | |
| 298 - 312 | N298 | C312 | MBD2b - SetD8 | −1.3444653 | 0.00285858 |
| 164 - 333 | N164 | C333 | SIR5_HUMAN - sc_RPD3 | −1.344854 | 0.01413019 |
| 68 - 106 | N68 | C106 | MBD3_HUMAN - | −1.3455357 | 0.01354673 |
| | | | KAT6B_HUMAN | | |
| 201 - 97 | N201 | C97 | KDM2B_HUMAN - | −1.3473168 | 0.03367627 |
| | | | CHD3_HUMAN | | |
| 9 - 199 | N9 | C199 | MECP2 - ING3_HUMAN | −1.3482367 | 0.02851579 |
| 306 - 272 | N306 | C272 | CobB - Sir2 | −1.3488961 | 5.86E−04 |
| 195 - 32 | N195 | C32 | SETD3_HUMAN - | −1.3495726 | 0.00156626 |
| | | | Gilbert_Lab_CRISPRi_KRAB | | |
| 112 - 298 | N112 | C298 | KDM5A_HUMAN - MBD2b | −1.3505795 | 0.00115342 |
| 111 - 215 | N111 | C215 | KDM5A_HUMAN - | −1.3507021 | 0.02745383 |
| | | | HDAC4_HUMAN | | |
| 5 - 306 | N5 | C306 | KDM1A - CobB | −1.3507436 | 0.00122878 |
| 312 - 39 | N312 | C39 | SetD8 - KDM3A | −1.3507911 | 0.02995332 |
| 38 - 298 | N38 | C298 | EHMT2 - MBD2b | −1.3509109 | 0.02788136 |
| 12 - 165 | N12 | C165 | KMT2A - MTA2_HUMAN | −1.3512343 | 0.01803467 |
| 55 - 12 | N55 | C12 | RBBP4_HUMAN - KMT2A | −1.3514977 | 3.48E−04 |
| 305 - 100 | N305 | C100 | NUE - TAF1_HUMAN | −1.3515013 | 0.02306934 |
| 334 - 298 | N334 | C298 | hs_RCOR1 - MBD2b | −1.3526637 | 7.82E−04 |
| 182 - 280 | N182 | C280 | MTA3_HUMAN - CBF1- | −1.3530852 | 0.0022332 |
| | | | Cterm | | |
| 9 - 255 | N9 | C255 | MECP2 - EED_HUMAN | −1.3535959 | 0.01905398 |
| 55 - 123 | N55 | C123 | RBBP4_HUMAN - | −1.353605 | 0.00136962 |
| | | | KMT2A_HUMAN | | |
| 196 - 69 | N196 | C69 | MTA1_HUMAN - | −1.3545039 | 0.02451018 |
| | | | SETB2_HUMAN | | |
| 105 - 310 | N105 | C310 | MBD2_HUMAN - TgSET8 | −1.3561597 | 0.00982321 |
| 79 - 105 | N79 | C105 | KAT6A_HUMAN - | −1.3561949 | 0.02711072 |
| | | | MBD2_HUMAN | | |
| 313 - 299 | N313 | C299 | SaII - HST2 | −1.3562604 | 9.33E−04 |
| 212 - 182 | N212 | C182 | HDAC8_HUMAN - | −1.3578342 | 0.01297195 |
| | | | MTA3_HUMAN | | |
| 63 - 298 | N63 | C298 | SMYD5_HUMAN - MBD2b | −1.3579015 | 0.00112881 |
| 196 - 59 | N196 | C59 | MTA1_HUMAN - | −1.3579797 | 0.00252628 |
| | | | INO80_HUMAN | | |
| 131 - 274 | N131 | C274 | PRDM2_HUMAN - UBE2A | −1.3582757 | 0.03328771 |
| 167 - 298 | N167 | C298 | EHMT1_HUMAN - MBD2b | −1.358472 | 0.00700411 |
| 182 - 271 | N182 | C271 | MTA3_HUMAN - GCN5 | −1.3595661 | 0.00585858 |
| 173 - 306 | N173 | C306 | SIR4_HUMAN - CobB | −1.3599469 | 0.01661123 |
| 105 - 127 | N105 | C127 | MBD2_HUMAN - | −1.3606759 | 2.42E−04 |
| | | | SETMR_HUMAN | | |
| 105 - 245 | N105 | C245 | MBD2_HUMAN - | −1.3619071 | 0.01175415 |
| | | | TYY1_HUMAN | | |
| 224 - 88 | N224 | C88 | KDM4C_HUMAN - | −1.3631457 | 0.03990732 |
| | | | ING4_HUMAN | | |
| 38 - 68 | N38 | C68 | EHMT2 - MBD3_HUMAN | −1.3634306 | 0.00110349 |
| 3 - 293 | N3 | C293 | HDAC3 - KRAB-MeCP2 | −1.3641532 | 0.00450441 |
| 317 - 116 | N317 | C116 | HDAC3 - ANM6_HUMAN | −1.3646068 | 0.01494278 |
| 293 - 25 | N293 | C25 | KRAB-MeCP2 - CTCFL | −1.364707 | 0.01451064 |
| 238 - 68 | N238 | C68 | NAT10_HUMAN - | −1.3653287 | 0.03300258 |
| | | | MBD3_HUMAN | | |
| 68 - 119 | N68 | C119 | MBD3_HUMAN - | −1.3656434 | 0.0169883 |
| | | | ACL6A_HUMAN | | |
| 298 - 146 | N298 | C146 | MBD2b - KDM4A_HUMAN | −1.3665163 | 0.03752935 |
| 131 - 320 | N131 | C320 | PRDM2_HUMAN - mMLL3- | −1.3669459 | 0.01491126 |
| | | | SET | | |
| 274 - 298 | N274 | C298 | UBE2A - MBD2b | −1.3670346 | 0.00448911 |
| 55 - 63 | N55 | C63 | RBBP4_HUMAN - | −1.3677118 | 0.00266573 |
| | | | SMYD5_HUMAN | | |
| 269 - 191 | N269 | C191 | PU.1 - CDYL_HUMAN | −1.3677827 | 0.00343321 |
| 315 - 68 | N315 | C68 | NCoR - MBD3_HUMAN | −1.3684914 | 0.00163603 |
| 106 - 293 | N106 | C293 | KAT6B_HUMAN - KRAB- | −1.3695215 | 0.01390429 |
| | | | MeCP2 | | |
| 196 - 346 | N196 | C346 | MTA1_HUMAN - | −1.3700524 | 0.03721287 |
| | | | MSX2_HUMAN | | |
| 48 - 341 | N48 | C341 | SETD5_HUMAN - | −1.3700708 | 0.00918611 |
| | | | NCOR2_HUMAN | | |
| 55 - 205 | N55 | C205 | RBBP4_HUMAN - | −1.3716911 | 7.40E−04 |
| | | | JMJD7_HUMAN | | |
| 268 - 105 | N268 | C105 | FOXA1-Cterm - | −1.3717322 | 0.00489244 |
| | | | MBD2_HUMAN | | |
| 96 - 346 | N96 | C346 | CHD3_HUMAN - | −1.3723834 | 0.03052 |
| | | | MSX2_HUMAN | | |
| 182 - 326 | N182 | C326 | MTA3_HUMAN - pf_Sir2A | −1.3728426 | 0.02135749 |
| 273 - 105 | N273 | C105 | PRDM9-CD - | −1.3733871 | 4.68E−04 |
| | | | MBD2_HUMAN | | |
| 131 - 113 | N131 | C113 | PRDM2_HUMAN - | −1.3739384 | 0.03764288 |
| | | | KDM5A_HUMAN | | |
| 298 - 194 | N298 | C194 | MBD2b - ING1_HUMAN | −1.3743326 | 0.00499149 |
| 68 - 246 | N68 | C246 | MBD3_HUMAN - | −1.3772678 | 4.65E−04 |
| | | | ATRX_HUMAN | | |
| 105 - 295 | N105 | C295 | MBD2_HUMAN - SET- | −1.3772867 | 0.01934434 |
| | | | TAF1B | | |
| 251 - 105 | N251 | C105 | HCFC1_HUMAN - | −1.3778386 | 0.04614832 |
| | | | MBD2_HUMAN | | |
| 95 - 311 | N95 | C311 | SETD6_HUMAN - Sin3a | −1.3782676 | 0.02841859 |
| 123 - 306 | N123 | C306 | KMT2A_HUMAN - CobB | −1.3782911 | 0.00156919 |
| 182 - 246 | N182 | C246 | MTA3_HUMAN - | −1.3791213 | 9.82E−04 |
| | | | ATRX_HUMAN | | |
| 298 - 99 | N298 | C99 | MBD2b - IN80C_HUMAN | −1.3792899 | 0.00245886 |
| 205 - 105 | N205 | C105 | JMJD7_HUMAN - | −1.381263 | 0.02587584 |
| | | | MBD2_HUMAN | | |
| 105 - 312 | N105 | C312 | MBD2_HUMAN - SetD8 | −1.3829733 | 0.00356295 |
| 269 - 165 | N269 | C165 | PU.1 - MTA2_HUMAN | −1.3839341 | 0.0215013 |
| 308 - 120 | N308 | C120 | HDT1 - RING1_HUMAN | −1.3840744 | 0.04929503 |
| 182 - 290 | N182 | C290 | MTA3_HUMAN - HP1aCS | −1.3846703 | 0.01182624 |
| 165 - 222 | N165 | C222 | MTA2_HUMAN - | −1.385719 | 0.01151984 |
| | | | IN80E_HUMAN | | |
| 3 - 27 | N3 | C27 | HDAC3 - EZH1 | −1.3863585 | 0.00953213 |
| 73 - 191 | N73 | C191 | KDM5D_HUMAN - | −1.3868697 | 0.00618048 |
| | | | CDYL_HUMAN | | |
| 196 - 17 | N196 | C17 | MTA1_HUMAN - EZH2 | −1.3870726 | 0.00537975 |
| 228 - 307 | N228 | C307 | HDAC7_HUMAN - KYP | −1.3873418 | 0.0417872 |
| 215 - 132 | N215 | C132 | HDAC4_HUMAN - | −1.3878255 | 0.02003509 |
| | | | PRDM2_HUMAN | | |
| 79 - 32 | N79 | C32 | KAT6A_HUMAN - | −1.3884495 | 0.04229349 |
| | | | Gilbert_Lab_CRISPRi_KRAB | | |
| 295 - 68 | N295 | C68 | SET-TAF1B - | −1.3896512 | 8.12E−04 |
| | | | MBD3_HUMAN | | |
| 107 - 55 | N107 | C55 | KAT6B_HUMAN - | −1.3911344 | 0.02245624 |
| | | | RBBP4_HUMAN | | |
| 293 - 191 | N293 | C191 | KRAB-MeCP2 - | −1.3924527 | 0.02080805 |
| | | | CDYL_HUMAN | | |
| 51 - 105 | N51 | C105 | UCHL5_HUMAN - | −1.3924732 | 5.87E−04 |
| | | | MBD2_HUMAN | | |
| 25 - 306 | N25 | C306 | CTCFL - CobB | −1.3965836 | 0.0033616 |
| 21 - 105 | N21 | C105 | KDM1B - MBD2_HUMAN | −1.3975862 | 0.0011573 |
| 8 - 151 | N8 | C151 | MECP2 - SIR7_HUMAN | −1.3978009 | 0.01429118 |
| 205 - 314 | N205 | C314 | JMJD7_HUMAN - MeCP2 | −1.3978319 | 0.03019206 |
| 199 - 191 | N199 | C191 | ING3_HUMAN - | −1.397944 | 0.00858181 |
| | | | CDYL_HUMAN | | |
| 87 - 68 | N87 | C68 | SETD7_HUMAN - | −1.3982399 | 0.03137278 |
| | | | MBD3_HUMAN | | |
| 293 - 7 | N293 | C7 | KRAB-MeCP2 - ZNF10 | −1.3983398 | 0.01019261 |
| 307 - 306 | N307 | C306 | KYP - CobB | −1.4001947 | 0.01903139 |
| 288 - 300 | N288 | C300 | SUV[SET] - SIRT6 | −1.4006896 | 0.01069008 |
| 55 - 47 | N55 | C47 | RBBP4_HUMAN - | −1.4023079 | 0.02523387 |
| | | | ANM2_HUMAN | | |
| 246 - 185 | N246 | C185 | ATRX_HUMAN - | −1.4025081 | 0.00160955 |
| | | | RBBP7_HUMAN | | |
| 55 - 249 | N55 | C249 | RBBP4_HUMAN - HAC1- | −1.4032442 | 0.02283769 |
| | | | HAT | | |
| 88 - 32 | N88 | C32 | ING4_HUMAN - | −1.4038739 | 0.01261637 |
| | | | Gilbert_Lab_CRISPRi_KRAB | | |
| 55 - 38 | N55 | C38 | RBBP4_HUMAN - EHMT2 | −1.4041085 | 0.01717707 |
| 228 - 233 | N228 | C233 | HDAC7_HUMAN - | −1.4045776 | 0.03242074 |
| | | | HINFP_HUMAN | | |
| 105 - 194 | N105 | C194 | MBD2_HUMAN - | −1.4059725 | 0.00225214 |
| | | | ING1_HUMAN | | |
| 298 - 287 | N298 | C287 | MBD2b - G9A[SET] | −1.406292 | 0.04405819 |
| 269 - 293 | N269 | C293 | PU.1 - KRAB-MeCP2 | −1.4074166 | 8.78E−04 |
| 68 - 345 | N68 | C345 | MBD3_HUMAN - | −1.4077955 | 0.01765396 |
| | | | MSX2_HUMAN | | |
| 298 - 197 | N298 | C197 | MBD2b - SUV91_HUMAN | −1.409479 | 0.02454339 |
| 182 - 8 | N182 | C8 | MTA3_HUMAN - MECP2 | −1.4096088 | 0.00234131 |
| 334 - 245 | N334 | C245 | hs_RCOR1 - TYY1_HUMAN | −1.4100576 | 0.01521445 |
| 226 - 334 | N226 | C334 | SETD2_HUMAN - | −1.4108762 | 0.00226276 |
| | | | hs_RCOR1 | | |
| 320 - 165 | N320 | C165 | mMLL3-SET - | −1.4123898 | 0.00879912 |
| | | | MTA2_HUMAN | | |
| 69 - 68 | N69 | C68 | SETB2_HUMAN - | −1.4133136 | 0.00161702 |
| | | | MBD3_HUMAN | | |
| 135 - 88 | N135 | C88 | KDM2A_HUMAN - | −1.4138913 | 0.04264226 |
| | | | ING4_HUMAN | | |
| 75 - 296 | N75 | C296 | KDM5D_HUMAN - HP1a | −1.4139215 | 0.03992301 |
| 105 - 73 | N105 | C73 | MBD2_HUMAN - | −1.4150021 | 0.00265572 |
| | | | KDM5D_HUMAN | | |
| 190 - 293 | N190 | C293 | ANM1_HUMAN - KRAB- | −1.4160756 | 0.03092579 |
| | | | MeCP2 | | |
| 3 - 313 | N3 | C313 | HDAC3 - SaII | −1.4168441 | 2.06E−04 |
| 317 - 164 | N317 | C164 | HDAC3 - SIR5_HUMAN | −1.4171184 | 0.00293925 |
| 162 - 32 | N162 | C32 | SIR6_HUMAN - | −1.4172587 | 3.71E−04 |
| | | | Gilbert_Lab_CRISPRi_KRAB | | |
| 277 - 306 | N277 | C306 | SUV39H1 - CobB | −1.4176025 | 0.01013477 |
| 149 - 68 | N149 | C68 | MBD1_HUMAN - | −1.4188597 | 0.00150054 |
| | | | MBD3_HUMAN | | |
| 344 - 118 | N344 | C118 | HDAC3_HUMAN - | −1.4189561 | 0.00759233 |
| | | | HDAC6_HUMAN | | |
| 331 - 298 | N331 | C298 | mm_G9a - MBD2b | −1.4193108 | 0.00114906 |
| 190 - 68 | N190 | C68 | ANM1_HUMAN - | −1.4205395 | 0.00312 |
| | | | MBD3_HUMAN | | |
| 68 - 154 | N68 | C154 | MBD3_HUMAN - | −1.4209156 | 0.00183073 |
| | | | NSD2_HUMAN | | |
| 302 - 202 | N302 | C202 | HDAC11 - | −1.4211179 | 0.04809194 |
| | | | KDM2B_HUMAN | | |
| 306 - 202 | N306 | C202 | CobB - KDM2B_HUMAN | −1.4212073 | 0.02991387 |
| 165 - 34 | N165 | C34 | MTA2_HUMAN - DNMT3A- | −1.4222437 | 0.00204577 |
| | | | 3L_JKNp112 | | |
| 77 - 202 | N77 | C202 | KDM5D_HUMAN - | −1.4243498 | 0.01297585 |
| | | | KDM2B_HUMAN | | |
| 38 - 165 | N38 | C165 | EHMT2 - MTA2_HUMAN | −1.4244697 | 0.02569778 |
| 298 - 91 | N298 | C91 | MBD2b - ANM5_HUMAN | −1.4253045 | 0.01043129 |
| 196 - 294 | N196 | C294 | MTA1_HUMAN - DIM5 | −1.4260538 | 0.02560031 |
| 182 - 269 | N182 | C269 | MTA3_HUMAN - PU.1 | −1.4262308 | 0.00114861 |
| 266 - 306 | N266 | C306 | GADD45A - CobB | −1.4262318 | 7.02E−04 |
| 306 - 9 | N306 | C9 | CobB - MECP2 | −1.4262574 | 9.12E−04 |
| 215 - 78 | N215 | C78 | HDAC4_HUMAN - | −1.4264047 | 0.02638801 |
| | | | KAT6A_HUMAN | | |
| 177 - 191 | N177 | C191 | KDM5C_HUMAN - | −1.4267987 | 0.01782111 |
| | | | CDYL_HUMAN | | |
| 327 - 55 | N327 | C55 | hs_NIPP1 - RBBP4_HUMAN | −1.4271433 | 0.00905923 |
| 105 - 133 | N105 | C133 | MBD2_HUMAN - | −1.4286408 | 0.00598968 |
| | | | KAT8_HUMAN | | |
| 17 - 105 | N17 | C105 | EZH2 - MBD2_HUMAN | −1.4287016 | 1.86E−04 |
| 317 - 332 | N317 | C332 | HDAC3 - hs_SIRT5 | −1.4289979 | 0.00215717 |
| 5 - 293 | N5 | C293 | KDM1A - KRAB-MeCP2 | −1.4290654 | 0.01849954 |
| 68 - 116 | N68 | C116 | MBD3_HUMAN - | −1.4294221 | 0.00941555 |
| | | | ANM6_HUMAN | | |
| 105 - 20 | N105 | C20 | MBD2_HUMAN - KDM1B | −1.4297648 | 8.10E−04 |
| 55 - 345 | N55 | C345 | RBBP4_HUMAN - | −1.4303146 | 0.00208989 |
| | | | MSX2_HUMAN | | |
| 197 - 105 | N197 | C105 | SUV91_HUMAN - | −1.4305329 | 0.00185559 |
| | | | MBD2_HUMAN | | |
| 165 - 20 | N165 | C20 | MTA2_HUMAN - KDM1B | −1.4305818 | 0.03953392 |
| 3 - 335 | N3 | C335 | HDAC3 - at_SUVR4 | −1.4311037 | 0.00410836 |
| 105 - 183 | N105 | C183 | MBD2_HUMAN - | −1.4324937 | 0.00477301 |
| | | | KMT2C_HUMAN | | |
| 317 - 32 | N317 | C32 | HDAC3 - | −1.4333993 | 0.00223512 |
| | | | Gilbert_Lab_CRISPRi_KRAB | | |
| 113 - 105 | N113 | C105 | KDM5A_HUMAN - | −1.4349031 | 0.00150761 |
| | | | MBD2_HUMAN | | |
| 165 - 305 | N165 | C305 | MTA2_HUMAN - NUE | −1.4349901 | 0.01029066 |
| 341 -66 | N341 | C66 | NCOR2_HUMAN - | −1.4356236 | 0.03575713 |
| | | | DOT1L_HUMAN | | |
| 182 - 229 | N182 | C229 | MTA3_HUMAN - | −1.4357424 | 0.00442021 |
| | | | NFRKB_HUMAN | | |
| 186 - 32 | N186 | C32 | RIOX2_HUMAN - | −1.4379468 | 4.46E−04 |
| | | | Gilbert_Lab_CRISPRi_KRAB | | |
| 182 - 68 | N182 | C68 | MTA3_HUMAN - | −1.4385345 | 0.00142525 |
| | | | MBD3_HUMAN | | |
| 68 - 45 | N68 | C45 | MBD3_HUMAN - | −1.4387473 | 0.01320901 |
| | | | KMT2D_HUMAN | | |
| 105 - 34 | N105 | C34 | MBD2_HUMAN - DNMT3A- | −1.4394557 | 0.00100189 |
| | | | 3L_JKNp112 | | |
| 274 - 191 | N274 | C191 | UBE2A - CDYL_HUMAN | −1.4405006 | 0.03682924 |
| 32 - 191 | N32 | C191 | Gilbert_Lab_CRISPRi_KRAB - | −1.4406911 | 0.00134765 |
| | | | CDYL_HUMAN | | |
| 346 - 165 | N346 | C165 | MSX2_HUMAN - | −1.440715 | 0.00146134 |
| | | | MTA2_HUMAN | | |
| 162 - 298 | N162 | C298 | SIR6_HUMAN - MBD2b | −1.4412342 | 0.00130463 |
| 105 - 294 | N105 | C294 | MBD2_HUMAN - DIM5 | −1.4413015 | 7.14E−05 |
| 228 - 312 | N228 | C312 | HDAC7_HUMAN - SetD8 | −1.4419809 | 0.02908662 |
| 116 - 105 | N116 | C105 | ANM6_HUMAN - | −1.4426095 | 0.00132878 |
| | | | MBD2_HUMAN | | |
| 105 - 56 | N105 | C56 | MBD2_HUMAN - | −1.4428277 | 0.00127318 |
| | | | SUV92_HUMAN | | |
| 131 - 314 | N131 | C314 | PRDM2_HUMAN - MeCP2 | −1.443305 | 7.95E−04 |
| 105 - 116 | N105 | C116 | MBD2_HUMAN - | −1.4436162 | 0.0016288 |
| | | | ANM6_HUMAN | | |
| 216 - 298 | N216 | C298 | ING5_HUMAN - MBD2b | −1.4439951 | 5.87E−04 |
| 24 - 173 | N24 | C173 | CTCFL - SIR4_HUMAN | −1.444201 | 0.03511658 |
| 133 - 186 | N133 | C186 | KAT8_HUMAN - | −1.4455098 | 0.04419971 |
| | | | RIOX2_HUMAN | | |
| 334 - 215 | N334 | C215 | hs_RCOR1 - | −1.445781 | 0.03181152 |
| | | | HDAC4_HUMAN | | |
| 135 - 298 | N135 | C298 | KDM2A_HUMAN - MBD2b | −1.4462135 | 0.00914657 |
| 182 - 63 | N182 | C63 | MTA3_HUMAN - | −1.4465996 | 0.00198451 |
| | | | SMYD5_HUMAN | | |
| 23 - 274 | N23 | C274 | TET1 - UBE2A | −1.4468555 | 0.03010238 |
| 69 - 55 | N69 | C55 | SETB2_HUMAN - | −1.4479524 | 0.02300465 |
| | | | RBBP4_HUMAN | | |
| 23 - 105 | N23 | C105 | TET1 - MBD2_HUMAN | −1.4484156 | 0.02386389 |
| 67 - 191 | N67 | C191 | PRDM9_HUMAN - | −1.4490016 | 0.03889068 |
| | | | CDYL_HUMAN | | |
| 3 - 173 | N3 | C173 | HDAC3 - SIR4_HUMAN | −1.4491913 | 0.00540295 |
| 215 - 216 | N215 | C216 | HDAC4_HUMAN - | −1.4494013 | 0.00776304 |
| | | | ING5_HUMAN | | |
| 173 - 293 | N173 | C293 | SIR4_HUMAN - KRAB- | −1.4502824 | 0.00657288 |
| | | | MeCP2 | | |
| 298 - 113 | N298 | C113 | MBD2b - KDM5A_HUMAN | −1.4512518 | 0.00701482 |
| 162 - 293 | N162 | C293 | SIR6_HUMAN - KRAB- | −1.4512975 | 0.00698116 |
| | | | MeCP2 | | |
| 18 - 32 | N18 | C32 | EZH2 - | −1.4521011 | 0.00349301 |
| | | | Gilbert_Lab_CRISPRi_KRAB | | |
| 215 - 5 | N215 | C5 | HDAC4_HUMAN - KDM1A | −1.452489 | 0.04285392 |
| 344 - 55 | N344 | C55 | HDAC3_HUMAN - | −1.4525222 | 0.00543402 |
| | | | RBBP4_HUMAN | | |
| 215 - 56 | N215 | C56 | HDAC4_HUMAN - | −1.4537966 | 0.00345827 |
| | | | SUV92_HUMAN | | |
| 275 - 32 | N275 | C32 | DOT1L-DOT1_domain - | −1.4543743 | 9.16E−04 |
| | | | Gilbert_Lab_CRISPRi_KRAB | | |
| 128 - 233 | N128 | C233 | HAIR_HUMAN - | −1.4543819 | 0.04659986 |
| | | | HINFP_HUMAN | | |
| 55 - 67 | N55 | C67 | RBBP4_HUMAN - | −1.454538 | 0.0218015 |
| | | | PRDM9_HUMAN | | |
| 254 - 298 | N254 | C298 | MEN1_HUMAN - MBD2b | −1.4552431 | 0.02409466 |
| 77 - 105 | N77 | C105 | KDM5D_HUMAN - | −1.4553391 | 0.00301553 |
| | | | MBD2_HUMAN | | |
| 131 - 315 | N131 | C315 | PRDM2_HUMAN - NCoR | −1.4565835 | 0.03943505 |
| 108 - 255 | N108 | C255 | KAT6B_HUMAN - | −1.4567426 | 0.01668762 |
| | | | EED_HUMAN | | |
| 298 - 153 | N298 | C153 | MBD2b - KAT7_HUMAN | −1.4573176 | 0.02560699 |
| 151 - 306 | N151 | C306 | SIR7_HUMAN - CobB | −1.4577044 | 9.51E−04 |
| 315 - 131 | N315 | C131 | NCoR - PRDM2_HUMAN | −1.4579224 | 0.00912985 |
| 299 - 55 | N299 | C55 | HST2 - RBBP4_HUMAN | −1.4581027 | 0.04769023 |
| 308 - 105 | N308 | C105 | HDT1 - MBD2_HUMAN | −1.4585609 | 0.00135941 |
| 187 - 326 | N187 | C326 | MBD4_HUMAN - pf_Sir2A | −1.4598577 | 0.02410887 |
| 106 - 105 | N106 | C105 | KAT6B_HUMAN - | −1.4606753 | 4.42E−05 |
| | | | MBD2_HUMAN | | |
| 309 - 218 | N309 | C218 | HDAC8 - IN80B_HUMAN | −1.4609366 | 0.01212608 |
| 24 - 105 | N24 | C105 | CTCFL - MBD2_HUMAN | −1.4612117 | 3.51E−05 |
| 154 - 32 | N154 | C32 | NSD2_HUMAN - | −1.4634844 | 0.01540003 |
| | | | Gilbert_Lab_CRISPRi_KRAB | | |
| 220 - 299 | N220 | C299 | JARD2_HUMAN - HST2 | −1.4649816 | 0.00264047 |
| 237 - 298 | N237 | C298 | KAT5_HUMAN - MBD2b | −1.4653375 | 7.55E−04 |
| 306 - 136 | N306 | C136 | CobB - JMJD6_HUMAN | −1.4660728 | 0.01426655 |
| 24 - 271 | N24 | C271 | CTCFL - GCN5 | −1.4675154 | 0.00270947 |
| 298 - 56 | N298 | C56 | MBD2b - SUV92_HUMAN | −1.4675384 | 0.00900826 |
| 277 - 298 | N277 | C298 | SUV39H1 - MBD2b | −1.4677964 | 0.00228094 |
| 315 - 334 | N315 | C334 | NCoR - hs_RCOR1 | −1.4680421 | 0.00401285 |
| 87 - 95 | N87 | C95 | SETD7_HUMAN - | −1.4681076 | 0.03539549 |
| | | | SETD6_HUMAN | | |
| 165 - 56 | N165 | C56 | MTA2_HUMAN - | −1.4685293 | 0.04383368 |
| | | | SUV92_HUMAN | | |
| 299 - 248 | N299 | C248 | HST2 - GLYR1_HUMAN | −1.469764 | 0.01562272 |
| 105 - 99 | N105 | C99 | MBD2_HUMAN - | −1.4722582 | 0.00318641 |
| | | | IN80C_HUMAN | | |
| 136 - 191 | N136 | C191 | JMJD6_HUMAN - | −1.4733811 | 0.0017388 |
| | | | CDYL_HUMAN | | |
| 300 - 293 | N300 | C293 | SIRT6 - KRAB-MeCP2 | −1.4739969 | 0.00234045 |
| 182 - 136 | N182 | C136 | MTA3_HUMAN - | −1.474101 | 0.02509433 |
| | | | JMJD6_HUMAN | | |
| 105 - 300 | N105 | C300 | MBD2_HUMAN - SIRT6 | −1.4769381 | 6.87E−05 |
| 299 - 47 | N299 | C47 | HST2 - ANM2_HUMAN | −1.4773963 | 0.02632064 |
| 174 - 214 | N174 | C214 | HDAC1_HUMAN - | −1.4774319 | 0.03431556 |
| | | | HDAC4_HUMAN | | |
| 105 - 94 | N105 | C94 | MBD2_HUMAN - | −1.4780368 | 0.00483805 |
| | | | KDM3B_HUMAN | | |
| 165 - 274 | N165 | C274 | MTA2_HUMAN - UBE2A | −1.4787689 | 9.77E−04 |
| 314 - 107 | N314 | C107 | MeCP2 - KAT6B_HUMAN | −1.478942 | 0.04174119 |
| 105 - 314 | N105 | C314 | MBD2_HUMAN - MeCP2 | −1.4794314 | 0.00402375 |
| 162 - 68 | N162 | C68 | SIR6_HUMAN - | −1.4805386 | 0.00235818 |
| | | | MBD3_HUMAN | | |
| 85 - 32 | N85 | C32 | SMYD3_HUMAN - | −1.4807117 | 0.00227969 |
| | | | Gilbert_Lab_CRISPRi_KRAB | | |
| 222 - 215 | N222 | C215 | IN80E_HUMAN - | −1.4808689 | 0.0218761 |
| | | | HDAC4_HUMAN | | |
| 123 - 191 | N123 | C191 | KMT2A_HUMAN - | −1.4810782 | 0.00186587 |
| | | | CDYL_HUMAN | | |
| 105 - 215 | N105 | C215 | MBD2_HUMAN - | −1.4811986 | 0.01420648 |
| | | | HDAC4_HUMAN | | |
| 275 - 105 | N275 | C105 | DOT1L-DOT1_domain - | −1.4818684 | 7.74E−05 |
| | | | MBD2_HUMAN | | |
| 345 - 32 | N345 | C32 | MSX2_HUMAN - | −1.4819849 | 0.02664506 |
| | | | Gilbert_Lab_CRISPRi_KRAB | | |
| 310 - 182 | N310 | C182 | TgSET8 - MTA3_HUMAN | −1.4832045 | 0.03282199 |
| 38 - 105 | N38 | C105 | EHMT2 - MBD2_HUMAN | −1.4836232 | 8.34E−05 |
| 182 - 69 | N182 | C69 | MTA3_HUMAN - | −1.4862279 | 0.01275282 |
| | | | SETB2_HUMAN | | |
| 3 - 87 | N3 | C87 | HDAC3 - SETD7_HUMAN | −1.4873203 | 0.00440884 |
| 329 - 298 | N329 | C298 | pbcv1_vSET - MBD2b | −1.4877335 | 1.95E−04 |
| 3 - 198 | N3 | C198 | HDAC3 - JMJD8_HUMAN | −1.4890973 | 0.00951466 |
| 268 - 334 | N268 | C334 | FOXA1-Cterm - hs_RCOR1 | −1.4891229 | 0.0015488 |
| 100 - 32 | N100 | C32 | TAF1_HUMAN - | −1.48923 | 0.00603353 |
| | | | Gilbert_Lab_CRISPRi_KRAB | | |
| 188 - 334 | N188 | C334 | SMYD4_HUMAN - | −1.4893079 | 0.0191203 |
| | | | hs_RCOR1 | | |
| 151 -32 | N151 | C32 | SIR7_HUMAN - | −1.4917631 | 0.0059674 |
| | | | Gilbert_Lab_CRISPRi_KRAB | | |
| 307 - 196 | N307 | C196 | KYP - MTA1_HUMAN | −1.4928924 | 0.03919388 |
| 143 - 32 | N143 | C32 | KDM4B_HUMAN - | −1.4929837 | 0.01397837 |
| | | | Gilbert_Lab_CRISPRi_KRAB | | |
| 105 - 308 | N105 | C308 | MBD2_HUMAN - HDT1 | −1.4934079 | 0.00237223 |
| 130 - 322 | N130 | C322 | PRDM2_HUMAN - | −1.4938397 | 0.04851983 |
| | | | MINT_HUMAN | | |
| 317 - 289 | N317 | C289 | HDAC3 - FOG1 | −1.4939297 | 0.00532047 |
| 55 - 165 | N55 | C165 | RBBP4_HUMAN - | −1.4940788 | 0.03670672 |
| | | | MTA2_HUMAN | | |
| 324 - 215 | N324 | C215 | LBR_HUMAN - | −1.4941931 | 0.00181654 |
| | | | HDAC4_HUMAN | | |
| 128 - 315 | N128 | C315 | HAIR_HUMAN - NCoR | −1.4976623 | 0.02452855 |
| 267 - 298 | N267 | C298 | FOXA1-Nterm - MBD2b | −1.497724 | 6.35E−04 |
| 25 - 256 | N25 | C256 | CTCFL - LSD1-ORF | −1.4977886 | 0.04410713 |
| 272 - 127 | N272 | C127 | Sir2 - SETMR_HUMAN | −1.499124 | 0.03293159 |
| 105 - 270 | N105 | C270 | MBD2_HUMAN - ENL | −1.4997702 | 0.00222759 |
| 317 - 246 | N317 | C246 | HDAC3 - ATRX_HUMAN | −1.500439 | 2.58E−04 |
| 293 - 241 | N293 | C241 | KRAB-MeCP2 - | −1.5008154 | 0.00586427 |
| | | | SIR2_HUMAN | | |
| 88 - 298 | N88 | C298 | ING4_HUMAN - MBD2b | −1.500952 | 0.00174573 |
| 3 - 55 | N3 | C55 | HDAC3 - RBBP4_HUMAN | −1.5015892 | 0.02642142 |
| 3 - 204 | N3 | C204 | HDAC3 - HIF1N_HUMAN | −1.5020639 | 0.00434244 |
| 105 - 280 | N105 | C280 | MBD2_HUMAN - CBF1- | −1.5024696 | 0.00326985 |
| | | | Cterm | | |
| 146 - 105 | N146 | C105 | KDM4A_HUMAN - | −1.5030222 | 0.0078225 |
| | | | MBD2_HUMAN | | |
| 341 - 246 | N341 | C246 | NCOR2_HUMAN - | −1.505154 | 0.01543445 |
| | | | ATRX_HUMAN | | |
| 131 - 73 | N131 | C73 | PRDM2_HUMAN - | −1.5058032 | 4.38E−04 |
| | | | KDM5D_HUMAN | | |
| 217 - 191 | N217 | C191 | CHRC1_HUMAN - | −1.5068201 | 0.0266692 |
| | | | CDYL_HUMAN | | |
| 215 - 12 | N215 | C12 | HDAC4_HUMAN - KMT2A | −1.5081641 | 0.00312664 |
| 123 - 105 | N123 | C105 | KMT2A_HUMAN - | −1.5084745 | 3.49E−04 |
| | | | MBD2_HUMAN | | |
| 222 - 191 | N222 | C191 | IN80E_HUMAN - | −1.5091046 | 0.0044341 |
| | | | CDYL_HUMAN | | |
| 3 - 97 | N3 | C97 | HDAC3 - CHD3_HUMAN | −1.5091102 | 0.00854381 |
| 68 - 5 | N68 | C5 | MBD3_HUMAN - KDM1A | −1.5100915 | 0.01513469 |
| 111 - 293 | N111 | C293 | KDM5A_HUMAN - KRAB- | −1.5110471 | 0.01293786 |
| | | | MeCP2 | | |
| 69 - 202 | N69 | C202 | SETB2_HUMAN - | −1.5115875 | 0.01173844 |
| | | | KDM2B_HUMAN | | |
| 105 - 163 | N105 | C163 | MBD2_HUMAN - | −1.5118376 | 0.0026488 |
| | | | ANM7_HUMAN | | |
| 205 - 333 | N205 | C333 | JMJD7_HUMAN - sc_RPD3 | −1.5119511 | 0.02040196 |
| 328 - 298 | N328 | C298 | hs_PHF19 - MBD2b | −1.5119801 | 0.00876459 |
| 226 - 105 | N226 | C105 | SETD2_HUMAN - | −1.5120441 | 0.00405239 |
| | | | MBD2_HUMAN | | |
| 175 - 218 | N175 | C218 | RUVB2_HUMAN - | −1.512397 | 0.03251661 |
| | | | IN80B_HUMAN | | |
| 105 - 85 | N105 | C85 | MBD2_HUMAN - | −1.5124577 | 7.59E−04 |
| | | | SMYD3_HUMAN | | |
| 130 - 97 | N130 | C97 | PRDM2_HUMAN - | −1.5126052 | 0.01956861 |
| | | | CHD3_HUMAN | | |
| 55 - 106 | N55 | C106 | RBBP4_HUMAN - | −1.513927 | 2.65E−04 |
| | | | KAT6B_HUMAN | | |
| 293 - 108 | N293 | C108 | KRAB-MeCP2 - | −1.514382 | 0.04587446 |
| | | | KAT6B_HUMAN | | |
| 131 - 294 | N131 | C294 | PRDM2_HUMAN - DIM5 | −1.5146486 | 0.0353811 |
| 298 - 75 | N298 | C75 | MBD2b - KDM5D_HUMAN | −1.5149728 | 0.00464009 |
| 129 - 105 | N129 | C105 | HAIR_HUMAN - | −1.5155539 | 0.00249576 |
| | | | MBD2_HUMAN | | |
| 105 - 303 | N105 | C303 | MBD2_HUMAN - EZH2 | −1.5158002 | 0.0136604 |
| 213 - 190 | N213 | C190 | HBAP1_HUMAN - | −1.515822 | 0.03074989 |
| | | | ANM1_HUMAN | | |
| 201 - 293 | N201 | C293 | KDM2B_HUMAN - KRAB- | −1.5164452 | 0.01958011 |
| | | | MeCP2 | | |
| 241 - 334 | N241 | C334 | SIR2_HUMAN - hs_RCOR1 | −1.5167025 | 0.02683367 |
| 202 - 289 | N202 | C289 | KDM2B_HUMAN - FOG1 | −1.5174989 | 0.01000875 |
| 3 - 332 | N3 | C332 | HDAC3 - hs_SIRT5 | −1.5193879 | 1.80E−04 |
| 298 - 328 | N298 | C328 | MBD2b - hs_PHF19 | −1.5197249 | 0.00384227 |
| 256 - 269 | N256 | C269 | LSD1-ORF - PU.1 | −1.5199914 | 0.01208931 |
| 306 - 303 | N306 | C303 | CobB - EZH2 | −1.5215049 | 0.00466464 |
| 144 - 298 | N144 | C298 | UTY_HUMAN - MBD2b | −1.5217196 | 0.02084244 |
| 200 - 32 | N200 | C32 | ARP5_HUMAN - | −1.5221757 | 0.01320819 |
| | | | Gilbert_Lab_CRISPRi_KRAB | | |
| 55 - 8 | N55 | C8 | RBBP4_HUMAN - MECP2 | −1.5222608 | 2.13E−04 |
| 105 - 155 | N105 | C155 | MBD2_HUMAN - | −1.522908 | 4.01E−04 |
| | | | NSD2_HUMAN | | |
| 105 -210 | N105 | C210 | MBD2_HUMAN - | −1.5233208 | 0.01806123 |
| | | | SIR3_HUMAN | | |
| 105 - 45 | N105 | C45 | MBD2_HUMAN - | −1.5241247 | 2.36E−04 |
| | | | KMT2D_HUMAN | | |
| 108 - 8 | N108 | C8 | KAT6B_HUMAN - MECP2 | −1.5255338 | 0.02258125 |
| 105 - 167 | N105 | C167 | MBD2_HUMAN - | −1.5257214 | 0.02429358 |
| | | | EHMT1_HUMAN | | |
| 344 - 332 | N344 | C332 | HDAC3_HUMAN - | −1.5270354 | 0.00433238 |
| | | | hs_SIRT5 | | |
| 197 - 293 | N197 | C293 | SUV91_HUMAN - KRAB- | −1.52713 | 0.03098591 |
| | | | MeCP2 | | |
| 66 - 298 | N66 | C298 | DOT1L_HUMAN - MBD2b | −1.5271699 | 3.71E−04 |
| 306 - 151 | N306 | C151 | CobB - SIR7_HUMAN | −1.5273948 | 0.00886292 |
| 306 - 7 | N306 | C7 | CobB - ZNF10 | −1.5278554 | 1.19E−04 |
| 55 - 77 | N55 | C77 | RBBP4_HUMAN - | −1.5287575 | 0.00235358 |
| | | | KDM5D_HUMAN | | |
| 164 - 105 | N164 | C105 | SIR5_HUMAN - | −1.5290389 | 2.14E−04 |
| | | | MBD2_HUMAN | | |
| 3 - 345 | N3 | C345 | HDAC3 - MSX2_HUMAN | −1.5305986 | 0.00141218 |
| 165 - 132 | N165 | C132 | MTA2_HUMAN - | −1.5315359 | 0.02624577 |
| | | | PRDM2_HUMAN | | |
| 298 - 198 | N298 | C198 | MBD2b - JMJD8_HUMAN | −1.5322569 | 0.04758382 |
| 215 - 344 | N215 | C344 | HDAC4_HUMAN - | −1.5323864 | 0.01365395 |
| | | | HDAC3_HUMAN | | |
| 105 - 92 | N105 | C92 | MBD2_HUMAN - | −1.5327174 | 0.00304728 |
| | | | SMYD1_HUMAN | | |
| 68 - 315 | N68 | C315 | MBD3_HUMAN - NCoR | −1.5333766 | 4.06E−04 |
| 182 - 105 | N182 | C105 | MTA3_HUMAN - | −1.5344677 | 2.83E−04 |
| | | | MBD2_HUMAN | | |
| 182 - 164 | N182 | C164 | MTA3_HUMAN - | −1.5359855 | 0.00104904 |
| | | | SIR5_HUMAN | | |
| 3 - 233 | N3 | C233 | HDAC3 - HINFP_HUMAN | −1.5386376 | 6.39E−05 |
| 87 - 105 | N87 | C105 | SETD7_HUMAN - | −1.5393979 | 0.01356 |
| | | | MBD2_HUMAN | | |
| 25 - 215 | N25 | C215 | CTCFL - HDAC4_HUMAN | −1.54068 | 0.00116367 |
| 32 - 215 | N32 | C215 | Gilbert_Lab_CRISPRi_KRAB - | −1.5411741 | 0.03449299 |
| | | | HDAC4_HUMAN | | |
| 249 - 293 | N249 | C293 | MTL5_HUMAN - KRAB- | −1.5412465 | 0.00470719 |
| | | | MeCP2 | | |
| 150 - 32 | N150 | C32 | HDA11_HUMAN - | −1.5416997 | 4.56E−04 |
| | | | Gilbert_Lab_CRISPRi_KRAB | | |
| 105 - 158 | N105 | C158 | MBD2_HUMAN - | −1.5427961 | 0.01747048 |
| | | | SETB1_HUMAN | | |
| 216 - 105 | N216 | C105 | ING5_HUMAN - | −1.5430775 | 2.63E−05 |
| | | | MBD2_HUMAN | | |
| 24 - 298 | N24 | C298 | CTCFL - MBD2b | −1.5440097 | 1.85E−04 |
| 196 - 56 | N196 | C56 | MTA1_HUMAN - | −1.544922 | 0.00486273 |
| | | | SUV92_HUMAN | | |
| 167 - 164 | N167 | C164 | EHMT1_HUMAN - | −1.5458399 | 0.02416352 |
| | | | SIR5_HUMAN | | |
| 250 - 298 | N250 | C298 | HCFC1_HUMAN - MBD2b | −1.5461085 | 0.02431457 |
| 12 - 298 | N12 | C298 | KMT2A - MBD2b | −1.5463801 | 8.86E−05 |
| 68 - 313 | N68 | C313 | MBD3_HUMAN - SaII | −1.5465558 | 5.48E−04 |
| 206 - 105 | N206 | C105 | SETD9_HUMAN - | −1.5473976 | 6.01E−04 |
| | | | MBD2_HUMAN | | |
| 298 - 275 | N298 | C275 | MBD2b - DOT1L- | −1.5480803 | 0.00443389 |
| | | | DOT1_domain | | |
| 154 - 334 | N154 | C334 | NSD2_HUMAN - hs_RCOR1 | −1.5482714 | 0.00130974 |
| 222 - 68 | N222 | 268 | IN80E_HUMAN - | −1.5485965 | 1.25E−04 |
| | | | MBD3_HUMAN | | |
| 293 - 345 | N293 | C345 | KRAB-MeCP2 - | −1.5485985 | 0.00146311 |
| | | | MSX2_HUMAN | | |
| 17 - 298 | N17 | C298 | EZH2 - MBD2b | −1.5494771 | 1.59E−05 |
| 217 - 7 | N217 | C7 | CHRC1_HUMAN - ZNF10 | −1.5497809 | 0.01670789 |
| 136 - 51 | N136 | C51 | JMJD6_HUMAN - | −1.5504112 | 0.02329556 |
| | | | UCHL5_HUMAN | | |
| 165 - 233 | N165 | C233 | MTA2_HUMAN - | −1.5510688 | 0.00287181 |
| | | | HINFP_HUMAN | | |
| 298 - 270 | N298 | C270 | MBD2b - ENL | −1.5516204 | 2.89E−04 |
| 344 - 12 | N344 | C12 | HDAC3_HUMAN - KMT2A | −1.5517322 | 0.01554923 |
| 149 - 105 | N149 | C105 | MBD1_HUMAN - | −1.5523112 | 1.56E−04 |
| | | | MBD2_HUMAN | | |
| 312 - 105 | N312 | C105 | SetD8 - MBD2_HUMAN | −1.5524938 | 2.58E−04 |
| 217 - 298 | N217 | C298 | CHRC1_HUMAN - MBD2b | −1.5534334 | 1.34E−04 |
| 135 - 191 | N135 | C191 | KDM2A_HUMAN - | −1.5548023 | 0.04367548 |
| | | | CDYL_HUMAN | | |
| 105 - 293 | N105 | C293 | MBD2_HUMAN - KRAB- | −1.554885 | 0.01590004 |
| | | | MeCP2 | | |
| 123 - 298 | N123 | C298 | KMT2A_HUMAN - MBD2b | −1.554904 | 0.00106478 |
| 165 - 88 | N165 | C88 | MTA2_HUMAN - | −1.5557347 | 0.00591082 |
| | | | ING4_HUMAN | | |
| 299 - 191 | N299 | C191 | HST2 - CDYL_HUMAN | −1.5561699 | 0.0444721 |
| 85 - 331 | N85 | C331 | SMYD3_HUMAN - mm_G9a | −1.5571898 | 0.02286424 |
| 185 - 299 | N185 | C299 | RBBP7_HUMAN - HST2 | −1.5576389 | 8.37E−04 |
| 170 - 105 | N170 | C105 | NSD1_HUMAN - | −1.5594698 | 0.02772646 |
| | | | MBD2_HUMAN | | |
| 344 - 190 | N344 | C190 | HDAC3_HUMAN - | −1.5594698 | 0.02772646 |
| | | | ANM1_HUMAN | | |
| 119 - 253 | N119 | C253 | ACL6A_HUMAN - | −1.5607654 | 0.01709049 |
| | | | RBBP5_HUMAN | | |
| 346 - 215 | N346 | C215 | MSX2_HUMAN - | −1.5617567 | 0.00102869 |
| | | | HDAC4_HUMAN | | |
| 110 - 298 | N110 | C298 | JMJD4_HUMAN - MBD2b | −1.5627301 | 0.00325018 |
| 317 - 210 | N317 | C210 | HDAC3 - SIR3_HUMAN | −1.5632175 | 0.01041552 |
| 215 - 320 | N215 | C320 | HDAC4_HUMAN - mMLL3- | −1.5638958 | 0.03010412 |
| | | | SET | | |
| 293 - 135 | N293 | C135 | KRAB-MeCP2 - | −1.5641856 | 0.00825643 |
| | | | KDM2A_HUMAN | | |
| 165 - 99 | N165 | C99 | MTA2_HUMAN - | −1.5645011 | 0.0193658 |
| | | | IN80C_HUMAN | | |
| 1 - 265 | N1 | C265 | SETD1A - EZH2 | −1.5646854 | 0.02338345 |
| 3 - 255 | N3 | C255 | HDAC3 - EED_HUMAN | −1.5656092 | 0.00125286 |
| 334 - 214 | N334 | C214 | hs_RCOR1 - | −1.5672112 | 0.04490514 |
| | | | HDAC4_HUMAN | | |
| 306 - 152 | N306 | C152 | CobB - CDY1_HUMAN | −1.5715453 | 5.04E−04 |
| 289 - 298 | N289 | C298 | FOG1 - MBD2b | −1.5720041 | 0.00101534 |
| 12 - 105 | N12 | C105 | KMT2A - MBD2_HUMAN | −1.5725606 | 7.55E−05 |
| 317 - 25 | N317 | C25 | HDAC3 - CTCFL | −1.5737024 | 0.00109859 |
| 204 - 191 | N204 | C191 | HIF1N_HUMAN - | −1.5739069 | 0.02867925 |
| | | | CDYL_HUMAN | | |
| 105 - 118 | N105 | C118 | MBD2_HUMAN - | −1.5744875 | 2.97E−04 |
| | | | HDAC6_HUMAN | | |
| 312 - 55 | N312 | C55 | SetD8 - RBBP4_HUMAN | −1.5765779 | 0.01769547 |
| 298 - 266 | N298 | C266 | MBD2b - GADD45A | −1.5766278 | 8.03E−04 |
| 105 - 241 | N105 | C241 | MBD2_HUMAN - | −1.5773052 | 0.01483836 |
| | | | SIR2_HUMAN | | |
| 280 - 298 | N280 | C298 | CBF1-Cterm - MBD2b | −1.5773812 | 5.60E−05 |
| 212 - 215 | N212 | C215 | HDAC8_HUMAN - | −1.5781383 | 0.00144208 |
| | | | HDAC4_HUMAN | | |
| 162 -105 | N162 | C105 | SIR6_HUMAN - | −1.5784693 | 3.09E−04 |
| | | | MBD2_HUMAN | | |
| 298 - 2 | N298 | C2 | MBD2b - SETD1A | −1.5796868 | 0.01378234 |
| 324 - 298 | N324 | C298 | LBR_HUMAN - MBD2b | −1.5815887 | 0.00110677 |
| 298 - 47 | N298 | C47 | MBD2b - ANM2_HUMAN | −1.582243 | 0.00755423 |
| 143 - 68 | N143 | C68 | KDM4B_HUMAN - | −1.5822912 | 0.01205189 |
| | | | MBD3_HUMAN | | |
| 344 - 206 | N344 | C206 | HDAC3_HUMAN - | −1.582494 | 0.02132743 |
| | | | SETD9_HUMAN | | |
| 105 - 70 | N105 | C70 | MBD2_HUMAN - | −1.5827501 | 0.00235541 |
| | | | SETB2_HUMAN | | |
| 312 - 298 | N312 | C298 | SetD8 - MBD2b | −1.5832522 | 1.75E−04 |
| 105 - 278 | N105 | C278 | MBD2_HUMAN - TET2CD | −1.5837603 | 8.46E−04 |
| 3 - 190 | N3 | C190 | HDAC3 - ANM1_HUMAN | −1.5846249 | 0.00219674 |
| 167 - 68 | N167 | C68 | EHMT1_HUMAN - | −1.5846502 | 0.0019446 |
| | | | MBD3_HUMAN | | |
| 95 - 332 | N95 | C332 | SETD6_HUMAN - hs_SIRT5 | −1.5848129 | 0.01597805 |
| 88 - 105 | N88 | C105 | ING4_HUMAN - | −1.5849423 | 8.79E−05 |
| | | | MBD2_HUMAN | | |
| 254 - 34 | N254 | C34 | MEN1_HUMAN - DNMT3A- | −1.5850395 | 0.01033726 |
| | | | 3L_JKNp112 | | |
| 313 - 68 | N313 | C68 | SaII - MBD3_HUMAN | −1.5855589 | 0.00165367 |
| 257 - 105 | N257 | C105 | c-Myb-TAD - | −1.5867136 | 0.00168449 |
| | | | MBD2_HUMAN | | |
| 182 - 32 | N182 | C32 | MTA3_HUMAN - | −1.5893947 | 0.01841587 |
| | | | Gilbert_Lab_CRISPRi_KRAB | | |
| 131 - 299 | N131 | C299 | PRDM2_HUMAN - HST2 | −1.5894802 | 0.01457139 |
| 196 - 324 | N196 | C324 | MTA1_HUMAN - | −1.5894955 | 0.01803363 |
| | | | LBR_HUMAN | | |
| 266 - 298 | N266 | C298 | GADD45A - MBD2b | −1.5895562 | 5.61E−05 |
| 24 - 343 | N24 | C343 | CTCFL - NCOR2_HUMAN | −1.5906734 | 0.03892218 |
| 108 - 68 | N108 | C68 | KAT6B_HUMAN - | −1.5914048 | 0.00484546 |
| | | | MBD3_HUMAN | | |
| 293 - 68 | N293 | C68 | KRAB-MeCP2 - | −1.5923949 | 0.00012435 |
| | | | MBD3_HUMAN | | |
| 317 - 233 | N317 | C233 | HDAC3 - HINFP_HUMAN | −1.5928398 | 0.01226108 |
| 215 - 17 | N215 | C17 | HDAC4_HUMAN - EZH2 | −1.5929974 | 0.00858138 |
| 105 - 8 | N105 | C8 | MBD2_HUMAN - MECP2 | −1.5933243 | 0.00464101 |
| 295 - 306 | N295 | C306 | SET-TAF1B - CobB | −1.5941622 | 0.00213682 |
| 298 - 63 | N298 | C63 | MBD2b - SMYD5_HUMAN | −1.5962905 | 0.00146166 |
| 206 - 298 | N206 | C298 | SETD9_HUMAN - MBD2b | −1.5963869 | 4.04E−05 |
| 298 - 246 | N298 | C246 | MBD2b - ATRX_HUMAN | −1.5965571 | 0.00505911 |
| 186 - 68 | N186 | C68 | RIOX2_HUMAN - | −1.5967437 | 0.00117035 |
| | | | MBD3_HUMAN | | |
| 165 - 250 | N165 | C250 | MTA2_HUMAN - | −1.59697 | 0.01367641 |
| | | | HCFC1_HUMAN | | |
| 3 - 192 | N3 | C192 | HDAC3 - JHD2C_HUMAN | −1.5983577 | 0.02453805 |
| 55 - 189 | N55 | C189 | RBBP4_HUMAN - | −1.5984246 | 0.0014827 |
| | | | ING2_HUMAN | | |
| 55 - 217 | N55 | C217 | RBBP4_HUMAN - | −1.5991823 | 1.68E−04 |
| | | | CHRC1_HUMAN | | |
| 55 - 69 | N55 | C69 | RBBP4_HUMAN - | −1.5992598 | 3.26E−05 |
| | | | SETB2_HUMAN | | |
| 298 - 218 | N298 | C218 | MBD2b - IN80B_HUMAN | −1.5996029 | 0.00716082 |
| 3 - 111 | N3 | C111 | HDAC3 - KDM5A_HUMAN | −1.6001062 | 5.71E−04 |
| 78 - 293 | N78 | C293 | KAT6A_HUMAN - KRAB- | −1.6019278 | 0.0012595 |
| | | | MeCP2 | | |
| 131 - 213 | N131 | C213 | PRDM2_HUMAN - | −1.6022441 | 0.02696891 |
| | | | HBAP1_HUMAN | | |
| 320 - 298 | N320 | C298 | mMLL3-SET - MBD2b | −1.6034044 | 0.00513292 |
| 150 - 105 | N150 | C105 | HDA11_HUMAN - | −1.60416 | 5.23E−04 |
| | | | MBD2_HUMAN | | |
| 138 - 68 | N138 | C68 | TFPT_HUMAN - | −1.6048584 | 0.02202212 |
| | | | MBD3_HUMAN | | |
| 73 - 68 | N73 | C68 | KDM5D_HUMAN - | −1.6069611 | 0.00270211 |
| | | | MBD3_HUMAN | | |
| 196 - 320 | N196 | C320 | MTA1_HUMAN - mMLL3- | −1.6071151 | 2.69E−04 |
| | | | SET | | |
| 253 - 7 | N253 | C7 | RBBP5_HUMAN - ZNF10 | −1.6074045 | 0.01466138 |
| 239 - 105 | N239 | C105 | KDM8_HUMAN - | −1.6091679 | 2.49E−04 |
| | | | MBD2_HUMAN | | |
| 329 - 105 | N329 | C105 | pbcv1_vSET - | −1.6108533 | 6.74E−04 |
| | | | MBD2_HUMAN | | |
| 120 - 306 | N120 | C306 | RING1_HUMAN - CobB | −1.6110622 | 0.00387116 |
| 309 - 27 | N309 | C27 | HDAC8 - EZH1 | −1.6134154 | 0.02536678 |
| 3 - 298 | N3 | C298 | HDAC3 - MBD2b | −1.6136149 | 1.26E−04 |
| 174 - 32 | N174 | C32 | HDAC1_HUMAN - | −1.6140704 | 0.02319837 |
| | | | Gilbert_Lab_CRISPRi_KRAB | | |
| 105 - 276 | N105 | C276 | MBD2_HUMAN - | −1.6143782 | 0.0163313 |
| | | | EHMT2(G9a)-Cterm | | |
| 194 - 152 | N194 | C152 | ING1_HUMAN - | −1.6144256 | 0.02035681 |
| | | | CDY1_HUMAN | | |
| 301 - 177 | N301 | C177 | SIRT3 - KDM5C_HUMAN | −1.6149111 | 0.01863025 |
| 332 - 105 | N332 | C105 | hs_SIRT5 - MBD2_HUMAN | −1.6155864 | 2.86E−04 |
| 314 - 94 | N314 | C94 | MeCP2 - KDM3B_HUMAN | −1.6158677 | 0.01981182 |
| 219 - 306 | N219 | C306 | JARD2_HUMAN - CobB | −1.6185787 | 0.02826827 |
| 88 - 167 | N88 | C167 | ING4_HUMAN - | −1.6190697 | 0.0142021 |
| | | | EHMT1_HUMAN | | |
| 15 - 191 | N15 | C191 | EP300 - CDYL_HUMAN | −1.6196587 | 0.02089733 |
| 182 - 202 | N182 | C202 | MTA3_HUMAN - | −1.6199428 | 0.0028329 |
| | | | KDM2B_HUMAN | | |
| 173 - 105 | N173 | C105 | SIR4_HUMAN - | −1.6221762 | 2.97E−06 |
| | | | MBD2_HUMAN | | |
| 273 - 32 | N273 | C32 | PRDM9-CD - | −1.6226431 | 0.00184145 |
| | | | Gilbert_Lab_CRISPRi_KRAB | | |
| 106 - 165 | N106 | C165 | KAT6B_HUMAN - | −1.6232041 | 8.48E−04 |
| | | | MTA2_HUMAN | | |
| 246 - 105 | N246 | C105 | ATRX_HUMAN - | −1.6234905 | 7.44E−05 |
| | | | MBD2_HUMAN | | |
| 306 - 313 | N306 | C313 | CobB - SaII | −1.6238461 | 7.47E−06 |
| 57 - 105 | N57 | C105 | ANM8_HUMAN - | −1.6246175 | 9.59E−05 |
| | | | MBD2_HUMAN | | |
| 298 - 334 | N298 | C334 | MBD2b - hs_RCOR1 | −1.6247149 | 0.00398937 |
| 269 - 298 | N269 | C298 | PU.1 - MBD2b | −1.6252969 | 9.44E−06 |
| 3 - 322 | N3 | C322 | HDAC3 - MINT_HUMAN | −1.6259005 | 0.02663124 |
| 182 - 26 | N182 | C26 | MTA3_HUMAN - EZH1 | −1.6260278 | 2.88E−05 |
| 237 - 127 | N237 | C127 | KAT5_HUMAN - | −1.6267479 | 0.03237688 |
| | | | SETMR_HUMAN | | |
| 298 - 111 | N298 | C111 | MBD2b - KDM5A_HUMAN | −1.6272556 | 4.40E−04 |
| 55 - 149 | N55 | C149 | RBBP4_HUMAN - | −1.6281931 | 0.00574739 |
| | | | MBD1_HUMAN | | |
| 298 - 212 | N298 | C212 | MBD2b - HDAC8_HUMAN | −1.6288425 | 8.50E−04 |
| 267 - 105 | N267 | C105 | FOXA1-Nterm - | −1.6296159 | 0.00287507 |
| | | | MBD2_HUMAN | | |
| 105 - 219 | N105 | C219 | MBD2_HUMAN - | −1.6307815 | 0.00259128 |
| | | | JARD2_HUMAN | | |
| 68 - 187 | N68 | C187 | MBD3_HUMAN - | −1.6315285 | 0.01260479 |
| | | | MBD4_HUMAN | | |
| 190 - 191 | N190 | C191 | ANM1_HUMAN - | −1.6315803 | 0.01332131 |
| | | | CDYL_HUMAN | | |
| 18 - 191 | N18 | C191 | EZH2 - CDYL_HUMAN | −1.6318309 | 0.00369497 |
| 3 - 186 | N3 | C186 | HDAC3 - RIOX2_HUMAN | −1.6329916 | 1.53E−04 |
| 9 - 134 | N9 | C134 | MECP2 - KDM2A_HUMAN | −1.6335226 | 0.0297053 |
| 165 - 15 | N165 | C15 | MTA2_HUMAN - EP300 | −1.6335982 | 0.0238358 |
| 138 - 293 | N138 | C293 | TFPT_HUMAN - KRAB- | −1.6336823 | 0.00147132 |
| | | | MeCP2 | | |
| 68 - 252 | N68 | C252 | MBD3_HUMAN - | −1.6339001 | 0.00444697 |
| | | | HCFC1_HUMAN | | |
| 55 - 288 | N55 | C288 | RBBP4_HUMAN - | −1.6339552 | 7.25E−04 |
| | | | SUV[SET] | | |
| 254 - 68 | N254 | C68 | MEN1_HUMAN - | −1.6356559 | 0.01484048 |
| | | | MBD3_HUMAN | | |
| 205 - 189 | N205 | C189 | JMJD7_HUMAN - | −1.6357523 | 0.01549219 |
| | | | ING2_HUMAN | | |
| 293 - 205 | N293 | C205 | KRAB-MeCP2 - | −1.6365113 | 0.01017227 |
| | | | JMJD7_HUMAN | | |
| 55 - 222 | N55 | C222 | RBBP4_HUMAN - | −1.6367544 | 1.42E−04 |
| | | | IN80E_HUMAN | | |
| 158 - 133 | N158 | C133 | SETB1_HUMAN - | −1.6375752 | 0.02843787 |
| | | | KAT8_HUMAN | | |
| 9 - 215 | N9 | C215 | MECP2 - HDAC4_HUMAN | −1.637943 | 0.01325898 |
| 155 - 105 | N155 | C105 | NSD2_HUMAN - | −1.6381396 | 3.38E−04 |
| | | | MBD2_HUMAN | | |
| 277 - 105 | N277 | C105 | SUV39H1 - MBD2_HUMAN | −1.6389282 | 0.00117136 |
| 138 - 215 | N138 | C215 | TFPT_HUMAN - | −1.6390795 | 0.03151708 |
| | | | HDAC4_HUMAN | | |
| 3 - 85 | N3 | C85 | HDAC3 - SMYD3_HUMAN | −1.6407901 | 0.00543388 |
| 135 - 32 | N135 | C32 | KDM2A_HUMAN - | −1.6411401 | 5.78E−04 |
| | | | Gilbert_Lab_CRISPRi_KRAB | | |
| 105 - 246 | N105 | C246 | MBD2_HUMAN - | −1.6414918 | 8.97E−05 |
| | | | ATRX_HUMAN | | |
| 300 - 298 | N300 | C298 | SIRT6 - MBD2b | −1.6419835 | 0.00617146 |
| 18 - 298 | N18 | C298 | EZH2 - MBD2b | −1.64214 | 2.40E−05 |
| 228 - 313 | N228 | C313 | HDAC7_HUMAN - SaII | −1.6422538 | 0.0019882 |
| 30 - 105 | N30 | C105 | KDM3A - MBD2_HUMAN | −1.6426112 | 4.46E−04 |
| 105 - 331 | N105 | C331 | MBD2_HUMAN - mm_G9a | −1.6434849 | 2.68E−05 |
| 3 - 45 | N3 | C45 | HDAC3 - KMT2D_HUMAN | −1.6443655 | 0.00303085 |
| 105 - 248 | N105 | C248 | MBD2_HUMAN - | −1.6450389 | 0.00170948 |
| | | | GLYR1_HUMAN | | |
| 196 - 73 | N196 | C73 | MTA1_HUMAN - | −1.6459223 | 0.00230273 |
| | | | KDM5D_HUMAN | | |
| 196 - 185 | N196 | C185 | MTA1_HUMAN - | −1.6460251 | 0.00487218 |
| | | | RBBP7_HUMAN | | |
| 178 - 293 | N178 | C293 | KMT5C_HUMAN - KRAB- | −1.6464616 | 2.08E−04 |
| | | | MeCP2 | | |
| 341 - 73 | N341 | C73 | NCOR2_HUMAN - | −1.6479198 | 0.00119195 |
| | | | KDM5D_HUMAN | | |
| 298 - 107 | N298 | C107 | MBD2b - KAT6B_HUMAN | −1.6480052 | 2.04E−04 |
| 220 - 105 | N220 | C105 | JARD2_HUMAN - | −1.6486131 | 4.06E−04 |
| | | | MBD2_HUMAN | | |
| 233 - 68 | N233 | C68 | HINFP_HUMAN - | −1.6488532 | 6.35E−05 |
| | | | MBD3_HUMAN | | |
| 165 - 111 | N165 | C111 | MTA2_HUMAN - | −1.6520583 | 4.24E−04 |
| | | | KDM5A_HUMAN | | |
| 298 - 118 | N298 | C118 | MBD2b - HDAC6_HUMAN | −1.6539712 | 7.59E−04 |
| 149 - 191 | N149 | C191 | MBD1_HUMAN - | −1.6549764 | 0.01564288 |
| | | | CDYL_HUMAN | | |
| 275 - 298 | N275 | C298 | DOT1L-DOT1_domain - | −1.6550466 | 9.78E−05 |
| | | | MBD2b | | |
| 189 - 32 | N189 | C32 | ING2_HUMAN - | −1.6560237 | 2.62E−04 |
| | | | Gilbert_Lab_CRISPRi_KRAB | | |
| 5 - 32 | N5 | C32 | KDM1A - | −1.6567778 | 0.01997851 |
| | | | Gilbert_Lab_CRISPRi_KRAB | | |
| 165 - 272 | N165 | C272 | MTA2_HUMAN - Sir2 | −1.6575585 | 0.01138377 |
| 196 - 130 | N196 | C130 | MTA1_HUMAN - | −1.6575921 | 0.00158798 |
| | | | PRDM2_HUMAN | | |
| 240 - 134 | N240 | C134 | ANM3_HUMAN - | −1.6598066 | 0.02832334 |
| | | | KDM2A_HUMAN | | |
| 105 - 103 | N105 | C103 | MBD2_HUMAN- | −1.6624444 | 0.01412154 |
| | | | B4DKA4_HUMAN | | |
| 327 - 68 | N327 | C68 | hs_NIPP1 - MBD3_HUMAN | −1.6629094 | 4.60E−04 |
| 228 - 299 | N228 | C299 | HDAC7_HUMAN - HST2 | −1.6635389 | 0.01497198 |
| 166 - 293 | N166 | C293 | EHMT1_HUMAN - KRAB- | −1.6640223 | 0.01981775 |
| | | | MeCP2 | | |
| 99 - 298 | N99 | C298 | IN80C_HUMAN - MBD2b | −1.6650269 | 2.17E−05 |
| 293 - 55 | N293 | C55 | KRAB-MeCP2 - | −1.6652288 | 2.30E−04 |
| | | | RBBP4_HUMAN | | |
| 74 - 105 | N74 | C105 | KDM5D_HUMAN - | −1.6662065 | 3.97E−04 |
| | | | MBD2_HUMAN | | |
| 196 - 280 | N196 | C280 | MTA1_HUMAN - CBF1- | −1.6672005 | 0.00576042 |
| | | | Cterm | | |
| 165 - 218 | N165 | C218 | MTA2_HUMAN - | −1.6677546 | 0.02075175 |
| | | | IN80B_HUMAN | | |
| 317 - 191 | N317 | C191 | HDAC3 - CDYL_HUMAN | −1.6685521 | 0.01812756 |
| 313 - 298 | N313 | C298 | SaII - MBD2b | −1.6691778 | 0.00188424 |
| 339 - 306 | N339 | C306 | SPOC1_HUMAN - CobB | −1.6694029 | 0.02901233 |
| 105 - 79 | N105 | C79 | MBD2_HUMAN - | −1.6699155 | 0.00118921 |
| | | | KAT6A_HUMAN | | |
| 164 - 256 | N164 | C256 | SIR5_HUMAN - LSD1-ORF | −1.6702568 | 0.0328352 |
| 298 - 311 | N298 | C311 | MBD2b - Sin3a | −1.6720163 | 1.12E−04 |
| 294 - 36 | N294 | C36 | DIM5 - TET1CD_JKNp84 | −1.672399 | 0.04294134 |
| 105 - 346 | N105 | C346 | MBD2_HUMAN - | −1.6729966 | 5.45E−04 |
| | | | MSX2_HUMAN | | |
| 255 - 105 | N255 | C105 | EED_HUMAN - | −1.6755841 | 2.64E−05 |
| | | | MBD2_HUMAN | | |
| 105 - 91 | N105 | C91 | MBD2_HUMAN - | −1.6787634 | 0.00208052 |
| | | | ANM5_HUMAN | | |
| 298 - 257 | N298 | C257 | MBD2b - c-Myb-TAD | −1.6807646 | 0.01808918 |
| 6 - 293 | N6 | C293 | KDM1A - KRAB-MeCP2 | −1.680942 | 0.00465954 |
| 345 - 293 | N345 | C293 | MSX2_HUMAN - KRAB- | −1.6811355 | 0.00561266 |
| | | | MeCP2 | | |
| 306 - 126 | N306 | C126 | CobB - SETMR_HUMAN | −1.6816162 | 0.00124616 |
| 317 - 69 | N317 | C69 | HDAC3 - SETB2_HUMAN | −1.6825462 | 3.68E−05 |
| 105 - 140 | N105 | C140 | MBD2_HUMAN - | −1.6830873 | 0.02603307 |
| | | | KDM6B_HUMAN | | |
| 45 - 289 | N45 | C289 | KMT2D_HUMAN - FOG1 | −1.6856469 | 0.0424863 |
| 73 - 298 | N73 | C298 | KDM5D_HUMAN - MBD2b | −1.6857345 | 1.24E−04 |
| 105 - 206 | N105 | C206 | MBD2_HUMAN - | −1.6858227 | 0.00845705 |
| | | | SETD9_HUMAN | | |
| 310 -32 | N310 | C32 | TgSET8 - | −1.6858742 | 1.06E−05 |
| | | | Gilbert_Lab_CRISPRi_KRAB | | |
| 313 - 334 | N313 | C334 | SaII - hs_RCOR1 | −1.6863548 | 1.28E−04 |
| 215 - 25 | N215 | C25 | HDAC4_HUMAN - CTCFL | −1.6872308 | 7.93E−04 |
| 202 - 68 | N202 | C68 | KDM2B_HUMAN - | −1.6889765 | 0.00383496 |
| | | | MBD3_HUMAN | | |
| 105 - 3 | N105 | C3 | MBD2_HUMAN - HDAC3 | −1.6930612 | 0.01232821 |
| 105 - 187 | N105 | C187 | MBD2_HUMAN - | −1.6936206 | 0.00131516 |
| | | | MBD4_HUMAN | | |
| 298 - 149 | N298 | C149 | MBD2b - MBD1_HUMAN | −1.6951311 | 3.93E−04 |
| 346 - 191 | N346 | C191 | MSX2_HUMAN - | −1.6960547 | 9.27E−05 |
| | | | CDYL_HUMAN | | |
| 61 - 306 | N61 | C306 | HAT1_HUMAN - CobB | −1.6961224 | 0.00120548 |
| 165 - 7 | N165 | C7 | MTA2_HUMAN - ZNF10 | −1.698744 | 0.00215597 |
| 151 - 298 | N151 | C298 | SIR7_HUMAN - MBD2b | −1.6990446 | 4.55E−05 |
| 256 - 105 | N256 | C105 | LSD1-ORF - | −1.6994665 | 0.00203783 |
| | | | MBD2_HUMAN | | |
| 182 - 87 | N182 | C87 | MTA3_HUMAN - | −1.7007037 | 0.01857656 |
| | | | SETD7_HUMAN | | |
| 346 - 105 | N346 | C105 | MSX2_HUMAN - | −1.7010553 | 1.39E−04 |
| | | | MBD2_HUMAN | | |
| 205 - 298 | N205 | C298 | JMJD7_HUMAN - MBD2b | −1.7013989 | 2.89E−04 |
| 310 - 306 | N310 | C306 | TgSET8 - CobB | −1.7015671 | 9.51E−04 |
| 306 - 143 | N306 | C143 | CobB - KDM4B_HUMAN | −1.7019792 | 0.01171555 |
| 225 - 298 | N225 | C298 | SETD2_HUMAN - MBD2b | −1.704093 | 0.01037515 |
| 25 - 55 | N25 | C55 | CTCFL - RBBP4_HUMAN | −1.7075498 | 1.66E−04 |
| 187 - 334 | N187 | C334 | MBD4_HUMAN - | −1.7084238 | 0.01845954 |
| | | | hs_RCOR1 | | |
| 182 - 12 | N182 | C12 | MTA3_HUMAN - KMT2A | −1.7089565 | 1.92E−04 |
| 78 - 68 | N78 | C68 | KAT6A_HUMAN - | −1.709457 | 1.65E−04 |
| | | | MBD3_HUMAN | | |
| 300 - 334 | N300 | C334 | SIRT6 - hs_RCOR1 | −1.7103347 | 0.03466858 |
| 55 - 227 | N55 | C227 | RBBP4_HUMAN - | −1.7105403 | 0.01342292 |
| | | | SIR1_HUMAN | | |
| 8 - 185 | N8 | C185 | MECP2 - RBBP7_HUMAN | −1.7127799 | 0.00294058 |
| 298 - 190 | N298 | C190 | MBD2b - ANM1_HUMAN | −1.7131698 | 1.71E−04 |
| 148 - 293 | N148 | C293 | NSD3_HUMAN - KRAB- | −1.7137969 | 0.00163077 |
| | | | MeCP2 | | |
| 215 - 288 | N215 | C288 | HDAC4_HUMAN - | −1.7139182 | 1.11E−04 |
| | | | SUV[SET] | | |
| 201 - 63 | N201 | C63 | KDM2B_HUMAN - | −1.7139562 | 0.01525073 |
| | | | SMYD5_HUMAN | | |
| 131 - 25 | N131 | C25 | PRDM2_HUMAN - CTCFL | −1.7141941 | 7.17E−04 |
| 306 - 186 | N306 | C186 | CobB - RIOX2_HUMAN | −1.7185457 | 1.26E−05 |
| 165 - 87 | N165 | C87 | MTA2_HUMAN - | −1.7189447 | 0.0189022 |
| | | | SETD7_HUMAN | | |
| 194 - 68 | N194 | C68 | ING1_HUMAN - | −1.7194599 | 1.54E−04 |
| | | | MBD3_HUMAN | | |
| 341 - 313 | N341 | C313 | NCOR2_HUMAN - SaII | −1.719712 | 2.48E−05 |
| 9 - 182 | N9 | C182 | MECP2 - MTA3_HUMAN | −1.7208572 | 0.00947334 |
| 20 - 216 | N20 | C216 | KDM1B - ING5_HUMAN | −1.7211987 | 0.01745138 |
| 167 - 105 | N167 | C105 | EHMT1_HUMAN - | −1.7213657 | 5.16E−04 |
| | | | MBD2_HUMAN | | |
| 3 - 289 | N3 | C289 | HDAC3 - FOG1 | −1.7217623 | 8.25E−04 |
| 344 - 298 | N344 | C298 | HDAC3_HUMAN - MBD2b | −1.721777 | 1.24E−04 |
| 329 - 191 | N329 | C191 | pbcv1_vSET - | −1.7223764 | 8.69E−04 |
| | | | CDYL_HUMAN | | |
| 297 - 105 | N297 | C105 | MesoLo - MBD2_HUMAN | −1.7240573 | 2.09E−05 |
| 190 - 32 | N190 | C32 | ANM1_HUMAN - | −1.7260307 | 1.68E−04 |
| | | | Gilbert_Lab_CRISPRi_KRAB | | |
| 222 - 32 | N222 | C32 | IN80E_HUMAN - | −1.7260658 | 0.00103216 |
| | | | Gilbert_Lab_CRISPRi_KRAB | | |
| 200 - 105 | N200 | C105 | ARP5_HUMAN - | −1.7265487 | 6.72E−04 |
| | | | MBD2_HUMAN | | |
| 68 - 214 | N68 | C214 | MBD3_HUMAN - | −1.7270721 | 0.00314718 |
| | | | HDAC4_HUMAN | | |
| 239 - 298 | N239 | C298 | KDM8_HUMAN - MBD2b | −1.727188 | 0.0280994 |
| 105 - 290 | N105 | C290 | MBD2_HUMAN - HP1aCS | −1.7273761 | 5.32E−05 |
| 204 - 69 | N204 | C69 | HIF1N_HUMAN - | −1.7276374 | 0.0064521 |
| | | | SETB2_HUMAN | | |
| 1 - 293 | N1 | C293 | SETD1A - KRAB-MeCP2 | −1.7297444 | 1.69E−04 |
| 165 - 290 | N165 | C290 | MTA2_HUMAN - HP1aCS | −1.7303898 | 1.45E−04 |
| 105 - 1 | N105 | C1 | MBD2_HUMAN - SETD1A | −1.7303901 | 0.0011958 |
| 212 - 165 | N212 | C165 | HDAC8_HUMAN - | −1.7322666 | 0.00452766 |
| | | | MTA2_HUMAN | | |
| 298 - 57 | N298 | C57 | MBD2b - ANM8_HUMAN | −1.732775 | 9.41E−04 |
| 215 - 266 | N215 | C266 | HDAC4_HUMAN - | −1.7340837 | 5.71E−05 |
| | | | GADD45A | | |
| 106 - 215 | N106 | C215 | KAT6B_HUMAN - | −1.7343685 | 1.27E−04 |
| | | | HDAC4_HUMAN | | |
| 154 - 293 | N154 | C293 | NSD2_HUMAN - KRAB- | −1.7345978 | 0.00131797 |
| | | | MeCP2 | | |
| 298 - 320 | N298 | C320 | MBD2b - mMLL3-SET | −1.7363746 | 6.40E−06 |
| 105 - 275 | N105 | C275 | MBD2_HUMAN - DOT1L- | −1.7374523 | 1.04E−05 |
| | | | DOT1_domain | | |
| 105 - 268 | N105 | C268 | MBD2_HUMAN - FOXA1- | −1.7411298 | 1.49E−04 |
| | | | Cterm | | |
| 295 - 298 | N295 | C298 | SET-TAF1B - MBD2b | −1.7423416 | 0.00485995 |
| 69 - 191 | N69 | C191 | SETB2_HUMAN - | −1.7425259 | 6.97E−06 |
| | | | CDYL_HUMAN | | |
| 3 - 241 | N3 | C241 | HDAC3 - SIR2_HUMAN | −1.7430297 | 4.40E−04 |
| 215 - 313 | N215 | C313 | HDAC4_HUMAN - SaII | −1.7448429 | 0.00100839 |
| 105 - 110 | N105 | C110 | MBD2_HUMAN - | −1.7468311 | 0.00954122 |
| | | | JMJD4_HUMAN | | |
| 222 - 298 | N222 | C298 | IN80E_HUMAN - MBD2b | −1.7476244 | 4.10E−05 |
| 170 - 32 | N170 | C32 | NSD1_HUMAN - | −1.7504031 | 0.00362098 |
| | | | Gilbert_Lab_CRISPRi_KRAB | | |
| 341 - 99 | N341 | C99 | NCOR2_HUMAN - | −1.7523751 | 0.0033306 |
| | | | IN80C_HUMAN | | |
| 3 - 118 | N3 | C118 | HDAC3 - HDAC6_HUMAN | −1.7526799 | 2.36E−04 |
| 55 - 190 | N55 | C190 | RBBP4_HUMAN - | −1.7527385 | 0.00250211 |
| | | | ANM1_HUMAN | | |
| 189 - 298 | N189 | C298 | ING2_HUMAN - MBD2b | −1.7527825 | 3.28E−06 |
| 298 - 154 | N298 | C154 | MBD2b - NSD2_HUMAN | −1.7539593 | 0.00455586 |
| 55 - 7 | N55 | C7 | RBBP4_HUMAN - ZNF10 | −1.7558686 | 1.93E−05 |
| 194 - 32 | N194 | C32 | ING1_HUMAN - | −1.7560811 | 3.60E−05 |
| | | | Gilbert_Lab_CRISPRi_KRAB | | |
| 339 - 105 | N339 | C105 | SPOC1_HUMAN - | −1.756589 | 0.00672608 |
| | | | MBD2_HUMAN | | |
| 182 - 130 | N182 | C130 | MTA3_HUMAN - | −1.7588659 | 0.00608218 |
| | | | PRDM2_HUMAN | | |
| 105 - 297 | N105 | C297 | MBD2_HUMAN - MesoLo | −1.7593258 | 1.14E−05 |
| 3 - 63 | N3 | C63 | HDAC3 - SMYD5_HUMAN | −1.7605722 | 5.59E−05 |
| 116 - 298 | N116 | C298 | ANM6_HUMAN - MBD2b | −1.7625006 | 1.28E−05 |
| 269 - 105 | N269 | C105 | PU.1 - MBD2_HUMAN | −1.7628334 | 7.78E−06 |
| 346 - 321 | N346 | C321 | MSX2_HUMAN - | −1.7637697 | 0.01076756 |
| | | | MINT_HUMAN | | |
| 69 - 341 | N69 | C341 | SETB2_HUMAN - | −1.7641418 | 0.00435045 |
| | | | NCOR2_HUMAN | | |
| 315 - 105 | N315 | C105 | NCoR - MBD2_HUMAN | −1.7644037 | 1.73E−04 |
| 68 - 327 | N68 | C327 | MBD3_HUMAN - hs_NIPP1 | −1.7659127 | 1.13E−05 |
| 136 - 341 | N136 | C341 | JMJD6_HUMAN - | −1.7662806 | 0.01840171 |
| | | | NCOR2_HUMAN | | |
| 298 - 87 | N298 | C87 | MBD2b - SETD7_HUMAN | −1.7668418 | 2.31E−04 |
| 298 - 85 | N298 | C85 | MBD2b - SMYD3_HUMAN | −1.7687979 | 0.00242415 |
| 182 - 233 | N182 | C233 | MTA3_HUMAN - | −1.7695164 | 0.00145079 |
| | | | HINFP_HUMAN | | |
| 294 - 22 | N294 | C22 | DIM5 - TET1 | −1.770059 | 0.03073939 |
| 197 - 298 | N197 | C298 | SUV91_HUMAN - MBD2b | −1.7708926 | 0.00635594 |
| 148 - 298 | N148 | C298 | NSD3_HUMAN - MBD2b | −1.7717119 | 4.80E−05 |
| 136 - 293 | N136 | C293 | JMJD6_HUMAN - KRAB- | −1.7721291 | 2.95E−04 |
| | | | MeCP2 | | |
| 105 - 267 | N105 | C267 | MBD2_HUMAN - FOXA1- | −1.774195 | 2.86E−04 |
| | | | Nterm | | |
| 332 - 68 | N332 | C68 | hs_SIRT5 - MBD3_HUMAN | −1.774316 | 3.34E−04 |
| 3 - 231 | N3 | C231 | HDAC3 - KDM5B_HUMAN | −1.7754366 | 0.02116604 |
| 222 - 55 | N222 | C55 | IN80E_HUMAN - | −1.775884 | 0.00133817 |
| | | | RBBP4_HUMAN | | |
| 307 - 293 | N307 | C293 | KYP - KRAB-MeCP2 | −1.7779229 | 0.00281174 |
| 105 - 217 | N105 | C217 | MBD2_HUMAN - | −1.7796568 | 0.00728676 |
| | | | CHRC1_HUMAN | | |
| 105 -262 | N105 | C262 | MBD2_HUMAN - EZH2 | −1.781203 | 0.00214045 |
| 341 - 63 | N341 | C63 | NCOR2_HUMAN - | −1.7834875 | 0.02067537 |
| | | | SMYD5_HUMAN | | |
| 118 - 105 | N118 | C105 | HDAC6_HUMAN - | −1.7840365 | 4.16E−04 |
| | | | MBD2_HUMAN | | |
| 165 - 217 | N165 | C217 | MTA2_HUMAN - | −1.7862504 | 5.29E−04 |
| | | | CHRC1_HUMAN | | |
| 165 - 186 | N165 | C186 | MTA2_HUMAN - | −1.7862652 | 0.00573336 |
| | | | RIOX2_HUMAN | | |
| 111 - 191 | N111 | C191 | KDM5A_HUMAN - | −1.7874439 | 0.00118982 |
| | | | CDYL_HUMAN | | |
| 215 - 269 | N215 | C269 | HDAC4_HUMAN - PU.1 | −1.7879938 | 5.70E−05 |
| 215 - 98 | N215 | C98 | HDAC4_HUMAN - | −1.7902461 | 0.00197237 |
| | | | A4FU79_HUMAN | | |
| 105 - 105 | N105 | C105 | MBD2_HUMAN - | −1.7904977 | 2.92E−06 |
| | | | MBD2_HUMAN | | |
| 3 - 134 | N3 | C134 | HDAC3 - KDM2A_HUMAN | −1.7909602 | 0.01611343 |
| 346 - 341 | N346 | C341 | MSX2_HUMAN - | −1.7916758 | 4.00E−04 |
| | | | NCOR2_HUMAN | | |
| 138 - 32 | N138 | C32 | TFPT_HUMAN - | −1.7917328 | 0.00611771 |
| | | | Gilbert_Lab_CRISPRi_KRAB | | |
| 215 - 332 | N215 | C332 | HDAC4_HUMAN - | −1.7917535 | 0.02545074 |
| | | | hs_SIRT5 | | |
| 322 - 298 | N322 | C298 | MINT_HUMAN - MBD2b | −1.7921445 | 0.00145583 |
| 87 - 202 | N87 | C202 | SETD7_HUMAN - | −1.7925498 | 0.0225164 |
| | | | KDM2B_HUMAN | | |
| 310 - 68 | N310 | C68 | TgSET8 - MBD3_HUMAN | −1.7931945 | 0.00292839 |
| 18 - 105 | N18 | C105 | EZH2 - MBD2_HUMAN | −1.7947621 | 9.01E−06 |
| 105 -320 | N105 | C320 | MBD2_HUMAN - mMLL3- | −1.7960244 | 6.20E−06 |
| | | | SET | | |
| 151 - 68 | N151 | C68 | SIR7_HUMAN - | −1.7974605 | 1.49E−06 |
| | | | MBD3_HUMAN | | |
| 165 - 289 | N165 | C289 | MTA2_HUMAN - FOG1 | −1.7985615 | 7.34E−04 |
| 302 - 298 | N302 | C298 | HDAC11 - MBD2b | −1.7992054 | 0.00949413 |
| 3 - 185 | N3 | C185 | HDAC3 - RBBP7_HUMAN | −1.8018703 | 2.51E−04 |
| 196 - 349 | N196 | C349 | MTA1_HUMAN - | −1.8029409 | 0.00548681 |
| | | | DAM_N132A | | |
| 55 - 255 | N55 | C255 | RBBP4_HUMAN - | −1.8032086 | 4.33E−05 |
| | | | EED_HUMAN | | |
| 298 - 296 | N298 | C296 | MBD2b - HP1a | −1.8036233 | 3.69E−05 |
| 310 - 293 | N310 | C293 | TgSET8 - KRAB-MeCP2 | −1.8039059 | 9.75E−04 |
| 55 - 299 | N55 | C299 | RBBP4_HUMAN - HST2 | −1.8078453 | 2.83E−06 |
| 105 - 69 | N105 | C69 | MBD2_HUMAN - | −1.8088303 | 6.18E−06 |
| | | | SETB2_HUMAN | | |
| 154 - 68 | N154 | C68 | NSD2_HUMAN - | −1.8100674 | 3.56E−04 |
| | | | MBD3_HUMAN | | |
| 298 - 299 | N298 | C299 | MBD2b - HST2 | −1.8103663 | 9.65E−05 |
| 105 - 226 | N105 | C226 | MBD2_HUMAN - | −1.810724 | 5.26E−05 |
| | | | SETD2_HUMAN | | |
| 68 - 298 | N68 | C298 | MBD3_HUMAN - MBD2b | −1.8110932 | 0.00259162 |
| 5 - 191 | N5 | C191 | KDM1A - CDYL_HUMAN | −1.8116465 | 9.31E−05 |
| 38 - 55 | N38 | C55 | EHMT2 - RBBP4_HUMAN | −1.8117574 | 0.01184303 |
| 298 - 345 | N298 | C345 | MBD2b - MSX2_HUMAN | −1.8121053 | 9.51E−05 |
| 182 - 270 | N182 | C270 | MTA3_HUMAN - ENL | −1.8156232 | 0.0034193 |
| 263 - 306 | N263 | C306 | HDAC8 - CobB | −1.8162475 | 3.99E−04 |
| 85 - 68 | N85 | C68 | SMYD3_HUMAN - | −1.8162846 | 0.00135101 |
| | | | MBD3_HUMAN | | |
| 341 - 27 | N341 | C27 | NCOR2_HUMAN - EZH1 | −1.8182086 | 0.0090925 |
| 105 - 55 | N105 | C55 | MBD2_HUMAN - | −1.8182423 | 0.00294624 |
| | | | RBBP4_HUMAN | | |
| 256 - 1 | N256 | C1 | LSD1-ORF - SETD1A | −1.8196161 | 7.35E−05 |
| 131 - 313 | N131 | C313 | PRDM2_HUMAN - SaII | −1.8222366 | 0.00493293 |
| 3 - 130 | N3 | C130 | HDAC3 - PRDM2_HUMAN | −1.8230989 | 4.90E−05 |
| 55 - 314 | N55 | C314 | RBBP4_HUMAN - MeCP2 | −1.8237042 | 0.0014026 |
| 105 - 164 | N105 | C164 | MBD2_HUMAN - | −1.8260049 | 1.39E−04 |
| | | | SIR5_HUMAN | | |
| 167 - 32 | N167 | C32 | EHMT1_HUMAN - | −1.8264081 | 4.61E−04 |
| | | | Gilbert_Lab_CRISPRi_KRAB | | |
| 3 - 7 | N3 | C7 | HDAC3 - ZNF10 | −1.8272905 | 4.71E−04 |
| 3 - 246 | N3 | C246 | HDAC3 - ATRX_HUMAN | −1.8280097 | 4.32E−06 |
| 189 - 105 | N189 | C105 | ING2_HUMAN - | −1.8285478 | 1.66E−06 |
| | | | MBD2_HUMAN | | |
| 85 - 191 | N85 | C191 | SMYD3_HUMAN - | −1.8318818 | 0.00532482 |
| | | | CDYL_HUMAN | | |
| 6 - 298 | N6 | C298 | KDM1A - MBD2b | −1.8406659 | 2.76E−05 |
| 245 - 191 | N245 | C191 | TYY1_HUMAN - | −1.8425679 | 0.00324622 |
| | | | CDYL_HUMAN | | |
| 196 - 68 | N196 | C68 | MTA1_HUMAN - | −1.8437045 | 0.00114779 |
| | | | MBD3_HUMAN | | |
| 268 - 152 | N268 | C152 | FOXA1-Cterm - | −1.8438268 | 7.29E−04 |
| | | | CDY1_HUMAN | | |
| 289 - 341 | N289 | C341 | FOG1 - NCOR2_HUMAN | −1.84383 | 0.00341678 |
| 324 - 55 | N324 | C55 | LBR_HUMAN - | −1.8443883 | 0.01598442 |
| | | | RBBP4_HUMAN | | |
| 341 - 26 | N341 | C26 | NCOR2_HUMAN - EZH1 | −1.8471271 | 0.0015354 |
| 3 - 251 | N3 | C251 | HDAC3 - HCFC1_HUMAN | −1.8512402 | 0.00252777 |
| 105 - 37 | N105 | C37 | MBD2_HUMAN - KDM6A | −1.8520786 | 0.0059323 |
| 298 - 26 | N298 | C26 | MBD2b - EZH1 | −1.8530373 | 1.19E−05 |
| 74 - 298 | N74 | C298 | KDM5D_HUMAN - MBD2b | −1.8537245 | 8.64E−04 |
| 267 - 32 | N267 | C32 | FOXA1-Nterm - | −1.8548285 | 8.97E−04 |
| | | | Gilbert_Lab_CRISPRi_KRAB | | |
| 88 - 293 | N88 | C293 | ING4_HUMAN - KRAB- | −1.855084 | 3.11E−05 |
| | | | MeCP2 | | |
| 165 - 332 | N165 | C332 | MTA2_HUMAN - hs_SIRT5 | −1.8554856 | 3.92E−04 |
| 165 - 187 | N165 | C187 | MTA2_HUMAN - | −1.8561063 | 3.73E−04 |
| | | | MBD4_HUMAN | | |
| 186 - 306 | N186 | C306 | RIOX2_HUMAN - CobB | −1.8563131 | 5.39E−05 |
| 182 - 1 | N182 | C1 | MTA3_HUMAN - SETD1A | −1.8566471 | 3.36E−05 |
| 67 - 51 | N67 | C51 | PRDM9_HUMAN - | −1.8572931 | 0.01207509 |
| | | | UCHL5_HUMAN | | |
| 196 - 290 | N196 | C290 | MTA1_HUMAN - HP1aCS | −1.8603005 | 7.54E−05 |
| 174 - 299 | N174 | C299 | HDAC1_HUMAN - HST2 | −1.8606484 | 0.00945952 |
| 3 - 47 | N3 | C47 | HDAC3 - ANM2_HUMAN | −1.8613124 | 0.01465587 |
| 298 - 138 | N298 | C138 | MBD2b - TFPT_HUMAN | −1.8617129 | 9.30E−04 |
| 241 - 105 | N241 | C105 | SIR2_HUMAN - | −1.8623703 | 2.02E−04 |
| | | | MBD2_HUMAN | | |
| 215 - 222 | N215 | C222 | HDAC4_HUMAN - | −1.8627296 | 2.82E−04 |
| | | | IN80E_HUMAN | | |
| 345 - 298 | N345 | C298 | MSX2_HUMAN - MBD2b | −1.8630981 | 1.02E−05 |
| 327 - 298 | N327 | C298 | hs_NIPP1 - MBD2b | −1.8637975 | 0.00455913 |
| 297 - 298 | N297 | C298 | MesoLo - MBD2b | −1.8643211 | 2.27E−05 |
| 341 - 272 | N341 | C272 | NCOR2_HUMAN - Sir2 | −1.8655745 | 0.00128855 |
| 3 - 32 | N3 | C32 | HDAC3 - | −1.8677207 | 2.46E−05 |
| | | | Gilbert_Lab_CRISPRi_KRAB | | |
| 105 - 239 | N105 | C239 | MBD2_HUMAN - | −1.8677477 | 1.67E−04 |
| | | | KDM8_HUMAN | | |
| 252 - 298 | N252 | C298 | HCFC1_HUMAN - MBD2b | −1.868141 | 0.00211183 |
| 207 - 298 | N207 | C298 | KDM1B_HUMAN - MBD2b | −1.8700982 | 0.00701019 |
| 126 - 314 | N126 | C314 | SETMR_HUMAN - MeCP2 | −1.8710376 | 0.00293877 |
| 105 - 313 | N105 | C313 | MBD2_HUMAN - SaII | −1.871533 | 5.78E−04 |
| 245 - 105 | N245 | C105 | TYY1_HUMAN - | −1.8716108 | 1.32E−06 |
| | | | MBD2_HUMAN | | |
| 280 - 105 | N280 | C105 | CBF1-Cterm - | −1.8717164 | 1.20E−05 |
| | | | MBD2_HUMAN | | |
| 155 - 298 | N155 | C298 | NSD2_HUMAN - MBD2b | −1.8725842 | 1.59E−04 |
| 268 - 68 | N268 | C68 | FOXA1-Cterm - | −1.8754 | 5.09E−07 |
| | | | MBD3_HUMAN | | |
| 6 - 105 | N6 | C105 | KDM1A - MBD2_HUMAN | −1.8760702 | 6.09E−04 |
| 306 - 331 | N306 | C331 | CobB - mm_G9a | −1.876501 | 0.01091222 |
| 119 - 97 | N119 | C97 | ACL6A_HUMAN - | −1.878277 | 0.0135804 |
| | | | CHD3_HUMAN | | |
| 148 - 306 | N148 | C306 | NSD3_HUMAN - CobB | −1.8784594 | 4.39E−04 |
| 182 - 313 | N182 | C313 | MTA3_HUMAN - SaII | −1.8790123 | 1.25E−06 |
| 55 - 214 | N55 | C214 | RBBP4_HUMAN - | −1.8809083 | 6.91E−05 |
| | | | HDAC4_HUMAN | | |
| 57 - 298 | N57 | C298 | ANM8_HUMAN - MBD2b | −1.8813024 | 0.00266648 |
| 151 - 105 | N151 | C105 | SIR7_HUMAN - | −1.8814835 | 8.20E−06 |
| | | | MBD2_HUMAN | | |
| 32 - 131 | N32 | C131 | Gilbert_Lab_CRISPRi_KRAB - | −1.8821154 | 3.92E−04 |
| | | | PRDM2_HUMAN | | |
| 64 - 165 | N64 | C165 | HDAC2_HUMAN - | −1.8824843 | 0.00558604 |
| | | | MTA2_HUMAN | | |
| 189 - 215 | N189 | C215 | ING2_HUMAN - | −1.8828481 | 6.58E−04 |
| | | | HDAC4_HUMAN | | |
| 322 - 68 | N322 | C68 | MINT_HUMAN - | −1.8829068 | 1.35E−05 |
| | | | MBD3_HUMAN | | |
| 257 - 191 | N257 | C191 | c-Myb-TAD - | −1.8831507 | 3.96E−05 |
| | | | CDYL_HUMAN | | |
| 3 - 315 | N3 | C315 | HDAC3 - NCoR | −1.8853702 | 1.60E−07 |
| 245 - 293 | N245 | C293 | TYY1_HUMAN - KRAB- | −1.8865063 | 4.58E−04 |
| | | | MeCP2 | | |
| 327 - 105 | N327 | C105 | hs_NIPP1 - MBD2_HUMAN | −1.8875142 | 1.94E−04 |
| 105 - 25 | N105 | C25 | MBD2_HUMAN - CTCFL | −1.891833 | 4.61E−05 |
| 341 - 267 | N341 | C267 | NCOR2_HUMAN - FOXA1- | −1.8925434 | 0.00584845 |
| | | | Nterm | | |
| 68 - 151 | N68 | C151 | MBD3_HUMAN - | −1.8945 | 0.00265178 |
| | | | SIR7_HUMAN | | |
| 142 - 32 | N142 | C32 | SMRC1_HUMAN - | −1.8960112 | 0.00109223 |
| | | | Gilbert_Lab_CRISPRi_KRAB | | |
| 105 - 68 | N105 | C68 | MBD2_HUMAN - | −1.8963447 | 1.81E−04 |
| | | | MBD3_HUMAN | | |
| 298 - 68 | N298 | C68 | MBD2b - MBD3_HUMAN | −1.8969515 | 1.09E−05 |
| 78 - 105 | N78 | C105 | KAT6A_HUMAN - | −1.897362 | 1.01E−04 |
| | | | MBD2_HUMAN | | |
| 196 - 25 | N196 | C25 | MTA1_HUMAN - CTCFL | −1.8977252 | 0.00731339 |
| 55 - 315 | N55 | C315 | RBBP4_HUMAN - NCoR | −1.8994357 | 1.64E−07 |
| 272 - 105 | N272 | C105 | Sir2 - MBD2_HUMAN | −1.9038751 | 0.0101961 |
| 17 - 32 | N17 | C32 | EZH2 - | −1.9047861 | 0.00130615 |
| | | | Gilbert_Lab_CRISPRi_KRAB | | |
| 314 - 306 | N314 | C306 | MeCP2 - CobB | −1.904982 | 6.58E−05 |
| 3 - 201 | N3 | C201 | HDAC3 - KDM2B_HUMAN | −1.9100751 | 0.00291319 |
| 295 - 105 | N295 | C105 | SET-TAF1B - | −1.9115055 | 2.43E−07 |
| | | | MBD2_HUMAN | | |
| 245 - 298 | N245 | C298 | TYY1_HUMAN - MBD2b | −1.9144838 | 1.07E−05 |
| 300 - 152 | N300 | C152 | SIRT6 - CDY1_HUMAN | −1.9153103 | 0.00478733 |
| 233 - 191 | N233 | C191 | HINFP_HUMAN - | −1.9169232 | 5.06E−04 |
| | | | CDYL_HUMAN | | |
| 55 - 25 | N55 | C25 | RBBP4_HUMAN - CTCFL | −1.9171611 | 4.14E−06 |
| 25 - 105 | N25 | C105 | CTCFL - MBD2_HUMAN | −1.9178454 | 1.65E−05 |
| 48 - 55 | N48 | C55 | SETD5_HUMAN - | −1.9211877 | 0.00608182 |
| | | | RBBP4_HUMAN | | |
| 105 - 7 | N105 | C7 | MBD2_HUMAN - ZNF10 | −1.9212554 | 1.48E−05 |
| 110 - 105 | N110 | C105 | JMJD4_HUMAN - | −1.9277906 | 0.00217615 |
| | | | MBD2_HUMAN | | |
| 215 - 328 | N215 | C328 | HDAC4_HUMAN - | −1.9288735 | 0.00210614 |
| | | | hs_PHF19 | | |
| 105 - 299 | N105 | C299 | MBD2_HUMAN - HST2 | −1.9290039 | 1.46E−07 |
| 105 - 190 | N105 | C190 | MBD2_HUMAN - | −1.9298206 | 2.04E−06 |
| | | | ANM1_HUMAN | | |
| 165 - 73 | N165 | C73 | MTA2_HUMAN - | −1.9304884 | 1.71E−05 |
| | | | KDM5D_HUMAN | | |
| 246 - 298 | N246 | C298 | ATRX_HUMAN - MBD2b | −1.9309873 | 3.84E−07 |
| 3 - 268 | N3 | C268 | HDAC3 - FOXA1-Cterm | −1.9328004 | 0.00468812 |
| 165 - 205 | N165 | C205 | MTA2_HUMAN - | −1.9352289 | 0.00804628 |
| | | | JMJD7_HUMAN | | |
| 5 - 298 | N5 | C298 | KDM1A - MBD2b | −1.9361787 | 1.32E−06 |
| 32 - 105 | N32 | C105 | Gilbert_Lab_CRISPRi_KRAB - | −1.9374643 | 4.93E−05 |
| | | | MBD2_HUMAN | | |
| 190 - 298 | N190 | C298 | ANM1_HUMAN - MBD2b | −1.9382499 | 0.00209184 |
| 329 - 131 | N329 | C131 | pbcv1_vSET - | −1.9392633 | 0.00316252 |
| | | | PRDM2_HUMAN | | |
| 182 - 311 | N182 | C311 | MTA3_HUMAN - Sin3a | −1.9396987 | 0.00193314 |
| 64 - 152 | N64 | C152 | HDAC2_HUMAN - | −1.9405868 | 0.00772641 |
| | | | CDY1_HUMAN | | |
| 68 - 69 | N68 | C69 | MBD3_HUMAN - | −1.9410448 | 6.81E−06 |
| | | | SETB2_HUMAN | | |
| 165 - 320 | N165 | C320 | MTA2_HUMAN - mMLL3- | −1.9416752 | 7.67E−06 |
| | | | SET | | |
| 105 - 106 | N105 | C106 | MBD2_HUMAN - | −1.9446683 | 3.39E−08 |
| | | | KAT6B_HUMAN | | |
| 32 - 256 | N32 | C256 | Gilbert_Lab_CRISPRi_KRAB - | −1.9458572 | 0.00218686 |
| | | | LSD1-ORF | | |
| 105 - 332 | N105 | C332 | MBD2_HUMAN - hs_SIRT5 | −1.9472305 | 2.72E−04 |
| 165 - 8 | N165 | C8 | MTA2_HUMAN - MECP2 | −1.9505133 | 0.00811368 |
| 85 - 105 | N85 | C105 | SMYD3_HUMAN - | −1.9505455 | 1.95E−04 |
| | | | MBD2_HUMAN | | |
| 196 - 106 | N196 | C106 | MTA1_HUMAN - | −1.9572083 | 4.62E−05 |
| | | | KAT6B_HUMAN | | |
| 196 - 308 | N196 | C308 | MTA1_HUMAN - HDT1 | −1.9572367 | 0.00180054 |
| 165 - 315 | N165 | C315 | MTA2_HUMAN - NCoR | −1.9586337 | 3.88E−04 |
| 15 - 105 | N15 | C105 | EP300 - MBD2_HUMAN | −1.9591003 | 2.12E−05 |
| 215 - 301 | N215 | C301 | HDAC4_HUMAN - SIRT3 | −1.9606659 | 6.87E−05 |
| 105 - 153 | N105 | C153 | MBD2_HUMAN - | −1.9619113 | 1.19E−04 |
| | | | KAT7_HUMAN | | |
| 32 - 298 | N32 | C298 | Gilbert_Lab_CRISPRi_KRAB - | −1.9672005 | 1.62E−05 |
| | | | MBD2b | | |
| 105 - 12 | N105 | C12 | MBD2_HUMAN - KMT2A | −1.9682314 | 1.56E−06 |
| 164 - 298 | N164 | C298 | SIR5_HUMAN - MBD2b | −1.9687205 | 1.33E−05 |
| 298 - 1 | N298 | C1 | MBD2b - SETD1A | −1.9708924 | 1.76E−04 |
| 215 - 149 | N215 | C149 | HDAC4_HUMAN - | −1.9740108 | 3.79E−04 |
| | | | MBD1_HUMAN | | |
| 298 - 294 | N298 | C294 | MBD2b - DIM5 | −1.9751086 | 6.21E−06 |
| 55 - 327 | N55 | C327 | RBBP4_HUMAN - hs_NIPP1 | −1.9751995 | 3.06E−06 |
| 64 - 105 | N64 | C105 | HDAC2_HUMAN - | −1.9774131 | 2.98E−06 |
| | | | MBD2_HUMAN | | |
| 3 - 8 | N3 | C8 | HDAC3 - MECP2 | −1.9782591 | 2.65E−06 |
| 202 - 327 | N202 | C327 | KDM2B_HUMAN - | −1.9785724 | 4.01E−04 |
| | | | hs_NIPP1 | | |
| 3 - 165 | N3 | C165 | HDAC3 - MTA2_HUMAN | −1.9835554 | 0.00469714 |
| 78 - 298 | N78 | C298 | KAT6A_HUMAN - MBD2b | −1.9844355 | 3.80E−07 |
| 298 - 61 | N298 | C61 | MBD2b - HAT1_HUMAN | −1.9847232 | 9.04E−04 |
| 257 - 298 | N257 | C298 | c-Myb-TAD - MBD2b | −1.9903244 | 6.23E−05 |
| 182 - 310 | N182 | C310 | MTA3_HUMAN - TgSET8 | −1.9905717 | 1.19E−04 |
| 105 - 38 | N105 | C38 | MBD2_HUMAN - EHMT2 | −1.9916421 | 3.13E−04 |
| 105 - 185 | N105 | C185 | MBD2_HUMAN - | −1.9934366 | 2.12E−04 |
| | | | RBBP7_HUMAN | | |
| 75 - 70 | N75 | C70 | KDM5D_HUMAN - | −1.9938195 | 0.01107021 |
| | | | SETB2_HUMAN | | |
| 182 - 55 | N182 | C55 | MTA3_HUMAN - | −1.9940704 | 5.90E−04 |
| | | | RBBP4_HUMAN | | |
| 77 - 68 | N77 | C68 | KDM5D_HUMAN - | −1.9952461 | 1.31E−05 |
| | | | MBD3_HUMAN | | |
| 173 - 298 | N173 | C298 | SIR4_HUMAN - MBD2b | −1.99574 | 6.10E−04 |
| 310 - 105 | N310 | C105 | TgSET8 - MBD2_HUMAN | −1.9970482 | 3.60E−07 |
| 69 - 293 | N69 | C293 | SETB2_HUMAN - KRAB- | −1.9991525 | 5.60E−04 |
| | | | MeCP2 | | |
| 315 - 298 | N315 | C298 | NCoR - MBD2b | −2.0000418 | 1.71E−06 |
| 195 - 105 | N195 | C105 | SETD3_HUMAN - | −2.0002009 | 1.13E−04 |
| | | | MBD2_HUMAN | | |
| 215 - 268 | N215 | C268 | HDAC4_HUMAN - FOXA1- | −2.0022858 | 2.20E−04 |
| | | | Cterm | | |
| 165 - 123 | N165 | C123 | MTA2_HUMAN - | −2.0028676 | 0.00576643 |
| | | | KMT2A_HUMAN | | |
| 105 - 128 | N105 | C128 | MBD2_HUMAN - | −2.0038167 | 4.98E−04 |
| | | | HAIR_HUMAN | | |
| 3 - 68 | N3 | C68 | HDAC3 - MBD3_HUMAN | −2.0086647 | 8.47E−08 |
| 344 - 239 | N344 | C239 | HDAC3_HUMAN - | −2.0103825 | 0.00448274 |
| | | | KDM8_HUMAN | | |
| 182 - 346 | N182 | C346 | MTA3_HUMAN - | −2.0120241 | 7.90E−05 |
| | | | MSX2_HUMAN | | |
| 182 - 332 | N182 | C332 | MTA3_HUMAN - hs_SIRT5 | −2.0130506 | 6.11E−05 |
| 61 - 105 | N61 | C105 | HAT1_HUMAN - | −2.0142415 | 2.26E−04 |
| | | | MBD2_HUMAN | | |
| 233 - 215 | N233 | C215 | HINFP_HUMAN - | −2.0145665 | 1.52E−05 |
| | | | HDAC4_HUMAN | | |
| 138 - 298 | N138 | C298 | TFPT_HUMAN - MBD2b | −2.0149919 | 0.00162101 |
| 105 - 63 | N105 | C63 | MBD2_HUMAN - | −2.0213373 | 1.01E−04 |
| | | | SMYD5_HUMAN | | |
| 241 - 68 | N241 | C68 | SIR2_HUMAN - | −2.0216606 | 4.33E−06 |
| | | | MBD3_HUMAN | | |
| 298 - 233 | N298 | C233 | MBD2b - HINFP_HUMAN | −2.0253903 | 2.08E−05 |
| 298 - 308 | N298 | C308 | MBD2b - HDT1 | −2.0275443 | 4.64E−05 |
| 68 - 314 | N68 | C314 | MBD3_HUMAN - MeCP2 | −2.0278708 | 5.44E−07 |
| 186 - 105 | N186 | C105 | RIOX2_HUMAN - | −2.028462 | 2.26E−05 |
| | | | MBD2_HUMAN | | |
| 298 - 204 | N298 | C204 | MBD2b - HIF1N_HUMAN | −2.02942 | 2.65E−05 |
| 55 - 298 | N55 | C298 | RBBP4_HUMAN - MBD2b | −2.0321874 | 7.42E−08 |
| 12 - 293 | N12 | C293 | KMT2A - KRAB-MeCP2 | −2.0322965 | 6.06E−04 |
| 5 - 341 | N5 | C341 | KDM1A - NCOR2_HUMAN | −2.0327574 | 0.0012255 |
| 293 - 32 | N293 | C32 | KRAB-MeCP2 - | −2.0345597 | 9.08E−06 |
| | | | Gilbert_Lab_CRISPRi_KRAB | | |
| 298 - 116 | N298 | C116 | MBD2b - ANM6_HUMAN | −2.0372938 | 6.71E−04 |
| 3 - 105 | N3 | C105 | HDAC3 - MBD2_HUMAN | −2.0381299 | 1.09E−05 |
| 105 - 186 | N105 | C186 | MBD2_HUMAN - | −2.040693 | 1.02E−05 |
| | | | RIOX2_HUMAN | | |
| 106 - 32 | N106 | C32 | KAT6B_HUMAN - | −2.0414366 | 7.71E−05 |
| | | | Gilbert_Lab_CRISPRi_KRAB | | |
| 233 - 105 | N233 | C105 | HINFP_HUMAN - | −2.0484098 | 4.04E−06 |
| | | | MBD2_HUMAN | | |
| 165 - 68 | N165 | C68 | MTA2_HUMAN - | −2.0491263 | 2.79E−04 |
| | | | MBD3_HUMAN | | |
| 219 - 298 | N219 | C298 | JARD2_HUMAN - MBD2b | −2.0497392 | 4.54E−04 |
| 263 - 298 | N263 | C298 | HDAC8 - MBD2b | −2.0508585 | 2.77E−05 |
| 27 - 105 | N27 | C105 | EZH1 - MBD2_HUMAN | −2.0514323 | 6.52E−05 |
| 298 - 130 | N298 | C130 | MBD2b - PRDM2_HUMAN | −2.051775 | 0.00331916 |
| 1 - 298 | N1 | C298 | SETD1A - MBD2b | −2.0524522 | 1.36E−05 |
| 3 - 149 | N3 | C149 | HDAC3 - MBD1_HUMAN | −2.0525566 | 4.99E−05 |
| 298 - 69 | N298 | C69 | MBD2b - SETB2_HUMAN | −2.0536159 | 7.75E−04 |
| 68 - 78 | N68 | C78 | MBD3_HUMAN - | −2.0550407 | 8.73E−04 |
| | | | KAT6A_HUMAN | | |
| 111 - 298 | N111 | C298 | KDM5A_HUMAN - MBD2b | −2.0557073 | 7.15E−06 |
| 154 - 105 | N154 | C105 | NSD2_HUMAN - | −2.057568 | 1.76E−06 |
| | | | MBD2_HUMAN | | |
| 196 - 222 | N196 | C222 | MTA1_HUMAN - | −2.058457 | 0.00109409 |
| | | | IN80E_HUMAN | | |
| 73 - 105 | N73 | C105 | KDM5D_HUMAN - | −2.0586244 | 4.23E−06 |
| | | | MBD2_HUMAN | | |
| 306 - 215 | N306 | C215 | CobB - HDAC4_HUMAN | −2.0595322 | 2.30E−04 |
| 339 - 298 | N339 | C298 | SPOC1_HUMAN - MBD2b | −2.0643639 | 0.00118319 |
| 112 - 275 | N112 | C275 | KDM5A_HUMAN - DOT1L- | −2.0650804 | 0.00131473 |
| | | | DOT1_domain | | |
| 332 - 202 | N332 | C202 | hs_SIRT5 - | −2.0663771 | 0.00400583 |
| | | | KDM2B_HUMAN | | |
| 105 - 9 | N105 | C9 | MBD2_HUMAN - MECP2 | −2.0718366 | 1.83E−04 |
| 196 - 164 | N196 | C164 | MTA1_HUMAN - | −2.0821965 | 0.00361066 |
| | | | SIR5_HUMAN | | |
| 55 - 105 | N55 | C105 | RBBP4_HUMAN - | −2.0838447 | 2.29E−08 |
| | | | MBD2_HUMAN | | |
| 165 - 12 | N165 | C12 | MTA2_HUMAN - KMT2A | −2.0847835 | 1.28E−04 |
| 196 - 315 | N196 | C315 | MTA1_HUMAN - NCoR | −2.0850156 | 1.70E−05 |
| 194 - 105 | N194 | C105 | ING1_HUMAN - | −2.0857433 | 1.91E−04 |
| | | | MBD2_HUMAN | | |
| 246 - 293 | N246 | C293 | ATRX_HUMAN - KRAB- | −2.0866057 | 4.93E−04 |
| | | | MeCP2 | | |
| 306 - 196 | N306 | C196 | CobB - MTA1_HUMAN | −2.0867307 | 0.00417878 |
| 106 - 298 | N106 | C298 | KAT6B_HUMAN - MBD2b | −2.0887381 | 8.96E−08 |
| 341 - 8 | N341 | C8 | NCOR2_HUMAN - MECP2 | −2.0909555 | 9.40E−06 |
| 280 - 341 | N280 | C341 | CBF1-Cterm - | −2.0919403 | 0.00381003 |
| | | | NCOR2_HUMAN | | |
| 196 - 310 | N196 | C310 | MTA1_HUMAN - TgSET8 | −2.0951695 | 0.00194682 |
| 166 - 105 | N166 | C105 | EHMT1_HUMAN - | −2.0953492 | 3.57E−04 |
| | | | MBD2_HUMAN | | |
| 69 - 105 | N69 | C105 | SETB2_HUMAN - | −2.0955902 | 1.23E−07 |
| | | | MBD2_HUMAN | | |
| 215 - 130 | N215 | C130 | HDAC4_HUMAN - | −2.0981368 | 2.89E−04 |
| | | | PRDM2_HUMAN | | |
| 215 - 1 | N215 | C1 | HDAC4_HUMAN - SETD1A | −2.099548 | 1.68E−04 |
| 196 - 1 | N196 | C1 | MTA1_HUMAN - SETD1A | −2.1019872 | 1.10E−07 |
| 257 - 293 | N257 | C293 | c-Myb-TAD - KRAB-MeCP2 | −2.112451 | 1.67E−07 |
| 306 - 47 | N306 | C47 | CobB - ANM2_HUMAN | −2.1126245 | 0.00164965 |
| 196 - 149 | N196 | C149 | MTA1_HUMAN - | −2.1197293 | 8.58E−04 |
| | | | MBD1_HUMAN | | |
| 298 - 135 | N298 | C135 | MBD2b - KDM2A_HUMAN | −2.1209024 | 1.56E−04 |
| 215 -202 | N215 | C202 | HDAC4_HUMAN - | −2.1221753 | 2.80E−04 |
| | | | KDM2B_HUMAN | | |
| 297 - 215 | N297 | C215 | MesoLo - HDAC4_HUMAN | −2.1228409 | 3.42E−04 |
| 218 - 105 | N218 | C105 | IN80B_HUMAN - | −2.1326237 | 4.03E−05 |
| | | | MBD2_HUMAN | | |
| 212 - 298 | N212 | C298 | HDAC8_HUMAN - MBD2b | −2.1338436 | 1.88E−04 |
| 55 - 204 | N55 | C204 | RBBP4_HUMAN - | −2.1361922 | 9.91E−05 |
| | | | HIF1N_HUMAN | | |
| 255 - 298 | N255 | C298 | EED_HUMAN - MBD2b | −2.1366265 | 1.55E−05 |
| 25 - 298 | N25 | C298 | CTCFL - MBD2b | −2.1424299 | 2.42E−06 |
| 227 - 68 | N227 | C68 | SIR1_HUMAN - | −2.1449952 | 0.00326434 |
| | | | MBD3_HUMAN | | |
| 68 - 1 | N68 | C1 | MBD3_HUMAN - SETD1A | −2.1456674 | 1.91E−04 |
| 182 - 116 | N182 | C116 | MTA3_HUMAN - | −2.1536282 | 0.00261348 |
| | | | ANM6_HUMAN | | |
| 298 - 148 | N298 | C148 | MBD2b - NSD3_HUMAN | −2.1556426 | 4.43E−05 |
| 105 - 233 | N105 | C233 | MBD2_HUMAN - | −2.1564961 | 1.34E−05 |
| | | | HINFP_HUMAN | | |
| 182 - 214 | N182 | C214 | MTA3_HUMAN - | −2.1644647 | 5.21E−04 |
| | | | HDAC4_HUMAN | | |
| 165 - 164 | N165 | C164 | MTA2_HUMAN - | −2.1645229 | 4.27E−04 |
| | | | SIR5_HUMAN | | |
| 165 - 116 | N165 | C116 | MTA2_HUMAN - | −2.1654076 | 2.57E−04 |
| | | | ANM6_HUMAN | | |
| 215 - 110 | N215 | C110 | HDAC4_HUMAN - | −2.1673818 | 0.00122431 |
| | | | JMJD4_HUMAN | | |
| 341 - 290 | N341 | C290 | NCOR2_HUMAN - HP1aCS | −2.1686797 | 7.44E−06 |
| 105 - 134 | N105 | C134 | MBD2_HUMAN - | −2.1687271 | 5.28E−05 |
| | | | KDM2A_HUMAN | | |
| 25 - 293 | N25 | C293 | CTCFL - KRAB-MeCP2 | −2.1697219 | 3.02E−04 |
| 241 - 191 | N241 | C191 | SIR2_HUMAN - | −2.1702955 | 0.00194052 |
| | | | CDYL_HUMAN | | |
| 341 - 32 | N341 | C32 | NCOR2_HUMAN - | −2.172743 | 3.35E−05 |
| | | | Gilbert_Lab_CRISPRi_KRAB | | |
| 215 - 20 | N215 | C20 | HDAC4_HUMAN - KDM1B | −2.1784094 | 6.57E−04 |
| 299 - 105 | N299 | C105 | HST2 - MBD2_HUMAN | −2.178972 | 4.95E−08 |
| 77 - 298 | N77 | C298 | KDM5D_HUMAN - MBD2b | −2.1802591 | 7.54E−06 |
| 73 - 165 | N73 | C165 | KDM5D_HUMAN - | −2.1810421 | 4.21E−05 |
| | | | MTA2_HUMAN | | |
| 298 - 295 | N298 | C295 | MBD2b - SET-TAF1B | −2.1852432 | 6.00E−04 |
| 212 - 105 | N212 | C105 | HDAC8_HUMAN - | −2.1886048 | 2.11E−05 |
| | | | MBD2_HUMAN | | |
| 306 - 298 | N306 | C298 | CobB - MBD2b | −2.1937623 | 3.05E−08 |
| 165 - 311 | N165 | C311 | MTA2_HUMAN - Sin3a | −2.1957418 | 0.00157958 |
| 298 - 15 | N298 | C15 | MBD2b - EP300 | −2.1975726 | 8.78E−05 |
| 298 - 307 | N298 | C307 | MBD2b - KYP | −2.1982983 | 3.81E−06 |
| 299 - 215 | N299 | C215 | HST2 - HDAC4_HUMAN | −2.2005603 | 0.00110841 |
| 196 - 217 | N196 | C217 | MTA1_HUMAN - | −2.2027484 | 3.35E−04 |
| | | | CHRC1_HUMAN | | |
| 32 - 165 | N32 | C165 | Gilbert_Lab_CRISPRi_KRAB - | −2.2037265 | 6.83E−06 |
| | | | MTA2_HUMAN | | |
| 164 - 293 | N164 | C293 | SIR5_HUMAN - KRAB- | −2.2046852 | 8.17E−04 |
| | | | MeCP2 | | |
| 188 - 298 | N188 | C298 | SMYD4_HUMAN - MBD2b | −2.2065404 | 1.13E−05 |
| 196 - 88 | N196 | C88 | MTA1_HUMAN - | −2.2069533 | 0.00184046 |
| | | | ING4_HUMAN | | |
| 322 - 105 | N322 | C105 | MINT_HUMAN - | −2.2094368 | 2.00E−06 |
| | | | MBD2_HUMAN | | |
| 105 - 202 | N105 | C202 | MBD2_HUMAN - | −2.2129549 | 2.92E−05 |
| | | | KDM2B_HUMAN | | |
| 165 - 346 | N165 | C346 | MTA2_HUMAN - | −2.2169236 | 1.33E−05 |
| | | | MSX2_HUMAN | | |
| 25 - 191 | N25 | C191 | CTCFL - CDYL_HUMAN | −2.2189909 | 3.33E−06 |
| 165 - 334 | N165 | C334 | MTA2_HUMAN - | −2.219871 | 7.27E−05 |
| | | | hs_RCOR1 | | |
| 55 - 306 | N55 | C306 | RBBP4_HUMAN - CobB | −2.2219641 | 2.89E−06 |
| 105 - 334 | N105 | C334 | MBD2_HUMAN - | −2.2242821 | 4.42E−07 |
| | | | hs_RCOR1 | | |
| 174 - 298 | N174 | C298 | HDAC1_HUMAN - MBD2b | −2.2292442 | 1.96E−05 |
| 228 - 332 | N228 | C332 | HDAC7_HUMAN - | −2.230855 | 0.0022546 |
| | | | hs_SIRT5 | | |
| 215 - 246 | N215 | C246 | HDAC4_HUMAN - | −2.2321704 | 3.05E−05 |
| | | | ATRX_HUMAN | | |
| 55 - 293 | N55 | C293 | RBBP4_HUMAN - KRAB- | −2.23664 | 4.89E−04 |
| | | | MeCP2 | | |
| 68 - 306 | N68 | C306 | MBD3_HUMAN - CobB | −2.2386403 | 6.28E−06 |
| 105 - 182 | N105 | C182 | MBD2_HUMAN - | −2.2431825 | 9.44E−05 |
| | | | MTA3_HUMAN | | |
| 165 - 105 | N165 | C105 | MTA2_HUMAN - | −2.2469327 | 1.67E−07 |
| | | | MBD2_HUMAN | | |
| 1 - 105 | N1 | C105 | SETD1A - MBD2_HUMAN | −2.2490233 | 1.97E−06 |
| 332 - 215 | N332 | C215 | hs_SIRT5 - | −2.2491345 | 0.00139411 |
| | | | HDAC4_HUMAN | | |
| 322 - 32 | N322 | C32 | MINT_HUMAN - | −2.2518052 | 4.40E−06 |
| | | | Gilbert_Lab_CRISPRi_KRAB | | |
| 69 - 298 | N69 | C298 | SETB2_HUMAN - MBD2b | −2.2526909 | 2.29E−07 |
| 306 - 105 | N306 | C105 | CobB - MBD2_HUMAN | −2.2540561 | 4.65E−09 |
| 68 - 105 | N68 | C105 | MBD3_HUMAN - | −2.2583141 | 1.95E−08 |
| | | | MBD2_HUMAN | | |
| 165 - 69 | N165 | C69 | MTA2_HUMAN - | −2.260498 | 4.17E−05 |
| | | | SETB2_HUMAN | | |
| 78 - 32 | N78 | C32 | KAT6A_HUMAN - | −2.2630791 | 4.76E−05 |
| | | | Gilbert_Lab_CRISPRi_KRAB | | |
| 215 - 190 | N215 | C190 | HDAC4_HUMAN - | −2.2646455 | 8.84E−04 |
| | | | ANM1_HUMAN | | |
| 306 - 182 | N306 | C182 | CobB - MTA3_HUMAN | −2.2677323 | 4.07E−05 |
| 15 - 298 | N15 | C298 | EP300 - MBD2b | −2.2717177 | 4.46E−04 |
| 313 - 306 | N313 | C306 | SaII - CobB | −2.280951 | 1.49E−08 |
| 298 - 315 | N298 | C315 | MBD2b - NCoR | −2.2814258 | 2.85E−04 |
| 196 - 8 | N196 | C8 | MTA1_HUMAN - MECP2 | −2.2863473 | 2.53E−05 |
| 298 - 176 | N298 | C176 | MBD2b - ARP8_HUMAN | −2.2866391 | 0.00116264 |
| 215 - 73 | N215 | C73 | HDAC4_HUMAN - | −2.2942005 | 4.31E−04 |
| | | | KDM5D_HUMAN | | |
| 298 - 346 | N298 | C346 | MBD2b - MSX2_HUMAN | −2.2949378 | 2.98E−07 |
| 233 - 32 | N233 | C32 | HINFP_HUMAN - | −2.2966958 | 8.67E−05 |
| | | | Gilbert_Lab_CRISPRi_KRAB | | |
| 105 - 214 | N105 | C214 | MBD2_HUMAN - | −2.3057108 | 2.25E−04 |
| | | | HDAC4_HUMAN | | |
| 268 - 298 | N268 | C298 | FOXA1-Cterm - MBD2b | −2.3069994 | 6.23E−08 |
| 293 - 105 | N293 | C105 | KRAB-MeCP2 - | −2.3078913 | 4.80E−06 |
| | | | MBD2_HUMAN | | |
| 5 - 215 | N5 | C215 | KDM1A - HDAC4_HUMAN | −2.3083667 | 4.66E−04 |
| 105 - 17 | N105 | C17 | MBD2_HUMAN - EZH2 | −2.310123 | 4.74E−05 |
| 182 - 106 | N182 | C106 | MTA3_HUMAN - | −2.3103974 | 1.24E−05 |
| | | | KAT6B_HUMAN | | |
| 185 - 306 | N185 | C306 | RBBP7_HUMAN - CobB | −2.3140941 | 6.67E−05 |
| 118 - 293 | N118 | C293 | HDAC6_HUMAN - KRAB- | −2.3164315 | 2.79E−05 |
| | | | MeCP2 | | |
| 196 - 105 | N196 | C105 | MTA1_HUMAN - | −2.3197933 | 0.00156363 |
| | | | MBD2_HUMAN | | |
| 105 - 81 | N105 | C81 | MBD2_HUMAN - | −2.3209988 | 3.41E−04 |
| | | | NCOA3_HUMAN | | |
| 215 - 290 | N215 | C290 | HDAC4_HUMAN - HP1aCS | −2.329752 | 7.34E−06 |
| 215 - 280 | N215 | C280 | HDAC4_HUMAN - CBF1- | −2.3309558 | 2.05E−04 |
| | | | Cterm | | |
| 165 - 106 | N165 | C106 | MTA2_HUMAN - | −2.3316808 | 8.38E−05 |
| | | | KAT6B_HUMAN | | |
| 165 - 301 | N165 | C301 | MTA2_HUMAN - SIRT3 | −2.3334809 | 8.42E−04 |
| 256 - 186 | N256 | C186 | LSD1-ORF - | −2.3349822 | 0.00202194 |
| | | | RIOX2_HUMAN | | |
| 55 - 245 | N55 | C245 | RBBP4_HUMAN - | −2.3438901 | 6.89E−04 |
| | | | TYY1_HUMAN | | |
| 298 - 105 | N298 | C105 | MBD2b - MBD2_HUMAN | −2.3482267 | 4.23E−10 |
| 298 - 186 | N298 | C186 | MBD2b - RIOX2_HUMAN | −2.3601249 | 2.94E−05 |
| 215 -26 | N215 | C26 | HDAC4_HUMAN - EZH1 | −2.3658051 | 1.31E−07 |
| 215 - 315 | N215 | C315 | HDAC4_HUMAN - NCoR | −2.3676095 | 8.00E−05 |
| 105 - 120 | N105 | C120 | MBD2_HUMAN - | −2.3676896 | 3.31E−08 |
| | | | RING1_HUMAN | | |
| 165 - 313 | N165 | C313 | MTA2_HUMAN - SaII | −2.3689369 | 6.81E−04 |
| 196 - 5 | N196 | C5 | MTA1_HUMAN - KDM1A | −2.3711979 | 8.97E−04 |
| 287 - 131 | N287 | C131 | G9A[SET] - | −2.3729757 | 0.00167481 |
| | | | PRDM2_HUMAN | | |
| 298 - 280 | N298 | C280 | MBD2b - CBF1-Cterm | −2.374048 | 5.76E−04 |
| 346 - 293 | N346 | C293 | MSX2_HUMAN - KRAB- | −2.3836629 | 9.17E−08 |
| | | | MeCP2 | | |
| 68 - 32 | N68 | C32 | MBD3_HUMAN - | −2.3911476 | 4.79E−06 |
| | | | Gilbert_Lab_CRISPRi_KRAB | | |
| 166 - 298 | N166 | C298 | EHMT1_HUMAN - MBD2b | −2.3934243 | 9.87E−06 |
| | | | MBD2_HUMAN - | | |
| 105 - 78 | N105 | C78 | KAT6A_HUMAN | −2.401373 | 1.27E−08 |
| 313 - 215 | N313 | C215 | SaII - HDAC4_HUMAN | −2.4041577 | 5.35E−04 |
| 298 - 128 | N298 | C128 | MBD2b - HAIR_HUMAN | −2.416245 | 5.98E−06 |
| 196 - 241 | N196 | C241 | MTA1_HUMAN - | −2.4166364 | 4.41E−05 |
| | | | SIR2_HUMAN | | |
| 332 - 32 | N332 | C32 | hs_SIRT5 - | −2.4207498 | 1.57E−05 |
| | | | Gilbert_Lab_CRISPRi_KRAB | | |
| 88 - 341 | N88 | C341 | ING4_HUMAN - | −2.4347575 | 6.59E−05 |
| | | | NCOR2_HUMAN | | |
| 182 - 327 | N182 | C327 | MTA3_HUMAN - hs_NIPP1 | −2.4350487 | 1.07E−04 |
| 149 - 32 | N149 | C32 | MBD1_HUMAN - | −2.4355124 | 3.45E−07 |
| | | | Gilbert_Lab_CRISPRi_KRAB | | |
| 298 - 55 | N298 | C55 | MBD2b - RBBP4_HUMAN | −2.444322 | 8.07E−06 |
| 298 - 9 | N298 | C9 | MBD2b - MECP2 | −2.4501308 | 2.23E−05 |
| 64 - 298 | N64 | C298 | HDAC2_HUMAN - MBD2b | −2.4509012 | 1.31E−05 |
| 196 - 289 | N196 | C289 | MTA1_HUMAN - FOG1 | −2.4515801 | 5.41E−05 |
| 174 - 105 | N174 | C105 | HDAC1_HUMAN - | −2.4521282 | 4.55E−06 |
| | | | MBD2_HUMAN | | |
| 215 - 289 | N215 | C289 | HDAC4_HUMAN - FOG1 | −2.453865 | 1.83E−06 |
| 3 - 217 | N3 | C217 | HDAC3 - CHRC1_HUMAN | −2.4551324 | 1.26E−05 |
| 105 - 306 | N105 | C306 | MBD2_HUMAN - CobB | −2.4693319 | 1.53E−12 |
| 182 - 289 | N182 | C289 | MTA3_HUMAN - FOG1 | −2.4701994 | 6.13E−05 |
| 68 - 191 | N68 | C191 | MBD3_HUMAN - | −2.4718359 | 1.31E−05 |
| | | | CDYL_HUMAN | | |
| 228 - 111 | N228 | C111 | HDAC7_HUMAN - | −2.4782473 | 6.47E−05 |
| | | | KDM5A_HUMAN | | |
| 18 - 293 | N18 | C293 | EZH2 - KRAB-MeCP2 | −2.478558 | 6.30E−05 |
| 306 - 68 | N306 | C68 | CobB - MBD3_HUMAN | −2.4796141 | 5.03E−08 |
| 73 - 293 | N73 | C293 | KDM5D_HUMAN - KRAB- | −2.4899231 | 8.75E−07 |
| | | | MeCP2 | | |
| 151 - 293 | N151 | C293 | SIR7_HUMAN - KRAB- | −2.4932891 | 5.33E−08 |
| | | | MeCP2 | | |
| 268 - 32 | N268 | C32 | FOXA1-Cterm - | −2.4936815 | 1.24E−07 |
| | | | Gilbert_Lab_CRISPRi_KRAB | | |
| 135 - 68 | N135 | C68 | KDM2A_HUMAN - | −2.498604 | 2.49E−04 |
| | | | MBD3_HUMAN | | |
| 341 - 334 | N341 | C334 | NCOR2_HUMAN - | −2.4986048 | 1.61E−04 |
| | | | hs_RCOR1 | | |
| 298 - 332 | N298 | C332 | MBD2b - hs_SIRT5 | −2.5049211 | 7.25E−05 |
| 215 - 297 | N215 | C297 | HDAC4_HUMAN - MesoLo | −2.5054616 | 4.86E−05 |
| 241 - 293 | N241 | C293 | SIR2_HUMAN - KRAB- | −2.5070304 | 1.32E−04 |
| | | | MeCP2 | | |
| 298 - 214 | N298 | C214 | MBD2b - HDAC4_HUMAN | −2.5151914 | 1.74E−07 |
| 165 - 288 | N165 | C288 | MTA2_HUMAN - SUV[SET] | −2.530224 | 2.09E−05 |
| 328 - 293 | N328 | C293 | hs_PHF19 - KRAB-MeCP2 | −2.534127 | 7.90E−05 |
| 298 - 289 | N298 | C289 | MBD2b - FOG1 | −2.5395449 | 4.77E−05 |
| 298 - 290 | N298 | C290 | MBD2b - HP1aCS | −2.5406477 | 5.15E−08 |
| 87 - 298 | N87 | C298 | SETD7_HUMAN - MBD2b | −2.5409602 | 2.68E−07 |
| 111 - 105 | N111 | C105 | KDM5A_HUMAN - | −2.5437818 | 7.84E−07 |
| | | | MBD2_HUMAN | | |
| 317 - 298 | N317 | C298 | HDAC3 - MBD2b | −2.5453389 | 6.92E−08 |
| 298 - 327 | N298 | C327 | MBD2b - hs_NIPP1 | −2.5479678 | 1.28E−07 |
| 182 -306 | N182 | C306 | MTA3_HUMAN - CobB | −2.5766944 | 6.13E−07 |
| 241 - 298 | N241 | C298 | SIR2_HUMAN - MBD2b | −2.5887108 | 1.14E−08 |
| 25 - 165 | N25 | C165 | CTCFL - MTA2_HUMAN | −2.6001659 | 5.15E−05 |
| 105 - 315 | N105 | C315 | MBD2_HUMAN - NCoR | −2.6006607 | 1.10E−07 |
| 196 - 269 | N196 | C269 | MTA1_HUMAN - PU.1 | −2.6035475 | 6.87E−05 |
| 252 - 105 | N252 | C105 | HCFC1_HUMAN - | −2.6243908 | 1.46E−05 |
| | | | MBD2_HUMAN | | |
| 182 - 78 | N182 | C78 | MTA3_HUMAN - | −2.6434623 | 1.51E−05 |
| | | | KAT6A_HUMAN | | |
| 165 - 327 | N165 | C327 | MTA2_HUMAN - hs_NIPP1 | −2.664428 | 1.07E−06 |
| 55 - 32 | N55 | C32 | RBBP4_HUMAN - | −2.6666527 | 6.87E−07 |
| | | | Gilbert_Lab_CRISPRi_KRAB | | |
| 313 - 105 | N313 | C105 | SaII - MBD2_HUMAN | −2.67587 | 1.17E−08 |
| 313 - 191 | N313 | C191 | SaII - CDYL_HUMAN | −2.6991404 | 4.50E−06 |
| 313 - 55 | N313 | C55 | SaII - RBBP4_HUMAN | −2.6996559 | 4.23E−05 |
| 298 - 7 | N298 | C7 | MBD2b - ZNF10 | −2.7073988 | 3.18E−06 |
| 298 - 313 | N298 | C313 | MBD2b - SaII | −2.7419301 | 6.88E−06 |
| 313 - 256 | N313 | C256 | SaII - LSD1-ORF | −2.7634799 | 5.00E−06 |
| 222 - 293 | N222 | C293 | IN80E_HUMAN - KRAB- | −2.7712317 | 2.30E−06 |
| | | | MeCP2 | | |
| 327 - 32 | N327 | C32 | hs_NIPP1 - | −2.782958 | 3.00E−08 |
| | | | Gilbert_Lab_CRISPRi_KRAB | | |
| 105 - 298 | N105 | C298 | MBD2_HUMAN - MBD2b | −2.785728 | 6.03E−06 |
| 215 - 68 | N215 | C68 | HDAC4_HUMAN - | −2.7903857 | 2.84E−07 |
| | | | MBD3_HUMAN | | |
| 185 - 191 | N185 | C191 | RBBP7_HUMAN - | −2.8012281 | 4.44E−06 |
| | | | CDYL_HUMAN | | |
| 25 - 341 | N25 | C341 | CTCFL - NCOR2_HUMAN | −2.8088122 | 9.07E−07 |
| 131 - 105 | N131 | C105 | PRDM2_HUMAN - | −2.8337782 | 1.98E−09 |
| | | | MBD2_HUMAN | | |
| 306 - 32 | N306 | C32 | CobB - | −2.8641021 | 3.01E−10 |
| | | | Gilbert_Lab_CRISPRi_KRAB | | |
| 108 - 298 | N108 | C298 | KAT6B_HUMAN - MBD2b | −2.8785147 | 2.25E−05 |
| 298 - 293 | N298 | C293 | MBD2b - KRAB-MeCP2 | −2.8807611 | 1.52E−08 |
| 105 - 32 | N105 | C32 | MBD2_HUMAN - | −2.8901637 | 8.13E−09 |
| | | | Gilbert_Lab_CRISPRi_KRAB | | |
| 212 - 32 | N212 | C32 | HDAC8_HUMAN - | −2.8987062 | 3.58E−06 |
| | | | Gilbert_Lab_CRISPRi_KRAB | | |
| 74 - 293 | N74 | C293 | KDM5D_HUMAN - KRAB- | −2.9246829 | 7.72E−07 |
| | | | MeCP2 | | |
| 105 - 327 | N105 | C327 | MBD2_HUMAN - hs_NIPP1 | −2.9322523 | 1.80E−11 |
| 268 - 191 | N268 | C191 | FOXA1-Cterm - | −2.9355377 | 9.94E−07 |
| | | | CDYL_HUMAN | | |
| 215 - 345 | N215 | C345 | HDAC4_HUMAN - | −3.0575968 | 2.50E−08 |
| | | | MSX2_HUMAN | | |
| 298 - 5 | N298 | C5 | MBD2b - KDM1A | −3.080805 | 2.14E−09 |
| 341 - 105 | N341 | C105 | NCOR2_HUMAN - | −3.1917665 | 1.07E−08 |
| | | | MBD2_HUMAN | | |
| 215 - 105 | N215 | C105 | HDAC4_HUMAN - | −3.4746905 | 4.25E−11 |
| | | | MBD2_HUMAN | | |

1. A method of determining that a candidate polypeptide in a library of polypeptides has editing activity, the method comprising:
(a) expressing in a plurality of cells:
(i) a library of first expression cassettes each comprising a nucleic acid encoding a polypeptide wherein each polypeptide in the library comprises, in order, a first editing domain, a first linker, a nuclease-deficient RNA guided endonuclease domain, a second linker, and a second editing domain; and
(ii) a second expression cassette comprising a nucleic acid encoding a guide RNA directed to a target of interest, wherein said first and second expression cassettes are expressed in the plurality of cells;
(b) separating the plurality of cells based on the expression level of the target of interest;
(c) extracting genomic DNA (gDNA) from the plurality of cells; and
(d) sequencing the gDNA of cells that have a high level of expression or a low level of expression of the target of interest thereby determining that the candidate polypeptide has epigenetic editing activity.

2. The method of claim 1, wherein cells that have high a level of expression are in the top 25% of the plurality of cells that have the highest level of expression and cells that have low expression are bottom 25% of the plurality of cells that have the lowest expression.

3. The method of claim 1, wherein the target of interest is a gene operably linked with a sequence encoding a fluorescent protein.

4. The method of claim 1, wherein the target of interest is a gene that is not operably linked with a sequence encoding a fluorescent protein.

5. The method of claim 1, wherein the first expression cassettes do not comprise a nucleic acid encoding a barcode sequence.

6. The method of claim 1, wherein the plurality of cells is selected from the group consisting of a plurality of mammalian cells, a plurality of plant cells, a plurality of insect cells, a plurality of eukaryotic cells, a plurality of yeast cells, a plurality of bacterial cells, and a plurality of fungal cells.

7. The method of claim 1, wherein the library comprises first expression cassettes encoding at least 1000 unique polypeptides.

8. The method of claim 1, wherein the library comprises first expression cassettes encoding at least 10000 unique polypeptides.

9. The method of claim 1, wherein the library comprises first expression cassettes encoding at least 100000 unique polypeptides.

10. The method of claim 1, wherein the first editing domain is selected from the group consisting of an enzymatic domain of a polypeptide listed in Table 4.

11. The method of claim 1, wherein the second editing domain is selected from the group consisting of an enzymatic domain of a polypeptide listed in Table 4.

12. The method of claim 1, wherein the nuclease-deficient RNA guided endonuclease domain is a nuclease-deficient CRISPR/Cas effector polypeptide.

13. The method of claim 12, wherein the CRISPR/Cas effector polypeptide is a class 2 nuclease-deficient CRISPR/Cas effector polypeptide.

14. The method of claim 13, wherein the class 2 nuclease-deficient CRISPR/Cas effector polypeptide is a CRISPR/dCas9 effector polypeptide.

15. The method of claim 13, wherein the class 2 nuclease-deficient CRISPR/Cas effector polypeptide is a CRISPR/dCas12 effector polypeptide.

16. The method of claim 1, wherein the guide RNA is a single guide RNA.

17. The method of claim 1, wherein the first expression cassette is contained on a viral vector.

18. The method of claim 1, wherein the second expression cassette is contained on a viral vector.

19. The method of claim 17, wherein the first and second expression cassettes are contained on the same viral vector.

20. The method of claim 17, wherein the viral vector is a lentivirus expression vector.

21. The method of claim 1, wherein the sequencing reaction is a long read sequence reaction.

22. The method of claim 1, wherein the genomic DNA comprises the target of interest.

23. The method of claim 1, wherein the first and second linker are an XTEN linker.

24. The method of claim 1, further comprising sequencing the second expression cassette.

25. The method of claim 1, wherein the editing domain is an epigenetic editing domain.

26. The method of claim 1, wherein the editing domain is a transcriptional editing domain.

27. The method of claim 1, wherein the separating comprises sorting the cells using flow cytometry.

28. A library, the library comprising:
a plurality of expression cassettes each comprising a nucleic acid encoding a polypeptide wherein each polypeptide comprises, in order, a first editing domain, a first linker, a nuclease-deficient RNA guided endonuclease domain, a second linker, and a second editing domain;
wherein the plurality of expression cassettes comprise at least 1000 different combinations of first and second editing domain combinations.

29. The library of claim 28, wherein the first editing domain is any one of N1-N349 as defined by Table 4.

30. The library of claim 28, wherein the second editing domain is any one of C1-C349 as defined by Table 4.

31. The library of claim 28, wherein the first and second linked is an XTEN linker.

32. The library of claim 28, wherein the nuclease-deficient RNA guided endonuclease domain is a nuclease-deficient CRISPR/Cas effector polypeptide.

33. The library of claim 32, wherein the CRISPR/Cas effector polypeptide is a class 2 nuclease-deficient CRISPR/Cas effector polypeptide.

34. The library of claim 33, wherein the class 2 nuclease-deficient CRISPR/Cas effector polypeptide is a CRISPR/dCas9 effector polypeptide.

35. The library of claim 32, wherein the class 2 nuclease-deficient CRISPR/Cas effector polypeptide is a CRISPR/dCas12 effector polypeptide.

36. The library of claim 28, wherein plurality of expression cassettes comprise at least 10000 different combinations of first and second editing domain combinations.

37. The library of claim 28, wherein plurality of expression cassettes comprise at least 100000 different combinations of first and second editing domain combinations.
